(19) United States
(12) Patent Application Publication
(10) Pub. No.: US 2016/0130663 A1
(43) Pub. Date: May 12, 2016

(54) METHOD FOR PREDICTING RESPONSE TO CANCER TREATMENT

(71) Applicants: NATIONAL CANCER CENTER, Tokyo (JP); DAIICHI SANKYO COMPANY, LIMITED, Tokyo (JP)

(72) Inventors: Takashi Kohno, Tokyo (JP); Koji Tsuta, Tokyo (JP); Yuichi Tominaga, Tokyo (JP); Kentaro Ito, Tokyo (JP)

(73) Assignees: National Cancer Center, Chuo-ku, Tokyo (JP); Daiichi Sankyo Company, Limited, Chuo-ku, Tokyo (JP)

(21) Appl. No.: 14/904,188

(22) PCT Filed: Jul. 11, 2014
(86) PCT No.: PCT/JP2014/068572

§ 371 Date: Jan. 11, 2016

(30) Foreign Application Priority Data
Jul. 12, 2013 (JP) 2013-147061

(51) Int. Cl. C12Q 1/68; G01N 33/50; C12N 15/113; G01N 33/574

(52) U.S. Cl. CPC C12Q 1/6886; G01N 33/57496; G01N 33/5038; C12N 15/1137; C12N 2310/14; C12N 2310/531; C12Q 2600/106; C12Q 2600/158; C12Q 2600/156; G01N 2333/914; G01N 2500/10; G01N 2500/04; C12N 2320/30

(57) ABSTRACT

It has been found that BRG1 and BRM have a synthetic-lethal relationship, and that a treatment for inhibiting BRM is a promising approach for treating a BRG1-deficient cancer having no mutation in known therapeutic target genes. Moreover, it has also been revealed that, in this therapeutic strategy, a BRM inhibitor may be administered to a cancer patient after the selection based on BRG1 function suppression, thus enabling an efficient treatment based on the companion diagnosis.

### TECHNICAL FIELD

The present invention relates to a method for predicting responsiveness to a cancer treatment with a compound capable of inhibiting BRM and a method for selecting a cancer treatment target on the basis of BRG1 function suppression. Moreover, the present invention relates to a method for treating a cancer having a suppressed BRG1 function by using a compound capable of inhibiting BRM. Further, the present invention relates to a reagent, used in these methods, for detecting whether or not a BRG1 function is suppressed. Furthermore, the present invention relates to a screening method for a compound on the basis of BRM inhibition, the compound used in a treatment against a cancer having a suppressed BRG1 function.

### BACKGROUND ART

Tyrosine kinase inhibitors are effective against solid tumors with activating mutations in tyrosine kinase genes (for example, EGFR mutations and ALK fusions observed in lung adenocarcinomas) (NPL 1). The present inventors and other researchers have recently identified RET oncogene fusions in lung adenocarcinoma (NPL 2), which supports the importance of tyrosine kinase genes as therapeutic targets. Two other tyrosine kinase gene alterations, the FGFR1 amplification and the DDR2 mutation, have been identified as therapeutic targets in squamous-cell carcinoma, which corresponds to another major type of non-small-cell lung carcinoma (NSCLC) (NPLs 3, 4).

Meanwhile, inactivating somatic mutations of genes that encode subunits of the SWI/SNF chromatin-remodeling complex, such as BRG1/SMARCA4, PBRM1/BAF180, ARID1A/BAF250A, and ARID2/BAF200, have attracted much interest. This is because these mutations have been identified by genome-wide sequencing analyses of cancer cells (NPL 5). These mutations are thought to interfere with the functions of the SWI/SNF in transcription (NPL 5) and DNA double-strand break repair (NPL 6), and believed to be critical for cancer development and progression. In addition, there is a report that BRG1 and BRM play complementary roles in this complex (NPL 7).

However, no therapeutic strategies have yet been developed for specifically targeting cancer cells harboring these inactivating SWI/SNF mutations.

### CITATION LIST

### Non Patent Literatures

[NPL 1] Lovly C M, Carbone D P. Nat Rev Clin Oncol. 2011; 8: 68-70.
[NPL 2] Kohno T, et al. Nat Med. 2012; 18: 375-7.
[NPL 3] Hammerman P S, et al. Cancer Discov. 2011; 1: 78-89.
[NPL 4] Weiss J, et al. Sci Transl Med. 2010; 2: 62ra93.
[NPL 5] Wilson B G, Roberts C W. Nat Rev Cancer. 2011; 11: 481-92.
[NPL 6] Ogiwara H, et al. Oncogene. 2011; 30: 2135-46.
[NPL 7] Reyes J C, et al. EMBO J. 1998; 17: 6979-91.

### SUMMARY OF INVENTION

### Technical Problem

The present invention has been made in view of such circumstances as described above. An object of the present invention is to develop a therapeutic strategy for specifically targeting cancer cells harboring inactivating SWI/SNF mutations. More specifically, an object of the present invention is to develop a therapeutic strategy for specifically targeting cancer cells in which a normal BRG1 function is suppressed, preferably cancer cells in which normal BRG1 is lost.

### Solution to Problem

Synthetic-lethality therapy is a very promising cancer treatment method. For example, the BRCA1 and BRCA2 genes have a synthetic-lethal relationship with the PARP1 gene. The growth of BRCA1-deficient or BRCA2-deficient cancer cells is dependent on PARP1 protein function. These findings have been translated to the clinic through the development of PARP inhibitors to treat BRCA1/BRCA2-deficient tumors (Chan D A, et al. Nat Rev Drug Discov. 2011; 10: 351-64). Besides, a synthetic-lethality therapy has also been proposed for treatment of cancers deficient in genes involved in DNA mismatch repair and cell metabolism (Muller F L, et al. Nature. 2012; 488: 337-42, Chan D A, et al. Sci Transl Med. 2011; 3: 94ra70, Martin S A, et al. Cancer Cell. 2010; 17: 235-48). The present inventors have earnestly studied in order to apply synthetic-lethality therapy to a therapeutic strategy for specifically killing cancer cells harboring inactivating SWI/SNF mutations.

As a result, the present inventors have found that suppressing BRM expression in BRG1-deficient cancer cells remarkably suppresses the cancer cell growth. This growth suppression is complemented with the introduction of the wild-type BRG1 gene, but not complemented with the introduction of a BRG1 gene with a disrupted ATP-binding pocket. Hence, it has been revealed that a loss of the ATPase activity of a SWI/SNF complex particularly contributes to the growth suppression. Moreover, it has been found that cancer cells whose growth is suppressed by suppressing the BRM expression exhibit a flattened morphology, and that the proportion of cells exhibiting G1 arrest in the cell cycle is increased. Further, expression of a senescence marker p21 has also been observed. These facts have revealed that senescence is induced. On the other hand, other driver gene mutations (for example, EGFR mutations, ALK fusions, and so on) serving as targets of molecularly targeted therapies have not been found in BRG1-deficient cancer cells. This suggests that current molecularly targeted therapies based on tyrosine kinase inhibitors are not suitable.

From the above results, the present inventors have found that BRG1 and BRM have a synthetic-lethal relationship, and that a treatment for inhibiting BRM (particularly its ATPase activity) is a promising approach for treating a BRG1-deficient cancer having no mutation in known therapeutic target genes. Additionally, it has also been revealed that, in this therapeutic strategy, a BRM inhibitor may be administered to a cancer patient after the selection based on BRG1 function suppression, thus enabling an efficient treatment based on the companion diagnosis.

Further, the present inventors have also successfully developed a novel screening system based on ATPase activity inhibition in order to develop a BRM inhibitor useful in this therapeutic strategy. These have led to the completion of the present invention.

Thus, the present invention relates to a synthetic-lethality therapy for specifically targeting cancer cells harboring inactivating SWI/SNF mutations, and a companion diagnosis for the synthetic-lethality therapy.

More specifically, the present invention provides the following inventions.

(1) A method for evaluating a cancer, the method comprising detecting whether or not a BRG1 function is suppressed in a biological sample derived from a cancer patient.

(2) A method for predicting responsiveness to a cancer treatment with a compound capable of inhibiting BRM, the method comprising:

detecting whether or not a BRG1 function is suppressed in a biological sample derived from a cancer patient; and

determining that the patient, from which the BRG1 function suppression is detected, responds to the cancer treatment with the compound capable of inhibiting BRM.

(3) A method for selecting a target of a cancer treatment with a compound capable of inhibiting BRM, the method comprising:

detecting whether or not a BRG1 function is suppressed in a biological sample derived from a cancer patient; and

selecting the patient, from which the BRG1 function suppression is detected, as the target of the cancer treatment with the compound capable of inhibiting BRM.

(4) A method for treating a cancer having a suppressed BRG1 function, the method comprising administering a compound capable of inhibiting BRM to a patient from which BRG1 function suppression is detected.

(5) A method for treating a cancer having a suppressed BRG1 function, the method comprising:

detecting whether or not a BRG1 function is suppressed in a biological sample derived from a cancer patient; and

administering a compound capable of inhibiting BRM to the patient from which the BRG1 function suppression is detected.

(6) A reagent for detecting whether or not a BRG1 function is suppressed in the method according to any one of (1) to (3) and (5), the reagent comprising as an active ingredient any one of the following molecules (a) to (c):

(a) an oligonucleotide primer capable of specifically binding to a BRG1 gene;

(b) an oligonucleotide probe capable of specifically binding to a BRG1 gene; and

(c) an antibody capable of specifically binding to a BRG1 protein.

(7) A screening method for a compound used in a treatment against a cancer having a suppressed BRG1 function, the method comprising the step of selecting the compound based on whether or not BRM is inhibited.

### Advantageous Effects of Invention

The present invention makes it possible to efficiently evaluate a cancer and predict responsiveness to a cancer treatment with a BRM inhibitor on the basis of BRG1 function suppression. According to the present invention, a cancer treatment with a BRM inhibitor would be performed for a cancer patient having a suppressed BRG1 function after the patient is selected by detecting whether or not a BRG1 function is suppressed (for example, inactivating mutation or expression reduction) in a biological sample derived from the patient. This makes it possible to greatly improve the treatment outcome of the cancer patient. The uses of the probe and primer for the BRG1 gene and the antibody against BRG1 enable such an efficient companion diagnosis by detecting whether or not a BRG1 function is suppressed.

### BRIEF DESCRIPTION OF DRAWINGS

FIG. 1 is a figure for illustrating BRG1 and BRM expression in surgically resected non-small-cell lung carcinomas. A: shows the strategy of this study. B: shows proportions of specimens expressing the BRG1 and BRM proteins. C: representative images of immunohistochemical staining. Upper panel: BRG1-negative/BRM-positive specimens. Lower panel: BRG1/BRM-positive specimens. D: BRG1/BRM protein expression and other clinicopathological factors.

FIG. 2 is a figure for illustrating differential sensitivity of BRG1-deficient and -proficient cell lines to BRM knockdown. A: shows BRG1 protein and BRM protein expression in human cancer cell lines: BRG1-deficient (left panel) and BRG1-proficient (right panel). B: shows survival of the BRG1-deficient and -proficient cancer cells after the BRM knockdown: BRG1-deficient (left panel) and -proficient (right panel). Data are shown as mean±SD. Asterisks; significant differences in surviving fraction between siBRM-treated cells and siControl-treated cells, as determined by Student's t test (*, P<0.05; **, P<0.001). C: shows BRM protein expression in the cancer cells after the BRM knockdown. D: shows surviving fraction of the cancer cell lines according to BRG1 and BRM expression status.

FIG. 3 is a figure for illustrating the effect of the BRM knockdown on cell growth in noncancerous cells and in NCI-H1299 cells complemented with wild-type BRG1 cDNA. A: BRG1 protein and BRM protein expression in HFL-1 cells and MRC-5 fibroblasts. B: shows surviving fraction of the HFL-1 cells and the MRC-5 cells after the BRM knockdown. Data are shown as mean±SD. C, D: illustrate BRG1 cDNA complementation assay. WT; wild-type, KA; ATPase mutant. P values were calculated using Student's t test. Data are shown as mean±SD of three replicates per treatment condition.

FIG. 4 is a figure for illustrating the effect of the BRM knockdown on growth inhibition in BRG1-deficient cancer cells. A: cell-cycle profile. Upper panel; immunoblot analysis of BRM and α-tubulin. Bottom panel: cell-cycle profiles determined by flow cytometry. Asterisk: a significant difference in the fraction of cells in G1-phase between dox-treated cells and -untreated cells as determined by Student's t test (P<0.01). B: shows expression of senescence-associated (SA)-β-gal and p21. Upper panel; immunoblot analysis of BRM, p21, and α-tubulin. Middle panel; percentage of SA-β-gal-positive cells. Asterisk; a significant difference in the percentage of SA-β-gal-positive cells between dox-treated cells and -untreated cells as determined by Student's t test (P<0.001). Bottom panel; representative micrographs of cells stained for SA-β-gal (blue). Scale bars, 100 μm. C: shows senescence-associated heterochromatic foci (SAHF) and mitotic catastrophe (MC) as revealed by nuclear DAPI staining. Upper panel; representative images showing normal nuclear (Normal), SAHF, and MC patterns in dox-treated NCI-H1299-shBRM cells. Scale bars, 10 μm. Bottom panel; percentage of cells exhibiting SAHF and MC. Asterisk; a significant difference in the percentage of SAHF-positive cells between dox-treated cells and -untreated cells as determined by Student's t test (P<0.001). D: shows p21 expression and clonogenic survival of NCI-H1299 cells after BRM knockdown followed by reexpression of BRM. A schematic of the experimental protocol is shown at the top of this panel in the figure. NCI-H1299-shBRM cells were cultured in the presence of dox for 7 days (BRM KD) and then cultured in the absence of dox for an additional 5 days (KD→Exp). Bottom left panel; shows immunoblot analysis of BRM, p21, and α-tubulin. Bottom right panel; shows clonogenic survival assay. Data are shown as mean±SD. Asterisks; significant decreases in the surviving fraction relative to that of control cells as determined by Student's t test (P<0.001). E to G: BRG1-deficient (NCI-H1299, A549, and NCI-H157) and BRG1-proficient (NCI-H520, LK2, and NCI-H460) non-small-cell lung cancer cells were transfected twice (days 1 and 3) with BRM-targeting siRNA (siBRM) or control siRNA (siControl). E; immunoblot analysis of BRM and α-tubulin (loading control). F; positivity for SA-β-gal staining. Asterisks; significant differences in the percentage of SA-β-gal-positive cells between siBRM-treated cells and siControl-treated cells as determined by Student's t test (P<0.005). G; cell-cycle profiles determined by flow cytometry. Asterisks; significant differences in the fraction of cells in G1-phase between siBRM-treated cells and siControl-treated cells, as determined by Student's t test (P<0.005).

FIG. 5 is a figure for illustrating suppression of BRG1-deficient cancer cell growth by BRM knockdown both in vitro and in vivo. A: shows long-term proliferation of NCI-H1299 cells and HeLa cells after BRM knockdown. B: shows growth of NCI-H1299 xenografts in mice. Data represent mean±SD. Asterisks; significant differences in the tumor volume between the dox-fed mice and the controls, as determined by Student's t test (P<0.05).

### DESCRIPTION OF EMBODIMENTS

<Method for Evaluating Cancer, Method for Predicting Cancer Treatment Responsiveness, Method for Selecting Cancer Treatment Target>

In the present invention, tumor, malignant tumor, cancer, malignant neoplasm, carcinoma, sarcoma, and the like are collectively referred to as “tumor” or “cancer”. The present invention has revealed that BRG1 and BRM have a synthetic-lethal relationship in cancer cells, and that inhibiting BRM in cancer cells having a suppressed BRG1 function can suppress the cancer cell growth. Based on these findings, it is possible to evaluate responsiveness to a cancer treatment with a compound capable of inhibiting BRM on the basis of the BRG1 function suppression. Thus, the present invention provides a method for predicting responsiveness to a cancer treatment with a compound capable of inhibiting BRM, the method comprising:

detecting whether or not a BRG1 function is suppressed in a biological sample derived from a cancer patient; and

determining that the patient, from which the BRG1 function suppression is detected, responds to the cancer treatment with the compound capable of inhibiting BRM.

In addition, a cancer treatment with a compound capable of inhibiting BRM is suitable for a patient from which BRG1 function suppression is detected as described above. On the basis of BRG1 function suppression, a patient who should receive the cancer treatment with the compound capable of inhibiting BRM and a patient who should not are selected, hence enabling an efficient treatment. Thus, the present invention also provides a method for selecting a target of a cancer treatment with a compound capable of inhibiting BRM, the method comprising:

detecting whether or not a BRG1 function is suppressed in a biological sample derived from a cancer patient; and

selecting the patient, from which the BRG1 function suppression is detected, as the target of the cancer treatment with the compound capable of inhibiting BRM.

In the present invention, the “cancer patient” may be not only a person who has a cancer, but also a person who is suspected of a cancer. In the method of the present invention, the cancer patient to be targeted for the detection of whether or not a BRG1 function is suppressed is not particularly limited, and all cancer patients can be the target. As cancers from which BRG1 function suppression is observed, for example, lung cancer, medulloblastoma, clear cell renal carcinoma, liver cancer, rhabdoid tumor, and the like have been reported. Nevertheless, such cancers are not limited to these, and may be, for example, breast cancer, colon cancer, prostate cancer, stomach cancer, ovarian cancer, cervical cancer, endometrial cancer, uterine cancer, kidney cancer, thyroid cancer, esophageal cancer, squamous-cell carcinoma, leukemia, osteosarcoma, melanoma, glioblastoma, neuroblastoma, ovarian cancer, head and neck cancer, testicular cancer, colorectal cancer, blood cancer, retinoblastoma, pancreatic cancer, and the like. Since it is known that a BRG1 function is suppressed in 5 to 10% of cases of many cancers including lung cancer, the method of the present invention makes it possible to select a cancer patient with such a frequency as a “treatment target.”

The “biological sample” used in the present invention is not particularly limited, as long as it is possible to detect whether or not a BRG1 function is suppressed. Nevertheless, the “biological sample” is preferably a cancer biopsy specimen, and may be protein extracts and nucleic acid extracts (such as mRNA extracts, cDNA preparations and cRNA preparations prepared from mRNA extracts) obtained from such samples.

In the present invention, “BRG1” and “BRM” are subunits of a SWI/SNF chromatin-remodeling complex. SEQ ID NO: 1 shows a typical base sequence of a human-derived naturally occurring BRG1 genomic DNA, SEQ ID NO: 2 shows a typical base sequence of a human-derived naturally occurring BRG1 cDNA, and SEQ ID NO: 3 shows a typical amino acid sequence of a human-derived naturally occurring BRG1 protein. Moreover, SEQ ID NO: 4 shows a typical base sequence of a human-derived naturally occurring BRM genomic DNA, SEQ ID NO: 5 shows a typical base sequence of a human-derived naturally occurring BRM cDNA, and SEQ ID NO: 6 shows a typical amino acid sequence of a human-derived naturally occurring BRM protein. It should be understood that even if BRG1 or BRM has no mutation, there may be a difference in the sequence case by case due to polymorphism or the like.

In the present invention, the phrase “BRG1 function suppression” includes both BRG1 inactivation and expression reduction. The “BRG1 inactivation” is typically caused by an inactivating mutation in BRG1 (including BRG1 deficiency). The inactivating mutation may occur, for example, by a missense mutation in a BRG1 helicase domain (positions 766 to 931 or positions 1084 to 1246 of the amino acid sequence of SEQ ID NO: 3), a missense mutation in an ATP-binding domain (positions 779 to 786 of the amino acid sequence of SEQ ID NO: 3) in the helicase domain, a nonsense mutation over the entire region, a complete or partial deletion of the gene, or the like. However, the cause is not limited to these, as long as BRG1 is inactivated. The BRG1 mutations have been reported in the literature (Rodriguez-Nieto S, et al. Hum Mutat. 2011; 32: E1999-2017) and so forth. For example, many of the BRG1 genome abnormalities are homozygous truncating mutations and deletions, and normal BRG1 expression is lost in many cases. Moreover, the loss and reduction in BRG1 expression are also observed frequently. In the present invention, this two are collectively referred to as “BRG1 deficiency.”

In the present invention, the phrase “BRG1 expression reduction” includes both expression reduction at a transcription level and expression reduction at a translation level.

In the present invention, the method for “detecting BRG1 function suppression” is not particularly limited, but examples thereof include the following methods.

—Detection of BRG1 Mutation—

In the present invention, the phrase “detecting a mutation” basically means detecting a mutation in the genomic DNA. In a case where a mutation in the genomic DNA reflects a change in a base of a transcription product or a change in an amino acid of a translation product, the phrase also means to include detecting the changes in these transcription product and translation product (i.e., indirect detection).

A preferable embodiment of the method of the present invention is a method for detecting a mutation by directly determining a base sequence of a BRG1 gene region of a cancer patient. In the present invention, the term “BRG1 gene region” means a certain region of the genomic DNA including the BRG1 gene. This region includes an expression control region (for example, a promoter region, an enhancer region) of the BRG1 gene and a 3′ untranslated region of the BRG1 gene, and the like. A mutation in these regions may influence, for example, the transcription activity of the BRG1 gene.

In this method, first, a DNA sample is prepared from a biological sample derived from a cancer patient. Examples of the DNA sample include genomic DNA samples, and cDNA samples prepared from RNAs by reverse transcription.

The method for extracting a genomic DNA or an RNA from a biological sample is not particularly limited, and a known method can be selected as appropriate for use. Examples of the method for extracting a genomic DNA include: an SDS phenol method (in which a tissue is stored in ethanol or a solution containing urea, a protein of the tissue is denatured with a protease (proteinase K), a surfactant (SDS), and phenol, and a DNA is precipitated and extracted from the tissue with ethanol); and a DNA extraction method using Clean Columns (registered trademark, manufactured by NexTec), AquaPure (registered trademark, manufactured by Bio-Rad), ZR Plant/Seed DNA Kit (manufactured by Zymo Research), AquaGenomic Solution (registered trademark, manufactured by Mo Bi Tec), prepGEM (registered trademark, manufactured by ZyGEM), and BuccalQuick (registered trademark, manufactured by TrimGen). Meanwhile, examples of the method for extracting an RNA include: an extraction method using phenol and a chaotropic salt (more concretely, which is an extraction method using a commercially available kit of Trizol (manufactured by Invitrogen), ISOGEN (manufactured by Wako Pure Chemical Industries, Ltd.), or the like); and methods using other commercially available kits (RNAPrep Total RNA Extraction Kit (manufactured by Beckman Coulter), RNeasyMini (manufactured by QIAGEN), RNA Extraction Kit (manufactured by Pharmacia Biotech) or the like). Further, the reverse transcriptase used to prepare a cDNA from the extracted RNA is not particularly limited, and examples thereof include reverse transcriptases derived from retroviruses such as RAV (Rous associated virus) and AMV (Avian myeloblastosis virus), and reverse transcriptases derived from mouse retroviruses such as MMLV (Moloney murine leukemia virus).

In this embodiment, a DNA containing a mutation site of the BRG1 gene region is then isolated, and the base sequence of the isolated DNA is determined. The DNA can be isolated, for example, by PCR or the like using a pair of oligonucleotide primers designed to flank a mutation in the BRG1 gene region with the genomic DNA or the RNA as a template. The base sequence of the isolated DNA can be determined by a method known to those skilled in the art such as the Maxam-Gilbert method or the Sanger method.

The base sequence of DNA or cDNA thus determined is compared with that of a control (for example, the base sequence of DNA or cDNA derived from a non-cancerous tissue of the same patient). Thereby, whether or not the BRG1 gene region is mutated in the cancer cells of the cancer patient can be determined.

As the method for detecting a mutation of the BRG1 gene region, various methods capable of detecting a mutation can be employed, besides the method by which the base sequence of DNA or cDNA is directly determined.

For example, in the present invention, a mutation may be detected by a method as follows. First, a DNA or cDNA sample is prepared from a biological sample. Then, an oligonucleotide probe is prepared which has a base sequence complementary to abase sequence containing a BRG1 gene region mutation, and which is labeled with a reporter fluorescent dye and a quencher fluorescent dye. Subsequently, the oligonucleotide probe is hybridized to the DNA sample, and the base sequence containing the BRG1 gene region mutation is amplified using, as a template, the DNA sample with the oligonucleotide probe hybridized thereto. Thereafter, fluorescence is detected which is emitted by the reporter fluorescent dye through degradation of the oligonucleotide probe associated with the amplification. Then, the detected fluorescence is compared with that of a control. Examples of such a method include a double-dye probe method, and so-called TaqMan (registered trademark) probe method.

In still another method, a DNA or cDNA sample is prepared from a biological sample. Then, in a reaction system including an intercalator which emits fluorescence when inserted between DNA double strands, a base sequence containing the BRG1 gene region mutation is amplified using the DNA sample as a template. Subsequently, the temperature of the reaction system is changed to detect a variation in the intensity of fluorescence emitted by the intercalator. The detected variation in the intensity of the fluorescence due to the change in the temperature is compared with that of a control. An example of such a method includes a HRM (high resolution melting) method.

In still another method, first, a DNA or cDNA sample is prepared from a biological sample. Then, a DNA containing a mutation site of a BRG1 gene region is amplified. Subsequently, the amplified DNAs are cleaved with a restriction enzyme. Thereafter, the DNA fragments are separated on the basis of the size. After that, the size of the detected DNA fragments is compared with that of a control. Examples of such a method include methods utilizing Restriction Fragment Length Polymorphism (RFLP), PCR-RFLP method, and the like.

In still another method, first, a DNA or cDNA sample is prepared from a biological sample. Then, a DNA containing a mutation site of a BRG1 gene region is amplified. Subsequently, the amplified DNAs are each dissociated into single-stranded DNAs. Thereafter, the dissociated single-stranded DNAs are separated on a non-denaturing gel. The mobility of the separated single-stranded DNAs on the gel is compared with that of a control. An example of such a method includes the PCR-SSCP (single-strand conformation polymorphism) method.

In still another method, first, a DNA or cDNA sample is prepared from a biological sample. Then, a DNA containing a mutation site of a BRG1 gene region is amplified. Subsequently, the amplified DNAs are separated on a gel where the concentration of a DNA denaturant is gradually increased. Thereafter, the mobility of the separated DNAs on the gel is compared with that of a control. An example of such a method includes the denaturant gradient gel electrophoresis (DGGE) method.

Still another method is a method that uses a DNA containing a mutation site of a BRG1 gene region prepared from a biological sample, and a substrate having an oligonucleotide probe which is fixed thereto and capable of hybridizing to the DNA. Examples of such a method include the DNA array method, and the like.

In still another method, first, a DNA or cDNA sample is prepared from a biological sample. Moreover, prepared is an “oligonucleotide primer having a base sequence complementary to a base located on the 3′ side of, and away by a distance of one base from, a base at a mutation site of a BRG1 gene region and a base sequence on the 3′ side therefrom.” Then, using the DNA as a template and the primer, a ddNTP primer extension reaction is carried out. Subsequently, the primer extension reaction product is subjected to a mass spectrometer for the mass measurement. Thereafter, the genotype is determined from the mass measurement result. After that, the determined genotype is compared with that of a control. An example of such a method includes the MALDI-TOF/MS method.

In still another method, first, a DNA or cDNA sample is prepared from a biological sample. Then, prepared is an oligonucleotide probe having 5′-“a base sequence complementary to a base at a mutation site of a BRG1 gene region and a base sequence on the 5′ side therefrom”-“a base sequence incapable of hybridizing to a base located on the 3′ side of, and away by a distance of one base from, the mutation site of the BRG1 gene region and a base sequence on the 3′ side therefrom”-3′ (flap). Moreover, prepared is an “oligonucleotide probe having a base sequence complementary to the base at the mutation site of the BRG1 gene region and a base sequence on the 3′ side therefrom.” Subsequently, the two types of the oligonucleotide probes are hybridized to the prepared DNA. Thereafter, the hybridized DNA is cleaved with a single-stranded DNA cleavage enzyme to release the flap. In the present invention, the single-stranded DNA cleavage enzyme is not particularly limited, and an example thereof includes cleavase. In this method, an oligonucleotide probe having a sequence complementary to that of the flap and labeled with reporter fluorescence and quencher fluorescence is then hybridized to the flap. Subsequently, the intensity of emitted fluorescence is measured. Thereafter, the measured fluorescence intensity is compared with that of a control. An example of such a method includes the Invader method.

In still another method, first, a DNA or cDNA sample is prepared from a biological sample. Then, a DNA containing a mutation site of a BRG1 gene region is amplified. Subsequently, the amplified DNAs are each dissociated into single strands. Only one of the dissociated single-stranded DNAs is separated. Thereafter, an extension reaction is carried out on each base from one near the base at the mutation site of the BRG1 gene region. Pyrophosphoric acid generated in this event is caused to emit light enzymatically, and the intensity of the luminescence is measured. After that, the measured fluorescence intensity is compared with that of a control. An example of such a method includes the Pyrosequencing method.

In still another method, first, a DNA or cDNA sample is prepared from a biological sample. Then, a DNA containing a mutation site of a BRG1 gene region is amplified. Subsequently, prepared is an “oligonucleotide primer having a base sequence complementary to a base located on the 3′ side of, and away by a distance of one base from, a base at the mutation site of the BRG1 gene region and a base sequence on the 3′ side therefrom.” Thereafter, a single base extension reaction is carried out using the amplified DNAs as a template and the prepared primer in the presence of a fluorescence-labeled nucleotide. After that, the polarization of the fluorescence is measured. Then, the measured fluorescence polarization is compared with that of a control. An example of such a method includes the AcycloPrime method.

In still another method, first, a DNA or cDNA sample is prepared from a biological sample. Then, a DNA containing a mutation site of a BRG1 gene region is amplified. Subsequently, prepared is an “oligonucleotide primer having a base sequence complementary to a base located on the 3′ side of, and away by a distance of one base from, a base at the mutation site of the BRG1 gene region and a base sequence on the 3′ side therefrom.” Thereafter, a single base extension reaction is carried out using the amplified DNAs as a template and the prepared primer in the presence of a fluorescence-labeled nucleotide. After that, the type of base used in the single base extension reaction is determined.

Then, the determined base type is compared with that of a control. An example of such a method includes the SNuPE method. Note that if a mutation occurs together with a change (for example, substitution, deletion, insertion) in an amino acid in a BRG1 protein, a sample prepared from a biological sample may be a protein. To detect a mutation in such a case, it is possible to utilize a method using a molecule (for example, antibody) capable of specifically binding to a site where an amino acid changes due to the mutation. A method for detecting a protein using an antibody will be described later.

—Detection of BRG1 Expression Reduction—

In the present invention, the term “BRG1 expression reduction” generally means that the expression level is low in comparison with a control (for example, an expression level of a non-cancerous tissue from the same patient or a healthy person).

In the method for detecting the amount of BRG1 expressed at a transcription level, first, an RNA or a cDNA is prepared from a biological sample derived from a cancer patient by the above-described method. Then, an oligonucleotide primer or an oligonucleotide probe is each used in an amplification reaction or a hybridization reaction, and the amplification product or the hybrid product is detected.

As such a method, it is possible to utilize, for example, an RT-PCR method, a northern blotting method, a dot blot method, a DNA array method, an in situ hybridization method, an RNase protection assay method, mRNA-seq, or the like.

Those skilled in the art can design an oligonucleotide primer and an oligonucleotide probe suitable for each method on the basis of the base sequence of BRG1 cDNA (for example, SEQ ID NO: 2) by conventional methods.

In the method for detecting the amount of BRG1 expressed at a translation level, first, a protein sample is prepared from a biological sample derived from a cancer patient. Then, an antibody specific to a BRG1 protein is used in a antigen-antibody reaction, and the binding of the antibody to the BRG1 protein is detected. In a case where the antibody specific to BRG1 is labeled, the BRG1 protein can be detected directly. Meanwhile, in a case where the antibody is not labeled, a labeled molecule (for example, secondary antibody or Protein A) capable of recognizing the antibody is additionally caused to act thereon so that the BRG1 protein can be detected indirectly by utilizing the label of the molecule.

As such a method, it is possible to utilize, for example, an immunohistochemical (immunity staining) method, a western blotting method, an ELISA method, flow cytometry, imaging cytometry, a radioimmunoassay, an immunoprecipitation method, an analysis method using an antibody array, or the like. The immunohistochemistry also has such advantages that additional information such as the morphology and distribution state of cancer cells in tissues can be obtained at the same time.

The type, origin, and so forth of the antibody used are not particularly limited, but the antibody is preferably a monoclonal antibody. As long as BRG1 can be detected with sufficient specificity, it is also possible to use an oligoclonal antibody (a mixture of several types or several tens of types of antibodies) or a polyclonal antibody.

Moreover, it is also possible to use a functional fragment of the antibody such as Fab, Fab′, F(ab′)2, Fv, scFv, sc(Fv)2, dsFv, and a diabody, or a multimer (for example, dimer, trimer, tetramer, polymer) of the antibody. The anti-BRG1 antibody may be a commercially available product, for example, Clone EPNCIR111A (Epitomics 2822-1) or Clone G-7 (SantaCruz sc-17796).

The BRG1 protein can also be detected using a mass spectrometry method (MS). Particularly, the analysis with a mass spectrometer coupled to liquid chromatography (LC/MS) is advantageous because of its sensitivity. The measurement according to the mass spectrometry method can be performed, for example, by: preparing a protein from a biological sample, labeling the protein, fractionating the protein, subjecting the fractionated proteins to mass spectrometry, and identifying the BRG1 protein from the mass spectrometry values. As the label, an isotope labeling reagent known in the technical field can be used, and an appropriate labeling reagent can be obtained as a commercially available product. Moreover, the fractionation can also be performed by a method known in the technical field. For example, the fractionation may be performed using a commercially-available strongly cationic column and so forth.

—Others—

It is known in the technical field that promoter hypermethylation is one main cause of gene expression reduction. Thus, in detecting whether or not a BRG1 function is suppressed, it is also conceivable to perform the detection on the basis of a methylation of a BRG1 gene promoter. To detect the promoter methylation, known methods can be utilized such as, for example, a method in which a change in a base sequence is directly detected by base sequencing after a treatment with bisulfite having an activity to convert methylated cytosine to uracil, or an indirect detection method utilizing a restriction endonuclease capable of recognizing (capable of cleaving) a base sequence before a bisulfite treatment but incapable of recognizing (incapable of cleaving) a base sequence after the bisulfite treatment.

After BRG1 function suppression is evaluated as described above, in a case where the BRG1 function suppression, for example, an inactivating mutation ablating the BRG1 function, is detected, in a case where BRG1 expression reduction is detected, or in a case where another phenomenon (for example, excessive promoter methylation) causing BRG1 inactivation or expression reduction is detected, it is possible to determine that the cancer patient responds to the cancer treatment with the compound capable of inhibiting BRM, and the cancer patient can be selected as the target of the cancer treatment with the compound capable of inhibiting BRM. Herein, the “responsiveness to a cancer treatment” and related phrases mean indicators that are used to evaluate whether or not the compound capable of inhibiting BRM can exhibit a therapeutic effect against a cancer. The determination of the responsiveness may include not only whether a patient responds or not, but also an evaluation of the degree of the responsiveness in the case where a patient responds (for example, evaluations such as that a high responsiveness can be expected, a moderate responsiveness can be expected, and so on). Thus, depending on the degree of BRG1 function suppression, a patient who may become a treatment target can be selected, for example, at such a level that a moderate responsiveness can be expected.

On the other hand, in a case where no BRG1 function suppression is observed in a cancer patient, it is possible to determine that the patient does not respond to the cancer treatment with the compound capable of inhibiting BRM, and the patient can be excluded from the target of the cancer treatment with the compound capable of inhibiting BRM. This makes it possible to improve the responsiveness rate of the treatment.

It should be noted that in a case where BRM, which is the target of the present treatment, is not properly expressed, the cancer treatment with the compound capable of inhibiting BRM may not be performed effectively. Thus, in evaluating a cancer, in predicting a responsiveness to a cancer treatment, and in selecting a cancer patient, it is also possible to further add normal expression of BRM as the indicator. The method for detecting BRM expression is the same as the above-described detection BRG1 of expression.

<Cancer Treatment Method>

Moreover, the present invention provides a method for treating a cancer having a suppressed BRG1 function, the method comprising administering a compound capable of inhibiting BRM to a patient from which BRG1 function suppression is detected. BRG1 in a patient may be detected by a person other than a doctor, or may be detected by a doctor himself/herself.

The “compound capable of inhibiting BRM” used in the treatment method of the present invention is not particularly limited, and may be a known compound or may be a compound identified by screening which will be described later.

The compound capable of inhibiting BRM can be administered in various forms such as tablets, powders, granules, capsules, and liquids, depending on the characteristics of the compound, by oral administration or parenteral administration (for example, intravenous administration, intraarterial administration, local administration) to a cancer patient. The dose is not particularly limited, as long as the amount is effective for inhibiting BRM to treat the cancer. The dose should be selected as appropriate in accordance with the age, weight, symptom, and health state of a cancer patient, the progression of the cancer, and so forth, in addition to the properties of the compound. How often the compound is administered is not particularly limited, either, and can be selected as appropriate in accordance with the purpose. For example, as the dose administered in a day, the compound may be administered once a day, or may be administered separately multiple times a day. When the compound capable of inhibiting BRM is administered to human, the range of the dose per day is approximately 0.01 mg/kg body weight to approximately 500 mg/kg body weight, preferably approximately 0.1 mg/kg body weight to approximately 100 mg/kg body weight. When administered to human, the compound is administered preferably once a day, or preferably separately two to four times a day such that the administration is repeated at appropriate intervals. Meanwhile, the amount in a day may exceed the above-described amount in accordance with the diagnosis of the doctor as necessary.

This makes it possible to further inhibit BRM in cancer cells having a suppressed BRG1 function in a cancer patient, and to treat the cancer owing to the synthetic-lethal effect.

Examples of the cancer as the treatment target include breast cancer, colon cancer, prostate cancer, lung cancer, stomach cancer, ovarian cancer, cervical cancer, endometrial cancer, uterine cancer, kidney cancer, hepatocellular carcinoma, thyroid cancer, esophageal cancer, squamous-cell carcinoma, leukemia, osteosarcoma, melanoma, glioblastoma, neuroblastoma, ovarian cancer, head and neck cancer, testicular cancer, colorectal cancer, blood cancer, retinoblastoma, pancreatic cancer, and the like, but are not limited to these cancers.

<Reagent for Detecting Whether or not BRG1 Function is Suppressed>

Further, the present invention provides a reagent for detecting whether or not a BRG1 function is suppressed in the above-described methods, the reagent comprising as an active ingredient any one of the following molecules (a) to (c):

(a) an oligonucleotide primer capable of specifically binding to a BRG1 gene;

(b) an oligonucleotide probe capable of specifically binding to a BRG1 gene; and

(c) an antibody capable of specifically binding to a BRG1 protein.

The polynucleotide primer should be designed on the basis of base sequence information on the BRG1 genomic DNA and cDNA (for example, SEQ ID NOs: 1 and 2) in such a manner as to make the primer appropriate for the aforementioned methods and amplification region, and to prevent an amplification product of genes other than the BRG1 gene from being generated as much as possible. Those skilled in the art can design such an oligonucleotide primer by conventional methods. The length of the oligonucleotide primer is normally 15 to 50 bases long, preferably 15 to 30 bases long, but may be longer depending on the method and the purpose.

The polynucleotide probe should be designed on the basis of the base sequence information on the BRG1 genomic DNA and cDNA (for example, SEQ ID NOs: 1 and 2) in such a manner as to make the primer appropriate for the aforementioned methods and hybridization region, and to prevent hybridization to genes other than the BRG1 gene from occurring as much as possible. Those skilled in the art can design such an oligonucleotide probe by conventional methods. The length of the oligonucleotide probe is normally 15 to 200 bases long, preferably 15 to 100 bases long, and furthermore preferably 15 to 50 bases long, but may be longer depending on the method and the purpose.

When used, the oligonucleotide probe is preferably labeled as appropriate. Examples of the labeling method include: a method in which the 5′ end of an oligonucleotide is phosphorylated and labeled with ³²P using a T4 polynucleotide kinase; and a method in which a substrate base labeled with an isotope such as ³²P, a fluorescence dye, biotin, or the like is incorporated using a DNA polymerase such as Klenow enzyme, and random hexamer oligonucleotides as primers (random priming method and the like).

The oligonucleotide primer and the oligonucleotide probe of the present invention can be prepared, for example, using a commercially-available oligonucleotide synthesizer. The oligonucleotide probe can also be prepared as a double-stranded DNA fragment obtained by restriction enzyme treatment or the like. Meanwhile, the oligonucleotide primer and the oligonucleotide probe of the present invention may be composed only of naturally-occurring nucleotides (deoxyribonucleotides (DNA) or ribonucleotides (RNA)), or part or whole thereof may be composed of artificial nucleotides. Examples of the artificial nucleotides include PNA (polyamide nucleic acids), LNA (registered trademark, locked nucleic acids), ENA (registered trademark, 2′-O,4′-C-Ethylene-bridged nucleic acids), and complexes of these.

In the case where the antibody capable of specifically binding to a BRG1 protein is a polyclonal antibody, the polyclonal antibody can be obtained as follows. Concretely, an animal is immunized with an antigen (a BRG1 protein, a partial peptide thereof, cells expressing any of these, or the like). Then, an antiserum from the animal is purified by conventional means (for example, salting-out, centrifugation, dialysis, column chromatography, or the like) to obtain the polyclonal antibody. Meanwhile, a monoclonal antibody can be prepared by a hybridoma method or a recombinant DNA method.

A typical example of the hybridoma method includes a method by Kohler and Milstein (Kohler & Milstein, Nature, 1975; 256: 495). Antibody-producing cells used in the cell fusion process of this method are spleen cells, lymph node cells, peripheral blood leukocytes, or the like of an animal (for example, mouse, rat, hamster, rabbit, monkey, goat) immunized with an antigen (a BRG1 protein, a partial peptide thereof, cells expressing any of these, or the like). It is also possible to use antibody-producing cells obtained by causing the antigen to act, in a medium, on the above-described types of cells, lymphocytes, or the like, which are isolated from non-immunized animals in advance. As myeloma cells, various known cell lines can be used. The antibody-producing cells and the myeloma cells may be originated from different animal species, as long as they can be fused. However, the antibody-producing cells and the myeloma cells are preferably originated from the same animal species. Hybridomas can be produced, for example, by cell fusion between mouse myeloma cells and spleen cells obtained from a mouse immunized with the antigen. By the subsequent screening, a hybridoma which produces a monoclonal antibody specific to a BRG1 protein can be obtained. The monoclonal antibody against the BRG1 protein can be obtained by culturing the hybridoma, or from the ascitic fluid of a mammal to which the hybridoma is administered.

The recombinant DNA method is a method by which the antibody of the present invention is produced as a recombinant antibody as follows. Concretely, a DNA encoding the aforementioned antibody is cloned from a hybridoma, B cells, or the like. The cloned DNA is incorporated into an appropriate vector, which is then introduced into host cells (for example, a mammalian cell line, Escherichia coli, yeast cells, insect cells, plant cells, or the like) for the production (for example, P. J. Delves, Antibody Production: Essential Techniques, 1997 WILEY, P. Shepherd and C. Dean Monoclonal Antibodies, 2000 OXFORD UNIVERSITY PRESS, Vandamme A. M. et al., Eur. J. Biochem. 1990; 192: 767-775). For the expression of the DNA encoding the antibody, DNAs encoding the heavy chain or the light chain may be incorporated separately into expression vectors to transform the host cells. Alternatively, the DNAs encoding the heavy chain and the light chain may be incorporated into a single expression vector to transform the host cells (see WO94/11523). The antibody can be obtained in a substantially pure and homogeneous form by culturing the host cells, followed by separation and purification in the host cells or from the culture liquid. For the separation and purification of the antibody, an ordinary method used for polypeptide purification can be employed. Once a transgenic animal (cattle, goat, sheep, pig, or the like) incorporating the antibody gene is prepared using a transgenic animal production technique, a large amount of monoclonal antibodies derived from the antibody gene can also be obtained from milk of the transgenic animal.

From the antibody obtained in the above described manner or the gene, it is possible to prepare a functional fragment of the antibody such as Fab, Fab′, F(ab′)2, Fv, scFv, sc(Fv)2, dsFv, and a diabody, and a multimer (for example, dimer, trimer, tetramer, polymer) of the antibody.

In a case where the amount of the antibody bound to the BRG1 protein is directly detected, the obtained anti-BRG1 antibody is directly labeled with an enzyme, a radioisotope, a fluorescence dye, an avidin-biotin system, or the like for use. On the other hand, in a case where an indirect detection method is performed to detect the amount of the antibody bound to the BRG1 protein by utilizing a secondary antibody or the like, the obtained anti-BRG1 antibody (primary antibody) does not necessarily have to be labeled; in the detection, it is only necessary to use a labeled molecule (for example, secondary antibody or Protein A) capable of recognizing the antibody.

The reagent of the present invention may comprise, as necessary, other ingredients acceptable as a reagent such as sterile water, a physiological saline, a buffer, and a preservative, in addition to the above-described molecules as the active ingredients.

<Screening Method for Compound Used in Cancer Treatment>

Furthermore, the present invention provides a screening method for a compound used in a treatment against a cancer having a suppressed BRG1 function, the method comprising the step of selecting the compound based on whether or not BRM is inhibited.

A test compound used in the screening system of the present invention is not particularly limited. Examples thereof include synthetic low-molecular-weight compound libraries, expression products from gene libraries, peptide libraries, siRNAs, antibodies, substances released from bacteria, liquid extracts and culture supernatants of cells (microorganisms, plant cells, animal cells), purified or partially purified polypeptides, extracts derived from marine organisms, plants, or animals, and random phage peptide display libraries.

In the present invention, “BRM inhibition” and related phrases mean to include both BRM activity inhibition and expression inhibition. As revealed in the present Examples, particularly a loss in the ATPase activity of BRM contributes to the synthetic lethality of BRG1 and BRM. Thus, the BRM inhibition, on which the screening is based, is preferably inhibition of the ATPase activity of BRM.

The screening system based on the ATPase activity inhibition is preferably an in vitro system containing a SWI/SNF chromatin-remodeling complex including BRM, and an ATPase reaction solution, from the viewpoint that specific detection is possible with high sensitivity. This in vitro system does not necessarily have to have all proteins constituting the SWI/SNF chromatin-remodeling complex, but the complex preferably contains BAF170, BAF155, and INI1 in addition to BRM as described in the present Examples.

In the screening, a test compound is added, and then the ATP consumption is detected. The ATPase consumption can be easily detected using a commercially available product, for example, ADP-glo (manufactured by Promega). As a result of the detection, if the consumption is reduced in comparison with ATPase consumption of a control (for example, a case where no test compound is added), it is possible to evaluate that the ATPase activity is inhibited.

In the case where BRM expression inhibition is detected, for example, a test compound is caused to act on cells expressing BRM, and then the BRM expression should be detected at a transcription level or translation level by the aforementioned method. Alternatively, it is also possible to use a reporter assay system utilizing an expression construct in which a reporter gene is ligated downstream of a BRM promoter. As a result of the detection, if the expression is reduced in comparison with BRM expression (in the reporter system, it is reporter expression instead of the BRM expression) of a control (for example, a case where no test compound is added), it is possible to evaluate that the BRM expression is inhibited.

The compound thus identified by the screening of the present invention may be mixed with a pharmacologically acceptable carrier and formulated into a drug by known pharmaceutical methods. Examples of the pharmacologically acceptable carrier include sterile water, physiological saline, vegetable oils, solvents, bases, emulsifiers, suspensions, surfactants, stabilizers, flavors, aromatic substances, excipients, vehicles, antiseptics, binders, diluents, isotonic agents, soothing agents, bulking agents, disintegrators, buffers, coating agents, lubricants, colorants, sweeteners, viscous agents, flavor modifiers, solubilizers, other additives, and the like, but are not limited to these.

### EXAMPLES

### Example 1

### Development of Therapeutic Strategy Based on Synthetic Lethality of BRG1/SMARCA4 Chromatin-Remodeling Gene Deficient Cancer

1. Materials and Methods

(1) Non-Small-Cell Lung Carcinoma (NSCLC) Cohort

Surgical specimens from 103 patients with NSCLC (51 adenocarcinomas and 53 squamous cell carcinomas), who received surgical resection at the National Cancer Center Hospital (Tokyo, Japan) between 1997 to 2007, were used for the analysis. The subjects were randomly selected from a cohort of 318 cases including similar numbers of lung adenocarcinoma and squamous-cell carcinoma cases of three differentiation grades (well differentiated, moderately differentiated, and poorly differentiated) (Tsuta K, et al. J Thorac Oncol. 2011; 6: 1190-9). The 103 specimens analyzed in this study include similar numbers of specimens representing the three differentiation grades: 18 well, 18 moderately, and 14 poorly differentiated adenocarcinomas, as well as 20 well, 16 moderately, and 17 poorly differentiated squamous-cell carcinomas. Detailed information regarding the 318-case cohort and their pathological characteristics has been reported previously (Tsuta K, et al. J Thorac Oncol. 2011; 6: 1190-9). The histological diagnoses were determined according to the World Health Organization (WHO) classification (Pathology and Genetics, Tumours of Lung, Pleura, Thymus and Heart. In Travis, W D, Brambilla, E, Muller-Hermelink, H K and Harris, C C (eds), World Health Organization Classification of Tumors; 2004: 1-344). Table 1 shows the characteristics of the patients. This study was approved by the Institutional Review Board of the National Cancer Center.

TABLE 1
Patient characteristics and their association with lost/reduced BRG1/SMARCA4 expression

| | | BRG1 | BRG1 | | |
|---|---|---|---|---|---|
| | All | lost/reduced | retained | P value | |
| Variant | No. (%) | No. (%) | No. (%) | Univariate* | Multivariate^{†} |
| Total patients | 103 (100.0) | 16 (100.0) | 87 (100.0) | NT | NT |
| Age | | | | | |
| Median [range] ± SD | 67 [44-82] ± 8 | 60 [48-78] ± 8 | 67 [44-82] ± 8 | 0.094 | 0.13 |
| >median | 52 (50.5) | 5 (31.3) | 47 (54.0) | | |
| Gender | | | | | |
| Male | 73 (70.9) | 16 (100.0) | 57 (65.5) | 0.0053 | NT |
| Female | 30 (29.1) | 0 (0.0) | 30 (34.5) | | |
| Histological cell | | | | | |
| type | | | | | |
| Adenocarcinoma | 50 (48.5) | 8 (50.0) | 42 (48.3) | 0.90 | NT |
| Squamous-cell | 53 (51.5) | 8 (50.0) | 45 (51.7) | | |
| carcinoma | | | | | |
| Differentiation | | | | | |
| Well | 38 (36.9) | 2 (12.5) | 36 (41.4) | 0.0011 | 0.022 |
| differentiated | | | | | |
| Moderately | 34 (33.0) | 3 (18.8) | 31 (35.6) | | |
| differentiated | | | | | |
| Poorly | 31 (30.1) | 11 (68.8) | 20 (23.0) | | |
| differentiated | | | | | |
| Pathological stage | | | | | |
| I | 42 (40.8) | 5 (31.3) | 37 (42.5) | 0.078 | 0.20 |
| II | 28 (27.2) | 8 (50.0) | 20 (23.0) | | |
| III and IV | 33 (32.0) | 3 (18.8) | 30 (34.5) | | |
| Smoking habit | | | | | |
| Never-smoker | 26 (25.2) | 1 (6.3) | 25 (38.7) | 0.057 | 0.17 |
| Ever-smoker | 77 (74.8) | 15 (93.8) | 62 (71.3) | | |
| EGFR mutation | | | | | |
| Mutant | 27 (26.2) | 0 (0.0) | 27 (31.0) | 0.0095 | NT |
| Wild-type | 76 (73.8) | 16 (100.0) | 60 (69.0) | | |
| KRAS mutation | | | | | |
| Mutant | 10 (9.7) | 0 (0.0) | 10 (11.5) | 0.15 | NT |
| Wild-type | 93 (90.3) | 16 (100.0) | 77 (88.5) | | |
| ALK fusion | | | | | |
| Positive | 1 (1.0) | 0 (0.0) | 1 (1.1) | 0.67 | NT |
| Negative | 102 (99.0) | 16 (100.0) | 86 (98.9) | | |
| FGFR1 amplification | | | | | |
| Positive | 1 (1.0) | 0 (0.0) | 1 (1.1) | 0.67 | NT |
| Negative | 68 (99.0) | 12 (100.0) | 56 (98.9) | | |
| BRM expression | | | | | |
| Lost or reduced | 40 (38.8) | 6 (37.5) | 34 (39.1) | 0.91 | NT |
| Retained | 63 (61.2) | 10 (62.5) | 53 (60.9) | | |

Abbreviation:
NT, not tested
*Association was examined by Fisher's exact test.
^{†}Association was examined by logistic regression test. Variables showing associations with P < 0.1 in the univariate analysis were subjected to this analysis. Gender and EGFR mutation were not included; they are not suitable for this analysis because no females and EGFR mutants were present in the “BRG lost/reduced” group.

(2) Immunohistochemical Analysis of BRG1 and BRM

Immunohistochemical analysis was performed on tissue microarray sections obtained from anonymized NSCLC samples. Formalin-fixed paraffin-embedded (FFPE) tissue sections were de-waxed in xylene, rinsed well in ethanol, and then washed in tap water. Antigen retrieval was performed by pressure cooking in 0.01 M citric acid (pH 6.0) (for BRM), or by immersion in TRS (target-retrieval solution) (manufactured by Dako) in a water bath at 95° C. (for BRG1). The sections were allowed to cool for 40 minutes at room temperature, and were then blocked in 1% hydrogen peroxide for 10 minutes, rinsed in tap water, placed in a humid staining chamber, and covered with TBS buffer (pH 7.6) for 5 minutes. The TBS buffer was drained off, and the sections were incubated with normal rabbit serum (NRS; Dako) diluted 1:5 in TBS. The sections were then incubated with a rabbit anti-BRG1 monoclonal antibody (1:200; Epitomics, 2822-1) for 1 hour at room temperature, or incubated with a rabbit anti-BRM polyclonal antibody (1:800; manufactured by Sigma, HPA029981) for 30 minutes at room temperature. The sections were then rinsed and incubated with Envision rabbit/HPP (manufactured by Dako) for 20 minutes. Finally, the sections were rinsed in running tap water, counter-stained as required, dehydrated, and mounted.

Salivary-gland tissue specimens exhibiting strong staining for both BRG1 and BRM in the nuclei were used as positive controls in each tissue microarray. The staining intensity of the BRG1 and BRM antibodies in the nuclei of each lung-cancer sample was expressed according to the intensity scores described by Fukuoka et al. (Fukuoka J, et al. Clin Cancer Res. 2004; 10: 4314-24) with some modifications: 0 (no signal), 1 (weak), 2 (moderate), and 3 (strong, i.e., the level found in salivary-gland cells). In addition, the intratumoral fractions of regions with each score (0 to 3) were determined. Six non-cancerous lung tissues (n=6) were also subjected to the same protocol. The expression levels of BRM and BRG1 in each tissue were compared according to the total score (TS), which was calculated as the sum of [intensity scores×fractions] for the scored regions. A tumor sample was judged to be positive when the TS was higher than the average TS calculated for normal lung epithelial tissues (n=6). Specific staining for BRG1 and BRM was confirmed by immunoblot analysis of cancer cell lines.

(3) Examination of Driver Gene Mutations

Genomic DNA obtained from tumor tissues was analyzed for somatic mutations in the EGFR and KRAS genes using the high-resolution melting method (Takano T, et al. J Clin Oncol. 2005; 23: 6829-37). Mutations in all DDR2 coding regions in squamous cell lung carcinoma samples were examined by targeted genome capture and massively parallel sequencing using an Ion PGM sequencing system and the Ion Target Seq Custom Enrichment Kit (manufactured by Life Technologies). EML4-ALK and KIF5B-ALK fusions were screened by immunohistochemistry using an anti-ALK antibody (Abcam; 5A4), followed by confirmation via reverse-transcriptase polymerase chain reaction and/or fluorescence/chromogenic in situ hybridization (Yoshida A, et al. Am J Surg Pathol. 2011; 35: 1226-34). RET and ROS1 fusions (in cases for which RNA samples were available) were examined by RT-PCR (Kohno T, et al. Nat Med. 2012; 18: 375-7, Takeuchi K, et al. Nat Med. 2012; 18: 378-81). FGFR1 amplification was examined by performing fluorescence in situ hybridization (FISH) analysis on 69 NSCLC samples that were available for analysis. This procedure was performed on formalin-fixed, paraffin-embedded, 4-μm-thick tumor tissues that had been sampled in duplicate from representative regions in each tumor. The present inventors used FISH probes for FGFR1 (RP11-513DS and RP11-100B16, which were labeled by Spectrum Orange, Chromosome Science Labo, Inc; Sapporo, Japan) and FISH probes for chromosome 8 centromere (CEN8) (RP11-837P5 and RP11-11P6, which were labeled by Spectrum Green, Chromosome Science Labo, Inc). FISH analysis and signal capture were performed through a fluorescence microscope (AXIO, manufactured by Zeiss) coupled to ISIS FISH Imaging System (manufactured by Metasystems). At least 60 inter-phase nuclei from each tumor were scored, and FGFR1 amplification was defined if one of the following criteria was fulfilled: (I) FGFR1/CEN8 ratio was 2.0, (II) average gene copy per nucleus was ≧6.0, and (III) percentage of tumor cells containing ≧15 gene copies or large cluster was ≧10% (Schildhaus H U, et al. Mod Pathol. 2012; 25: 1473-80).

(4) Cell Lines

A427, A549, NCI-H157, NCI-H460, NCI-H520, NCI-H522, NCI-H661, NCI-H1299, NCI-H1703, NCI-H1819, LK2, HCC515, II-18 (NSCLC), U2OS (osteosarcoma), HT1080 (fibrosarcoma), HeLa (cervical cancer), WiDr, HCT116 (colorectal cancer), HFL-1, and MRC-5 (fibroblast) cells were cultured in RPMI-1640 (Gibco) supplemented with 10% fetal bovine serum (FBS). Then, 293FT cells were cultured in DMEM supplemented with 10% FBS. A549, NCI-H1819, NCI-H661, NCI-H157, NCI-H1299, A427, NCI-H522, and NCI-H1703 were lung cancer cell lines harboring truncating BRG1 mutations, whereas NCI-H520, HeLa, LK2, NCI-H460, U2OS, and HT1080 carried wild-type BRG1 (Medina P P, et al. Hum Mutat. 2008; 29: 617-22, Blanco R, et al. Hum Mutat. 2009; 30: 1199-206). Alterations in BRG1 and other cancer-related genes were verified in the cell lines the present inventors examined. Some of the results were previously reported (Blanco R, et al. Hum Mutat. 2009; 30: 1199-206).

Mutations in BRG1 and BRM coding regions in HCC515 and II-18 cells, which did not express BRG1, and 16 primary tumors negative for BRG staining were examined by targeted genome capture and massively parallel sequencing of genomic DNA, as described above. Mutations detected were verified by Sanger sequencing of genomic PCR products. The effect of single-nucleotide variants resulting in amino-acid changes in the BRG1 ATPase domain, expressed in HCT116 (L1163P) and WiDr (D1284N) colorectal cancer cells (Medina P P, et al. Genes Chromosomes Cancer. 2004; 41: 170-7), was estimated using the Polyphen and Polyphen2 programs. The substitutions in HCT116 cells are thought to abrogate ATPase activity, but those in WiDr cells are benign.

(5) Short Interfering (si)RNA

ON-TARGET plus SMARTpool siRNA (manufactured by Dharmacon) was used in this study. To knock down the BRM protein, cells were transfected with siBRM (Cat No: L-017253-00: SMARCA2) using Lipofectamine RNAiMAX (manufactured by Invitrogen). Non-targeting siRNA (L-001810-10) was used as a negative control.

(6) Establishment of Cells Expressing Doxycycline-Inducible Short Hairpin (sh)BRM

To achieve doxycycline (dox)-inducible knockdown of BRM, a lentiviral vector-based shRNA-mediated conditional gene-expression system (Wiznerowicz M, Trono D. J Virol. 2003; 77: 8957-61) was used. In this system, tTRKRAB (a fusion of the tetracycline repressor [tTR] and the Krueppel-associated box [KRAB] proteins) binds to the Tet operator (TetO), which is juxtaposed with the H1 promoter, to create a local heterochromatic state extending over 2 to 3 kb. This leads to suppressed shRNA expression downstream of the H1 promoter. In the presence of doxycycline (dox), tTRKRAB does not bind to TetO, allowing expression of the shRNA, which then down-regulates expression of the target genes. Thus, both up-regulation and down-regulation of the target gene can be reversibly controlled by dox.

shBRM oligos were annealed and cloned into the Mlu1-Cla1 site downstream of the H1 promoter juxtaposed with TetO in the pLV-THM retrovirus plasmid (Addgene) to yield pLV-THM-shBRM. The sequences for the shBRM oligos are: “5′-cgcgtccccGGAGGTGCTAAGACACTTATGttcaagaCATAAGTGTCTTA GCACCTCCtttttggaaat-3′ (SEQ ID NO: 7)” and “5′-cgatttccaaaaaGGAGGTGCTAAGACACTTATGtctcttgaaCATAAGTGT CTTAGCACCTCCGGGGa-3′” (SEQ ID NO: 8) (Yamamichi N, et al. Cancer Res. 2007; 67: 10727-35). pLV-tTRKRAB, a plasmid vector carrying tTRKRAB, was purchased from Addgene. 293FT cells were co-transfected with pLV-THM-shBRM, pLV-THsi/Control carrying a shRNA targeting green fluorescent protein (GFP) (Bruno T, et al. Cancer Cell. 2010; 18: 122-34), or pLV-tTR-KRAB, together with the packaging plasmids psPAX2 and pMD2.G (Addgene), using Lipofectamine 2000 (Invitrogen), to produce lentiviruses for transduction of LV-THM-shBRM, LV-TH si/Control, or LV-tTRKRAB DNAs, respectively. NCI-H1299 cells were infected with LV-tTRKRAB, and a single clone was isolated. This clone exhibited high tTRKRAB protein expression, as revealed by immunoblot analysis using an antibody specific for tTR (manufactured by MobiTec, TET01) This NCI-H1299 clone was then subjected to retroviral infection with either LV-THM-shBRM or LV-TH si/Control (to yield NCI-H1299-shBRM or NCI-H1299-shControl cells, respectively), and then mass-cultured. HeLa-shBRM and HeLa-shControl cells were also established in the same manner. For the in vitro knockdown experiments, cells were cultured in growth medium containing 0.1 μg/ml dox. The medium was changed every 3 days.

(7) Immunoblot Analysis

Immunoblot analysis was performed as previously described (Ogiwara H, et al. Oncogene. 2011; 30: 2135-46), using antibodies specific for the following proteins: BRG1/SMARCA4 (manufactured by Epitomics; 2776-1), BRM/SMARCA2 (manufactured by Abcam; ab15597), SNF5/SMARCB1 (manufactured by Epitomics; 5542-1), ARID1A/SMARCF1 (manufactured by Bethyl; A301-041A-1), ARID2 (manufactured by Bethyl; A302-230A-1), BAF180 (manufactured by Bethyl; A301-591A), SNF2H/SMARCA5 (manufactured by Abcam; ab3749-100), p21/CDKN1A (manufactured by Cell Signaling Technologies; 29475S), α-tubulin (manufactured by Calbiochem; CP06), and β-actin (manufactured by Cell Signaling Technologies; 3700P). The expression level of BRM protein was quantified using the MultiGauge software (manufactured by FUJIFILM Co., Ltd.).

(8) Cell Survival Assay

The effect of BRM knockdown on the survival of BRG1-deficient (undetectable by immunoblot analysis) and BRG1-proficient (detectable by immunoblot analysis) cancer cells was evaluated using clonogenic survival assays. For this purpose, cultured cancer cells were trypsinized, counted, seeded in specified numbers in 6-well culture dishes, and cultured for a further 10 days to allow colony formation. The cells were then fixed for 5 minutes in a solution containing 50% (v/v) methanol/0.01% (w/v) crystal violet. Colonies containing at least 50 cells were counted.

Since non-cancerous fibroblasts do not form colonies on culture plates, the viability of these cells was determined by examining cellular ATP levels using the CellTiter-Glo Luminescent Cell Viability Assay kit (manufactured by Promega). For this purpose, cultured fibroblasts were trypsinized, counted, seeded in 96-well plates (2000 cells/well), and cultured for an additional 8 hours to allow the cells to attach. This assay was performed every 2 days (up to Day 6).

(9) Complementation Assay

The complementary role of BRG1 was examined in BRM knockdown cancer cells by transfecting cDNAs encoding wild-type and mutant BRG1 (BRG1K^{785A}, this mutation disrupts the ATP-binding pocket in BRG1; Zhang J, et al. Mol Cell Biol. 2006; 26: 7942-52) into NCI-H1299 cells. The human BRG1 cDNA clone was obtained from the Biological Resource Center (NBRC) (National Institute of Technology and Evaluation: NITE) (Kisarazu, Japan). Complementary DNAs encoding FLAG-tagged wild-type BRG1 and BRG1^{K785A} were subcloned into the pcDNA3.1 expression vector (manufactured by Invitrogen). The BRG1^{K785A} mutation was introduced by changing bases 2353 to 2355 of the human BRG1 cDNA to GCC by site-directed mutagenesis. Control siRNAs (D-001810-02-05) and siRNAs targeting BRM (LQ-017253-00-0002) were obtained from Thermo Scientific.

siRNAs (5 nM) were transfected into NCI-H1299 cells using Lipofectamine RNAiMAX (manufactured by Invitrogen). Twenty-four hours after the transfection, the cells were re-transfected with 2.5 μg of plasmid DNA using Lipofectamine LTX (manufactured by Invitrogen). Another 24 hours later (defined as Day 0), the cells were seeded into a 96-well plate (1000 cells/100 μl/well). On Day 2, the cells were re-transfected with 5 nM of siRNAs. To measure cell viability, ATPlite 1 step (manufactured by Perkin Elmer) was added to the cells, and the fluorescence was measured in an Wallac1420 ARVO SX multilabel counter (manufactured by Perkin Elmer). An anti-BRG1 monoclonal antibody (E1), an anti-β-actin monoclonal antibody (AC-15) (manufactured by Santa Cruz Biotechnology), and an anti-BRM polyclonal antibody (manufactured by Cell Signaling Technology) were used for immunoblot analysis.

(10) Cell-Cycle Analysis

Cells were trypsinized, centrifuged, washed in PBS, and fixed in ice-cold 70% ethanol. The cells were then centrifuged again, incubated with PBS containing 200 μg/ml RNase A and 5 μg/ml propidium iodide, and analyzed for cell-cycle distribution by Guava flow cytometry (manufactured by Millipore).

(11) Senescence-Associated β-Galactosidase (SA-β-Gal) Staining

SA-β-gal staining was performed using the Senescence β-galactosidase Staining Kit (Cell Signaling Technologies) The percentage of SA-β-gal-positive cells was determined from three different fields per sample.

(12) Fluorescence Microscopy

Cells were grown on glass coverslips, fixed with 2% (w/v) paraformaldehyde in PBS for 15 minutes at room temperature, and permeabilized with 0.3% (v/v) Triton X-100 for 15 minutes at room temperature. After the fixation, the cells were washed three times with PBS and stained with DAPI (0.4 μg/ml diluted in PBS) to visualize the DNA. The coverslips were mounted on glass slides using Prolong Gold mounting agent (manufactured by Invitrogen). Confocal images were taken using an inverted microscope (manufactured by Carl Zeiss) equipped with a 100× oil lens. The images were acquired using the ZEN Software (2008) (manufactured by Carl Zeiss). The percentage of cells containing senescence-associated heterochromatic foci (SAHF) or undergoing mitotic catastrophe was quantified in three different fields per sample. SAHF were judged as previously described (Di Micco R, et al. Nat Cell Biol. 2011; 13: 292-302). Cells having two or more distinct nuclear lobes were scored as positive for mitotic catastrophe (Russo A L, et al. Clin Cancer Res. 2009; 15: 607-12).

(13) Long-Term In Vitro Cell-Proliferation Assay

NCI-H1299-shBRM cells and HeLa-shBRM cells were seeded in 60-mm culture dishes (1×10⁵ cells/dish) in media with or without 0.1 μg/ml dox, and then cultured. The cells were trypsinized and counted every three days, and after each count, 1×10⁵ cells were re-seeded in new dishes with fresh medium. Accumulated cell numbers were calculated at each passage, based on the total number of cells and dilution ratio at the time of passage. The experiment was performed in duplicate.

(14) Mouse Xenograft Model

The effect of BRM ablation on the growth of NCI-H1299-shBRM tumor cell xenograft and NCI-H1299-shControl tumor cell xenograft was examined in BALB/c-nu/nu mice. NCI-H1299-shBRM cells and H1299-shControl cells (4×10⁶ cells/mouse in 50% Matrigel; manufactured by BD Biosciences) were injected subcutaneously into 40-day-old female BALB/c-nu/nu mice (manufactured by CLEA Japan, Inc.). After 3 weeks, when the tumors reached 100 mm³, the mice were randomly divided into two groups and fed either a diet containing dox (200 ppm) or a control diet. Tumor growth was measured twice per week using calipers. Tumor volume (TV) was calculated as previously described (Bruno T, et al. Cancer Cell. 2010; 18: 122-34) using the formula TV=a×(b²)/2 (in the formula, a and b are the tumor length and width, respectively). At the end of the experiment, the mice were sacrificed in accordance with standard protocols. All the experiments were approved by the Ethical Committee on Animal Experiments at the National Cancer Center.

(15) Statistical Analysis

Logistic regression analysis was performed using JMP (ver 5.1). Fisher's exact test and Student's t test were performed using Statmate III (ver 3.17). P<0.05 was considered statistically significant. Experiments were performed in triplicate unless otherwise stated.

2. Results

(1) Characteristics of BRG1-Deficient NSCLCs

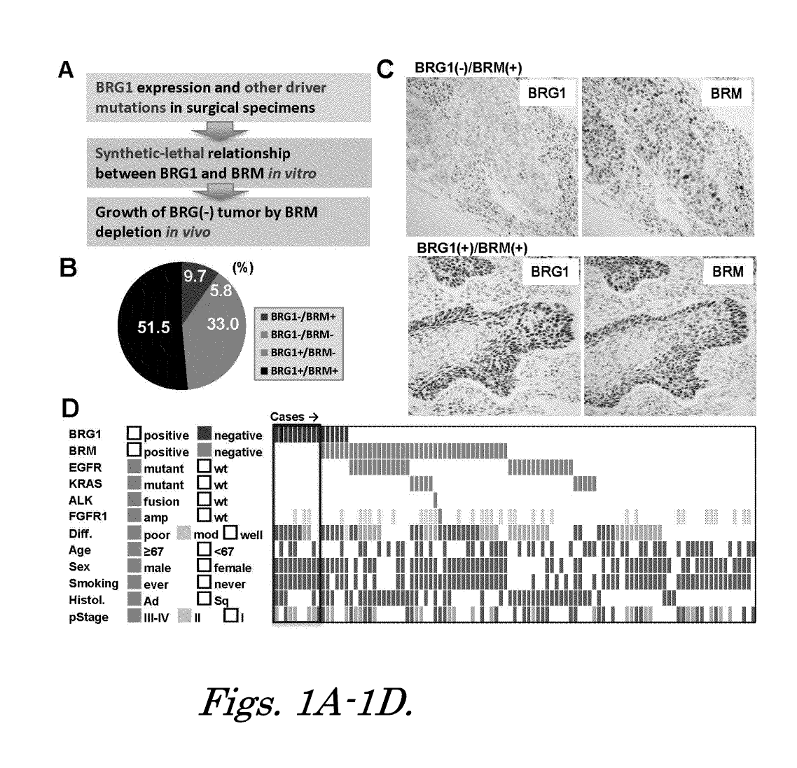

The present inventors first of all examined the characteristics of BRG1-deficient NSCLCs. Since BRG1 has been reported to play a role in cell differentiation (Romero O A, et al. EMBO Mol Med. 2012; 4: 603-16), the examined cases were selected to include similar numbers of adenocarcinomas and squamous-cell carcinomas of the three different differentiation grades (well differentiated; moderately differentiated, and poorly differentiated) (Table 1). This approach allowed an efficient analysis of the association between BRG1 expression and tumor differentiation. Immunohistochemical analysis revealed that BRG1 protein expression was low or absent in 16/103 (15.5%) NSCLCs (FIG. 1B, C). Such low or absent BRG1 expression was more prevalent in poorly differentiated tumors (Table 1 and FIG. 1D), and was also prevalent in males and smokers. Mutational analysis of BRG1 and BRM in 16 cases negative for BRG1 staining revealed that one case had nonsense BRG1 mutations, whereas the remaining 15 cases did not. This suggests that both genetic alterations and epigenetic silencing of the BRG1 gene can cause BRG1 deficiency, as previously reported (Rodriguez-Nieto S, et al. Hum Mutat. 2011; 32: E1999-2017). On the other hand, deleterious BRM mutations were not detected in these 16 cases including five that were negative for BRM staining.

(2) Driver Gene Mutations in BRG1-Deficient NSCLCs

Next, the present inventors analyzed representative therapeutically relevant gene mutations (EGFR and KRAS gene mutations and ALK fusions) in the 103 NSCLC cases. The 16 BRG1-deficient cases were negative for mutations in all three driver genes, including two therapeutic targets (EGFR mutation and ALK fusion) (FIG. 1D, Table 1). The present inventors also analyzed other driver gene mutations, RET and ROS1 fusions (Kohno T, et al. Nat Med. 2012; 18: 375-7, Takeuchi K, et al. Nat Med. 2012; 18: 378-81, Lipson D, et al. Nat Med. 2012; 18: 382-4), in the nine BRG1-deficient cases for which tumor tissue RNA was available, but no fusions were detected. Recent whole-genome analyses have also suggested that BRG1 mutations are prevalent in tumors without EGFR mutations or ALK fusions (Seo J S, et al. Genome Res. 2012; 22: 2109-19, Imielinski M, et al. Cell. 2012; 150: 1107-20). In addition, all the BRG1-deficient cases were also negative for two other alterations, FGFR1 amplifications and DDR2 mutations (Hammerman P S, et al. Cancer Discov. 2011; 1: 78-89, Weiss J, et al. Sci Transl Med. 2010; 2: 62ra93) (FIG. 1D, Table 1). These findings suggest that most BRG1-deficient NSCLCs are not suitable for current molecularly targeted therapies based on tyrosine kinase inhibitors (Lovly C M, Carbone D P. Nat Rev Clin Oncol. 2011; 8: 68-70). Of the 16 BRG1-deficient cases, 10 (62.5%, 9.7% of all cases studied) expressed BRM.

(3) BRM-Dependent Growth of BRG1-Deficient Cancer Cells

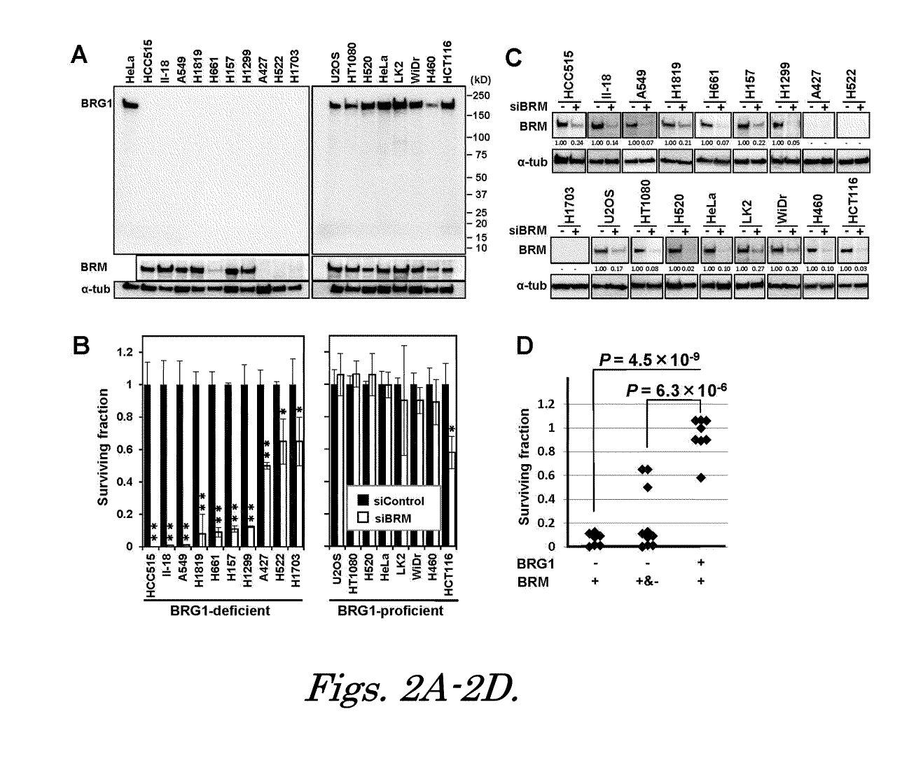

The present inventors then compared the effect of siRNA-mediated BRM knockdown on cell growth of BRG1-deficient (n=10) and BRG1-proficient (n=8) cancer cell lines, using a clonogenic survival assay (Clonogenic SURVIVAL assay) (FIG. 2A). Eight out of ten BRG1-deficient cell lines used in the study carry truncating BRG1 mutations/deletions (Medina P P, et al. Hum Mutat. 2008; 29: 617-22, Blanco R, et al. Hum Mutat. 2009; 30: 1199-206). Of the remaining two BRG1-deficient cell lines, the present inventors detected a homozygous nonsense mutation in one (HCC515) by targeted genome capture sequencing, but no BRG1 mutation in the other (II-18). On the other hand, BRG1-proficient cancer cells did not harbor such deleterious BRG1 mutations (except HCT116 cells, which harbor a heterozygous missense mutation that is likely to disrupt the BRG1 ATPase domain; Wong A K, et al. Cancer Res. 2000; 60: 6171-7).

The present inventors observed suppression of colony formation by BRM knockdown in all BRG1-deficient cells tested, but not in BRG1-proficient cell lines other than HCT116 (FIG. 2B). The cell surviving fractions of cancer cell lines are summarized according to BRG1 and BRM expression status in FIG. 2D. Survival of BRG1-deficient cells upon BRM knockdown was significantly lower than that of BRG1-proficient cell lines (P=6.3×10⁶ by Student's t test). The BRM protein was undetectable, or detectable only at trace levels, in three BRG1-deficient cell lines, A427, NCI-H522, and NCI-H1703 (FIG. 2A). These findings are consistent with a report showing that a fraction of lung-cancer cells lack, or express at low levels, both BRG1 and BRM (Reisman D N, et al. Cancer Res. 2003; 63: 560-6) (FIG. 1B). The effect of siRNA-mediated BRM knockdown on the cell growth and survival in these three cell lines was less evident than in the other seven cell lines. Thus, among BRG1-deficient cell lines, the suppression of colony formation by BRM knockdown was more prominent in cell lines that originally expressed the BRM protein (P=4.5×10⁻⁹ by Student's t test) (FIG. 2D).

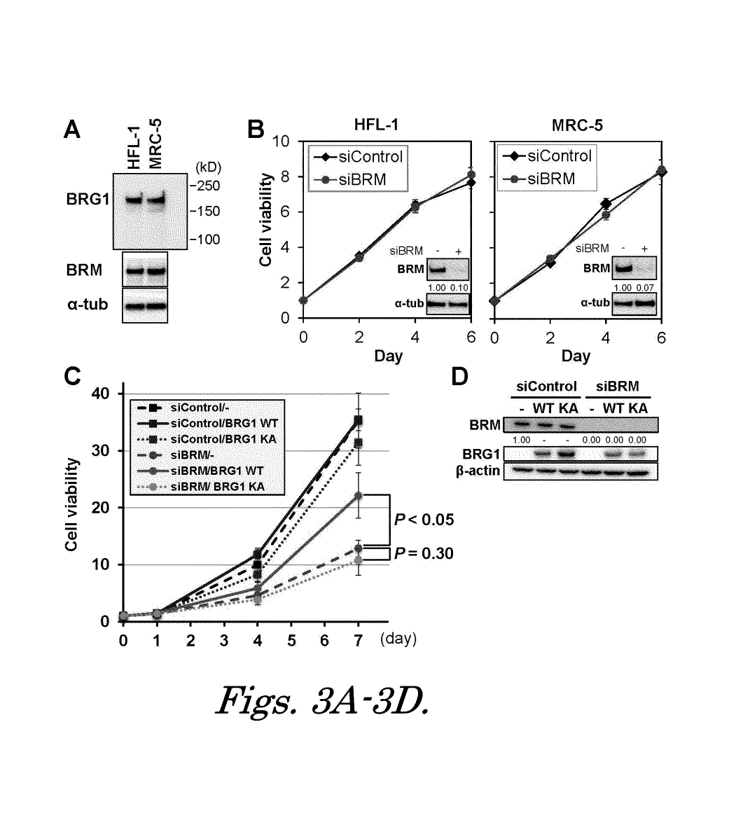

BRM knockdown did not affect the growth of the non-cancerous fibroblast cell lines HFL-1 and MRC-5, which expressed both BRG1 and BRM (FIG. 3A, B). Wild-type BRG1 cDNA complemented the sensitivity of NCI-H1299 cells to BRM knockdown-mediated growth inhibition (FIG. 3C, D). On the other hand, cDNA of an ATPase mutant with a disrupted ATP-binding pocket (BRG1^{K785A}) (Zhang J, et al. Mol Cell Biol. 2006; 26: 7942-52) did not complement the sensitivity (BRG1^{K785A} is indicated as “KA” in FIG. 3C, D). These results indicated that BRG1-deficient cancer cells depend on BRM for growth, and that this phenotype is caused by a lack of BRG1 ATPase activity.

(4) BRM Depletion Induces Senescence in BRG1-Deficient Cancer Cells

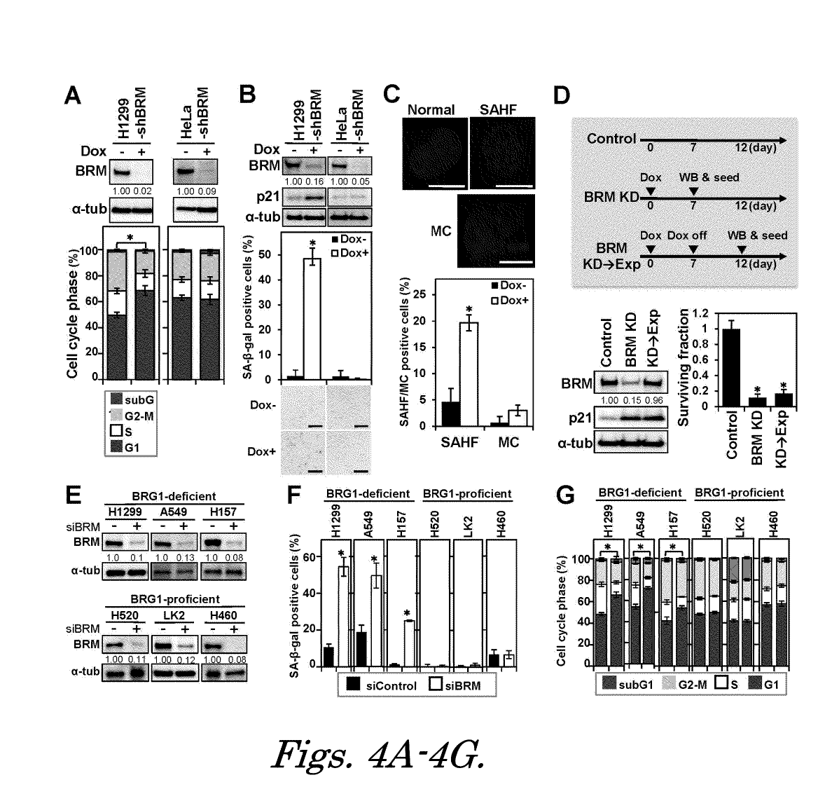

To examine the long-term effects of BRM depletion, the present inventors prepared NCI-H1299 (BRG1-deficient) cells and HeLa (BRG1-proficient) cells expressing either a BRM-targeting shRNA inducible by adding doxycycline (NCI-H1299-shBRM and HeLa-shBRM) (Wiznerowicz M, et al. J Virol. 2003; 77: 8957-61, Bruno T, et al. Cancer Cell. 2010; 18: 122-34) or a non-targeting shRNA (NCI-H1299-shControl and HeLa-shControl). To confirm the effects of BRM knockdown, the present inventors used a different BRM target site from the one used in the siRNA experiments. The results of the clonogenic survival assay were confirmed using this system. Next, examined was the mechanism underlying growth inhibition in NCI-H1299-shBRM cells. Seven days after dox treatment, NCI-H1299-shBRM cells, but not HeLa-shBRM cells, exhibited G1 arrest with a flattened morphology, suggesting senescence (FIG. 4A). Biomarkers of senescence (Roninson I B. Cancer Res. 2003; 63: 2705-15, Zhang R, Adams P D. Cell Cycle. 2007; 6: 784-9) (p21/CDKN1A expression, SA-β-gal staining, and SAHF) were observed in dox-treated NCI-H1299-shBRM cells, but not in HeLa-shBRM cells (FIG. 4B, C). Dox treatment did not affect the proportion of cells in sub-G1-phase or the number of cells exhibiting distinct abnormal nuclear lobation. This suggests that ablation of BRM does not induce apoptosis or mitotic catastrophe (Al-Ejeh F, et al. Oncogene. 2010; 29: 6085-98) (FIG. 4A, C). Senescence is an irreversible state of growth arrest that is maintained by p21 (Ohtani N, et al. Nature. 2001; 409: 1067-70); therefore, the present inventors examined p21 expression in NCI-H1299-shBRM cells in which BRM was re-expressed after dox removal (FIG. 4D). In these cells, p21 expression was maintained at the level observed in BRM knockdown cells. Consistent with this, cells re-expressing BRM exhibited suppressed clonogenic growth activity. Meanwhile, the BRM knockdown caused significant increases in SA-β-gal positivity and the number of cells in G1-phase in three BRG1-deficient NSCLC cell lines (NCI-H1299, A549, and NCI-H157), but not in three BRG1-proficient NSCLC cell lines (NCI-H520, LK2, and NCI-H460) (FIG. 4E, supplementary FIG. S7). These results suggest that BRM knockdown specifically suppresses the growth of BRG1-deficient cells by inducing senescence.

(5) BRM-Dependent Growth of BRG1-Deficient Cancer Cells In Vivo

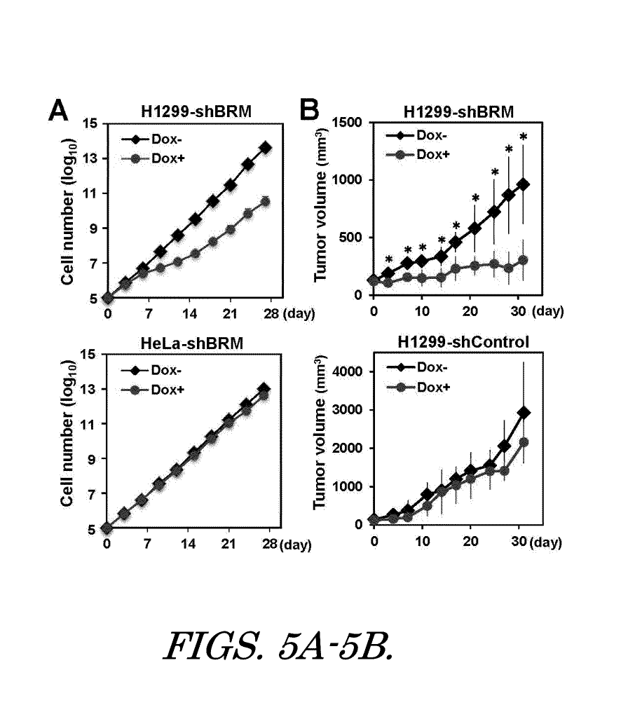

The number of BRM-knockdown NCI-H1299-shBRM cells was approximately 1,000 times as small as that of non-depleted cells after 4 weeks of culture, whereas the number of HeLa-shBRM cells did not change (FIG. 5A). This observation suggests that inhibition of BRM specifically suppresses the long-term growth of BRG1-deficient cells (i.e., over at least one month). To further confirm these data under more physiologically relevant conditions, the present inventors introduced NCI-H1299-shBRM cells and NCI-H1299-shControl cells into an in vivo conditional RNAi model (FIG. 5B). BALB/c-nu/nu mice were injected subcutaneously with NCI-H1299-shBRM cells or NCI-H1299-shControl cells. After 3 weeks, when the injected cells had formed tumor nodules measuring approximately 100 mm³, the animals were randomly divided into two groups and fed a diet containing dox (200 ppm) or a control diet for 1 month. In dox-fed mice, the NCI-H1299-shBRM xenografts were significantly smaller than the NCI-H1299-shControl xenografts.

### Example 2

### Development of Screening Method for BRM Inhibitor

1. Preparation of BRM/BRG1 Protein Complex Baculovirus

Recombinant baculovirus were prepared using HighFive cells with a flashBAC ULTRA system, and the human BRM gene (Kazusa Clone pFN21AE0797), the human BRG1 gene (NBRC clone BRHIP3033650), the human BAF170 gene (Kazusa Clone FXC01767), the human BAF155 gene (Kazusa Clone FHC11034), and the human INI1 gene (Kazusa Clone FXC03158).

2. Purification of BRM/BRG1 Protein Complex

HighFive cells were infected with the baculoviruses and cultured at 21° C. The culture liquid was centrifuged at 3000 rpm for 15 minutes at 4° C., and the cells were recovered. Then, washing was performed twice with a phosphate buffer containing Complete EDTA free protease inhibitor (manufactured by Roche). After the washing, the cells were suspended in a dissolution buffer (20 mM HEPES, pH 7.5, 200 mM NaCl, 1 mM DTT, 10% glycerol, Complete EDTA free protease inhibitor), and subjected to a ultrasonic disruption treatment (SoniFier (manufactured by BRANSON): ½ inch tip, power 5, on/off: 15 seconds/30 seconds, four times on ice). After centrifugation at 35000 rpm for 60 minutes at 4° C., the supernatant was recovered as a cell lysate. To confirm the expression of each protein complex, SDS-PAGE was performed using the cell lysate.

Utilizing the His-tag and FLAG-tag added to the recombinant protein, a protein complex was purified by the following method. To the cell lysate, Ni-NTA agarose (manufactured by Invitrogen) was added and allowed to react at 4° C. overnight. The carrier was washed with a wash buffer (25 mM Tris-HCl, pH 8.0, 600 mM NaCl, 0.05% Tween-20, 10% glycerol), and the protein was eluted using an elution buffer 1 (25 mM Tris-HCl, pH 8.0, 138 mM NaCl, 0.05% Tween-20, 10% glycerol, 500 mM imidazole). Subsequently, ANTI-FLAG M2 Agarose Affinity Gel (manufactured by Sigma-Aldrich) was added to the His-tag purified sample, and allowed to react at 4° C. for 6 hours. Thereafter, the carrier was washed with a wash buffer, and the carrier was then suspended in an elution buffer 2 (25 mM Tris-HCl, pH 8.0, 138 mM NaCl, 0.05% Tween-20, 10% glycerol, 300 μg/mL 3×FLAG peptide) and allowed to react at 4° C. overnight. The reacted sample was centrifuged, and the supernatant was recovered as a BRM/BRG1 protein complex. To confirm the recovery of the purified product of each protein complex, SDS-PAGE was performed.

3. ATPase Assay

Twenty five nM (calculated through the concentration analysis by SDS-PAGE) of each BRM/BRG1 protein complex purified by the above-described method was incubated in an ATPase reaction solution (final concentration: 32.5 mM Tris-HCl, pH 7.5, 118 mM NaCl, 5 mM MgCl₂, 60 μg/mL BSA, 8% glycerol, 0.025% Tween-20, 56 nM pcDNA3.1, 10 nM ATP) for 3 hours at room temperature. The reaction volume was set to 20 μL. The ATP consumed in this reaction was detected using ADP-glo (manufactured by Promega) and measured with 2104 Envision Multilabel Counter (manufactured by PerkinElmer).

### INDUSTRIAL APPLICABILITY

The present invention provides a therapeutic strategy for specifically targeting cancer cells harboring inactivating SWI/SNF mutations. The present invention has revealed that BRG1 and BRM have a synthetic-lethal relationship, and that a treatment for inhibiting BRM is a promising approach for treating a BRG1-deficient cancer having no mutation in known therapeutic target genes. Moreover, it has also been revealed that, in this therapeutic strategy, a BRM inhibitor may be administered to a cancer patient after the selection based on BRG1 function suppression, hence enabling an efficient treatment based on the companion diagnosis. Thus, the present invention can greatly contribute to the medical field, particularly, in the field of cancer treatment.

### [Sequence Listing Free Text]

### SEQ ID NOs: 7 and 8

<223> shBRM oligo sequences

1. A method for evaluating a cancer, the method comprising detecting whether or not a BRG1 function is suppressed in a biological sample derived from a cancer patient.

2. A method for predicting responsiveness to a cancer treatment with a compound capable of inhibiting BRM, the method comprising:
detecting whether or not a BRG1 function is suppressed in a biological sample derived from a cancer patient; and
determining that the patient, from which the BRG1 function suppression is detected, responds to the cancer treatment with the compound capable of inhibiting BRM.

3. A method for selecting a target of a cancer treatment with a compound capable of inhibiting BRM:
detecting whether or not a BRG1 function is suppressed in a biological sample derived from a cancer patient; and
selecting the patient, from which the BRG1 function suppression is detected, as the target of the cancer treatment with the compound capable of inhibiting BRM.

4. A method for treating a cancer having a suppressed BRG1 function, the method comprising administering a compound capable of inhibiting BRM to a patient from which BRG1 function suppression is detected.

5. A method for treating a cancer having a suppressed BRG1 function, the method comprising:
detecting whether or not a BRG1 function is suppressed in a biological sample derived from a cancer patient; and
administering a compound capable of inhibiting BRM to the patient from which the BRG1 function suppression is detected.

6. A reagent for detecting whether or not a BRG1 function is suppressed, the reagent comprising as an active ingredient any one of the following molecules (a) to (c):
(a) an oligonucleotide primer capable of specifically binding to a BRG1 gene;
(b) an oligonucleotide probe capable of specifically binding to a BRG1 gene; and
(c) an antibody capable of specifically binding to a BRG1 protein.

7. A screening method for a compound used in a treatment against a cancer having a suppressed BRG1 function, the method comprising the step of selecting the compound based on whether or not BRM is inhibited.


SEQUENCE LISTING (8 sequences)

SEQ ID NO: 1 - DNA, 77665 bp, Homo sapiens
  CDS (1)..(222)
atgtccactccagacccacccctgggcggaactcctcggccaggtccttccccgggccctggcccttcccctggagccatgctgggccctagcccgggtccctcgccgggctccgcccacagcatgatggggcccagcccagggccgccctcagcaggacaccccatccccacccaggggcctggagggtaccctcaggacaacatgcaccagatgcacaaggtagggatccctgtgcccgcctcgcacctgcggcctctgcccactagggctgcaggcagcctctggaccgagggccttacttggaggatggggggaagccttcttgttggaggtgtcctgccttggctcagccccctaccccagggcccacggccatgaacagaaggttcagctcgtcagaccccagcctgtgctggcgcatgatctgggccccgcgggcacctgccccaccgtttcccgctcccttgctttctgcatgtgaaatttgggaatatcactacaaagttttcttttggtaatgaagaaaaacatccaaattcctaaaaaggcttttagccacgtggtttcccccgttctggctgtcaggctcacctgtgcagctcgcagagccgagcagcgggttccctttcctccaaggcgtgcccctcagccactgtctttacctcacctgcgctgagcagggccttcagtcagcaaggtggagaaggctctttatggtcacaagtctcttttgcatgtccctggatgtggttagaggacatattcccttatacatacagcatctgaaaggaaaggtcatattagcatccatgagaaaaggtgacatttctaagtgtcacgggccacaggaggtcccgggtggtagaagatgaggattgcttgaccacagtctcccatatgctcgtgctccactgggcgacatttattttgtgtacctgtctcgaggcctaagtaggtgtcagaaccttgccttggagtcatgctggggactggggagatgcgcgtcatcttcgggtggctgttctcggtgccctcgagcttctctcgggcagcgcatagctgcgctgccacctcacgttccacatgctgaccctgccttgccatggtccctctcgcagcccatggagtccatgcatgagaagggcatgtcggacgacccgcgctacaaccagatgaaaggaatggggatgcggtcagggggccatgctgggatggggcccccgcccagccccatggaccagcactcccaaggtacagaactgcgttccttcctgccttgtgtttgtcatactccagagtcctcagatcattttcctcccctgggttcccacaggatggattcagggagtacctaggatgatgtagccgggtgggtggcccgccacagagagctgtctggcatggcgtggctggtgctctgttctggccacctcgtctcggagcagccctggaaggggaaatgctggttggggggcagaccatgttcagcatgggtgatagaggagggctgtgcagggcagcagccccgtgccacccaggaacttactggaaatacagattcctggggcctgggactctgctttaatgagcccagcaggtgccttttctgtacgaggacaattgagaagtgatatggtgggtgactttccaaggccttgctgtcatcaggggtgggaagctcaggggctgcactgggaatggaagtgttgggggtgggcctggcgagcccgaggatgtggaccccgcctgtggacaggcaggcggaagccgggagagagccgtgatttcatgcacaggttctcagcattaaccagaagcaccttactgagacgtgggccaaaccaaatccaccaggtcggctgctgggctgaggtgccttcagcatgtgccctccaggtgcccctggttgactcaagagaaggatgccatttggctgtgccctgtctcagagtaggagctggtgtagggggaagaggctgtaaaaatcacagacatatgctgccgagtgaccagtgggctgacctttctctgcaggttacccctcgcccctgggtggctctgagcatgcctctagtccagttccagccagtggcccgtcttcggggccccagatgtcttccgggccaggaggtgccccgctggatggtgctgacccccaggccttggggcagcagaaccggggcccaaccccatttaaccagaaccagctgcaccagctcagagctcagatcatggcctacaagatgctggccagggggcagcccctccccgaccacctgcagatggcggtgcagggcaagcggccgatgcccgggatgcagcagcagatgccaacgctacctccaccctcggtgtccgcaacaggacccggccctggccctggccctggccccggcccgggtcccggcccggcacctccaaattacagcaggcctcatggtaagactggctgccctggccctcaggtgtctcagagcgaatggctggggcgtgggtggcggggtggacagaccgtgctctgttgccgactgggttccccggtttgggattgcacgggcccatactgcacttctgggtgctcgggtggtgggggcagctaaatgctgcttccccagctccccgcttcccctggggccgctggttaataggtgtatctgctgtgtcccctggagccagggctgcccacggggctgggcgcaggcataaacctgggacgcactgttttctcttttgtttctccctacatgtaggtatgggagggcccaacatgcctcccccaggaccctcgggcgtgccccccgggatgccaggccagcctcctggagggcctcccaagccctggcctgaaggtgagctccctcttctatggtggtgcacccgtgcccttactccccatctcaagcttgggtccttgagatgagctttgtcagggagaaagggccgagggtggtcaggctgaactgcagcctgtacttttcttgtggtggtccccgggtctcccctgtagccagtcagttcccaaaggctgtcgttcatccctcctctgacagcttgtggccttcacccagtccctgagccctgtatatgtaattgctcagtaagtgctgcaggctcccatctgttgggctcaaagaggcagtgggactcattacccatcctcttggagcctgaggtttagcgatgggcattgaggggcaaattgcttccactggatttagttggcactaggaggagatgacaggaaatgctgccatagagccacagacagaacaaaaaggccttgggagcctggggctggccccagtggagggtgtgaaggacgagggtgaggctgagatctggaggaggttggagcccaaggcaggtgtgaaggacacccctgggtgaaaggtgggagatgggcggggtctgcctgtccccagtgcctcaagcagctcagcagctttccatttccagcccgggatgggccccagagctcaacatgacgccctggccccttgccttctcccaggacccatggcgaatgctgctgcccccacgagcacccctcagaagctgattcccccgcagccaacgggccgcccttcccccgcgccccctgccgtcccacccgccgcctcgcccgtgatgccaccgcagacccagtcccccgggcagccggcccagcccgcgcccatggtgccactgcaccagaagcagagccgcatcacccccatccagaagccgcggggcctcgaccctgtggagatcctgcaggagcgcgagtacaggtgagggcggggcccagttgccaaggtcactgccctgtgtcccccatgtccccctggggaagccactcaactttctcccccactctagcaaacagtgccctgtgcccaccactgccacttggcctacacctccttcctcacctccctgctcccaccctggctgcggtctgtttcccacacagcagtttgagttctctctctttttttttttttgagacggagtttcactcttgttgcccacgctggagtgcaatggcacaatctcggttcactgcaacctgcatctcctggcttcaagcgattcttctgcctcagcctcctgagtagctgggattataggcatgcgccaccacacctggctaattttgtatttattttagtagaggcagggtttctccatgttggtcaggctggtctcgagctcctgacctcagatgctcccaaagtgctgggattacaggcatgagccaccgcacctggcatgaattctctttttatgattgatatcagatctactccgtggattaaaacctgcctgtagcttcaattatctgcccaataagctttgccttatgtccactcctgcccagcatccttcctcacccatcccagtctctctgttcctcttttcatttatttaacaagttagacttgttcctgccccaggacctttgcacaggctgtgcatggctccttctactcattcaggcctgattgctggggttccctcctcatggaggcccttcctgtcacttccagccactctctggaattgcagtggatattttattttttttaaatctcctttatggcctgtgttttctaatgagactgagagttccttgctttgttgttcttgtttagtgctaaggccctgcacttaaaatggtccctggcatacagttggtgctcaatgagtatgtgcagaaagactgattaagagaattagcccacataaccttcgcatgtaaccttcgtaaccttcagaccaagcctctagggtcactccgaggggttgcctcagttttgaggtgagaatgctgaggtgtgcagttgaaatagattcccaccgccctgcgcctcatccatgccagagctgcatgaaaaccttggcccgcctgacgccagaacctggctcacaccactgaaaaggcatgacctgggcgcgtctatcaggaatcttgtttgctgtcccctgaggcccggtcctgaggagtgccccgggctatctccctctctgctgtgccctgcggccagcatcctcttctgaggcatctcttgcctcatgactagtgcctgcctctctcgagggatgggtcccctgcagctccagctgtaactgggtgccctggcaaccctctcaccgcctttgctccttgtctctgctcccaggctgcaggctcgcatcgcacaccgaattcaggaacttgaaaaccttcccgggtccctggccggggatttgcgaaccaaagcgaccattgagctcaaggccctcaggctgctgaacttccagaggcaggtgggtgctggcatggccgcagctttccgaaaagggcctttgtcaccaacactgctgctaaggctccaaatacggcttgcctggctagtattacacctgcctgggctgtgaaaagcagccgtggccatccatgggcactcaatcactgcagagccttcctcttacggcctgcgcagtgctggggacgtggacctcactaccctaaagcacttgccgcctcaaagcagacttgtatgggcttctttcttcctttccctttccctttttttttttttttggaaacagtcttgctctatcacctaggctggagtgcagtggcataaactctgctcactgcaaccaacgcctcctgggatcaagcaattcttctgcttcagcctcccaagtagctgggattacaagcgtgtgccaccatgcctaatttttatatttttagtagagacagggtttcaccatattggccaggctggtctcaaactcttgacctcaaatgatcggcctgccttggcctcccaaagtgctgggattacaggcatgagccaccacgtgctgtcctgcatgggtctatttctgagggtactgtgcttagaccctggagtgcagtggagagatcacagctcactgcaaacttgacctcccaggctcatgtcctcctcccgcttcagcctcctgagtagctgggactataggcgtgcaccattacacctggctatatttttcttttttttgaaacagagtctcactctgtcacccaggctggagtgcagtggtgccatctcaccccactgtggcctctgcctctgtggttcaagtgattttcctgtctcagccactgagtagctgggattacaggtgtgcaccaccatgccgggctgatttttttgaagtgcagtggcacaatcttggctcactgcaacctctgccttccaggttcaagtgattctcccacctcagcctcctgagtagctgggattacaggcatgtgccccacgcctggctaatttttgtatttttagtagagacagtgtttcaccatgttggccaggctggtcttaaactcctgacctcaggtgatcctcctgcctcagcctcccaaagtgctgggattacaggcatgagccaccacgcaggcctggtttttgtatttttaatagacacagggtttcgccatgtcgcccaggctagtctcgaactcctgagctcaagccatccacctgtctcagcctcccaaagtgctgggattacaggcatgagccactgctcctggccgcctagttaatttttcttctagtttttgtagaagtgaggtctctccatgttgcccaggttggtctcaaactcctgggcttgagcaatcctcccaccttggcctcccaaagtgttgggattactggcgttaagccaccacacccagccaagagtgttatacttctgaatgactcctaagacgtgtgtgtggtagaggggaggcaagctggaggtccgagaggaccccctggatagatgtccagaagtgatcaccttctgtcctgtgctcgccctgcccgggggcacctgctagacgtcccctgcacacacggactgggtgtgtaaggcacttacaatgctcctttagcggtgaagcatgtgacatcaccatacgtgtttgtcattgtggatgccacagagctgtgcagtgcgcgggcttgtcctcttccctcctacagctgcgccaggaggtggtggtgtgcatgcggagggacacagcgctggagacagccctcaatgctaaggcctacaagcgcagcaagcgccagtccctgcgcgaggcccgcatcactgagaagctggagaagcagcagaagatcgagcaggagcgcaagcgccggcagaagcaccaggtacgctccggtggccccaaggccctgcagcccgcccacctggctgcctggcttgtccagcggttgccacggggctgtgtttgctttgttcctaaacatgctgcttgtctctctcttttgctcattgtaacagtattctaggtactgagagtgtattggtgtaaaaacccaatggtagggagaagatagtgatgaaatccacgcgtagcacgtctggaaggtgatagggcacaaagaaaaatcaagccaggatagagaggaaggaacgggctgttgtagaactgatgttgggggagttctttcttttggaggtgacatctgggcagacccccaaggaagggaggcacacagatctgtgggaaagagctttctgggaaaaggagaaggtgcaagggccctggggctggaatgtgcctgtgttttctctggaaagcaaggacctgcatgagccagagtagagtgagtggcggggagagctggaggaagggagcgcagggaggtgacgagcaggtgatgcagggccttgcgagctgcatggaggatttgggcttttgctctgagtaaagtgggagccatagagggttctgggcagtgcagggactgatctgactcagggtttcccaagatgaatcagtaagacggaaatcttcaaatctaaaatcaagtaaggatttggggatgattgtgctgatttggtcatttatagattttctagagattttctataattaatatcttattggtgtagtgtaaaataatctagacagtcaacacagcaatctctgttaacttttttttttttttttgagatggagtctcgctgtgtcactgaggctggagtgcagtggtgcaatctcggctcactgcaacctccatctcctgggttcaagcagttttcctgcctcagcctacctagtagctgggattacaggcacctgccaccatgcctggctaactttttttttgtatttttagtagagatggggtttcaccatgttggtcaggctggtttcaaattcctgacctcaagtgatctgtccgtcttggcctctcaaagtgctaagatggcaggcgtaatcccccgtgcctggccaacatttttttttttttttttttttgagatggagtttcactcttgtcacccggactggagtgcaatggcacgatcttggctcactgcaacctctgcctcctgggttcaagggattctcctgcctcagcctcccaagtagttgggattacagatgtgcgccgccacacctgtttgtttttttctttttttgagacagagtttcactcttgttgcccaggccggagtgcagtggtgcgatcttggttcgacacaacctctgcctcctgggttcaagcgattttcctgcctcagcctcccaagtagctgggactacaggcacctgccaccatgcctggctaattttttatatttttagtagagatgaggtttcactatgttggccaggctgctggtctcagactgttgacctcgtgatccgcctgcctcggcttcccaaagtgctgggattacagacgtgagccactgctcctgaccaccaacatttttttatatatattatttcagccttttttcttgtatattaatacaaggttaaatagccctttttaaattttttctaaaactttgctttttttttttttttttttgagtcagaatctcactctgttgcccaggctggagtgcagtggcacgatctccactcactgcagcctctgtcttctgggttcaagcgattctcctgccttagccacctgagcagctggaattacaggtgtgcaccaccacacctggctactttttgtacttttagtagagatggggattcaccctgttggccaggctggtcttgaactcctgacctcaagtgatccgcctgccttggccttccaaagtgctggaattacaggcgtgagccgctgctcctggccacatcgctttgtttttaagtatcatggaaacatgacttttgatgtctacactgtattccatttctatgtgtccaggatctaatcatttatgtcacctttttcctgtttgaggacatgcagcatgtttcagcttttgaaaattaaaaattatgctaggaccggaattcttggcttgaacattttggagctggtctctgatatttccttaaaataaatttgtagtaatggaataagttgggtgaagggttggaagcatttttgagacatttcacatccgtgccatgctgccctgccagtcgactgcactgtgtgcctacctgctgtgtttgagggcctttcccacccacagttgccggtgcttagtttgtttttttttttgagacggagtctcgctctgtcgcccacgctggagtgcagtggcgcgatctcggctcactgcaagctctgcctgctgggttcatgccattcttctgcctcggcctcctgagtagctgggactacaggtgccagccaccacgcccggctaattttttgtatttttagtagagacggggtttcaccatgttagccaggatggtatcgatctgaccttgtgatctgcccacttcggcctcccaaagtgctgggattacaggcgtgagccaccgcgccctgccgccagtgctgagttttatcatcagagagagaaacccaaccgcggcaggcaaagggtgtccctttattgttttcataatagtctcgtttttattttatgtattatatttatttttttgagatggagtctcactctgtcacccaggctggagtacagtggtgcgatctcagctcactgcaacctctgtctcctggattcaagcgattcccctgctttggcctcctgagtagctgggattacaggcatgtgccaccacacctggctaatattttttgtatttgtagtagagatggggtttcactatgttggctgggctggtctcgaactcctgacctcaggcattttgtcctcctcagcctcccaaagcgatattctaggtttttttttttttttttttttttgagacagcgtctcactctgttgcccaggctggagcacagtggcacgatctcggctcactgcaagctccgcctcccgggttcacgccattctcctgcctcagcctcccgagtagctgggactacaggcggcccctcaccacgcccggctgattttttgaattttttttgttagtagagatggggttttaccatggtctcgatctcctgacctcatgatccgcccgcctcagcctcccaaagtgctgggattacaggcatgagccactgtgcccagccctaatttttttatttttatttttagtagagacggggtttcaccgtgttagccaggatgatctcgatctcctgacctcgtgatctgccctcctctgcctcccaaagcgctgggattacaggccaatattctaggttttaaacaagccttgcggggagatgtgtccaccatgctgctgaagggtcagccttctcttttgtgctttcctgcaggaatacctcaatagcattctccagcatgccaaggatttcaaggaatatcacagatccgtcacaggcaaaatccagaagctgaccaaggcagtggccacgtaccatgccaacacggagcgggagcagaagaaagagaacgagcggatcgagaaggagcgcatgcggaggctcatggtatggtcctgccttcttgacgtgcgctcttctacatgtgtagctggtactgcgggctccaggggtcactccccagggttacgccaaggcatttcacagctcggagttagaggtgggacagagggaaaagagcaggcgcgggcttgggagtcagtcctgggctcggatatttgctgatctctctgatcatcagaatgactgtgtgacctcaggccacttagccggctcctctgccttctctggaaaatggaggtggtgcccccttccctcgttctgtgagagccaggtggtgtgatccagacccccgacccctagcataaacatctctgaccatttatttgtgactgagtcgctggttgggcagatattggcagcgtgttctgggccaggtgctgctctgtgtacggggacatggcccctgcctttgtgggggctcctggttgtgcttgaggggcagatacgcgaatgaagcaaacagagtagtggatggtgaccgctacagtgaggagggggaagtggagtggaaggggagggacagggtagtaagtaaaagccttgaaacagaggcccaaagggtggccctgatgcacaaacgcagagacccagggaaggagccccaggcagtgcggacaaaggttgaggtggcaggcagccatgtgccagggcagccggtagccctgggctcccagaacagcgttgtgtggggcaggaggtgaggtcagagcttacaggggcaggtgggaggtgggacctttcagaccattgggaggactctgggttgagttctgatcatgaaggggagcctcacccgggagggttttggctgtggggtgatgtgatctgatgtattcttagaaaggtggggggctgctgtaggaggtggtgggagggatgcaccttggacttggtggagagaaggcttcgggtttggggtgtatttcgtggacagagtgggtaggactggctcatggcctctgtaaatggctgctggcgggactgtctgcctagcgggtgcccttggaacctagcccttggtgggttttgaggaaatgattcctgaatgaggagtcgattgccgtgtgaagggctggtggcacggcacccgcgtgagctacgcgtgccctcagtgcgcttctggattgactggccatgggtgctcacagacatgcacattgtgccaccacattgcagtaacccccatgcttttgtaggctgaagatgaggaggggtaccgcaagctcatcgaccagaagaaggacaagcgcctggcctacctcttgcagcagacagacgagtacgtggctaacctcacggagctggtgcggcagcacaaggctgcccaggtcgccaaggagaaaaagaagaaaaagaaaaagaaggtgtgctgggcctggcatggtgcccgccgcgggtgggatgggagcagccgtcttcacgtgtgtggcctcagccttgtgggtcagggcctgaccgtgtctctctctatttccagaaggcagaaaatgcagaaggacagacgcctgccattgggccggatggcgaggtgaggaagcagggtttcttgtggaagtatcaagctagccctaaggcgttggtctgtttcagactttaaaacctgtgttcttttaaaattacgaacaatgatatgtgatgatggtgagaatcccagggtgtcctctgacgcccatcccacccgaccccacatcccacatgttgtgttttcttccagacctttttctatgcatgtgccacaggattcatctttcaggttttactttgttgtaacgtccgacttacaggagaggtgcaagaaccgtccagagatgtcctgtgtgttcttcacccagagtctccattttcacgttttgtgttttgttttgtttctttgagacagggtctctctgtcacccaggctggattgcagtggcggatggctcactgcagccttgacctcctgggctcaagtgatcctacccttcgagtagctgggattacggttatgccccaccacacccagctaagttgtttttttttttgagatggagttttgctcttttgcccaggctagagtaaagtggtgcaatcttggctcattgcagcctccgccccgcgagttcaatcaattgtcctgcctcagcctcctgagtagctgggattataggcatccaccaccacgcccatttaattttatttttatttatttatttatttattttagacggagtctcgttctgtcacacaggctggagtgcaatggcacaatctctgctcactgcaagctccgcctcctgggttcacgccattctcctgcctcagcctcccgagtagctgggactacaggcacctgctaccacgcccggctaattttttgtgtttttagtagagatgggatttcaccgtgttagccaggatggtttcaatctcctgacctcgtgaacatcggcctcccaaaatgctgggattacaggcttgatccaccatggccagccagtttttgtatttttagtagagacggggtttcaccatgttggccaggctggtcttgaactcctgatctcaagtgatccacccgccttggcctcccaaagtgccaggattacaggcataagccaccacgcctggcctaattttttaaaatttattttctatagagacgggggttccatatgtttttcaggctagtctccaattcctgggctcaggcatcctcctgcctcagcctcccaaagtgttaggattataggcatgagcccccgcacctggccttcacctgtgttttttcctaaactctttgagagtcaaatgcagacgtgacagcgccttacctccatgtaattcattatctacttcataaagacaaggttgtttctgcacactgtaccgaggtgacggacacaggaggtgaacattggtacagcactgttatccaatctataggccttcgtatgaggtttggccagtttttccagttatcttcacagcagcagagaatcctgggtcacatttagtggaggtccctttagtcagctttagtcttaaagagctccttgtgtgtgtgtttgtttttttgcagagatggggtctcatgttgcccaggctggtcttgaactcctgggctcaagtgatcctcccacctcggccttccaaaggctgtgattataggcgtgagccaccacgcttggcttcaggccagctatgtcatccagtggccccggactttggtttgtctgatgttccttgtggttggagttgggttatgtgtctctggcaggcagacccaacatggccctgagtccctctcagggtgtcatatcgagagtgcatggcggaggttagcaaataaggtcactggtgatgctgactttgaacccctgccaagggatctccactgtccagttactgtttttttcttttgtaatcagtaagtaccctgtagggagatacttgagactctgtaactatgttgtaactcctcagaattccagctactccttttagtatctttttttttttttttgcctgtatcagtttttgctctgatggctacaggatggtgacttttatttccatcgttctttctgcattgatttgtttgtttttctatagtaggaaaagctttctctttggtcctgttagtcctgtggccatggtctgccaacccctggtgtagacttacggattcttgtttagttgaatgaatggattccgatcctttaaaatgatttcttttggtcatttagctttagccagcaggagccccttgccattggctcctgtttgcttttggtacatgcccaccattcctggagcacttccttcctcatcagacttttcttctgctttccctgcctgagctctctggagccaactctttctccagggagtcctggttccttttttttttttttttgtctgtcaagacggggtctccctgtgtcacccaggccggagtgtgtggtggtgcgatcatggctccctgtagtctcaacctcctggactgaagcgatcctcccacctcagcctcctgagtacctggggctacaggcgcacgccaccatgcttggttaagtgttctgtatttttttgtagagacagggtttcaccatgttgcccaggctggtctcgaactcctgagctcaagcaatctacctgccttggcctcccaaagtgctaggattacagacatgagccactatgcctggctatatttcgtattattacagaagaatatttacatgccaagatctgactttgggggtgcttcctgctagtgggccatcactgcttctaggccctctgagcagccggatctgggaaatagatgtatgtacacatactcatctgcattgctgtttgtatatattaaaacttgtggctgggcatggtggctcatgcctgtattcccagtactttgggaggccaatatgggaggactacttgagcccaggagttcgagaccagtgtgggcaatatggcaaaaccttgtctttataaaaaattttaaaaattagttgggtgtagtggcatgcacctgtggtcccagctacttgagaggctgaggtgggaggatcacttgagtccaagagtttgaggctatagtgagctgtaatctcaccattgcactccagcctggtcaagacagtgagaccctatctcacacgcaaaaagaatcttgaggttatactgacacctccaattctaatccaacagcacagggttaatttttatcctttccgcctttccataattctaaaatcagcaacctgatttcccttagcaataataggcttacttacctgctccattttagaatacacagaaagtacgttccaaagtgctaatggacacctctatgcagaagtctcctagttagagttcagtatttatagagttcctctacctcaggatatgtgttccaaatactgtgtccagaagttactggggccgggtgcggtggctcatgcctgtaatcccagcactttgggaggccaaggtgggcggatcacctaaggtcaagagttcaagaccagcctgaccaacatagtgaaacctcgtctctgctaaaaatacaaaaaattagccaggtgtgggggcacacgcctgtggtcccagctacttgggaggctgaggcaggagaattggttgaacctgagaggtggaggttgcagtgagccaggttcatggccattgcactccagcctgggtgacagagtgagattctgtctcaaaaaaaaaaaaaaagaagttactttggaccaggtgtggtggctcatgcctataaccccagcagtttgggttgctaaggtgggtggatcgcttgagcccaggagttcgaaaccagcctgagcaacatggtgaaacctcatctctacaaaaaattaaccaggcttggtgcgcacgcctgtagtcccacctactcgagaggctgaggtagggggataacttagcccaggaggtccaggctgcagtgcactgtgtttgtaccactgcattccagcctggggagctttttattacttgagacagggtctctctctgtcagccagttctgtctcacagttctgaagccatctggaggactcaagaaaaaagtaatgggccaggtgcggtggctcacatctgtaatcccagcactttgggaggccgaggcaggcagatcatgaggtcaggagttcgagaccagtctggccagtatggtgaaacccagtctctactaaaaatacaaacattagccaggcgtggtggcaggcttctgtagtcccagctactgggaggctgaggcaggagaatcacttaaacccgggaggcggatcacaccactgcactccagcctgggcgacagagtgagactctgtctcaaaaaaaaaaaaaaaaagtcatgaagtgggaaagtgcatgaagtgggagcttgtgctcagtcctgctgtggctgcctgtcctgtggagtgttgagaacaggcaccggcacttgctgctgcttgtgggttttcttgttctcggacagtggtactcagcctctgttgtgtgttctagtcacctggggagctctctattattattactactattatttgagacagggtctctctgttgtccaggctggagtccagtgatgaggtcatggctcaccgcagccttgacgtcctgggctcaagtgatcctccgaggacctttttctaagtagtagctgagcatgaaggcatgtgcctgttgttgcagctgctcaggaggctgaggtgggaggattgctttagtccaggagttcaagaccaacctgggcaacatagggagactctgtctctccaaaaaataaaataattacctgggcatggtggtgtgcacttgcggtcccagctgattgggagccagaggtaggcggatcacttgagcccagggctttgaggctgtgatcacacctgtgaataaccatggtactgagcccgtctccaaaaaataaaaagtaaattaaaaatcttaaaaagcgaggatgcccaagcctcactccagaggagttcaatcagaattttgggtgtaggggaggcatgcagcaatcggcatctttgactgctcctcccagtgatcccagagaggatgtggagttgaaagtccctgactgaggcagcctgcagagcccattgttggggtaagaggttgctcttatggtggccgctctgtgggtgagcttttagcgcgtagagactgatatccctccgctgtgtggacggtattgacactgtgagcgcagcagttttgcaggtagttataaattcctggaagatgaaaaccaaaatttaaaccaataactttttctccatcctggaagttttttctaggaaatatacaagaattgatgagggagaaaatgtttatccattgaatctcaacaggaggagtttcatatttgttgctaagaggaattatttttgaaatatttcaattagctttttaacaaaaaagaatgaggacccttgatgagatctacaaatagagtataagtgggggaaaaaatcaaggtccaaagaatgtaaattatataaccgttagtgtaaaaaagagggagaaagtggaaacgtctaagtccttgtttcttatccatggaattgatctggaaggatggacaggaaactgataccagtggaaaactgtgaagagaaaaacttaggctggagcacagaagaatggggagagcccacatgatgctcttcagttttcgttaaaaaaataaaaatgggccttgggaggccgaggcgggcagatcacgaggtcaggagatcgagactgtcttggctaacacagtgaaaccccgtctccactaaaaatactaaaaattagctgggcatggtgatgggcacctgtagtcccagctactctggaggctgaggcaggagaatggcatgaacctgggaggtggagcttgcagtgagccgataccgcaccactgcactccagcctgggcaacagagtgagactccatctcaaaaaaataaataaataaaaataaaaaataaaaatgggccgagcatagtggctcacgcctggaatcccagcactttgggaggccaaggcgggtggatcacatgaggccagaagtttgagaccagcccggcctgaattttcagctatgagtgtctggactatttgaaaattacaatgtatatgccagttaccttaaagaaacaggtgttgggagactgaggcagagaattgcttgaacccaggaggcagaggttgtagtgagccgagagccgagatcacgccactgcactctagcctgggtgacagagcgagactccgtctcaaaaaaaaaaaaaaaaaaaaaagaagaaacaagtgtcagccaaggttgatgggttatatatttttactggattactgagtatacagtttacgcattttcccaccactggccatgtttgcctgccattttctgtgcaaacagttgagtgggtgcttcccaccttggcctctgtaagtgtttggtctggaggccctgcaacctcagtgtcacacgaatgactctttttcagcctctggacgagaccagccagatgagcgacctcccggtgaaggtgatccacgtggagagtgggaagatcctcacaggcacagatgcccccaaagccgggcagctggaggcctggctcgagatgaacccggggtgagttgggccttgcattccagatgcagtggggatccaagtcctcggtgggccttgttccagggaggtggcagccaggagcaattttacttctgtttgaattcccggcaggtttggtagggaaagtgaattctgctggctctgagcagatttgtatgaaagcccttacattttttctaggtatgaagtagctccgaggtctgatagtgaagaaagtggctcagaagaagaggaagaggtaagagtgcatttcctggctttcaaggctctcagtgcccactggcagtgacttccaccctgtgtgttgtgaccgtctcctgccctggctgggcatcttgtggggcagggaacagcagggcccaggcctgcccgaagtcggtgctttagagctgtcctatccaatagggcagcttctggcgacatgtgaccatttccattttaaattaaatcagtcacattacataaaatttaagactcattttctctttcacactggctgcatttcaggtgcttggcatcccttatctgggaatggtctgcctcatttgcccattttttttttttttttttgaaacggagtctcactctgtcacccaggctggagtgcactggtgcaatctcagctcattgcaacctccacctcccgggttcaagcaattctcctgcctcagcctcccaagtagctgggactacaggcgcctgccaccacgtccagctagtttttttatgtttagtagagatggagtttcatcatattggccaggctggtctcaaactcccgacctgaagtgatccacctgcctcagcctcccaaagtgctgggattacagacatgaacctccacgtccgacttatgatttttttgttgttgttttgaaatggagacttgctgtgttgcccaggctggagtgcagtggcgcgatgttggctcactgcagcctccacctcccgtgttcaagcgattctcctgcctcagcctccctagtagctgggacttcaggcacctgccaccatgcctggctaatttttgtatttttagtagagacagggttacaccatgttggccagtctggtcttgaactcttgactggtgattttttttttttttgagacagagtctcactctatagcccaggatggagtgcagtggcgcgatctcgctcactgcaacctccgcctcccgggttcaagtgattctcctgcctcagtctcctgagtagctgggattacaggcgcccgccaccatgccagctaatttttaaaatatttttagtagagatggggttccaccatgttggccaggctggtctcgaactgctgaccttaagcgatccacccacctgggcctcccaaagtattgggattacaggcacgaaccagtgcgtcaaagtctgttctgtgttagtcactctaaccctcttatgattattgttcacatggtacatctttttcccttcttttactttcaacctgaatctacaacagcatatgggttttgcattttcagctagtatgacagtgactgctttttgactgagtttttaagaccatttatatttactgtaatttctgatatgattgggcttacatttgctatttcttttctatgcctcctgtttttgttcctcctttactgccttttgtgttcaatgttttttttccctagatttttatttttatagtgccttttttttttttgagacggagtctcgctctgtcacccaggctggagtgcagtggagcaatctcggctcactgcaatctctgcctcccgggttcaagtgattctcctgcctcagcctcccgggactacaggtgcatgccaccaaacccggctaatttttttttgtatttttagtagagacagggtttcaccatgttggtcaggctggtctcgaactcctgacctcaggtgatctgcctgccttggcctcccaaagtgttgggattacaggcgtgagccaccacgcctggcctctcctatttttttgcttacctcttcatgtttagttgaagcgtactttagctaccctggagtctgacttttaaaactctgggaggttgtggtggcgtctagtctgtttagtaatttcctggggctcagtctgtgaaatctggttcctgcatagtgtgtatagcctctgctgtatttcctcaattcttttaaacattttttggttttatcttattcgtgatttttttcctggttcattaggggcaggcagtgtttctttgtcacttagtggtcatccaaagattggccacagtttgtgctcagacacgcgccttctttctcctgctgatggttcagttgtatgaggaggtgcattccacatttgggccattgtcgggttctccctggcacttatgctttgctgagtcctttggcgtctctttgcatatgcacagactcagcctcagccagtgatacgcttttccctaccgtggctgatctcacctccattagcagtgccactggctgtgggcattgcttccttctacaagtgactgagccccttccacctccctgccaagcctcatggcctgcagctctccagctgtaagaggttcatgtgtccagagctgaggcagaagaagtgaaagtggcccacagtaaaagtgctacaaactcccactcttcccacctgaagttcagttttttaaggttcaatgattctagtagacctttggtcaactcgctgagctctgaaatgctcactctgtcaatttgtccggctttatagttgcttttggggaggaaatttgcccacctcctcatttgggtttagccagaagtctcactcctattttaattccagcatgtctgtaccagctctgattttcttgcctaaaatgtcggtgttattaccacctgtttattccaagtgatctgtaaaatcattttttgtgacctaagggaaatcctactggcttttcactggaattatttaacagattaattgcagatcatatttttaaagctttgtagcagttccattatagatgagggaacaatcaactgtgttgtatttgtaggatttaaatggcaatacaagaatagtttaagataaggaaatgcattcttgtctatgttatcaatatgtgttatgcaaaatcgcagtaaaggctggaaagccattaaatgatgcaacatgaatgattaaacaagttcgtagataacttgcagtaaactgagatgcctcttgtcgccagtgttatcagacatattgtagatggttgagacttgtcagtaaagctggaagtaaattacaagcaataaatattgaaagttggttctaagatatttaaagccaggcgcggtgtctcacgcctgtaatcccagcattttgggagggcaagttgggtggatgatccaaggtcaggagttcgagaccagcctggccaacatgatgaaaccccgtctctactaaaaaatacaaaaattagccggggtgatggtgggctctaagatatttaatacctaggaaatccaagagaatcaactgataaactgctttagtaggtcagcagagaatagacgtgtaatgttcattgtataacaggggtaggagagatatggagtctacttctaattagtaatgaaagtccacaataccctgagatagccttaaaagaagcgtcggggccgggtgcagtggctcatacctgtaatcccagcactttgggaggctgaggcaggcagattcttgagctcaggagtttgagaccagcctgggcaacatagtgagtccctgtctctaccaaaaatgcaaaacaattagccaagtgtcatagtgcacgcctgtggtcccagctactcgagaggctgaggtgggaggatcgcttaagtccagtgggtggaggtagcagcgagctgagatagcaccactgccctccagccagagtgacagagcaagaccccatctcaacaataacaaaaaaaaggaagtgtcaggaatctgtggtggggaaaccaaaaacggtttactgggctgggtgcggtggctcacgcctgtaatcccagcactttgggaagccaaggcaggcggatcacgaggtcaggagttcaagaccagcctggccaacatagtgaaaccccgtctctactaaaaatacaaaaattagccaggcatggtggcgcatgcctgtaatcccagctacttgggaggttgaggcaggagaatcgcttgaacctgggagctggaggttgcactgcgccgagatcacgccactgcactccagcttgggcaacagagtgacacttcgtctcaaaaaaaaaaaaaaaaaaaaaaagatgtagccagtgtggtggcatttgcctgtagtcccagctacttgagaggctgaggcgagaggattgcttgagcccaggagtttgagaccagcccggggaatgtcacgagactacatatctacaaaaattcaaaacatttgagctgggtgtggtggtgcatagctgtagttctagctgctggggaggctgaggcaggatgattgcttgagctgaggagttcaaagccatctgggcaacatcctgagactcccatatattaaaaaaaataataataaatacataattttaaaacaaagatgtaaataataaacagcacacattccaggataggtagagggggtgagggctgtaagaagatcacttgcttctgagactgttctccccatgggagtcccgtcccccctctctgggggatgaactgaggtgacatgggcttgtctcttggtaggaggaggaggaagagcagccgcaggcagcacagcctcccaccctgcccgtggaggagaagaagaagattccagatccagacagcgatgacgtctctgaggtggacgcgcggcacatcattgagtaaggggtcccgacacaggttgttctgtgccagcttcctgtgaggtggcgctggtgggagtggctagaatcccagccatccctagctgacattcagcactgtccagccagctgctactgtggaactacagagaccaaatttatttacttcacatttcatgtattttatagggatttaatagagcaattgcattcgcatggttcaccatatgcttacgaggtacagtcaggtgtgcgggagaagccctcctcaccccgttctctgctccccggctccctccctttccactaccctccctggggaaactttccagccttgtgtgtcatcttccagagatgctgtctgcattagcaagcaaaatgtgcatatttttgtcttcatttgcatacatatctccagtgtacattggtgtgcaccttgttattttcactgaaccttccctcttggaggtcatcctgtgtccgtgcagagtgcttccagtctggtcctcagcgtgcagtgtcatactgcgtcacgttatattctcattttcgtagcctgtcctttggtgacaagcgtttaggttatatttgtcatcttttgctcctgtaacacatcacagttaacacagtaatctcttcattgcttatggtaaggaattaatagatcctgttaaaaagcaagtgagggctgggcgtggtggctcaggcctgtaatcccagcactttgggaggccaaggcaggtggatcacttgaggtcaggagttcaagaccagcctggccaacttggtgaaaccgcatctctactaaaaatacaaaaattagctgggcgtggtggcgtgcacctggagtcccagctacttgggaggctgagacaggagaatcgcttgaaatggggaggaggaggctgcagtgagctgagatagcgccactgcactctagcctgggcaacagagggagactccatctcaaaaaaaaaaaaaatgtaggtggataaaagtcacttttttttttttttttttttttttttttttttttgagatagagtcttgctcttcaccaggctggagtacagtggtgcgatcttggctcactgcaacctccacctcctgggttcgagtgattctcttgcctcggcctcctgagtagctggcacaacaggcgcgggccaccacgtccagctaatttttgtgtttttagtagagatggggtttcaccatgttggccaggatggtcttgatctcttgacctcatgatctacccactttgagctcccaaagtgctgggattacaggcgtgagccaccgcacctggccagagttactttttaaaagaaaaacattcagattagattacaaattgcctaagttttaacagctctggttttgtatgaacctgttcagtctgtgtttcttttcacctttaggctcatgaattgttcttagaaagggtgggttctatgatgatcagcatctggctaggtgcagagtgtctttctgtgctgtggcaagtgacagcattctatttgttgttttgagacagtcttgctcagtcgcccaggctggagtgcagtggctcgatctcttgactcactgcagcctctgcctccgggcttaagtgtttcttttacctcagcctcctgagtagctggggttacaggcatgcaccaccacgcccggctaactttttttttttttttttttgagacagagtcttgctctgtcgcccaggctagagtgcagtggcacgatctcggctctctgcaagctccgcctcccaggttcatgccattctcctgcctcagcctcccaagtagctgggactacaggcgcctgccaccacgcccggctaatttttgtatttttagtagagacggggtttcaccatgttggccaggctggtctagaattcctgacctcaggtgatccacatgccttggcctcccaaagtgctgggattacaggtgtaagccactgtgcccagcctaatttttgttttttgtagtagagatggggtttcaccatgttggccaggctggtcttgatctcctggcctcaagtgatccacctgcttcagcctcccaaagtgttgaaattacaggcgtgagccaccacgcccatcagagtgacagaattcttgtactgtggtggtgcaatcaggacccagagctcccaggagtccagtctggacccctcaggcttagtgcgggctgccctgcctggcctcacactgtccggctgcacctctatgtgtcttacagtgctggccacaggtcattcagcagaaagggccactccgcagagcttgtgtcaggagccagcacattgtcacagataggaatgtgtgtccttacccggcacctccatctcactcccaggaatgccaagcaagatgtcgatgatgaatatggcgtgtcccaggcccttgcacgtggcctgcagtcctactatgccgtggcccatgctgtcactgagagagtggacaagcagtcagcgcttatggtcaatggtgtcctcaaacagtaccaggtgaggtagggggtggggaggccaccgccacgtagctgcctcggtgcaggtgttcccaggtggtgcgggcttcagacctcaggcagacctgagttcaccttttgctctttgtgggcaatgcactcttcccctctgagcctcggtttccccatgtgtaaagcagagatgtggtctggtaggttagctgccaggaggacgtgcaacagcagaaagtcagtgtctactgggtgcccgatgtgccaggcagagtgggagcgatttgcactccagtggcctcacagaggagacggggccacctgaggagcagagaggtgcagcacctcctcagcatcgcaccgtgtgtaggactgaaccagggccggagctcagatctgggcatctgcatgctcaggacaggttacctgagctctgtgtggtgtgaaacggcgagcgtcaggagcttagatctaggagccggcagccctggactccgctctttcttctggggatgaggtagcaggtcctgtgtgagggtgaaaagttggtggctctgaagctcttcacctgggcctgacttgggacagtcacgaaacagagctgctcatgctcttaatgcggcctgccatccagagctcattacaaggccccacaagacaaccagtacaactccaaccaaagaacagctctctctggttttcaatttggttgtttcttttcttttcttttttttttttgaaacggagcctctctgttgcccaggctggagtgcaatggcgcgatctcagctcactgcaacctctgcctcccgggttcaagtgattctcctgcctcagactcctgagtagctgggactacaagtgcgtgccatcacgcccggctagtttttgtatttttagtagagacagagtttcaccatgtcggccaagctggtctcaaactcctgacctcaggtgatccgcccaccttggcctcccaaagtgctgggattacagatgtaagccagtgcacccagccccaactttctttaagacaactcttaaaccttttgtgctagtatcaacattcaaagctgcacatgcacctatagtcccagctacttaagaggctgagatgggaggatcgcatgagcccaggagttgaaagtgggcctgggcaacatagtgagacactgcctctacaaaaatattttaaaaattagctgggggtggtagcacacatctgtagtcccagatagttgggcggcttaggtgggaggagagcccgagcccaggacttctgagctgtagcgccctatgtcgatcaggtgtctgtactaagttcagcatgaatatgatgaccttccaggagtggggcaccaccaggtggcctaaggaggagcgaatctgcccaggtcagaaatggagcaggtcaaaactgatgtgctgatccgtagtgagatagtccctgtgaagagccactgcactccagcctgggtggcatagcgagaccccatctctataaaacaataatgaaacaaaggcaaagctcatggttagtggaattttccaagagtcccatgcaattaaaataattagaacttggctaggcgcagtggctcatggctgtgatgccagcactttgggagaccaaggcgggtggatcacctgaggtcaggagtttgagaccaggctggccaacatgatgagaccctgtctctaccgaaaatacaaaaaattagctgggcatggtggtgcatgcctgtaatcccagctacttgggaggctgaggcagaattgcttgaacctgggaggcagaggttgcagtgagccaagatcgggccactgcactccagcttgggtgagagtgagactcggtctcaaaaaaaaaaaaatgaattagaacttggccagaacttacttcctcaatccattacgaacatttccaaattttccttcataacgttatcttcagtttgcaaatagtcttctgtttaaggcctgtatttcagaaagctcccaggctttggaaatccctctcttaatcttgaaatgatgttcaggcagtgaattcctcacgaagtcgccctgaagccctcttggtttcctgagtgcaagtgtcccgtggtgtgggcaggtttttcctgtgtgctgtgtggggcatggacaggcagacaatgtaacaggtgggagtgagctgttgagatgtgtattagaaatgtcacacgtgtccatcgttcgcacagtcatcctgcagcttcacagacgttattgctaaggatgaggctaagcgataaagaatcagtttggttcagaggaaaaatccgtggtaaaggcagtagagggtgggatgtggcttaggcagaactcgtggctggcggtgtcttgactctctgaagtgggaggaccctctggtgtccgacccggccttcagtcctggcgtggccgcatctgtccttgcagatcaaaggtttggagtggctggtgtccctgtacaacaacaacctgaacggcatcctggccgacgagatgggcctggggaagaccatccagaccatcgcgctcatcacgtacctcatggagcacaaacgcatcaatgggcccttcctcatcatcgtgcctctctcgtgagtacccgctgccagcaacatcccacacgccgctcacacgctcctgtgtttgtttcctaagtttgccgcagtagaataccacaaacgaggtggcttaattacagaaatttgttttcctggaggccagaagtccagggtcaaggtgtagcaggttgcttcctcctgaggcctctcttcctgcttgcagggatacgtgcttacatgtatctcactgtgtcttcacatggtccttcctctgtgcgtgtctgtgtcctcatctcttcttcttagaaggacatgggtcaggttggatgagggtgcatcctaatggcctgctttaacttcattgcctctgtattggccctgtctccaggtacagtcgcatcctgagctgctgggggtcaggactgctccaccagcctcggggctgtgcacagctccacccgtcacagcctctgcctctgggtcccggatcaagtgggggcatgactgtgcatcccctccacccctggggaggcctggggtccctctcccgggatgtccttggtgtccttttcaagtcccacaaccagcaaagtccatgcaggggagtcgcaggagacaagaacctcccactagaaaagtagggtgaggccattgaggctccattgaacagtctctacttcctcactgtgcagtgccctgcaagggccaccagagccctcgggcacatcccagcatggcccaggtccttgggcatggtgggatccggacaccctgagaggctctcacttgccactgcctggctccaccggcctttttctccctgcaaacagagtccgcccccctgcccacctctctcgacatctctctacccagctcagcccctgcactaaagggggacctgcctagggttttcccagaaagccccagggccggctcttactggtccagttcaagtcacatgtccagcaagcagacagtcaccggcctgggggttagtctggtctgattgggcaaacttcagcatagcatttggtctggccctagagcacagcccctaactccaaccaggcactcattgggaccactacagccacgagtcagcagcggacccccaactcctggcaggctggggagcggcatgcccatgctccaaccccatgggaaaatgcccttctgcctataagacagccaggccccatgggtcacacacccatagggtaggcctgacgtcctgactgttgcagggaggactggaggaaaggtgaggaccccagaaccctcccaggtttactgtggaactgtttctttcttctatctccctgcttcctacgtggcgggtagaacagactgctctgaaggagcagggcgtccttgtgcacagcgctgcaggtagctgcacctcccctcgtggtcagacccagattgctacagcgttcctgctgcacctgcctgagctctgtcccctgtctgtgtctggaggctgttccttccggctctgcccacctcctggctcccctctgcccttcttcccctggaggctgatgggtgcagtggcagtcgccctctgcagttcccgattttccctcctttcacttacagacttctcagatcaccatctctgctggaacgccgctctccagggtgctgttgttttgcagccttggttcttagttgttaattttttgctttaaaaaatttttttaaacttttcattttgaaatgatctcatttatttttatttttttttattttttgaggagtctcgcactgttgaccagcctggagtgcagtggtgccatcttggttcactgcaacctctgcctcctgggttcaagcgattctcctgcctcagcctcccgagtagctgggattagaggcacgcgccaccatgtccggctaacttttttgcatttttagtagagacagggtttctccatgttggctaggctggtcttgaactcctgaccccatgatctgcccgccttggcctcccaaagtgctgggattacaggagtgagccactgtgcctggcgacctcattcttagaaaacattgtagagttagtgtaaagaattgctatgcacccttcacccatcttccccattctttcataaccccaagacaattatgaaaaccaggaagataacatggacgtaggactaatagcaaaccagaggccttatttaccaatcttgtcagttttctcacaagtggccttgctctggtccaggatcccacactgtcacgtctgtgtggcatgcctgtggcagttcctcatttcgtctttgtcctggtcgagcttgacgctgttgaatcctgatcggacctttggtagactctccttcaatttgggtttgtccgatgtgatcgcgtggagcaacaccacgccctgcttgatgtgccttagcggggaaggcatgagatgttgaggccttggtgtctcgggcaaggtgcctgctgggtttcccactgtaaacctaggagttacactttccgttaataactgtctcgtgggaacgtacttcgacacttgtgaatatctcatttctcttcctactttcctcaataatttcagcatctcttctactccatttcatgttgccataaaggaatacctgagactaggtgataaagaaaagaggtttagctgggcgcggtggctcactcctgtaatcccagcactttgggaggccgaggcaggcggatcacctgaggtcaggagtttgagaccagcctgaccaacatggtgaaaccctgtccctaccaaaaatacaaaaaaaaattagccaggcatggtggcgcatgcctgtaatcccagccactcaggaggctgagacaggagaatcacttgaacctgggagacgggggttgcagtgagccaatatcgtgccattgcattccagcctgggcaacaagaatgaaacgccttctcaaaaaaaaaaagaggtttatttggctcatgattctgctggctggaagactgggcacctggtgagggcttcaggcggattccactcctggtggaaggcaaaggggagccagtgtgtagagatcccatggggagggaggaggcgagggaggcgggggtgccgggctccttttaaccatagctcccacgagagttcagagagcaggaactcattcatcccccatccccagtacagtacaggccgttcacgagggttcacgaggccgttcacgagggaggcccccatgacacagatgcctcccaccaggccccacctccaacattggaaatcagacttcagcatgaggtttggaggtgccagatacccaaactatagcagcaaacgtcagataagtcttgcctgaaaaaataattacatgagagttgccaaatacatggttacctgtttctgtccgctcttctctacaaattaattggaattccaccgttaggaagagctttcccgtctcccccatgtgcttgtttgggtaattattatatcagtgtgggcgtgaacaaatattatttattttgttgcttcagtttttttcagatttggcctgggagaattctccctggaagaattgtgtcttctctatgtattcagtcatttgttaatatgagtatggacccacggatatttatttagtactccaggctacgcaccagtggctgggttttttttgttttgtggctcaaatccatccagctttggccactgggagtgctttcagttggctcttctgtcctttgacacgcccccagcatcgggtgaattttgaggactttcttgctttttggcaccaagagatgctgcaggctcatgtagtctatttcccgcccactccctcaaatcagccatttctccaaggagccctggtttcccttcttacagaatggttttgggaaccaagacctgggtcctggatgggcctgttgcttctggggtgtcattgctcctggggccctctcagtggtcagtgctaggagattcatgtgcgcgtgctgatgctttgtgcacgtatcgtctaaaaggccttctctgcctcggcatctgtttctgtgttaagctgaacgtgagttcataccactgtctgcggctctgagtgaggaccacaggttccttctcacacttccaccctgtgtgtgcgcccattctcagcttccgtggacaacagtttcagagtagtgagccggtacctccgggaagcatcttgagccactagagcgcaggccttggggccagttccttgtgtgttcagacctccagattgcagtcagattccatttccagggtgatagaggtcaccttcttcccctacttcagtgaggttgtgaggtgggtgctcctgcagtctttcccactgcgccccacctgggcaagcgtccatgctgtggaacgccagctcctgcaggccagaagagggcccgcgcgctaggaaatgtttacggcgtatgggcatccggctgtgccggccctctcactgggaattgctgcctcctgccatgagctgcttgtcctgcctccgtttggacctctgtctctgggacctgtcccctcagtcccctcctgacccttgctcctggctcttctccctcccagtgcagatgctccgcacacctccggcttctcccttggtgcgctgactgtccccatcactccagggaggtttcactgttcctgagtcagcggaagcacagggtcaggccctacgggccaggtccggtggctccaagatgccatcagggacccagcctcccccaggtttctgcccagtcattcttgggtgactccaaccctcaggctccttacctcatggttgcaggacggctgcactgcctccctcgcctcatgcaggagagtgggcaggaaagagttctaggagagtgggcagggcaaaaggcagcagaagcgcaaggctgctgagcctctgctccccacgcttcttcaggaactctccccagccttcaccacagccatgcgtccttctgtctattggctgtgccgcctggttggctgtggcttccagcggggctggagatgggttcatggtggacacagcacctccctcgtggagctgcttgggggcaaaccacgagtagtccctgggactccagcttcgagccctgtctttacggaccaccccgtctagactttctgagtggcttccctcccccactgaaaactccctgccccagagttcctcccagagcccgtcccctttgggactcagctttattaccgcctccaccctctcctcagtctctaggctgtcggtctcaggtgactcacccctccccactgcattctgcctgcccccatgtcctccctcattccagccctggtcatcccacaggggaacagccacgagttgccgctccctcccatccctgtcctcccgcctgccacctgagccaggccccgcctcgcccctcactgtcctcgttctctctcacagtcaggaaccttccgtggctggagggtggcagaaagactgtttggtgtctcctatatcctcaggtctccacagcactgttgctagcctggctctgggcgtgaagcctgtgctctgaagctgcattttcccggcagcaggtccaggcttccccagccctggcagcattgtttctgtcccccatcacatctctgtgtccctacttctcactctaaagggccttgccctcttttcccagtggcctcccttctgtcctctgaggcagagcccttcacgtcctcgccagtctctcttcctccctccctccctcagctgccctaaacacagtttctctgcccactcggagggctgtggccatgttggcctcgcccctgacagccagtggctatgggtttgcacagtgagccattgatgagagaccggcacttgactctcatttccttgttccatcagaacgctgtccaactgggcgtacgagtttgacaagtgggccccctccgtggtgaaggtgtcttacaaggtaggtcacagccactgaggtttcctctcttgctacggaggtgcaggcggtggtgggcaggacgtccacacatacctctggacagtgaacctgagaatgctgggtctccagtcgcatggagtctccaggacagcctggaactccagtcacatggatccgggagtttggactgggcagggacagggcagattagctcactggggaggagacaggagtgagcatggtggccagcactcagaggccagctcaggcgcctgagatggggacccaggaagaggggagcctgtcagccaccaggaatgtgcagatggcggtgcaggctgcgtggttccctcaggccccggccgccgctggcctgcactgcttcctcttccccctgcagcgcgtgttctgcgtggtgaggtctggggacgcgccagcggccctgccagcccctttccccactacccctgtgaggacgagccctcccgccgtgtcactgggcagttgcagggggtgcctgtgcccctcttgccacctggccacccggctccaaaagccgagctgtgcatcctgcttcccttgcagggatccccagcagcaagacgggcctttgtcccccagctccggagtgggaagttcaacgtcttgctgacgacgtacgagtacatcatcaaagacaagcacatcctcgccaaggtaacgtgtccctgtgggaaatgccaggccatgggccgagtgctcacacgtgggtcacgctgcccgtctcctccaaagcccctacaagtttcttcccggagtcccacctgcatgtgcgtgaagacagctgccctgtgtaggggaaaggcctaggtgggggcgacctcaggtttacctccctaactgtctccaggcaggccctgagtcaggcccagaagctggggccatctacacagcatgcgctctgcctcctctcgggccttctgcccagagagcctcagcaccaaggcgtctcctgacccgcctacagagtccccatggggccttggccagaccagtcttgtcctcctgtctcattgccttggccgtccctccctggggccctggcttctcaccacaggccatgggctgagattcagcatgtgggggagagctcgagggccaaataccatgactgcctgctggccttgggcagctggttcgggctgttctgtgagggtcctactgtcctggaagcctcttctgggacgaaactgtgaagggagtcccagcgtggccatgactgagaatacggcccagatagaaaacagacatagccctctgcctccaaaaactcatgttcctcatttgaggaggggtaaagatgtacctggtctcacccctaaacatccagctgagtgagtgttgaacagtagagagttttttgcgttttggtttttttttttttgagaccgagtctcgctctgttgcccatgctggagtgcagtgatgtgatcacagctcactgcaggcctgacctcctggcctcaagcgatcctcccacctcaccctcctgagtagctgggactacaggtgtgcgccaccactcccagctaacttttttaaatttattttttatggagatggggtcttgctgtactgcccaggctggtctcaaactcctggcctcaaggaatcctctcgccttggcctcacaaagcactgggcaactgtgtctggcctaacagtagagttcttgacccaggtatggggggatggaggagctgtttatgggtggctccgcctctcagtgcatggctggagtgacagtactggaatactggggctgtgaactgggtgttttcttttctttttctcttcttttttttttttattgagacagagtctcacagctgtcacccaggctggagtgcagtggagcgatctcagctcactgcaacaacctctgcctcctgggttcaagtgattctcctgcctcagccttccgaggactgggtgctttatcccggagccaacaagggaggaaactgaggcccagggagctgggtatcccacgtgcaggctcttacctcaggtccatgcttcaggtgcctcctttgtggctcacccctgctgactccttacaaagccaggccgttggcaggcacaggaaccaggctctgaaaccccgtgctcccctgccagtggctcagttgatgtaatcaccctgcagtgggagtgctctttgagtgcggccactgcgccccctcccgacccgcatggccctgtatgtagtggcgctggcccctaccccatggggcccacattctgaaccacaggctgctcattagaaatgtaccgtcaccaacccagttcccctgagaccctccacaccagcaggacaagtcccccagtcagcaagatgtgtcttctgccagaacgtgctcggcatttttctgtgctgtagattcatatgtgtgattttgcagggaagccagattgcttttttgcaatttgcattgttcacctgccaatagtcatggggtcacgggcctgtctctgcccaaggcttgcaagaggctacgttgtgtggctccaactcggtgagtcagccccggggcaggacgtcaggcctgtgctctccacccagctgcgctcttccacctggagccttcctggctgctgggcgcagagtgggagattctccccatgtgccgggccacctgctgccccctgccctgattgcccactctggggcccgcagatccgttggaagtacatgattgtggacgaaggtcaccgcatgaagaaccaccactgcaagctgacgcaggtgctcaacacgcactatgtggcaccccgccgcctgctgctgacgggcacaccgctgcagaacaagcttcccgagctctgggcgctgctcaacttcctgctgcccaccatcttcaagagctgcagcaccttcgagcagtggtttaacgcaccctttgccatgaccggggaaaaggtgggtttgcccagctgtgcccatgctgacggttccaggtgcggctggctttgctggttggaacgtgttgagcaccagctacagctggctgggcctgtgctgggtgcctggtgagagtctgcatctgcatggagcaggggagccctggaacccaaggctggggcagccacaagggccccaggggagcagggcagaggtcatggtggtgaatctcggaatgctcggggcgtctctgaccctatgctccaggtgcagagtggatggtgaggaagatggccctgaggcagctgtgggaccgcaagcgtgtggagagcacagctagaggctgtagcgtgctgtaggcccctttaggtgccctcagaactcaccctcagggcagtgggaaggaaacttggaagattccagttagcggggccacatggtcaggttctgtcttacgttgttttcaaaaagctcagtggccagtgtgtctcaatgtctgggtggcaccatcagccagactgtggcatggcagcatcatcattgatgagtggtcccacaccagcactgatccctgagcctagagctccttgctgttggaggttgaaactgtgggacagactgtgtttcctgctgaggggtttaagtaaccagatgctgaagtgtgggcagaactacagaggttacctcgaagtgccctgcccagcacggctgggcccacccccttcttcccaccttagacaggtccagggcagctggggccttcctggcctcatgcgtgagttccctgccctctcggctgctggtcaaggcagagactcttctgatggcgaaaccgaggcattccccaaagcctcgtgataaaggagtccccaatttttagactcccgggacccccacactaagggccaggatctgatgctatagttttgtcactttctgagccgagcaaaagctctgccaaaggattgccagggaagcaatagaagaaaagttttggcgtcgtctcattttcctttaacttttttaattaaaacattttaaaagttaattattagtagaggagtaggcgccgtgggaggtcagggtttctttcacctggttccacccaggctgaatgagagtcccttggggctcccggtggcagagccctccctgagaatcacccgctataagccccatgtggtcagcctggcccctgcagcgctcctggcatgagcttggcgtggggtgggtctgtggacactccagtttgtgttactcgtggagaagcagcatgcccgctgccgggggtctttctggtgaggcacagggagcagagtgctctgggtactgcggttccatgtcccaggaattacatggagggaaccagatcctcacagtttcctaaggaagagggtgccctgcctggctcccagaaagcataaagcaggctgaaaagccacgtgccaagggcaagatcaccccaggggaccccgtccaccctgtccccacatccgcaccttctagtgagacctctgtcgccctcctttggaggtaacgcttgcttctcctgtcttgggggcttccaggtggacctgaatgaggaggaaaccattctcatcatccggcgtctccacaaagtgctgcggcccttcttgctccgacgactcaagaaggaagtcgaggcccagttgcccgaaaaggtgatggagttttgaggggagccaccagtgaagcagcctcacgtgggggctttctccagggctgggcgtgctcagggcctctccccacagtcccaggcctgccctggtcaatccagcttgggggtggcgatgacgccactgggtctgtaaagccctgtgctgctgttgtagggattgaaaagcactcataggctcacatagctcacaataggttagagcacaaaatcccacttcctgtcagggggccaggtgcctgtgcgggctctcctggccacatcaggaggggccgtgggcttggggttgccccccggccccccatcatggctctgggttttcaaggcctgctgcagccacacccctgggcccagttcccacagcaagacccatggactcggggtccccggctgcagttgtccctcttggctctttgtttcctggctggctgtagagaggcccccagaagatggctggggggccctttctggcccagtaaccctctggtatctctgcctggtgcttcctctcattgcccaggtctcccctcttctccccagctgtgagggtctcctcacccacaccccagtgtgctctggtcagagctcatatgacagggccgagggggtcagagctgggttcggatggggggagtcaggcctcaagccaccttgggccctcgtgagcattatgtgtcccctgcaggtggagtacgtcatcaagtgcgacatgtctgcgctgcagcgagtgctctaccgccacatgcaggccaagggcgtgctgctgactgatggctccgagaaggacaagaaggtgggccccagagtcccccaactgcattccccactgggtgtccaaggccggcagcgtggcaggcagagcagagcgtgctctgaccatcgggtcatgatctggtcatgatccccagggcatctggccagccctggttatgatggctggtggcttgtgtcaggacacactgactcagctgccagtggcttctcctttgcctatgaaacactggctccttctgcaattggcagcctgggcccagcactaaccccagcagggttagagtgttctagaatgccccccttctctgttattaagtgaaaggaagggagcgggctgtctcctgcaggggctgctctgagaatcccaggccccaggccccacctgcctggctctcctcaccagcagtgaaattatcctcccaggaaatgcaccagaccccttgattctgcccccaaggccttgacctcggcctggcttccccgagacgttgtgtctgtgccccttctcccctgagctgagcagcagtgtgttccctagtccattcccagcccagagcatgcaccccccgccccgcccccacctccttcctcatcctggagcccagggagccctgagtcatggctcacccggctgaggccctcttgcccagctgagaccctctagggacccccacagtaacagtgcccagaacaaagctggcatcacaaagcatcggggactctgcctcagccctttgccagtcttgtttccgtcccgtcctgctggccctgtccctgccatctgccctctgctgtgccaggcagtcatttgtttcccagggcttgttgggggcctccgggcctccagcctgccatctcctcactcccaattgctgtgccaaaagccactcttccccactagagcgtccccatggcccttctcccaacacccacccatccaccaagcccaccccaccccaggagggcaagaccccatttgggtccctctcatctgccttccagggcaaaggcggcaccaagaccctgatgaacaccatcatgcagctgcggaagatctgcaaccacccctacatgttccagcacatcgaggtgagcccgccgcggctgggacggctcaggccctgctgtctgctgagctcctaggcagagctggcttctcctcgacagctctttaaaaacagactcgaatattttcattaggtaatgcctgtacatctgtctctcatttggtcttcgcttcaccagtgcttacgggaagaatcccagcagttagggcgcaacgtgcgataggaatatagggccctgaaagcttgccctcagagagcaggagcgtttggagacttctctggggatgtgtccccccacagtggggtcctgctgcgtctctttggctcactggcttttctcacgttgcggggcgtcaagagtcaccaccgtcttcagtgaagtgtgggggagtcagaaccctttttggtcatggtggctttgagaatctgggttcttagaagcatgcatatgttcgtgtttcttggtgtaaagtgtcagggtcgttggagaccgcctgctgccccgagagcccctggggctgtctcagcaccctgagcgtggctgtggccccatgacgtcctcgtgcattcagggtgtgaagagtgctgtatgggatgtcctttccttcgttggaggagtgtgagggaaaggatgtcagtggactaagaaagatggttttggcatctgtgtctggtctcagagccgccgtgggcccgtgccgagcacacagtgtgggggctttgtcctctgagcacgagcggtgtgtgcggaccgcagcggggcccggtggcctgctcctgcctgtcactgacccctctctccttgccttgcaggagtccttttccgagcacttggggttcactggcggcattgtccaagggtgagaagcttcccaactggatggggtgggcaggtggtccacccagaggttttctgtcgttttgttggctttattgctgctgttatgttgtccaatttcaaaggcagaaaattagaaaatacagaagaatccaaagcaaataatacaaatctttttttttttttttttttgaggtggagttttgctcttgttgcccaggctggagtgcagtgacacaatctcggctcactgcaacctctgcctcctgggttcaaatgattctcctgcctcagcctcccgagtagctgggattaccggcacctgccaccacccagctaattttttgtatttttagtagagatggggtttcaccatgttgaccaggctgatcttgaactcctgacctcaagtgatccacccgccttggcctcccaaagtgctggaattacaggtgtgagccaccgcgcccggccccattatacaaatctttagtgcttggagagctgaccactattaaccccaagagacaggattcctgaggcagacctggcttacagatcccactctgccgcctccaagctctgtgactctagacagctttcttaacctctctgagcctaaactctgctcactagtacagaggcactctttacatcatgtgggtgttttctatagcaccctttgtaaggggcagcccagactcctttaagcccctaatggatgtggatgctgtgtctagattttcaccattgcaaagaaccttgctaaaagcattcttccagctaaagcctccattcaattcccctggggtagtaagccatttgcaagaagagaggcgcctaatgaaaacatcacaaattctttttgatgtgtttttttatactgagttgtggaacagggtcctgtacacctggcttcaaggctgggaggcctggcttggacatgtggtcagccatatcacgtcgtccccctgccagcctcagtgtccacatctacaacacttggggtttggctggagagaacatgagaagccgaggtggcccttccttctgggtctagcagagcatgggtgtctccagtgtggcatttgctcctggggcttctttatcgggcaaggggacaaggtctggcttcccggagcatccctgttccagccccgtgttgtgccggccatgtgactcacagctcctctcattttcatgagtgtctgggtttgacgagaaatacatgtgccaccctggtgatacgcctccccagcgagggtggccgggcttatttcagttgggggaaatggctttgctgaagctttgggtgggtgacgtctgcttaggatgcatgtctgtgcccttgaacccggcgcctggcctggaggcgggcgatgcacctcctgccttacctgcctgcagggttccaggtttaacatcctgcgccttctctcctgcctcctccacactccaggctggacctgtaccgagcctcgggtaaatttgagcttcttgatagaattcttcccaaactccgagcaaccaaccacaaagtgctgctgttctgccaaatgacctccctcatgaccatcatggaagattactttgcgtatcgcggctttaaatacctcaggcttgatggtgagtatgagccagtgaggcgtttcttacagggttttgttgttgtggctgccacagaagctcctcactggcattccctcctgtgaggcagggtgaggcccaggcgctctgggtccagtggccagagtgggcctagccacaggctgagcatcttgggcactcggcactgggacaggccgtggattggacgccgggtgtcctgactcctaggctgacagtctttccacgacaatgctgggcgcactggggaaggggtgccctgtgcacttttagctggaagtgacagaattccataccatgtggacacaggaaagaaagcaccaataggcttacatagctctaaccccaggaggttaaacatccttcaggcacagctggatccaggcatgcaggtgatattggagagacttttctccagctctttgctctgccttcttccatcttggcttcctttccacctgctcagtgtccccaacaaaggcaggtcccaggctggctctcatttagctgctgtgagccgcatgcccatccctgggccattcgtagcagcccagtgactgccaccttgccacaggtcaggcctggacctggaatcagactctgtggtgagcacggagggagggaagcaagcactgccaggcaggagcagcagctgccgtagagaggctctcagcccaaccccctgccctgcggtccctgatgggaaaggtcaccgcctgtgtttctgcgcctgcctgccccagcacacgggagctcagggaggagaggctgtgagatgccgacagggcacgtgagacatgtctgtcagtcccagtgggtcagggagccctggagtgagttcttcgtgggagttactagaactgggcttacgagaaacacgagcatgagctgctgagacagacacggctcagccagtttgttgttccctacacgacctcccagagcctttgttgaagctcctgtgcaaggtctgtctcacagcaggacaccccagacctccataccccttgtcccgttgatcacttggacctgtgtttgctgggccaacctgcccaggtgccacccggagtgtgccctgtgtgtccctccaaactgcaggtcctccccggggttctgtccaggaccactctacagacacgagtctgttgcaggggtctccccggcttgccttcttccttgggggcttcccactttcagctttgaggctgaaaaccaaaatccttcagggtcctcatggccgggccaccccccaccctttcctctcctacggtctctccagcggcttctcccgtcctcgagagggccaaggcttcctgagggccatcccgtctgtgtcgtgtccctgactcccccaggagagagcctggttcccacccatcccagtgcccttctgcctactccataaggacaaggactttggtcagttgaactacagatgtgtcctctgcatgtgatgtggtcatggccacagggccagagaatccattgcgagtagaggaagggtaggagctcagtgaagtctcctgagactgcatctcccatgggtgggctggcatctgggagccgggtggcccctcacctcagcccctcctccgagctggagtagcaggggcacatctggtgcctgagagagggtgggacggcaggtggatggccaggctactgtgggcttctgcaccgtgcctgggcatgtgtaggctggtggcaggaaatggccagtttttatttcttcttttttttgagacggagtttcactcttgttgcccaggctggagtgcagtggcatgatctcagctcactgcaacctccgccgcctggattcaagcgattctcctgcctcatcctccctggtatctgggattacaggcgcccgccactacgcccagctaattttcttgtatttttagtagagacggggtttcaccatgttggccaggctggtctcgaactcctgtcctcaggcgatccacccacctcagcctcccaaagtgttgggattacaggcatgagtcaccgcgcccggcctgccagtttttatttcatcaccaaaatgacttcagtgttcaggggcacctctgtccgaactcccagcagcgcagggagtgttgacattgccttaatgcccacaacgctgagggcatcgtgggttgtcacccaggagacctgctctggctccttgacttgggttttctggggcatggggacactgatcctgccagaaaggacacgagccccctttataaggtctccatgcttttaccgtagaagattctctttgtgaaccacaaaactttttgagacagggctgaaggcaggtgcacccacagtcactgtccacccacccccaggtcccttgtgttgcgagcggcggtgttgccggcattggccgctgtgtcttccgctccccatggaatggcacagggtagagtagaggagagaatccagggctgtggtggtggtggctgtgctgacgctggacgctgtggcctagctgtgggctgggtgctggactctgtgctccaggcccacctcctctagttctcccagcggggcctgttttcatttctgggatgtgtggagagacgttttgtggtgaaacactggaaatccagttattcgccagtggcccacaggcccgtgtggagagtgcggagccagggatctggggatgctggcaggtgctgatcctgctcctgctctcagaagcaggtgttccttggtgtccccactctacccctgaggtcaccccgctgaccctgttctcctctgtgcccgtcaggaaccacgaaggcggaggaccggggcatgctgctgaaaaccttcaacgagcccggctctgagtacttcatcttcctgctcagcacccgggctggggggctcggcctgaacctccagtcggcagacactgtgatcatttttgacagcgactggaatcctcaccaggtaaaagcgggccgggccccaggtcgaggagaaggaagggggtgcctgcaaaacctcgaggagacggccctggcttgagggtcctccagcctcctccacattgtcttggaccccaggagccgggaggagctgcacccatatctccataggtcatcagggagaaaagaggcggggtcctcctgtttcccaaaatacaaacagcctttccctctgattgggaaagcagtgtataagccatccgtcggtttggtttttcttaaatcttggaatggcacccatgaggaggtggaaagtatcctgatcaatctgcgccgtcactgtggggcggcccctgcagtgtgccctgtgagcacacacactggcgcttgttcagacacaattgggggtaaactccatgccacttcgtttacttcctcctcttgttccataaccatgtacccctcatgggcctcggcatcggtggataccaggtcatgctgttacatagctacaagaatttgaaaatgagttaaacttttctctgtgtttcattataagactgataagttcttattgtttttaacatgtaaacaccagtgacagtcaggagtcccttttcccttcctctccaaccgtgctctcttctgagcctctgccagcccatctgccttcgcacatgtatgtactgagattaacacagatgcggcgtggcctagcatgtgcctcttccaccaagcagtgggtgtcaaccgccgagtgcacatcggaagctacccagccatctaatcggccaccaggcgtcccggcagatggccacccctggacgtctgctctcaaactctggttggcaggcatccgtcttttctgttgatcagcctggtcaccaacccagcagggtccctctttctctcagagtgcttgtgcaaacaccctaggtctgcgcagccctcagacatggggtcgctccaccccagacattgtcctcttcgtgttctggttgctactgccaaagtggtttcactcctacgctggggtgtgaatgtaccctgcccttccctgcagcgtgagggcagcagcgttctcagtgtgtaaattgtcgcttgtctgatgggtgaatgccgtggtggtcactggtggccccttttacgatcaggctggggtgtagcttctgcccagaccctggtgaccagccagtttcttaggaggtggttgagttgtgtgtccccgttggcaggccggcagcaggcggaagctgcacctcagggctgggggcgcgttggggagggctggccctgccaggaagtgtgctgacagggcaggagggctgagcggcagacacaaggcactcctggtccccaccagctgtggcctctgccaccccatgggtgccagtcttaagtttcagagcatcactggtactcaccctggtcctgcagctcctgggcccagagacaggatttcgtgttagcctcctcgtactgtgctggggtgatgataaaactaacaacccaggctggacgcagtggctcacacccgaaatcccaacattttgggaggccgaggtgggtggatcaactgaggtcaggagttcgagaccagcctggccaacatggcgaaaccctgtctctactaaaaatacaaaaattagccaggcatggtggtgcatgcctgtagccccagctactcaggaagctgaggcagaaaaatcgcttgaacttcgggaggcagaggttgcagtgagctgagatcgcaccactgcactccagcctgggcgacagagcgagactttgtctcaaaaaaaaaaaaaaagaaaaaaagcccatccacggcacctgcattgttgggagctaactgcaccagtcactgggccacactcctggtgcttattcactcattcgaggtatgcagtagccacgggttgggccccatgtaggctgagggagttttgaggctcacagaggtgagacctggttccggttctgtgtgcactgtgagagtggcagggcccacagccaaaccaagcactgccacaacacggccgagcttcggccccctgttgtaaatggtgctcgactgtgtaggtccacgctgtggggagctgtgccgctgccacgggagctgcttgagaatataatcccctggggggttggtgctttcttcccgaatatctgtggggtcccaataaggtagaaggtggcacttctggaggaacgtgatgagagtccccttcccccgagggacacatggcggcccaggctccaccagctctgttttcatgcggcggcaggtcaggctgggcagaattgtcaggccgagggtggcacgcacagcacacctctccagctagtgtcagaggccaccttcccttttatgacctcctgggctcctttgggactgactggcacctcttcccccaggacctgcaagcgcaggaccgagcccaccgcatcgggcagcagaacgaggtgcgtgtgctccgcctctgcaccgtcaacagcgtggaggagaagatcctagctgcagccaagtacaagctcaacgtggaccagaaggtgatccaggccggcatgttcgaccagaagtcctccagccatgagcggcgcgccttcctgcaggccatcctggagcacgaggagcaggatgaggtgagcccagcaccggccccgacccctccccagcgtgaatggtggacgcgtgagcggctttcatttttgtttttttaccttttttgcactcttattttttttgcatccctttggagtaaagggagtgtgggctgaacggaaagaggatgagtacttgctttttctttgaagtggtttttttttctaaactgctggtgaaagacgccggattgacagccctggagactgaagtcctctatttatccacagagcagacactgcagcacgggcagcggcagtgccagcttcgcccacactgcccctccgccagcgggcgtcaaccccgacttggaggagccacctctaaaggtgagaggggtagttcagtctccatgcccattcaatcctcggcttctcggctgagacggccagcaagggccctggtcccacggagcgtgcgtgtgcgtgtgcgtgtgtgtgcctttcgctgccgtgtgggtccccatccaccgcagccgtgccgggaccaccagctcattcccacggacgccgccgctcgcctctgagctcggccgccgcccaccccggcccctcctcagcggcactgacagtttgcaatcttataggaggaagacgaggtgcccgacgacgagaccgtcaaccagatgatcgcccggcacgaggaggagtttgatctgttcatggtaagcgctgcaggctggatggggcagttcaggcatcccactctgctgccaccaggagcaaagcagacgtcctagtgcccatggtggtatccctagcaggtcagggagccagggacagctcacagtgcagcccactcccacctccagactgacccgtcttccacccccagtctcctgaggatggcatcggagggcgagatgcacacccagccttctgcatgtgacccgagacctgccccaccagctctgttttctaacgggctctccagggcttcatgcactccctttcagagggagttcgccctatccaaggccaagggactgaccaggccttcagtcgcagagcccccttgcccctgggtgggaaacaggaaataagccacccaagcaggggccccttggcccgcaggcctcatgcctccaccaacgctgggccacgcagctgctgcccccctgctggggtctgcagccctcttgtgcaaccttccatcttttcgagtttcctctgcctcctgaggcagagcctctagtcagggtctgacggagccaggccaggtcagccactgaaaaatcgagagctactgtttaactctcgcagcagcgtggagccccacgggcagagaaaggcccttctgaactctcggtgttctggctctagcgtgcccctggtgcctgcatgctgatgcctctcccgttgcctccctgcccaccagcgcatggacctggaccgcaggcgcgaggaggcccgcaaccccaagcggaagccgcgcctcatggaggaggacgagctcccctcgtggatcatcaaggacgacgcggaggtggagcggctgacctgtgaggaggaggaggagaagatgttcggccgtggctcccgccaccgcaaggaggtggactacagcgactcactgacggagaagcagtggctcaaggtacatgctggagaggcccagcagctgccgcaggccagcgccaggcagggctggggagacaaagggcccaccgccaggactcaggcctgggtccaaaatgctttccttgggccactcctggccaggctccgcaggcagccgagagccttccgatgtgggccagggatggtcaggtccttttggctctgccttggaatgcaagaaggacccacggttcctgagcagctcaaaacctgctgctggttatggttggtctttcaagtaaaagggtttacctcttcccgaggttaaaaatcatgttgctgatctcagtggactgctggctttcccagtacctgccaggagggaggcacaagggtgaatcatggactattgaggcgccctgcaaagcgttgcactgctggctgcaggcagcctcactttgggcaagtccccccccatgagctggggagactcagtcatggggagggaggagagggcaagcccccaagggcctctgaacctggcaggtgctgcacggggctggcagttgcttgtggagctgacgtgagggcccgttctcagcagatggcagctggccctgcaacagcagctgcagatgtctttgagtcagtcgtgcggtgctcctggggcttcacccgacccaactttcccagcccaaggcacactgtgacagatgcctggcactgatgtcctcactggggttacggtgacagatcagaccccttcccagctcaggggctctgagttatagagacgatgattcagaatccatccctgaagttgtgtcataaaaatgtcctgaagcaataattccgagaaggcaggtggcagcttttggctatgccatgcaaagtgccacacccaggaaagagccatgtggcctagcccatgtggccaaacagccccggttccctcctgggacctcagcagcccagactcatgagcttgcctgccaagctgtgcaccagagggcgctgttgaaacacaccaggagcccaggtgacagacaggccatcctgttccccagccagcctcccaagcacagcgctccaggacccagaagagaaaccctgaaaagacagtgtgagccctgccacagacctgtgtgacagcagagctgtttggctgctgtatgagtgtcaccggccctgcatttttttcttttttaataaagacaaagtcttgctgtgttacccaggctggcctccagttcctgggggctcaagtgatcctcacacctcagcctcctgagtagttcagactacaggtacacaccaacacgcctggctaattttaaattttttgtaaagtgggggtctcactgtgtcactcaggctggtctcaaactcctgggctcaaacaatccacccgcctcggcctgcagagtagctgggattacgagcatgagccacggtgcccagctcagccctgcatatttgaggcagtgagtaccttttaaatttaagatcagttcatacgtggctgctctatgtacatttttttctaggtgtaatttacataccatacagttcacccttttggggtatatgatacaatcatcttcagtgtatccaagagttgtgcatctctcaccacagtccattttagaacattctcatcgcccccaaaagaagccctgccccgatttgccattcctggtaattcatggattctccccagccccaggcaaccactgatgtactttctgtccatatagttttacctgttctgggcgtttcctgtcagtggagtcctacagcgtgggatgctttggtgtcaggcttttttcacgcaatgtaacatgctgacccatgatgcagcaggtgtgttgcattctttgatggtgaaataatcctgcctcgtacagatttgtcacattcggtttttctgtccattcttgatggacacttgggttgtatccactttcgggctattacaaacaatactaccgtgaacattcacgtacacgtttttgcgtgcacgcgtttctctttaatatgtgatttctctttaatatctgatttctctttaatatgtgcctaggcgtagagttgccggaacaatgttagttctctatttaaccttttgagggaccccagcctggtttccttggtggctacgccagttcacagttccaccagctgtgtatgagaattcctctttctctacatcctcagcaacccttgctgttccctcgcccgctgattctagccatcccagcacgtatgaagtgccatctgagtgtggttggatttgcatttccctgatggctaatggtgtggagtatctgttcatatgcttcttggccctttgtgtatcctctagaaagagatgtctagtcacatcatcctttacccaaatttaaaatatatttatcttaatattgaattgtaagttatttacaagtgccttatcagatatcatttgtaaatattttgttccattctgcacattctcttttcgcttccgtgatatccgttgaagcacagaagtgtttcattgtacttacagtcgatttttctttttcacctgtgccttggtgttgtatctgagaaaccatcgcctatccaaggtcacgaagacttgcccctgtcttttctcctaggagggttataatttgatctcttccatgtagatccatgtaagatccattttgagttaaattttatacatgatgtgaggtaggggtccatcttcatccatccaactggaaatccagttgtcccagcagcatttgttgaagagactattttttcccattgagcccatcttattacacagtatattcaaattcacagttgtgaaaaatattatacaaaacactgtctccccactactgttggctgtggaattggcaccacttttctggcagccatctgttgccaagtgaactcaaagccttcaaaatgctcctatggccgccatcggacatggccatgactgtggcagcctaaagcaacagaactattttctctgcattctgggccagaagtctgaacccacggtgtgggcagggctgtgttcctttcgaggccctaggagaggacccttccttggcttttccagcttctgatggttccagaagcctcctggcttggggcagcgtcaccccatctctgcctctgtcttcatggggacttctcctctgtgtccccttctgtgtcttttaagaacaatttttaaaacttaatcccaaaagtaaagacctcatttgcaaagaaggtcccattccgaggttccaggtagacatgagtctggggggcacccgccgctccgccctgggtctgcagggggccttctcctggtgcctcctgcctgctgtgccctccatgctgcttctttgacccggttgccttccatcctcctgggcccctcaggctctcagcttcccatcttctctaagacatgtctgctccatcattctggtgtctctctcttccctccttcctggcacctgacacgtttgttaccccacttgtggggtgttgctgtctgatcaccatcttccttccaagggtaggggaaagggccctggctggttcaagtcacagctctgtcctcagcgccaggcacagtgcccagctcactggcacagcctcagattccctggacacctggctccccggctgcattttttaccattgggataagttattagtaccattgaaaattaatacctggggattctcttaatattgttcatattcatgttcttctcagcagttttaccatgaggaatctgtcttaaacaatgtcagttgtcaatagcgttatttaaaatagcaacaacaataaaaaaaggtggtcagacctggcagcttgtgcctgtaatcccagcacttggagaagccaaagcggggggatcatttcagctcaggagtttgagaccagtctggccaaaatagcaagacctttgtctctattaaaaaaaaaaatgccaggcacagtgacacatgcctgtaatcccaagcactttgggaggccgaggtgggcagatcacctgaggtcaggagctcaagaccagcctggccaacatggcaaaaacccgtttctactaaaaatacaaaaattagccaggcatggtggtgggcacctgtcatcccagctacttgggaggctgagtcaggagaatcactggaacctaggagttggagtttgtagtgagccaagatcgcaccactgcactccagcctgggcgacagaatgagaccctgtctctgaaaaaaagggaaatgacctaaatgcccaagaacaaggggaggctaaattagggcacgttgtgcactgaaacactaaacagacattaaaaaattttgttgtagaaaattacaggaaaagatatccatgacacagccagcagtgtggcacgtgggctacaattccagcgtggccttcagttctgcacacgtgcgtcaaaggtggggagagttctggtggtgggtggcgctgagggctgcacaacactggggacgtgcctaatgcccctgaactgtgtactaaaacatggctgctttggtaaattttatgttatatatgatttataatatatttttttaaatgcccagctactcaggaggctaagatgggaagatcacttgagcccaggagttaaagtccaaccagggcaatgtagcaagaccacatgtctaagaaaatttaaaaatcggctgggcgcggtggctcacgcctgtaatcccagcactttgagaggccgacatgggtggatcacgaggttaagagattaagaccatcctggccaacatggtaaaaccctgtctctactaaaaataccaaaattagctggacgtggtggtgggcgcctgtagtcccagctactcaggagggtgagggaggagaatcgcttgaaccaggaaggtggaggttgcagtgagctgagattgcaccactgcactccagcctggtgacagagcgagactgcgtctcaacaacaacaaaaaagaaaatttaaaaataatttttcatacaaaaattagccaggtgtggtggcacgtacctgtaatcccaggtacttgggaggctgaagcaggagaattgcttgaacccaggaggcggaggttgcagtgagccgagattgcaccactgcactccaacctcggtgacaagagcgaaactccgtctcaaaaatataaaaataatttttaaaatgtcttaaaaaggccgggtgtggtggctcacacctgtaatcccagcactttgggaggccgaggcgggcagatcacaaggtcaagagtttgagaccatcctgaccaacatggtgaaaccctgtctctactaaaaatacaaaaattagctaggcgtggtggtttgcgcctgtaatcccagctactcaggaggctgaggcaggagagttgcttgaacccaggaggcggaggttgcagtgagccaagatcatgccactgcactccagcctggtgacagagtgagactccgtctccaaaaaaaaaaaaaaaaaaatcttaaaaagaagaaatagatggaggccaggcccagtggcttatatctgtaatcctagcactttgggaggctaagatgggagcactgcttgagaccaggagttcaagaccagcctgggcaatattatgagaccccgtatctacaaaaaatttttttaattagctgagtgtggtgacacgtgcctgtagtcccagctacttaggaggctgaggtgggaggatctcttgagccctgggagttcaagactgcagtgagctatgatggtgccactgcactccagcctgggcaacaggtcgagaccctgtctctaaaaataaataagtaaataaaagaaatagatggggagagtgtggaacagaggtgtcaacatcacctccgggtgaagggatttcaggagcacgttcttttctgtacagagaagatagttctttttttttggtcaagaaattcaaccattagttttttaaagacaagcttgggtggggtgcctgctgggggcagtgctggtctttcactgcagcccaggcaccccttgagagtcccagtgtgtgttatgccccgagccagtcaagctgaagggagagcgggtgcgggggccctcctccgtgtcccagcccggcccctgggattgcgtcgcggcctctgcttgtcgacctgggtgctggctgtcctattttactactattgaccctgaaggccatcgaggagggcacgctggaggagatcgaagaggaggtccggcagaagaaatcatcacggaagcgcaagcgagacagcgacgccggctcctccaccccgaccaccagcacccgcagccgcgacaaggacgacgagagcaagaagcagaagaagcgcgggcggccgcctgccgagaaactctcccctaacccacccaacctcaccaagaagatgaagaagattgtggatgccgtgatcaagtacaaggacaggtaagcgaggaggcggggagggcgggggctgtaggggtccccgtgggagcaggcctggcatctgcactctgactctgcacactcaggcttgggccgctcactctttcactcatccacaaacactgactgaatctctgtgtctttgagcctggccctgaggaacatgctttctggaacagggaagcacacatgtatctgcggtgatgagagggaatgtcacatgtggtcgagaggagaggcaggggtgcgggtctcggagaaggggacattgcagcagagccttgagagaggggaggttggggagagtgcaccagcagaggccacagcaggcggagaggctctgccaaagttgtggcctggtggcctcaagggatgaggtgggatgaggggttatatggcagtaatgggtgctgcccgtgtggccaggaggttggggacaagaggctggcatgtctggagcagagaaagtggcagccctctcctgtccttgcaccctcccaggctacctgtgccatctctgtgttggtgagatggtggccacactcctgctctctgccaggccctgctccaagcctggagatcgctgtcctcacagacatgtcctatatacgaggcagcatgcggttccagctgtggccacggcccagcggcggatcataacgtgttggtggatcatgagtgccatttatatgtttttcatttactaatatccttttccgtgagcgactctatatgggtaccatttaaataagaagaaaggaatgttctcagcaactccccagggaggcatgtgcaccttctgccagcccagggcaggtcacagagctgggcaggagccagcaaaggtgtaaggcccatgcctggcccaggactcacccagtcttccactctccaaggtcttatttcacttcctggtacacactggccagtacggctgtccccgtaatgggagggttgtactgtttgattttaaataacagtttgctgttagtcatgaagcagagcgtgggggaattgtatctgttggaggggaaagtgaagggagcccacatgcatggtttggagagaatcttaccaagtgtgaagttttagaggatggtaacaattggagtgcctaaattaatgagtcaaaattaccgcgcaaggccagctgtggtggtccacacctgtcagcccagcactttgagaggctgaggtgggaggatcacttgaggtcaggagtttgagaccagcatggacaacatggtgaaaacccatctttactgaaaataggaaaacaattacctgggcgtggtggcacgctttttgggaggctgagacaggagaatcacttgaacccaggaagtggaggttgctgtggaccaagattgcaccactgcactccagccttgggtgacagagcaagacgcccactccaaaaacaaaatactgcttaaaaaggttcttttagccagatgcggtggctcatgcctgtaatcccagcactttaggaggccaaggcgggcggatcacttgaggtcaggagtttgaaaccagcatggccaatatggcgaaaccccctctctactaaaactacaaaaattcgccgagcgtagtggtgcatgcctgtaatcccagctactcgggaggctgaggtaggagaatcttgaagctgggaggcggaggttgcagtgacccaagatcacatcagtgcactccagcctgggggacagagtgagactccgtctcaaaaagagaaaaaaagaaaggttcttttattattggaagagaaacagatgatgaaggaaaggagacatattcaagttcaatgccagatctgaaaatgtcatgtgttgtcagtattgagaaaaaaatatatagactctaattcacaaacctaactccaaggctaagtcctcaaagaggaaagcaagtggaggacttaagttacttaaagtgcgggatacagctgcgggcagtagcacatgcctggggtccccactacttgggaggctgaggtgggaggatcacttgagcacagcaggtccaggctacagtgagccatgatcacacagctgcactccatcctgggtgatggaatgagaccctgtatcttaaaaaaatgaaataaatacagaatacttatttaaaacatgggccaggtatagtggctcgtgtaatcccagcactttgggaggctgaggtgggcggatcacttgaagttaggagtttgagaccagtctggccaacatggcaaaaccccatctctacaaaaaatacaaaaatagaaattaaactggcatggtgatgcgtgcttgagctcccagctcgggaggctgggcacgagaatcgcttgaacccagaaggcagagggtgcagtgagccaagattgcaccactgcactccagcctgggtgacagagtgagacaatacctgaaaaacaaaaaacaaaaacaaaaaaagggaattcgcagaaatctaatgacaatgacagaccctccccatgttttatttctaatgataatcttgtgagggaaacttaacattaccaacctaaagaagacccttgaagcgcagtggggaaagcacgggtgtctccatcacactggtctcctgggtatccacgtagagaggatgagcctcgaccacccccacacacgcacacaccatgcacttggatccacgtgacatggactgtggatgtaagaacgtgaaaggtaaaacaaaattctagaagaaagcacgggagaaaatcttcatgagtaggcatcgatttcttccacgggagccaaaagccactaatcacaaacaattgagaaattagacctcactgaaattaataacatctgtttattaatggacacgctgatggagacagtgaaaactcctcatagatgaggaaaagacaacacaatttaaagctggacaagagccgggaacagacgcctccttacacaggacatcccaggactaatcaatgtggggaaaggtgctcacgtcattgcattagcatcacaacttcaagaaaatgctcgttaaaaccagagtgacttgcaccagcacccgccccagaacggccgaatgcccatcagtgctggcaagtctgaagcagtctggcctcacaccctgcgtgtaggagtatgcagctctttggcaatctcttctgttttgctttgtttgtttttaaccatcgaactgcagaagataatattttctgaggctcagcctgtacataccctctgacccagcagcctgcagccctcgttctacccaggagagctgagtgcatctgtccacaccgacagacgtgtccatagccactttggtcataatctcccaaaacaagaatcagcccagtgtccatctacaggggcatggatcagtggaggcctaatcgtacagggccccaaacagctgtgaaaacagacatagtactgctacctggaagaagcttacattttgttgagtgaaagaagccagacactaaaaagggcatactggccgggcacggtggctcacgcctgtaatcccagcactttgggaggccgaggcgggcggatcacgaggtcaggagattgagaccatcctggctaacatggtgaaacaccgtctctactaaaaatacaaaaaattagccgggtgtggtggtgggcgcctgtagtcccagctactcgggaggctgaggcgggagaatggcatgaacccaggaggcggagcttgcagtgagccgagaccgcgccacggcactccagcctgggcgacagagcgggactccgtctaaaaaaaaaaagagggcctactgtgtgcctccagttctgtgagggtcagaagctgatccacagtaatggctgccaagcagtgcttaccttgagagagggaatagcaggaaagccacacgacgagccttctaggatactggggatattctctctatagatctggatgtgtatatgatgtgtacattcaccaaggtgtacacatgagatatgtacacttctctgtattccattacaccttggtttttgaaaagacagaaacagcatgctcagcttattaagaacctgaatattttcagacaaaaagtatgtctagtgttagtccacttttttttttttttagttttagactcacagagccctcttagggtcatatttagttgtatatcatctgacaaaagagggaatggctcacccagctgctacagtcagtcttgtagcctttgtaagatttgatgataaatcatacaataaattaaaactttcggctgggcacagtggctcacgcccataatcccagcactttgggaggccgaggcggaggatcacttgagcccaggagttcagatgagcctggccaacatagtgagactctgtctctatttaaaaaaaagaagaaaaaaaaagtctgggtgtggtggctcacgcctgtaatcccagcactttgggaggctgaggcgagcggatcacaaggccaggagatcaagaccatgctggctaacacagtgaaaccccgtctctactaaaaatacaaaaaattagccaggcgtggtggcgggcgcctgtagtcccagatactcgagaggctgaggcaggagaatggcgtgaacccgggaggcagagcttgcagtgagccgagattgtgccactgcactccagcctgggcgacagagcgagactccgtctcaaaaaaaaaaaaaaaaccccaaatcttaactcttactgctaagtgactgctttatcgaaactgttctgtttcgggacatttgattctggtagagattttgagacctgctttgaagaggacaccaacatttcagtcacacagcaatgctcctgtcttacctgggatccatgtgggaggatgcctcagtgggggatttgatctgtgttttcaatttaacctcacatcttactggaatggatggattcacagacctgggggtcgcccattggactgggagtaactacgttggaattacaagtaggacaacaaagatgactattaaagaaagcctcctgagtgctggaagcacagcaggcgtgggtctgtccactcactgtaatgtgatttgcacaggaaaacatcacatagggcacatcaactcaggaaagatagtcgttgaaaagctacctcagctgggcgtagtggtcacgcctgtaatcccagaactttgtaaggctgtgaggcttgtggatcccttgagaccaagagtacgagaccaacctgagcaaaacagggagaccccgcctatgcaaaaaatacaaaaattagccaggcgtggtggtgcactcctgttgactcagctattcaggaagctgaggcagaaggaccacaagaagtcaaggccgcagtggggtataatcacatcactgcactccaccatgggtgacagaggtgagaccctgttgcaaaaaaaaaaaaaaaaaaagcaaaagaaaagctgcctccatttagcaataatttcaaacatgatgtttgggtacaaaattggtgtacttaatggatatttctgatattcttaactaaatttgaagctgtgagggaagacaaactcagggtccacagggtgggaaaagtccacaagatcaccctcaagtgccgtcattctctaggaccccgagagccctagcggttgttacgcttgcagttatgatttattacagtgcaaggctacagattaaaatcaaccaaggcaagagatgtatggggcctcttgggggccaaggtgggcggatcacttgaggtgatctggacggttccagacacagggctttcctttgtctcctccctgtggagttgaagacagcgctgatgttcccggtgacgttgacacagagcactgccaaccagagaagcctccgtgtccagaggtttttttttttttttggagatgcagtctcactctgtcactcaggctggagtgcagtggcactatcttggctcactgtaacctctgcctcctgggttgaagtgattcttctgcctcagcctcctgagcaactgggattacaggctcgcgccaccacgcctggctaatttttgtattttttagtagagatggggtttcaccatgttggccaggctggtctcgaactcccgaccttaagtgatccgcccgcctcagcctcccgaagtgttgggattacaggcatgagccaccgtacccaccctgtgtccagcatttttactgggacttggtcacaaagacacagctggctacccacgtggctgacctccatctccagcccctctgcaggtgagcagaaactacatagcccaaagccccaacctaggactggtcccagaccccacgtaaacaaagacagccattaggtgtgacattctaaggactcagagatcacctcccaggagccccaggcaaaagccagacctcatttggggcgaagttacattcttttttattattattattatttgcccatcattgtctagaagttacattctgtatgacacatctgtcagatccctggaagcttcctcgccaccgactgcatggcaggtggtgctcatgtcactcggcccttccttggccccacacagctgcaccaggctgagaagagcttgaatggaataaagtcagacacagctaacagctcacttttttgtctccaactttgaattgctcctttccaagaaaagaaaaaaaacttctttttgtttaattggatttttttttttatttattttttgaggcagagtctcactctgtcacccaggctggagtgccatggcacaatctcggctccctgcaacctccaccttccaggttgaagtgattctcctgcctcagcctcccaagtagctgggattacaggtgcacaccaccatcatgcctggccggaatttttgttttttagttggctctccagaaccctctaagtagttcagctctgcaattgagttaaatgtgaagcctgaaaagagactggcatctgtgtagacctgagttctaacaattcaaagagtgaacacgggacgttgtcctctggattcggtgtcaggagcctgcctcgccgagctggagagtgtgcccatgactgcaggtgggagccacatggtccgtctaggtggcgacacctgaggacagaagtggtttagctgggcacccgctgctgtgttggggccagacccttttggagaggcagggacgcccttcctgtcaggtggaatcttttcaaatattgcttttgatcgttggagatacttgcagccccctagttcgtgttttattctggcagaattgtgtttgacgtgacctctgtgttgacctcatcatacgtgtacagttgtcttgcctgggaatcttatactctgttcttctgcactcagattcacagccatgaggccgctcctcccaaggccctggagtgtggcactgccagctgtcacagccacggtcctttacattgtatacacagagtgcagtgacatgcccctcctgctcaggggccacagtaggaacaggctggagtgttctgtggtaccatgccggcagtgctggcttctgctgtccaggggcttgtggtggctccagtgggaagggccacgtaagcagaacaggaagtgtgagcagtgtccactgggaggagagggacaagtgtcccaggcagagaggacacagtgcatgcagaggctggagggaggctaggggcttgagtggggctgcgccagcaccaggggcctcacaggggccctgggccacacacctgcccttagccaagggcgtttctccttggtggtttgaaatctcaaaccacattgaggaacctgccagaaatcgaaaccagaattaagaaattaagttacatgtaggagattttactttaaaatcagaacatgcagaaacgttctctgtggcttcgggtcaggttcgggcttgtggggctgtacactgccagttgataccttcttccctgggttctgtgtttttttttttttttttgagacagagtcttgctctgtagcccaggctggagtgcagtggcacagtcttggctcactgcaacctctgcctccctccctgggttctttatagcagtcagcagctggtggctgaggcatttggggcactgtgtccaagtgtcaaggccgcatgtgtgtcagggggcacacccttggtgtgcttggtaagggctcagagaagggcatctgctggtccatgtgggctcagccagcagcctgctaaagaccaccccgccagccaccagcctgcctgcgacagtgatagggcagagatgctgctacaataaagaccatgcgggccaggcgcaggcgctcacatctgtaatcccagcactttgggaggccaagccgggctgatcacttgagctcagaagtttgagaacagcttggccaacgtggcaaaaaccccgtctctattaaaatacaaaaattagccaggcatggtggcactcgcctgtaatcccagctacttgggaggctgaggcaggagaatcgcttgaacccaggaggaggtttcggtgagccaagattgcaccactgcactccagcctgggtgacagtgagactacgtctcaaaaaaaaagactgtgctagacggtggtgttgaacttgaacttgatgcttgtgctgaggtcaggagacacgtgcacagcttcagggctagaggggcacaatggctgcagactatgctcgggcagctcggaaagacggtgacgggtccaggctgccgcagctaagcctcacaagagctctggccccaccaggcgggccctctgggttccattcccactccccactgcctggaggcagcagccctgtggttactgtaggagggacctgaatgcagtgaagatctgcactccacacctgtctctgtgcctcagtctccacgtctgcagcacaggggcctgactgaaataacgtggccaggcacacagcccagacgccagcactgtgtgcactgtgggaggggacgatggtccctagcccagggcctgagctgctgccttgccaattcgtggtgacagctttggcctgggtctggcaggggctggctgctactgctgaaggcctgtctgcactgtttccctgcctttccacaagcactgtccctcctggctcttcagagttgagtggttataccttgtgcaagaaaaacaatgaaacagattgtctgaatgtttcagacctgcctgaaacattctgcaggagaggtcatcccaaggcttgaaagtgtttgtcacactccttcaagcccccagggcagggcccctgagggaagggtgcaggtggagggtgggcaggagtgggagccattccttggggacaggggggctcctcagggctgtctggcccttggcctgtggttccaggtctatggtctgcttggcacatagcctggaccctggtgctgtgcaggcactggctggatgagggcaccgagcagatgagccccaggcatgcccacttatgggcataggctgggtgcagggcactgggtccaacataaccatggcagagggctcagggggcctaaaagtcatggatggagttgcccacgcagcctgcggtcaggttccctgtggagccgtgctggggttctgcgcctgccccaggatttgctggaagaagctccccctcctctgtttgtgagtcgggccctacccccaagtctccccctcagtgatagagacctttacataacacaaataaacaaggcaactggtttagaagaatgagtctgaaaactagaatggcgtgtttaaggaaagcttaggacaggacaattagagacagaggtgcaggtgtgtggagttggcagcaggaggcctgggggagccgctcgtgcctttccctcctgccctggcctcccttggaactcggagcagccccctgccttgaggaggcccttcctttcagtgagcctgtgccgcagtcgtccgagggaacacagtggggacatatttccttttttttttttttttttgagacagagtctcactctgtcgccaggctggagtgcagtagcctgatctcagctcactgcaaattctgcctcctgggttcaagcaattctcctgtttcagcctcccgagtagctgggactacaggcatgcaccaccacgcccagctaatttttgtatttttagtagagatggggtttaaccacgttggctaggatggtctcgatctcttgacctcatgatccgccagcctcggcctcccaaagtgctgggattacaggagtgagccaccatgtccggccggaaatgtttattaaaatagcagatgaggccgggtgctgtgactcaggcctgtaatcccagcactttgggaggctgaggcgggtggatcacgaggtcaggagttcaagaccagcctgaccaagatggtgaaacccatctctactaaaagtacaaaaattagccgagcgtagtgagacttgcctataatcccagttacgagggaggctgaggcaggagaatcgcttgaacccaggaggcagaggttgcagtgagccgagattgtgccactgcactccagcctgggcaacagagcgagactccgtctcaaaaataaataaataaaaatagatgatcatagacaggagggatgaagtagaagattcaaagcagcttaaaaaaaggaagtaaaaaagaaataaggtcaggagacaagattgagtgactgtgaaacatcaacaataggcagatttaaaataaagtcaggaaatagctagatcaaagaatagaaaggtgaaccaggtgtgatatccacctgtagtcccagcttcttgggaggccaacgcgggaaaatcacgtgagcccaggagttggaggcaccgctgcactcaagcctgggtgacacagtgagaccctgtctcaaaaagcagcaggaagagaggctggcatgtggggctccagggggtgtgaaggagggtccacaccctccttcctccacctggccatactgagcagcacattctctgagccaacggcccctacaaacacccaccctttcctcctggtagcccagagggcgcccctgcctgctcacttcccaatgtcgggatcctgtgatttctgcctcttcagaaaggacttgaggcattttctgactcagagccactcaggattccaggacctcctgtggtgacaacagggacaccagtagtgatgaattcagaaaagagcgaacgcccctcaagctgccctcccccagccctgcagacagtggccatgggcctccctgcactcaggaccgactgcacctgcctgcggcgtaggggagctggggctggcctttccttttcatcttcccctggcactgggaaatgctctgggtggtgccatgtataacaccctttgtcacagtgctctcatttcgtcgtgtaaaaggccagtccccgcctcgcagtgttggggcagccagcacaggcacgtgagacaggagccacacctgggttctcatctggcatctgcctcttgccatgctcaggttcttgcctccagatatagggcatcaaactaagtggtttccaaacttgggttcttggtggggagtgagatcagggtttcttaaaaactgcacttggaggctgggcacggtggctcacgcctgtaattccagcactttgggaggccgaggcgggcggatcacctgaggtcaggagttacaaaaaacggaacctaggctcatgcctataatcccagcacttcgggaggccaaggctggcgaatcgctggaatttaagagttccagaccagcctggccaacgtggcaaaaccctgtttctactcaaaatacgtaagttagccgggcatggtggtgggtgcctgtaatcccagctactcgggaggcggaggtttcagtaagccaagattgcaccactgcactccagcctgggtgacagagtgagactccgtctcaaaaaaaaaaaaaaaaagaaagaaattcaaatctcagtgtcgataaatgaagttttgttggaatgcagccaaagcccaccgatttctgcgttgtctgtggttcccttcatggaacagtggcagaactgagcagttgcaacagaggcaggcccagcaagcctcaaatatttacactgagctgagcgcagtgactcacacctgtaattccagtgctttgggaggctgatgtgggaggatcccttgagcccaggagtttgagaccagcctgggcaacctagtgagaccccatctctacaaaatattttttaaattagccaggtgttgtggcgtgtgcctgtagtcctagctacttgggaggctgaagcagtaggatctcatgagcccaggagttggaggcttcagtgagctaggatggcaccactgcactctagcctgggcaacagaacaagatcgtgtctgtaaaaagagagaaaagaaacagacaattcctagactgcagcaggtgtttgcagtagtgcatccagcaaaggactccccagggaaggaaatgggccaaagatgggaacaagcgctgcttggaagagcctgtgaaacagtgcttggcttctctgcacaacaaggaaacgtgtttagggccacattgtagaatggctacaaggacaagcaccactcacgctggctcagtatctcccaagtagagcatttccccaccctgtgacctagagtatcttctcccagataaattcctaggagaactgcaaactaaacacttgttcctgagtgtccacagacacttgcttcctggtgtccaaccctgagcacagcccagatgcccggcagcgggaactgggcatggctggtgtgtgcccacctcgggacctgctgctgcactcggcaacagggacagcaaggaggcaggaggccaggccaggtttgggggctcccctgtgtgtttctgcatcatcaacttgaagaagcagccaacctcggcccaggatcatccctgggggaaggactgacaggagcgcatctttagcctcatgcttaagatctgtgcattgggccaggtgcggtggctcagcctgtgatcccagcacttttgggaggccaaggtgggtggatcaggaggtcaggagttcgagaccagcctgaccaacatggtgaaacccgtctctactaacaatacaaaaattagctaggtgtggtggtgcacctgtaatgccagctacttggaggctgaggcaggagaatcgcttgaaccctggaggcagaggttgcagtgagccgagatcacgccactgcatgatgtgtgcattggtgttaagtcagctattaagaggtgtgggctgggtgtgtgtgggggtcagaggaggtgatgtttgccctgagatgaggtcagagcccacattcccatgggggtggactcccctttgtaggccttgacacgagcatgggagcggagctaggggcactgacatcaaaggtggtggcgcctatccagaggactccgggtttcattccaagtgcggggaggctgcagcatgtccctctttaatatagaggtgagggaggctctcagcaccctgcctgttgcagggaagcctgaggacaggacctccagggaactctgcaccagagcggaatcctcagaccggcaactgggtggtcttgattcatccagaacgtaattctcctagaggtctgggaaggggtgctcttgtaccttggttttgtgttttgtttttgtttgttttttgagacagagtctcactctgtcgcccaggctagaatgcagtggtgcgatctcggctcactgcaacctctgccacccaggttcaggtgattctcctgcctcagcctcccgagtagctgggattgcaggtgcctgtcaccacgcctggctcatttttgtatttttagtagagacggggtatcactatcttggccaggctggtcttgaactccggacttcgtgatccacccacctcagcctcccaaagttctggggttacaggcgtgagccactgcgcccagccgcaccttgttttttttttgctgcctgtagctgtttctcattagcctgatggaggtggctgttggggtggtggggtgcttattataaagccttctgctgtcacagccctggggtctcacttcctgggctctctgcatagggagccgccctccagggcaagggccacagtgtgcagtaagccccccatggccacagggcagggcgtttcaagccaagagtctgcactgtttgctggccactaagggcattgaatggatcacaaaactggcttttttttcccccagtcaaatagaaatggggttttgccatgttgtccaggctggtctcaaactcctgggctcaagcaatcctcccacgttggcctcccaaagtgctgggattacaggcacgagccaccatatctttaaaaatgtaatagaatggaaaatggcaaagcgcgctgcttttcatagaagagactatcgcattggggttcttctgttgcccctgcctgtgctgcacgtcctgcttcacagtgtaaagcgcaactcttgctgtgccttattgtaacaggtatagggtggatgctgccatcatgaagagagaggagagctctttgcttataaaattccgtagagtggagaacaggatgtgccggcttaggagagggaagaggtacaggcccgctctgcccgagctgcagatgggacacacatttcccgactcagaggggagtcgtgtctttctccagggtcaccgagcagcaagcgcatccactgtgtgttcttattttctgagcctgtggactgggtttaccaacagttgcctgtggcagagccggccaccagcccgctcataggaaaaaggaagcctgtgcctccagggagggcccacgatgggaatgcagtctgagaagggtatgtgctcccatgctggaccggctggtcccacacccaggaccaaaaacacctcctggcaggagatggggagatgtacttcatgaacacacgttggggaaggacactcctggaaggcagaggctcaaggcgggctgtgtccaggccaggagattcactcccacctcaatttcctcctcagcacggctggcctggcactggtgcctgcttcagagtttagtgtgtgtaaagtgtggctggcccagcaagcaggggatgatggcggttgtcaaggccaggggaggtgcagggacaggattctggctcccgtgtgtaacgtctgcatctcaggaggctctcatgttagaagctgggtcaggggcttcgtgatgcacacaaacaccaaacaagcaggaggccttccgggatcctgctcacgagcagcttcctggaggcaagcctggcccgtctgccccttggtgacatcaggaccccagcaacagaaccagggaagccaccatgttgtcgctgccattttccagcgtggtaaacagaagcaagacaaggcgcactcaggacacccaaacagacggcggggccagccagtggcatgggcgggctgccctgccctctggaattttctgggaaagaacataaggaaagaagcggtgctgatgaggcagctggagttatcaggacccaagctcagctcctccttcccatcaggccccgttggccacttttaggcttcgtccgcctggtggctccctgcttggggccgttccctggctaggcccttcctctgggccaaagcagagaggagagcgcgtttgccatgcgttctcattttcccagcctgcgggctgggttctgtcagagcaaggtcagctctcagagcttctgtccccacccaccctgtcctgctgagccaggctcaggtatgggtaagagctgggagcgctgtgcacgaggcctggcagcatctcatcactgccgctgtaccgagtgcacaagggcgcttcctgtggtgttcagggggttagatcagtccccagtgaggacagagcagagatccagatgcatcccaggccggctgcagtggcttctgcctctaatcccagcacttcgggaggccaaggcaggcagatcacgaggtcaagagatcgagaccatcctgaccaacatggtgaaaccctgtctctactaaaaatacaaaaattagccgggtgtggtggctggcatctgtaatcccagctactcgggaggctgaggcaggagaattgcttgagcccaggaggcagaggttgcagtgagccgagatcattccattgcgctccagtctggcgacagaacaagactctgtctcaaaaaaaacaaaaaacaaaaaaatgtatcacaggcctgtttgtgtggttgtctctgggaaatgttccagaagctacaagaaagcacatgtgcaggccaggcatggtggctcacacctgtaatcccagcactttgggatgctgaggcgggctgatcacctgaggtcgggagtttgagaccagtctgaccaacatggagaaaccctgtctctactaaaaatacaaaattagcctggcatggtggtgcacgactgtaaaaaatcccagtgactccaaaggctgaggcaggagaatctcctgaacctgggaggtggaggttgcagtgagccgagatcgcaccattgcactccagcctgggcagcaagagcgaaactccgtctcaaaaaaaaaaaaagcgcatgtgcaagaaatagctcctgagctgagttggaatttctcatccttaaacaatgtgggaacctgctgaggtggggtgggggctcccgggtgggcggactcgggggtgatagccgccggttctgccttgcagcagcagtggacgtcagctcagcgaggtcttcatccagctgccctcgcgaaaggagctgcccgagtactacgagctcatccgcaagcccgtggacttcaagaagataaaggtaaccctgacgttgtacctgcgccccgcatgtgcccggaggggagtctgacccaggggcacccccatctgagagctgtggtgtgtgggcagaatgaccagaaaccacctaggcggtgccttgggctacctggttagggacctggtcgtgggcttttggggttccttgtaagggttgggggtgtcctgagggatgtcagtgggcagtcggtgttgggtgttccttcaaggtcccactcacttagtgctgggcctcagtcatgcagttcccatgcctagtgagccaggttaccgaggctggcgcttcggccacatcctcataggcacgaggaatcctagcccgtggggtctccagcacacagccaggcctgcgggcaggcgaggcggggtcctgaggtaagacctgctcctcccgtccactgcaggagcgcattcgcaaccacaagtaccgcagcctcaacgacctagagaaggacgtcatgctcctgtgccagaacgcacagaccttcaacctggagggctccctggtgagggcaccgctgggggttggggatgggccactcccacagctgggctttgacccaacccgcccctccttcccttctgaattgatgggttaaaaacaagtcccgctagctgtggtggttcgtgcctgtaatcccagcactttgggaggccaaggcgggaggatcacttgagaccaggagttcacgaccagcctgggcaacataacaagaccctgtctttaaaaaaaaaaaagaaaaaattaaaaatttccaacttgggatattttagcaattacaaaagcagtatgtatttaccgaagaaaataaagataggcacaacagaagaaatagactcagagtcctctgtctgttggaattttattttaaattccctggggcacattctcttcaaaggaatcctgtgggtttgttctttcctgtgatagaattgacacatacacagcctcaacttggcaaactttcttaagtgtctttgggattgattcctctcagaaaaaagacttgactttgatgagtttgggaaagatgctggcttcaacaaggggcatgtatttccaatgttattgcagacctcagagattgtgcagaaggagggaggcacctgcagctattcccaaaccagtgtgcctgacctttcaccctggagcatttcagaagtctgatgctcgggcccctatggaggaagattatggagagggccttccaccctgggtgggctgaagccccgacccgctgaggctcgcattggccactgatcagctgtccaggggcaacacagggctggggctggggctggggccagggccgggcaggcagccctccagtcgggcccatccactcaagcccctggtgtctctgcccagatctatgaagactccatcgtcttgcagtcggtcttcaccagcgtgcggcagaaaatcgagaaggaggatgacagtgaaggcgaggagagtgaggaggaggaagagggcgaggaggaaggctccgaatccgaatgtgagtcccgggggggttcaggacgccggggttcacgctggcccgagagcccccaaggccccagcttttcacagccctcccggctcccagacgccccttgctgtgggggtgctgcattcccagagctcaaggctgtctttccctcccggtcccctccagctcggtccgtcaaagtgaagatcaagcttggccggaaggagaaggcacaggaccggctgaagggcggccggcggcggccgagccgagggtcccgagccaagccggtcgtgagtgacgatgacagtgaggaggaacaagaggaggtgaggccgggcccccgagcaggcagagctggcatgtggcaggaggcatcccggggccctgatgggacagccctgtggggcggtgctcccacgcccccacgctgcaggtgggaaagctgaggcttgacctgccatggctgaccccaggtcacacagccagtatgtggcagggctgccactcaaagccaagcctgtgtgaccccggaaccagctctcagtacccgtgggtgtctggggactgggggacacgtgagtcctcaggacaggcagcaggcggtcccagggcaccagggctcctgggctgaggccgggcaggccagggcttgattagggttgggtaggaggtgctaggattggggacccaccagacagtgccccgtctcctcagtggcatagagaatgggcagaaacgccaagaccacagtggatagtttttggggtgcagtggggagccggagaagatccacttgctggctttaaataatctcacctctgtctctggcccttcctggctgtgggacctggcaggtcactttgcttctcaagcttcagtttcctcttatgcaaagcaaggaggctgcatggattagggggctgcccccaccgaagggcctggctcccgctgaagcccagcgaatgccattttgtcatcacagaagcagcaaatttcagtgcctgggaattagtctgacgaaagacgcacgtgacgtttgaggacattcatgaagtcctcatgtggaggggaggcccctgtgtcaggcaggagggtctgggctgtgatgctgtctctcttgcctaatccagagcagggtgttttatggcccatgccaggctgtttaaaaaggttttatgaggccaggtccagtggctcacacctgtaatcccagcactttgggaaactgaggcaggaggctcacttgagcctgggaggtcgaggctgcagtgagccgtgatagcaccactgcactccagcctgggcaacagagcaagaccctgtctttaaaaaaaaaaaaaaaaatatatatatatatatatatatatatatatatatatatgaaagagcaaagatctttcacagtcaggaagaaccaagatggtttggaagctgtaggtcaggggccaagggccaggcaggtgacatgggacagagcagggaaggcacgtggagcctcgaaggtcccgagagggccgagtgtggtcacagggactcgtcctgtagagaaacatgaagtttgggcatggccatggcgccatctcggctcactgcaagctccaccccccgggatcgcgccattctcctgcctcagcctcccaagtagctgggactacaggcgcccgccaccacgcccggctaattttttgtattttttagtagagacggggtttcaccgtgttagccaggatggtctcgatctcctgacctcgtgatccgcccgccttggccttccaaagtgctgggattacgggcgtgagccaccgtgcccggcccacatcagtgttttcttgagcaagtttatttaaagtttggcaggtccctggcaaggtgccctggcaggggtggccaacgcacactctctcctcctgtcccctctccaggaccgctcaggaagtggcagcgaagaagactga

SEQ ID NO: 2 - DNA, 4944 bp, Homo sapiens
  CDS (1)..(4941)
atgtccactccagacccacccctgggcggaactcctcggccaggtccttccccgggccctggcccttcccctggagccatgctgggccctagcccgggtccctcgccgggctccgcccacagcatgatggggcccagcccagggccgccctcagcaggacaccccatccccacccaggggcctggagggtaccctcaggacaacatgcaccagatgcacaagcccatggagtccatgcatgagaagggcatgtcggacgacccgcgctacaaccagatgaaaggaatggggatgcggtcagggggccatgctgggatggggcccccgcccagccccatggaccagcactcccaaggttacccctcgcccctgggtggctctgagcatgcctctagtccagttccagccagtggcccgtcttcggggccccagatgtcttccgggccaggaggtgccccgctggatggtgctgacccccaggccttggggcagcagaaccggggcccaaccccatttaaccagaaccagctgcaccagctcagagctcagatcatggcctacaagatgctggccagggggcagcccctccccgaccacctgcagatggcggtgcagggcaagcggccgatgcccgggatgcagcagcagatgccaacgctacctccaccctcggtgtccgcaacaggacccggccctggccctggccctggccccggcccgggtcccggcccggcacctccaaattacagcaggcctcatggtatgggagggcccaacatgcctcccccaggaccctcgggcgtgccccccgggatgccaggccagcctcctggagggcctcccaagccctggcctgaaggacccatggcgaatgctgctgcccccacgagcacccctcagaagctgattcccccgcagccaacgggccgcccttcccccgcgccccctgccgtcccacccgccgcctcgcccgtgatgccaccgcagacccagtcccccgggcagccggcccagcccgcgcccatggtgccactgcaccagaagcagagccgcatcacccccatccagaagccgcggggcctcgaccctgtggagatcctgcaggagcgcgagtacaggctgcaggctcgcatcgcacaccgaattcaggaacttgaaaaccttcccgggtccctggccggggatttgcgaaccaaagcgaccattgagctcaaggccctcaggctgctgaacttccagaggcagctgcgccaggaggtggtggtgtgcatgcggagggacacagcgctggagacagccctcaatgctaaggcctacaagcgcagcaagcgccagtccctgcgcgaggcccgcatcactgagaagctggagaagcagcagaagatcgagcaggagcgcaagcgccggcagaagcaccaggaatacctcaatagcattctccagcatgccaaggatttcaaggaatatcacagatccgtcacaggcaaaatccagaagctgaccaaggcagtggccacgtaccatgccaacacggagcgggagcagaagaaagagaacgagcggatcgagaaggagcgcatgcggaggctcatggctgaagatgaggaggggtaccgcaagctcatcgaccagaagaaggacaagcgcctggcctacctcttgcagcagacagacgagtacgtggctaacctcacggagctggtgcggcagcacaaggctgcccaggtcgccaaggagaaaaagaagaaaaagaaaaagaagaaggcagaaaatgcagaaggacagacgcctgccattgggccggatggcgagcctctggacgagaccagccagatgagcgacctcccggtgaaggtgatccacgtggagagtgggaagatcctcacaggcacagatgcccccaaagccgggcagctggaggcctggctcgagatgaacccggggtatgaagtagctccgaggtctgatagtgaagaaagtggctcagaagaagaggaagaggaggaggaggaagagcagccgcaggcagcacagcctcccaccctgcccgtggaggagaagaagaagattccagatccagacagcgatgacgtctctgaggtggacgcgcggcacatcattgagaatgccaagcaagatgtcgatgatgaatatggcgtgtcccaggcccttgcacgtggcctgcagtcctactatgccgtggcccatgctgtcactgagagagtggacaagcagtcagcgcttatggtcaatggtgtcctcaaacagtaccagatcaaaggtttggagtggctggtgtccctgtacaacaacaacctgaacggcatcctggccgacgagatgggcctggggaagaccatccagaccatcgcgctcatcacgtacctcatggagcacaaacgcatcaatgggcccttcctcatcatcgtgcctctctcaacgctgtccaactgggcgtacgagtttgacaagtgggccccctccgtggtgaaggtgtcttacaagggatccccagcagcaagacgggcctttgtcccccagctccggagtgggaagttcaacgtcttgctgacgacgtacgagtacatcatcaaagacaagcacatcctcgccaagatccgttggaagtacatgattgtggacgaaggtcaccgcatgaagaaccaccactgcaagctgacgcaggtgctcaacacgcactatgtggcaccccgccgcctgctgctgacgggcacaccgctgcagaacaagcttcccgagctctgggcgctgctcaacttcctgctgcccaccatcttcaagagctgcagcaccttcgagcagtggtttaacgcaccctttgccatgaccggggaaaaggtggacctgaatgaggaggaaaccattctcatcatccggcgtctccacaaagtgctgcggcccttcttgctccgacgactcaagaaggaagtcgaggcccagttgcccgaaaaggtggagtacgtcatcaagtgcgacatgtctgcgctgcagcgagtgctctaccgccacatgcaggccaagggcgtgctgctgactgatggctccgagaaggacaagaagggcaaaggcggcaccaagaccctgatgaacaccatcatgcagctgcggaagatctgcaaccacccctacatgttccagcacatcgaggagtccttttccgagcacttggggttcactggcggcattgtccaagggctggacctgtaccgagcctcgggtaaatttgagcttcttgatagaattcttcccaaactccgagcaaccaaccacaaagtgctgctgttctgccaaatgacctccctcatgaccatcatggaagattactttgcgtatcgcggctttaaatacctcaggcttgatggaaccacgaaggcggaggaccggggcatgctgctgaaaaccttcaacgagcccggctctgagtacttcatcttcctgctcagcacccgggctggggggctcggcctgaacctccagtcggcagacactgtgatcatttttgacagcgactggaatcctcaccaggacctgcaagcgcaggaccgagcccaccgcatcgggcagcagaacgaggtgcgtgtgctccgcctctgcaccgtcaacagcgtggaggagaagatcctagctgcagccaagtacaagctcaacgtggaccagaaggtgatccaggccggcatgttcgaccagaagtcctccagccatgagcggcgcgccttcctgcaggccatcctggagcacgaggagcaggatgagagcagacactgcagcacgggcagcggcagtgccagcttcgcccacactgcccctccgccagcgggcgtcaaccccgacttggaggagccacctctaaaggaggaagacgaggtgcccgacgacgagaccgtcaaccagatgatcgcccggcacgaggaggagtttgatctgttcatgcgcatggacctggaccgcaggcgcgaggaggcccgcaaccccaagcggaagccgcgcctcatggaggaggacgagctcccctcgtggatcatcaaggacgacgcggaggtggagcggctgacctgtgaggaggaggaggagaagatgttcggccgtggctcccgccaccgcaaggaggtggactacagcgactcactgacggagaagcagtggctcaaggccatcgaggagggcacgctggaggagatcgaagaggaggtccggcagaagaaatcatcacggaagcgcaagcgagacagcgacgccggctcctccaccccgaccaccagcacccgcagccgcgacaaggacgacgagagcaagaagcagaagaagcgcgggcggccgcctgccgagaaactctcccctaacccacccaacctcaccaagaagatgaagaagattgtggatgccgtgatcaagtacaaggacagcagcagtggacgtcagctcagcgaggtcttcatccagctgccctcgcgaaaggagctgcccgagtactacgagctcatccgcaagcccgtggacttcaagaagataaaggagcgcattcgcaaccacaagtaccgcagcctcaacgacctagagaaggacgtcatgctcctgtgccagaacgcacagaccttcaacctggagggctccctgatctatgaagactccatcgtcttgcagtcggtcttcaccagcgtgcggcagaaaatcgagaaggaggatgacagtgaaggcgaggagagtgaggaggaggaagagggcgaggaggaaggctccgaatccgaatctcggtccgtcaaagtgaagatcaagcttggccggaaggagaaggcacaggaccggctgaagggcggccggcggcggccgagccgagggtcccgagccaagccggtcgtgagtgacgatgacagtgaggaggaacaagaggaggaccgctcaggaagtggcagcgaagaagactga

SEQ ID NO: 34 - protein, 1647 aa, Homo sapiens
MSTPDPPLGGTPRPGPSPGPGPSPGAMLGPSPGPSPGSAHSMMGPSPGPPSAGHPIPTQGPGGYPQDNMHQMHKPMESMHEKGMSDDPRYNQMKGMGMRSGGHAGMGPPPSPMDQHSQGYPSPLGGSEHASSPVPASGPSSGPQMSSGPGGAPLDGADPQALGQQNRGPTPFNQNQLHQLRAQIMAYKMLARGQPLPDHLQMAVQGKRPMPGMQQQMPTLPPPSVSATGPGPGPGPGPGPGPGPAPPNYSRPHGMGGPNMPPPGPSGVPPGMPGQPPGGPPKPWPEGPMANAAAPTSTPQKLIPPQPTGRPSPAPPAVPPAASPVMPPQTQSPGQPAQPAPMVPLHQKQSRITPIQKPRGLDPVEILQEREYRLQARIAHRIQELENLPGSLAGDLRTKATIELKALRLLNFQRQLRQEVVVCMRRDTALETALNAKAYKRSKRQSLREARITEKLEKQQKIEQERKRRQKHQEYLNSILQHAKDFKEYHRSVTGKIQKLTKAVATYHANTEREQKKENERIEKERMRRLMAEDEEGYRKLIDQKKDKRLAYLLQQTDEYVANLTELVRQHKAAQVAKEKKKKKKKKKAENAEGQTPAIGPDGEPLDETSQMSDLPVKVIHVESGKILTGTDAPKAGQLEAWLEMNPGYEVAPRSDSEESGSEEEEEEEEEEQPQAAQPPTLPVEEKKKIPDPDSDDVSEVDARHIIENAKQDVDDEYGVSQALARGLQSYYAVAHAVTERVDKQSALMVNGVLKQYQIKGLEWLVSLYNNNLNGILADEMGLGKTIQTIALITYLMEHKRINGPFLIIVPLSTLSNWAYEFDKWAPSVVKVSYKGSPAARRAFVPQLRSGKFNVLLTTYEYIIKDKHILAKIRWKYMIVDEGHRMKNHHCKLTQVLNTHYVAPRRLLLTGTPLQNKLPELWALLNFLLPTIFKSCSTFEQWFNAPFAMTGEKVDLNEEETILIIRRLHKVLRPFLLRRLKKEVEAQLPEKVEYVIKCDMSALQRVLYRHMQAKGVLLTDGSEKDKKGKGGTKTLMNTIMQLRKICNHPYMFQHIEESFSEHLGFTGGIVQGLDLYRASGKFELLDRILPKLRATNHKVLLFCQMTSLMTIMEDYFAYRGFKYLRLDGTTKAEDRGMLLKTFNEPGSEYFIFLLSTRAGGLGLNLQSADTVIIFDSDWNPHQDLQAQDRAHRIGQQNEVRVLRLCTVNSVEEKILAAAKYKLNVDQKVIQAGMFDQKSSSHERRAFLQAILEHEEQDESRHCSTGSGSASFAHTAPPPAGVNPDLEEPPLKEEDEVPDDETVNQMIARHEEEFDLFMRMDLDRRREEARNPKRKPRLMEEDELPSWIIKDDAEVERLTCEEEEEKMFGRGSRHRKEVDYSDSLTEKQWLKAIEEGTLEEIEEEVRQKKSSRKRKRDSDAGSSTPTTSTRSRDKDDESKKQKKRGRPPAEKLSPNPPNLTKKMKKIVDAVIKYKDSSSGRQLSEVFIQLPSRKELPEYYELIRKPVDFKKIKERIRNHKYRSLNDLEKDVMLLCQNAQTFNLEGSLIYEDSIVLQSVFTSVRQKIEKEDDSEGEESEEEEEGEEEGSESESRSVKVKIKLGRKEKAQDRLKGGRRRPSRGSRAKPVVSDDDSEEEQEEDRSGSGSEED

SEQ ID NO: 4 - DNA, 163717 bp, Homo sapiens
  CDS (1)..(225)
atgtccacgcccacagaccctggtgcgatgccccacccagggccttcgccggggcctgggccttcccctgggccaattcttgggcctagtccaggaccaggaccatccccaggttccgtccacagcatgatggggccaagtcctggacctccaagtgtctcccatcctatgccgacgatggggtccacagacttcccacaggaaggcatgcatcaaatgcataaggtaagagtttgttctcccattcaaactcaacttctgataagtggatggctaatatttctgataaccccatccccctttctactgttgttaacaagaaaatcatgcaagatctttgtcttgtatatctcatatattattaaaaacgcatattgtgaggtacttttattgctacagatgttgcatcagttatgaagatcagatgttggctttttaaaatactgacatagactatgcagttctgataagtagatcctgtgatttgtgggttgaaggtatttttgtcttggtgaattagtacattagtctataagtcttattttttcctatctgtgttaagtaacttatccactaatgaacatactgaaaattattctttcctaaaaaggctgacatttattaacaaaaattagatcccaaagagtagaaggaaaaagtccttatctaaaggattttttggaaagggctttattgaaattaaatagtttctagggaatccttaaaatgcacattgctgtctctctgtgaagcacttttattctggttttggaagaaggttctgatttttgcagagctcacgtccagctgtttatttctggactaaggttgtaattatcctttatttatgaggcttggattgttgctgtggggatgatcatatctgtacaacttggcattatgccttcacaggagatgcaaagaagagagacagccttttgagaaagtaaacaagatcctctgtgacagtttgctttgaaagtaagcaatagccaagaaaaatctatgaggacattacatactacctgaaggttttgcctaatgagacacgtagaggttttacactccttgggatgatatcacaggtggatagggtggcagggctgcttcttgacattaagtaggtgtattcattaggattctccagagaaacggaaccaataggagatagagatatatgtaaaaagagacttattatgaggaattggctcacacagttatggaagctgagagttctattatctgcagtcaccaatctggatacccagcagaacctgtggtatagttccggtctgagtctacaggctcagacctgagcctgagaaccaggagttccaggagaactgatgatgtgagttccagtgatgtgagaaccaggagaaccaggagaactgatgatgtgagttccagtctgagtccaaagagccagaagagtcagtggagtaagttcaagtctgagggcaggagaagacccagcttaagcagtcaggcagagagaaagcttctcctttcctctagctttttttttttttttttttcttattcaggccctccatggattttgggtgaggcccaccgacattgctggaggcaatctgatttattcagtctaacaattcaaatgataatctcatccaggaataccctgacagacatacccagaaataatgtttaatcaaatatctaggcacctgaggcccagtcaagttgacacataaaatgaaccattacagtagacaagccgtggaagatcctggcttaaaacaaaaagattaccacagaaaagagcaagtctttcctccagttacgtaggactttaggtcagttttagggtagggcatcagagcccaaaagggaagatccttttgttaaaagtgttgtaatttgatgatttttcaaattaggaaagtaatatatggttattttgaaaaggaaaacaatactgagataaacagataatcttaccttcaccccaagtaacagcttgttgtacatgctttttctatcttgcttttctgtatgttcattacaaatactgtgtaagcacagatatgtatatattggtgtttttttttggttacttttcttttgctattatagaatcatgttgtctctgttaattactctgtaactttcttttttcatttaacaatgtattatggatatccctatgacattctctagttttctgtctcctctttttaacagatacatttgttagcttgtcagagatctagagaaataaagctgctcgttttccatgtcatatttttcaggggacctattgaacatattgacatccaagtcctgaaattgttttctgtttagattaaatgaatttgggagctatattctggatctgatcaatgacttgtaaaccttcctctaagtggcttacataatttcagaataatttgaaggtgtctcaccttcaaaatgaaagataaaatgtgagggaaatacttagctaaactttattgttctttagcagttaaaaattcaatgtgtgagtgttgggtcagtgttatgtttttctcctggattgtttttattaattgttatctcttctgatcccttcattttatgtcctagcatttttattacactttctcatattctctctaccctcttaaatactccctctttttgttttgctttgctcgcattctgtgagttttgcattttttgccatcctgcttctccttggccccagcccctcagatagttccctgcgtgaggacacgggtgagggtttagagctcacacctgctgaccagatgccttcacttttttctatgctttttataggcagttttatttcttctcttattcttccttcttcctctgaggcttcagctttttgcttttcaacattactggaaataaataaacaagtggcctaaatacatctgctgtgttgctttgatttcctctctattattactatttaacagactcagtgacactctttataccagattcttcccatttttcttccaaacatcctctttatgatacattttttcctcttttcaagtgcatacttttgactctgcagaaattaactttcttttctccaaaactgcaaacactcatactttcttgcttgaagtcatttggtttctataaactcctggaaatatttctttcaggctgttttgaaaattgctattacagtttggggaggaatagacttttcctttttcaaacacatgcttcattgcccaattctggattggattaatgtcgattcaatagctgtttattgcatgtctgtcacgtattatgcctcaagttttgggtccagcccctgtgttcgtttatttccaaactgtgcctttaaccttttccgttcctcctccctctgtagtacagctttatctattgcacacactttccttttcagtattcaccccccattcacggctctgtcctgtctgttcctctctttccaactgccatggaaccctgtggtttcaactttcaaactttccatggttccttcccttccaatgaagaaaaattaccaaaaactggatcatcacagatgtgtcaaacagccttccttcagacttcagactaacttcaacatataacaggtttcaagattacttaccagggtatggaaaaatagcctatatttgtctaccttgtgaccaggacttcattttcattgcctgttctgatattacaaaaaacaaacaaacaaacaaacaaacaaaaaaacaaccctccactgtttaaagacttaacatttgacttacagatagtttgctgatccttttggtctagtctctttttttaactctagaatgggtagtagatgatagtgccacacttttaaagaacttaggaaggggtctgaaaactccaaatagaaatattttaccttatagaggtgtctaactaatgtcctttaaatgtttcagcccatcgatggtatacatgacaaggggattgtagaagacatccattgtggatccatgaagggcactggtatgcgaccacctcacccaggcatgggccctccccagagtccaatggatcaacacagccaaggtttgtgtccgctgcacacctgatactggttccccagcatagtctttcagtctttcctgttggatattttaattatgaattccaaagaatgtgtctaaggaaggttgaacctagtaggtttcttttccttatggaaatctacctccgtcctcaccaatacaacctgatgatttatatgggaaattttatttccgaagtcctagtaattttactagccaccatgtgtccaagcagtctgcaaagagctctatgtactttatctagaatctttatcgtgatcttttagggtaaattaggttatcccagttttatagatagagaagataaagctggacaatttactttgttttgctaagtttacatgtccaatcggtgacagggccagaaagagaatctaggcctgcttcccagcaacagtgtagatgtgaaggtaaggtgaaccacctgtgtcccttgaattgcatgcttatccattgtctataaggcagctttatctggcagtgaaattatgtgtcatttctggtgggtggctgggcctgccatagcaaattaccacaaacagaaatgtattctcttatataattctggaggcaaagtctgaaatcaaagcatcagcagggccatgctctctctgaaggctttagagaagtatccttccttgctgcttcctagtttctggtggttgccagcaatccttggcattcctggacttgcagatgcatcactgcaatctctgccttagtatccacatgaaatttttcctgtgtgtctgtctgtgtccagattgtcttctttttatgggggccattcattggattagggtcttccctaatgcagtatggcctcatcttaatgtgattacatctacaaaaatactgtttcaggccgggcctggtggctgaagcctgtaatcccagcgctttgggaggctgagacgggcggatcacgagatcaagagatcgagaccatcctggtcaacatggtgaaaccccgtctctagtaaacatacaaaaatcagctgggcgtggtggcacatgcctgtagtctcagctacttgggaggctgaggcatgagaatcgcttgaacccgggggcagaggttgcagtgagccgagatcgcaccattgctctctagcctggcgacagagcaagactgcgtctcaaaaaaaaaaaaaaaaaaaaaaaactatttcaaagtaaggcacattcccaggtactgggggttaggacttcaacatatttggaggagggggaacacaattcaactcacaacaatggctaaatcagtatatggcatatttcttttcttcttcatacgtaacaagtcctctttcttttcttcactggtgaaaactattcacgtacctcaaagccttttaatacaatgtagcctgatttcagagtcattatgtggtcatttcaacatatgacggacatgagttaatctagcactaattaaaattaatatgtagttattccaattcaaggtttatggtttatgcgttgctatatataatcctgtgtaattaatgtgtgtgtttcctctatccatgatcagctatccatccactcgagggaagacttttggctgtgaggatttagagaagtctatgcacagttgttttctttttgaaatataaataggcaagtcatggaggcataggagtgcctccccactagtatttgaacaggcttttgaacatcccagactcttatctgaattttggtggcaggcaggcataattgttgcagataactcatagaatcttgaccatgtcagaagagcagcctaaaagcactgggaatttttgcagtacaagaagaacagatttgactcattcacggttatgaatgaagtaagacaatatttccaggcttatcatttacatttgaaggggccaagtatgttcatggagctaatataagcctttatttatttatttatttatttatttatttatttatttatttatatttttgaggcacagtctccctctgttgcccaggctggagtgcagtggcatgatctcagctcactgcaacctctacctcccgggttcaagcgattctcctgcctcagcctcctgagtagctgggactacaggcgtgtgccaccacacccagctaatttttgtatttttaatagagacagggtttcaccatgttgcccaggctggtctttgaactcctgacctcaggtgatccaccctcctcagcctcccacagtgctgggattacaggcatgagccaccatgcctggccttaaacctttatttttaatacctgggaggtatagatgagtaacgtttagtgattcttgaagccaggtgcttactttttctgctcctgttgttgcagtagctctaatagtggagtcctaggaaaacctgaaaactaaaaccactttaaatgcacaaatcataattgagagcatttttacttttatttctgtgatcacttggctcccctcttcaacttaagaatgatgtcttatttatcttggaatccacaacacttaggatgatatctagctagcacatggctagatagatattagtaaattttgttcaattaggtgtggaagtttgtgtattaaagagtcaacagtttaaaatgatctcaacttttatataatttcaatgaagtataataactttaaatgcaaaggtctgcattaaagggaatcagaattaacatgtaatattctctaaaatagtgaagtgcattgtaggctgaggaatgagttatttaagggtatatttttgaataaagcaatattagtataatgcagtcctattttatataatgatctaaaaagaatttagacttggtaatagcacgtgtctttaaaaaactcactgtgaaaagccatttattaatataaaaacacaggaacagagagatgtggtattgattggaaggctgcgagcttttaactacctagcaacctattaggttattttagggtattggtcaagaaggaaagccatttccttcaataaatactgaattgtgtaccttatggtcccaatgataacattgtttatttttagtccattggatagaaacccttcaaaagcaggtatatatttaaatagtgtgtgtgtgtgtgtgtgtgtgtgtttgtatgtgtgcgtgtagagaaaattcatgtagaattggcacttagaagttcaagattattaataggagagaggttattgcatctctctgaggcaaggtgaaactggggagataatagcccttgaagactagggaaagaaaactgccactttttgactactttcgaatgagcaaatcaaagaagattccaaaaagagcagaatctgcatcaacactgtcatcctacagaccaatgtatattcactctcaggcaatttcttcactctgttttctcaactaaagataattaccaaaatatgtgatgttgtatcacttagattctgtttttcctcaagttttatgggatgctattcatgtagaaataaaaatctctatttttaataccatacggtgacagaatgattctattcacatcccctacaaattctgggaaatgattcttctcactttcattttcttctgatcaaatttgaggtaaaattttgttgtattgactaactcagaaatgatttttatttccctctcaattttctgatcaaatttgaggtaggaatttttttgtattgattttccagtgaccaggttctttttttcagtttgaattcttatgtttaatgtaactgtctcaccttcataatgttgccttgttcatagtttaaaagagtgatttaagaaatagtgttatacagttgaatttagactaggttgcctaatattttggttcattgtattaggcatcttatgatttgccatgagtgagattgtaattgatgaccttaggaaataggaaagaccggaaatcaggaaacatatgactaattttaagccctggtcgtatctcttctactgtgaaaatgttagtagctttggtggaaagatttttcctcttatggtagactcagtgctggccagaaagacgtcataatgaacagactgccattttgtactagcgttacaactagggggttaatgtaagccttcttaaagaagaatgtatgctgaagttaagtgtctgttattgatattctctaagtgtttattgttataataacaacatgtcaggatttaaatttctgctagtcttgcaagctttcaagggaattttcttggctaatttatataaccaactaaaactgtaatcactgcatggtgtttgtgcttcctcacggagtagggatttgtggaatgaaatttagcaattcttctatttacctttagggcaggattctcctgaatagagttctacttctcatcagttggtggagcaaaacccattcttggtttaggggatcacgctaaatctgcctccctctccaaatgacaattggagataaaagtaactgaatcttttattttctgtggtcttgtgaattatgatggtgaatccccagttggtggcggtcatatattctccggcctctgtgattctgcagatgcacagagctctttcagttcaaatttttcttaggttcgggcatgcagcaggaatttaataaacacttatgtgatgacctttgtgagaactgtgacacgattacatttattaaagggcagagacttttcatctatgtatgtaatgccatattctgggacacctttgacttgattaattggcactttgtttcagctcaaggacaccttttggattgctttaggccactttgttctttccttcatcttggagctccctccaacaatgttcagtcatatgtctttctaggaattctatggccaagctatgcaggtcatctcctgaaataagtcccttgaggttttttgtgcattttgatttttcttccccagaaaagtgttcttcaatttcccatttagatctcttataaaccaaacagtttactctcatttttatgttatatcacaagtggttctagaaggtttttgaaactttcaagtccctaacttattttcatataaccaaatttgtattagatcatggaaagggcatatgcattagaatcagacagacctttgtttaaatattaaagctcccaaaagttaattgtgtgactttgggtaactggtttactctctgagcctcattttcctcattttaaaaaatgtggtaacagtatatatcttttaggttgttgtaaggattgaagaaataatgtatgttctacccatatcttcttgattccccttaccatttctatacccccaccagcctgatttcctgtgcccagaaccactgtcttggttcacttccgtctactgacacctacctttcccacataatggcaaagtccctggagaataataatctttagggcattagtcataaccagtggcttaaccagtgactgctgattgcaggagtataagtaccccagcttctttacccctttcctgaaataatttggaggcaagttatttactatatattctagagtttctgcactgaattaatcttcaggtacccactgcagcagctggcttgaaaataccccccctttactggctgccttcccttccttatattaccttttcatacccctattagtgtccccttaccttcccagtaaattactagtactcaatccttaaataagactctgctactagggagaacccaactgtgacagttgagtgcaagtcaactccttgcatagtacctacaacatgtaggcactcaataattgcaagttattcataacatgcataataataaaccaagtgttcgctcttcacctaagaaattctttattccccattgagatatatcaggttatgttctcgcagttggagctacttttaccattcagtactgtcccttggttagtggtgaacaacaaccactaacggtttaccttctaggacacatgtagctattgatcacttgaagtgtggttagttcgaattgagatgtcctttaagtgtaaaatatttagtggatttcaaagacttaatatgaaaaaaagaatgtagaatgtctcatcaacagtttttacattttaaaatgatgttttgggtatgttggtttaagtaaaatacatttaaattaactttacctgctttttttttttttaacactttaaagtggctaccagaaaattttaaattttacatatatataaagcacatattgatatgtaaagatcttaaactccatataattaataaatgatatgtcattcaaatttctgtcagacagtgttgctgtggacaattattagagtattcagggatatctctctttcagggttgtcaggggcagcctgtgatttccttttgtgttttattttaggttatatgtcaccacacccatctccattaggagccccagagcacgtctccagccctatgtctggaggaggcccaactccacctcagatgccaccaagccagccgggggccctcatcccaggtgatccgcaggccatgagccagcccaacagaggtccctcacctttcagtcctgtccagctgcatcagcttcgagctcagattttagcttataaaatgctggcccgaggccagcccctccccgaaacgctgcagcttgcagtccaggggaaaaggacgttgcctggcttgcagcaacaacagcagcagcaacagcagcagcagcagcagcagcagcagcagcagcagcagcaacagcagccgcagcagcagccgccgcaaccacagacgcagcaacaacagcagccggcccttgttaactacaacagaccatctggtaggttaatacgcaaccaaatgaataatgccatggtccaactcggataacaaagactgctcaccaaaacaccgggttgttaaaagcccggggctgacgtagccttttgttatacctcactggctctctatccttgctccacttagatggccaagattcttggtcttcccctgctgttgagacctagggcagtgtttcttaaccttaaaaggtggttgtgcaagctggagacccagggaaaaagcacctgtgattttgcgttctctttgaagcggttcaaggtttcttggaagcccatccacacacacaaggttaagaacctctggacttagtgcatttcatccccagaactcactgctcagatcagccccactagagccattttataatttgttgatttgtttatttctatccctttttgttatgaagaacatctggagaggcttacaagaagcatgcctgttgtaaaataattatgagtagcaaaatcagcaacaagggaaaatagaaaaaaaagtaagaaattgaggcaagaaagtagggctgcaaaagttctcatagttgctgtatttgcttcaaagatgacttgagtttcttgacaatcagagccaaaaggagacaaagtcctgtatctaagtctcgccctttggaaggaagttccttaccagtttctcagaaaaagccatttctagcatgagagtatagagttgatttcccaggtgagattattttttaaattattatcatcatcattattgtaagaggcactgcatgacagaaacatcaatagcatttgttcataatagactaacagattcaacatggcctatttcttatacaccttgggatgaagtctgaagctatgacattatacaatgggtagaaatgatacaatcctttgcatggcctcttggtcttctgtcttgatcccattgatagacttcagaggtgcaggggactgtgaagtgatcttccatgaaggttgttttctctaggaagcgttttgttaaagaactggaaaagcagagattgtatgggtctactctaatgaaaactttggattctagggaatgaggtacttccttgacgttgataccatttcctctgagtggtttccattcatttaggcaagttctgacatgcaggttgttgcaaacagctagtttgggatattggtactgttcctgggttttgggccttataccatgggagagagttcagattagtattttttcattaatattctgcctattgagtttgtatacgtttattttctggtagaggcagctccaagagtaggcctacctaagagatatcctcatcttattaattgttatagccttaagaatggatgtcttagtaagtctgagctgctgtaatgaaaggtcattgactgggtggcttaaaaacagcaaatttatttcttacagctctgaatgctgggaattccaacctcaaaacaccagcagattcagtgtctggtgagggcctgcttcctgattcatagatggcttccttctcactgtgtcctcacatggcagaagagacaaggcagctctctggagcctcttttacaagggcatgaatcccaattatgaggactctactctcatgaaatagtcacctcccaaagaccccaccttctaataccatctccctgggaattaggattttaacatatgaattttgaggggaccataaactttcagaccatggcagtgggttttaaaagggcaagaaagtatcatttcatctccagaatttctgtgtggctaccatggaggcatttggtaggtggaggatcatagagaactgagatttgggaacaggaaaggttatacaagaaactatgcagaaaatggcagtttgaatcctaaaagtatttagaggttttaaaactgcaaaactccagctcttgagaatgcagccagatagaagaaaaaagcacgtatgtatatgtgtgtgtggcttatcctccctgagtgcctgctggcctactcatgtgccattctcagtgataagtcctttgcatgtgtcacacaactaatgatggagagtcgggattcagaccccaggaatttgacttaagatctcatgttcttaaccactttcccatactggctccttcatggcaaaaaggctcatgacaaggaaaaaaacctatcacaaagtctgtgtaccaagatgcaagcattgtacttctgtgtgtaaagagacagtatctgcacaccttacttagatccttttccctcttactctgagacacaatatcaacaaaattggtaatctttatatcccatgtctcagtatgtgaagagtattaactaggagaatttcaagtagaggccagctggtaactaccagctacatgaaaggctgatggagaggagggatcagaacttgaactcccaaaaggtagtagtctgggatgatgtgttctgggtatgatctgctatacctcacagttggtcctgggatatcatcttgtcacagacatcaccaagcattggtatttttaagaacgagctagactcagtaataaaagattcgaagatatcagttaagaatgtaggaagcagaatgatttgtttgagaaacctttttcatccaaggttaaaataatgtaactgaacacagtagtaaaatttaaaggatgaagttccatttatctggatacctgtacacaataaatgcttcatgtcctaatcaacccaatcctgagtgccttttctggatcattttcaggagagggctctattttttttttttttttaacctggctccggtacctacatctttcgtcatcaggtgcctgtcattaaagggaattcactttattactcactgctcatgtctttttaaattttcaatagtcggagcttcattagaaatgacaatatttgggcaggtagggttttatggaagagtggaaaggagacgtgaatgaaggaggaaagaagatatctccttcaaatagaatgagcccatttctgtcactgagagggcactgccatttgacagcattgctgaacttgtccccacatctcaggtttcattagagaaacagataactttacttgccaaaaataattttttttttagattggcttttagaaaagataaaataaggcatgattttcttgttaaagttcatattagtctatttattgtttatctcctttttataaaataaattacttttaaacacccctgctaatatttcaggatattattcacttacgtagaggtagttgtgttcccattttgcttctgttccatttcctggtgttattcatgtgtgaagagaatctttttggctgggttggcctgcctcaaagctgctccaaacttataattcaccaaaagtgacatcatcccagccaccaccaaagaattatgattggagtcgaggcattgcccatggaacacataactctgcagtggtttcccctacaaaatgtcagccactcttacgatggaatgtgcccttgcctgctgtgggcaccgctcaaatctaacccatcatttttcttttcattttgattcacttttcctttcagagattcaaacaaaaagaaaaatgggaactgaaaaaatttggggtttttgaggggtgagggagagaccttatggtagaaaataaagtgttttttttgatatctgaaattttatatgctcaatttaaatgttttattgcgtttgttttttccaacctgtggaaaccccttaaattcttactgaatgcatagatcttttatttgcatgaatgtcctgtatgccggaagactaagtgggagcactgaatgctttgaagttctgtgcctgtgagacttgggtattttaatacattgttaccctattgcatcaaatcacgcctagggatgcattcaataaagactgccccctccccgcttagctgcgctgggactcccttctggagggccagagtttctactttgtttctactttgactcctaaaatgaggactgaagggaaagcagatggcattttgtacctctgacaatgaagcctaaattgtttacgttggagagggagtgggagataagctaaagaatgtgatttgtcttccatttgaagataccagggaaaagtcttgtcaagtagcaggccaccggtgtctagtgtagaggagacgatttctgtcgatagagagcaaagccagccaggcaaacgaacccgtaagccgcctgagggacagacaggcataaaacctaatgaatgggtaggtagactttgggcaaggctaattgcagcaagctgtgttcagttccccggcctctgttaagtagtcatttttatacaatctgtcggagggaagaggaaatagacccacacggtaaatgaacttgtggtgtttagctaggaagtggagtttgctctaagtagctctggtagcagactccagggagcacatggcaggcggactcatctgagtatctgtgctgaggagttgtgttaaactgttgaaacacttagaaattgctatagaaaaagagtactcccaaagttgccagctaatatggtaccgaatttcctgaaagacctttcttttgccatgtggtttttctataaagctgagtcaattaggcttctttatctccctgtccttggcaataaaacgacaaaagccgagggttgaacaggtagacactgttatctcactgaatcttcaccgctgccctatgaggtggctattgttactctccccattctatagatgaggacactgagccacagattggttgagtaacttgctcaggctcacacaactagcaggtgataacagctgaggtgaaagaacattttttctgggaagatacagggtgtttaaaggtacctgtttagtgcacagcaagttgcagtacttttaaattggagactggaatatgtaagtagatgtttgaatctctctctgtctttcagtgttacaggtgaatcaacaaaattagaatatctttctatttagtgactgaagaaggcggtccatgcatatatcagatctagtttggagtcacagactagattctggaccagagatgaaagatatgctttgggtacctctttttgattaaatgtagtttttaaaatagtgacttgactatacctcaaggagtcttcataatcttctgcatgggtatttataggcaataatgctaaagaagacagtgccagtacaagtgaaatgataacatgattgcagtgttacggcttttgttgatgaattatttgaactgggaggaaaaacactttgagaactaaatttaaagtaattcttccgatattatcagtaagacctaaagatggttgcttttagaggacctaaatgtattaaattcctgatttagcatgcttatagcaaggcttcaaatatgtttaattctaccagcttcttgagcttcttaaggagagtcatagaatcatagggaattaaaatgaatgttagaaatctgtatctcctctgacttgactggatcacacctagagcaacccaaatgaaaaatgtaccttgttttaaagctctccagggaaaaatattctaaaacttcccctgataacttactccaatgcatagtaaccctgagacttaatatttacaaattggtttcttatatatggatgaaaggaactctaaatgtatcgtattataaaaggtatgcattacataaaggaccttttatacaggaaaaaaagtatttagatgaaaaaacttcttaattatggaggtacatctacaatacctaaaattaaaaataaaatgaagtgcattttcctcctaagattttctcatcaaccccgagaaccactgctggaaattacctgaagaagaaatcttaccttgctctgtatctttaggaaatgttttacctaattttagaaggaggggaaaagaacgataacatcacagacacacacacacacacacacacacacacacacgacacaaacacaggcacacactgtagtttcctaagcagtttgttctaattcttcacatccccctgtcaaaatgtttgcttatagctttctttgtattgtcatttaagcccatttctaattggtgattgttggaaattgaataaatgtgttcagtttttcataatgtatgtcgttgtatggttaattattaaatcgctctgaagtatgtagactattatttttgttaccatttaatagctattttttcttagaaaggatttagaaaaaattggaaaaatgctctggactttaacatctgcttactgagattcttttaaaaaaatttaactcatctggtagcatcactaaaattataattttaaggcaactgaaaattcatctaggagaagaaatggaacacttaaaaatttaagtctgtacctagtaattaacttaaactcctgaagttcaatgcggaaaattattttaaattaacttaatcaatgcactttgacaagctttcagattacatattgttttattctggtaacatgggataatgctcccttgaatcctaatatttgccaatttttattaggtggaatgagcagattttttttggctaacaatgaatgttttaagaagtgttttctaatatgtagtaagaaaaaatttaagttcccagacttcactgattaaacctgaaatatgctttataggccttaaaagaaaaagtgtgtgctccttgtagttcacatcagaagactgataaactcgttgctctgtttgtaaagcatgggttctgacagcagggctcttccagattgcgatagaggtctgtcctaaaagggcttcagattgggatagaggtctgtcctaaaagggcttgaggtgcaatttgcaagatgtgattttaggaccatacccaaattgcatctgactgttgggtgactccctttcttttctgaactctcaatgcacctgtaattaaaaaaagaaaaagaaagaaacccattgcagagctacattaaaagctggcagtagtttccttaatcaattgtattaagtcaccgtcattttgggatcatttgcacccgtgttcgttagtgaatgccatttcccgatggggatagatgccagattcttttttctttctgtgtctttttccttctccgttttctttttccttttaccccgttccctgtcttgccctcctctccctccctcctcttccttcttgccctcctttttttttttttccttctcttccctcaggtgtttaagacacttaatggtccccagcactgggccccggggggcggcgggcaggccgggtgtggcccgcggggcgccgtcccgcccgagctgcccatgtcgctcttgtcccgcaggcccggggccggagctgagcggcccgagcaccccgcagaagctgccggtgcccgcgcccggcggccggccctcgcccgcgccccccgcagccgcgcagccgcccgcggccgcagtgcccgggccctcagtgccgcagccggccccggggcagccctcgcccgtcctccagctgcagcagaagcagagccgcatcagccccatccagaaaccgcaaggcctggaccccgtggaaattctgcaagagcgggaatacaggtaacgcaccccgccagcaaggggccccctgcggtgtgctagcacctgccgcccaagccgaggggggtgaggcgcctgcctccttggttggccaaactgtgattttcacccgtgcggtcggaaaactttcatccactcctggggccttcccggtgctgaggggaggcaccggggttttgaagatcaaaagccgacggggacttcctgtggacccgagagagtgatcatctcacactgcgactgtggaatacactgtccgtgtttgtgcttgtgcggttcggtgatgacggaggggctgggacgctgagtgtgttgcgctggcctgattttcatgttaactgtacgtttctactaaagcatccttcggtagcttggtataatctttgttttaattcacgtgtgtctatgcagtgatccagccttatgggtataccctgtgcgagtgtctttatacaaggtctatacttaaatcattagtccagaagcttaatttttacctgattcagccatttagcagaatcaggggttttggtaaactcttcctgtaaagagccagatagtcaatgttttatgcactctggggccataccgtctctgtcacaactcacctctacccttgcagtgtgaaaacagctgcagacaataatgggctggctgcctttcggtaaactttacaaaaacaggctggaggcagaatttggcctgcaggctgtgtttacgagacccctgatgtaaatggttaatgtcaacccaaaagggagtccggcaagtattaagtcaagataaaatgaagctaaggttgtggattctggagtctgccagaaagcatactgtgtcatcagtaacttcttaccgtggcgaagttatgtctcttttctgtgactcagtttcctcctaggctaaaagaagatacctctcatagtgttgggaagataaaatgatactgaaacattcaataaataatacctattattatttcccaaagactttaattttaaggaaaattagagtttaaaaattatagagaatgttggtaaacagtgtctgggcttccttttaaaaggttaccttaatctttattgaaaagcacatcactcatttcttatcagactaaaatactaagttaccagcctagagaggtaatctgaaagaaatcagaataacttgccattagatgtgtttatttttttggatgaagaatataactttattctcatcactattaataatcatctgttaagagattttatattgctatttccaccatttcatgtattcccagagagctagatactcattgtcaattgatatttcagtgctgataggcagattctgctgggtgaataattgctgtgtgttataaagtcaaatactagcatattcctctattcatgtgcaatagactaatgtgcagtacattctgtatgttactgttgagtttttaaaaagctaaaagtattggaaaaagtcagtaatagaatagagaaatacaatgtggatgctctaaaattctgacttcctaagagctgatattttttttcctaccctaggaaaggaaaaagctctgtacaagctccctagttaaaacattttttttttccatttgaattaccagtacctttgttaaactatgtgaactacccttagaagagagatgctttctatagctagaaatttttacatatgtctagacttctttcccttattcagcctggtatgtgtgtgtgcacaagtgtgttttaactttttattttgctaactcctaaatgttcttaatcattttttctggaattttcattgcaggactttcttgtaataagtttattttaggaacttttttggagattaccttagtggatggtcttccaaagtgcacagtattaagtagtactatttggcctattccaatgtgtaaaaagcttttgttctgccctcctatgtcaataaaaaatgctgtcagtatcttttaaattacatgcattacaaaatgtggagaatggcaatttttcatagtattattattgctttataaatgcctactcttttgtaaagccatgtggcttttgaaacgactgatctggacaaccctgaatccagaaaggtcatgggtcctttggtctgaattttctcatggccacctggagaagaagcacagtttggtggtgacagcattatggggcatgttttgtattctttggttacagagcaatagctctcttctgcctatttgaaggtaagttgttcaactgcagagattaaatttatcccttaaaagggttaaatgaagtcccttaggcacttgcaaaagacaacaaatgaaccattttttttcttcttttgaccctttgtgacagaatggcttaccagcctttagcattgataactgttaggatccagttcatctttatcatccgatcaaagattagagaggggtctaaaaaacctcttttacaatttgataaacttagtttcagcaaaaatcatctgtcagaaattcttgaacagatgttaatttctttgggtcttgtaatatcatttaccttgtctatgaaatttgacaggaagttttcagcatcttaaatttttttaacgtttaagtcagttggctgcttttccttgttcatcataaaatcagaacgttgacaaattgaccttctttttcattcatagcataaataaatgtctgcctatgagcactttgctgcagctctgtacaagctctctagttcaaacatttttcccacttaaattggatcctttctttttgaccatctttctcttataatggaactttattacttccatagtaagtgtcagagagatgtttccctgtactgctttttttttttcttttttttctttttggcacacttttgatgccttctcattgtgcctaacttcgctttcttctatttagagttagatgtctgatcaaaagtacctcaggaatcatcacattggcatgtaaactaattccacacatgtttgcctggaagatgtatttcacacttgaaggggtttatttgggaagaacatgtctgaattatatcagccctcttcggaataaagtcagactttagtatgaaaagagcggtatatcctctttgtgtcaggaagcaggcataggctgcttacctgtgatttttttttttttttgacctgcccttgtcgccattgcaatcctgaaaattccttctcaaaaattaccagtagctgctttaccagcagagttacaaaaagtcacacacgctctctttttaaagtgtcactcagctccttgaaggaacaaaatgtaccagtgtcattttctgttcagataactctcctacaggaatcataaacttctcctctggacctgaacaagtatctgttgcatttaatgcaactctttttcttgccagaacagtaacccctcgtgatttactgttcaatctgtatgaaaaacctgtgtgacctaattctgcatcaattttctgtactcgtgaggacgctaaatgagtcccctgcagttatgcagtattacatcttgttgttgtttggggaagtttagaatcctgaatctctttatcggactacctgctgaatctcactgatggggagatgaaattgtctggagaggaacattatttaaaatgaagatttctattaacaacaacaacatcaacaaagatataatcttctgtgttaaagaagcccctcctgtacctttgcttcttccccaccatgtgctgaatcaaatgcatataattttactatggagtctacctttcctcttctcctagagtttgcgctttgtacttcttgcctcccttccagaaaagggattcttttgtgccagggaatcagattcactgacaaataactgggctccttggagaaggagtcatttcagggagaggtgcacccccctcgctcccactctctctctccagtgcctaagtcacacagtgccccatctccccggggttcccaccccccatctcctcatttccgctccacttccctgcttcccacctcccagttcaatgtgatgtaatctctaccctatcaccccagttatctgacgattacttatttagtcctagttccatggttgtttggaagattaagtaggaattcctctaatttggcatctgacccactttgtcaaagagagtaaatcagaataccaagggatagtttgagttctaggatattcaaataagaaagatctgcaaggaatctagaaccaaggatgcggaataaccaaatcattggccatgggctcacgccactataatacttaattttcctttggtatggccttaacctgtgaaagtaaagttgtctcagttttattttttccctttaaattatatatttcaaaaaaaatcaatagtggatttttttctttacatcttaattgttaagatgcatcatctgttgctgtatccagagatttggggctacatagatgttgtagaaaatgagtaaatgaagtagatgtctgtttcatatagtgaaaattaaaaaccacggtaataatctcacgagtatcactagtacaaggaatgtggaagaaagttgggagaatttgaacaattttgccaaaagagtaaaatgatgagtttattattgttgcatgaaatacaaaactttcagtcttttgaatgattaaaccacttaaaaatgctattgtttgtgttggtatgcatagttaatagcacgggccaggcatggtggctcatgcctgtaatcccagcacttcgggaggccgatgcctgcggatcacctgaggtcaggagtttgagaccagcctggccaacatggcaaaaccccgtctctactaaaaatacaaaaattagctgggcgtggtggcacgtgcctatagccccagctacatgggaggctgagaggcaggagaatcgcttgaacctgggaggcggaggttgcagtgagcagagatagtgccactgcactccagcctggccgacagagcgagactccatctcaaacaaacaaagaaaaaacaataacgacagcacggtagcagtgataatattaacacattatgatgacaaatcatttaataaattagaacgaataacgtgcgtatagcaaataaaacaagaatcagtcttagatactatttttagtgaagtccctgaaaaatagttttccttaccaccatttttaaatttttaaaaatgatgacattttatttcccttccaaactaagcaactgtaggttttttaaaaaatcactcattttgatatttgggctatatgaagtattagatcttctacagcatttaggtccaggaggtttcactttttcacgttaatgaaaatgatggctgtcgtcatcagcatcttggcaaggaagacaaatggtctggttgccttaggacagaaccagaagttagaaagaaaaaagtctgtcctcccagctctgtactctggcaacatcatcatgtttggaaaaaactgtaatattcattttttaagacaatttcgacttttattttagattgaggggattcatgaataggtttcttaacatgggtatgttgcatgatgctgaggtttggggtgcagttgatcccatcattcagctaatgagcatagtacctaaaagtttttcatcccttcctgcgctccctccctcccccgtctagtagtccccagtgtgtattgttgccatctttatgtccatgaatacccagtgtttagctgccacttaaaagtgagaacatgcggtatttggttttctgttccggcattaatttgcttaggataatggcctccagctgcatccatgttgctgcaaaggacatgatttggttcttttttatggctgtgcataatttttaacagaattcttaattttcagcttttatagttgaggtccaggaaggttctcggaaagtgtatcttaattatctatcattttattgcagtggttttggtatcaggcaaattgaggttccaatcttggcccccttctttactagctctgtggtatctccaggtgtcttcagttttcctcatcagcaaaatgggaccattataacttttaccttacagaggcgtgtctgtgtgtgtaagagagagacaattaaataacattgtacattaaaggcacttagtgctgttaatattagcctactttaacttattacttattattattctttctttgttgaaatctagttcagtacagtttgcctcagatttaggggatgtagttctaaaaatagtcttaaatggcatataattattttgtcagaataggattcaggtgaactctaaaaccagcagtttatatatctaacagtaatagtatcccaagtgaatggctaaagtcagaggtcttttattttctactctgcctttattatgaagaagtacatgcagttatatttttgatggtgtgtaaaacgagaatcctcaacatccttttctagaaactcttggtttttgagtactatttactgctggccctgatttttttaaaacagcctcatttgtattttcttttctcaagcaagctctgctcacagaggaagtggttgtggctggcagagcgctttctgaatgtttagggttggctgatttctgtggcatcagcacctaaaactgttcttgctcactgcgctttaatggctgtcgtcatcagcatcttggcaaagaagacaaatggtctggttgccttaggacagaactagaagttcggaagaaaaaagtctgtgctctcagctctgcattctgtctggctgggagttagaaagcttgtcaattggttctgtctcttatattctctctctgctaagagtgagcaaggacatttgacttccccactagatgcagggtgaaccttctttccctagtgttagatagaggttatggaggaagccacattgttaagtaacatatacctgtctgcattaatgcaaaggtagcttttctctgcttaacatcaaaaattctagttgggtgttagagatcaactatcagtatctggaatatgttttgccccggacttaaacctaatgtcattgtttatcatttaagattaaattgtaatgtgtttttcccctgcccccttttccccattttattgtttcctttagacttcaggcccgcatagctcataggatacaagaactggaaaatctgcctggctctttgccaccagatttaagaaccaaagcaaccgtggaactaaaagcacttcggttactcaatttccagcgtcaggtaatacattttccccagtgaatctgagatgtaggaaataaatgtaattgttcctaaagtgttatctgtgttgcctttagtctattcaatattgttacaaagatataaagatataaatatagtaaaatagtgaatcttttagaccaacagcttacaactaaaaatgatgaagatgaactgcttattattctggttttctcttctgtatatgggaaagagtttttcctcctgtactatgggttacaaagtcagagttatggcgttttgttaccatggatgaaaaaccttgggaacaagtggggtagctcatgtttgcttttatgactccaaggaaaaaccagaaggtagtggattggagcttctttgtaagcacagatgtaaagtgcttgccacgactgtgaaaataattttaacaaatatggtagccacgctactgaattagtggttcatcatttatagaaatacataaagcaatgtcttatagcttatttattcaccgatgtctaagtttggagtgtttgatcacatcaggctcatgtattgactaatcagtaaattatctgctcttgtagtttacagcagttattaaaagccagtataatgacctattttctgatttctaaattagtaagagacaagtggaatgagttagagctaattttcactttggcgagttttgccttgtggactgaaaaggttcaagtttgctagtccgcagattgcctcactaacccaaaggggataaggaacgtttcattgttagtcagatgggtttgttttgttaaagtctggaacatgcgaaatgaataattaatgagtcaaagtagtttggaagtattgacttggtttgttggaggatgtgactaaaatcacaggccactccagttgctattgtggtaggaatttgggtgtatatttgccttaccggctataatctcacagatggacattagtggtgggtaggtgctacttggctcttggtttgtgagagtagatttcagcttaaattttaaaaagaaaagggaaaattctctgagttctgtttacttctttcttattggtgacctcataagatgaattaataaagtgctggataaaatcttactaaatgaatattaaccaagtatattttccggaatatgtttatgttcaaattagcgttgccatgacggactcttattgatttctgatttttgtttgatattattttaaaaatctgttatttatgaagcttcctaattaaattaactgtaactaatgatatttcagatgcagtggaatgattatttaatgtgtattctccagtccctaccccctaccaccacataacatgcaatacagataaaagtattacatttgtgatctgaaataagttcatgatataatataaaatgtagatctcttggaaagaagatcattattggagtgggaagaagccaaaactcatatttcccatcctttgtatgagactatgaattaacgttgtttgagtacctgctgctaccacacgtgaaagtaattcttttattctttttttggagcatcattaaaattaatctttaaacacaaagagaatgttaacatgacacttaaaacaaagcggaaaatttaaaaagcttatttagctaattggtatcattagaataattagagaaaatgaagttatttaaaaggcaagacaacatcttttctttcttttactgttacagtactataattgcttttgatttgaattatttggggagaaaaatcttgtaccgttcttgaaaatattgcatacatttggcatgattttagttccttcatttaatacaaaccaaaggtgattgagaagcttgtggagattccccgccccactctattccattaaatgcaaccgcgagaaggccagagttcaggaacctagcttctgttagggaaggctgtctaactgctctcttcttgacagctgagacaggaggtggtggcctgcatgcgcagggacacgaccctggagacggctctcaactccaaagcatacaaacggagcaagcgccagactctgagagaagctcgcatgaccgagaagctggagaagcagcagaagattgagcaggagaggaaacgccgtcagaaacaccaggttcttagaccctgggctttgctcaccctcactttggcagagctgtccaatgaattcatcaaatggggtcagaatgactgaaaaatggacccttgtgggtggtggggacatcacagaacagaacggttccttgacatgtacataatccaaccacatcattttatagacaaagacgcttaagccaggacgtataaatgactgcccaaggtcatacaattgcttagcagcacaatgaggtttagtattgtcttagtttgtctgggctgctataaaaaaatagcacaaactgggtggttgatgagcaacagaagtttatttttcaccgttctggaggctggaagcctgagatcagggtactagcatggctgggctctggtgagggtcttcttctgggttgcaaactgttaacttctccttgttttcttacgtggtggaagcaggccaagaaagctctccgggcttcctttataagggcactaatcccattcgtaagggctctaccctcatgaccttatcacctcccgaagaccccacctcctaataccattacattgggagttagcatttcaatatataaattttgggaggacacaaacattcagtccataacaggtgtcatgttcataatcccatgggaagtttttgaatgggtgggcataggtggatttcctagagcaaaattctgggagcaggaacttggaaaaggaaagtagaaagcaaaaagttattttcataacaaatataatagtgtcctggcttctctgaagggcctagttgaatgtttcacagaggcaaatattaaaaacattcctttctgggggtttggaaatattttgatgtaggatttgatttcctgtcatagtcctgtaaattgtatttgttttcctgtgcttaatttagtgcttagcttagtttccttttgaaaaaatgttggattttagtgtaaatagagtatatatttaaaaagtggatacaaattctgcatggcaattttaactttgtataaacttattttgtgtcatactctagggtagttagagcagaaaaacattacttttgtgacatactttttgctggaaatactcatgttcctcaggtttattttggagtgaatgagtggtttgctttacattttatcattgccattttgtaggtctctttcagtcttcgatctttccattctcatgtggccacaccctcaaggctaaggaggcttctcttgctaaagcttacaacaaaaggaacccgaagaggctatctttcccaccagccccatggcccacaccttaactgcagagacaccagggcacctatccccaaacagattctagtcttcctatgctgttaaacacacactgagagttttcttggacaacactgatagctcagaggacactggtcattggattttaagtatccttttcttccctttttggatcttctaggaatacctgaacagtattttgcaacatgcaaaagattttaaggaatatcatcggtctgtggccggaaagatccagaagctctccaaagcagtggcaacttggcatgccaacactgaaagagagcagaagaaggagacagagcggattgaaaaggagagaatgcggcgactgatggtaaggaactccctgcaggagcccaggaaactactcaacccacgtccgtctgcaatgagaccattaaatatggtggtagtagaaggaaaaaatgaaaactgcttatcaaaaattttagtaaatttctttgaaccttagggagatgaagtaaaaactttttctctttcctggagaatgcatgccaaaaatctagggggatgactgatgcgtatagtatttctgtgtcctggggtggcattgcgcggtacacattacagcttctctcataaaagcaggtactgtattttgcatgagtttgataaccagatctggaaggatggcaaatgcagttcttttaaaaatatggcctctttgagatgaatgaccacgttttttggaatgaataacacattaagtcgtgtcttcaaatctaagaaggtctgtgttttgaagcctctggtgcttatctttcttttttaatggcagaaattattctctaacagtgtaataagtgatttttaagttgattttgaaatgttgccttttttgtgatagccttgttatggtcaaaggctattcatggattattacatttgcactgttattttttaatgggcatataagcacagaagagataggtttatatcattggcctgagtatttgtttttattgaccatttatttttggaattggacctcagagcacactgtggattttagaaaagcgtgtgtgtgtgtgatgttataattataggagacctgcagattctatctaggacgtaatgcctgcatagatgaagataaggtcattagtctcagaccataattatgttcctggaaaattctaagtgacactgtcttcctggctggtattacagtttgcatttttttcattttttggattccagagagtaaacaccatgttttaaaagcctctgtgtgcaggtactactacatcttactaccctatcctgcctccctctccataccagggagagaggactgtccgagaggtttctgactagaccgtttttgtagtaatttgatcactaagcagtcaaatttgtctgactcaactgaagtgaaactatattagaacttcagtaacagattggtttgacactaatgattaggcatttgcaaatcattaatttttgcaattaagtgatggaatatacagataatagagaaactgaagttttttatcactctttcttcaagtgagttcaaccataaccgaaagcaattagagttataatagcatttgaggctcttgctcatccacagggcagacggcagctagactgagtgatgaagctgctgtcactcaggcagatgctagagtgacacccctggacaaaagatcccaagtttttgtttttgtttttgcaatggagttgcctcttaaagattttgcagcttgctccttttatcgtggcattacatgataaaaataatttggagtgtccttttattgggatacacaaagagatttctctcaaaatggcttagagtacatcagagttttattctgtttaacctcatcatgttgcttaatgggaggtggaggtgggaattactgtatctattttattgctcagcagtaccagacttcaggtgctaagtgacttgtctccagtcacctgggcccattactcagtgcagatctgtggccaaaaaaaaaaaaaaaaaaaaaaaaaaaaaaaatcccccaaatttgatctttcctgttaaccacaaagtttgtaatgccaaaaagaaagactttttaaaatataagacattatttgttaaaagcccatttgattccttgcctttagtagtactttttgaaatatttatgaatgaaaggatattatgtctggaatttgcttcaagacaattcagtttgggtttagaagaagagtagcaatgaaacaggagtcatcacaagtgaatttttgttgtttggggttggttataggagaaattaaactattctctctgcttttgattatatttgaaaatgtctaaaatatagagttaaagataaagtttagatcatgaattgtcaaattctaaaatctttttgtacagttaaagcaacacagaaaacaaagagcttggctcagagtgcttaatcacaccacacgtacagtgtggctttgtgcccggactttgctgtaaactccagtgtcctgggatgtaggcgtcacacttctgtgtgtttgtcccacttcagactctgtgcaatggcttgaacagcccaactcatccagtttgctacactcctaccagtcaaaatgcagactgtcaaggggaggacagagtggagagtggagttgtctgcacgcttatattatgctctcatcctgctcttctttccttaataggctgaagatgaggagggttatagaaaactgattgatcaaaagaaagacaggcgtttagcttaccttttgcagcagaccgatgagtatgtagccaatctgaccaatctggtttgggagcacaagcaagcccaggcagccaaagagaagaagaagaggaggaggaggaagaaggtgcgtatcctagtggtggtggctgagtccagggtgtatgggcagggataagtttttaaggcgagtattgctctttaagtactacttcttacactggtggaaggtttagatgattgaattgtgaaaattgcataaatgggtatggcatggaagtgttgattaggtacactagacaactattggaggtaagggcataataatgggatatactaaactaaaacaatgttgttcataaatcattgtttaatagaagattttaaattaagtacatacagtcaattgggaaaggaaaaacagacacaaataagtgttcgcttaagtttaatgctagggtggcaggggattactcaattaagaatgaaatttaagaagaaaatgcagtgtacaggttggaagaagccagaggaagatataagcaatagttacttggacagatggcagtgccaaagctgttcattcagttctgctgtccctgtgtcgcagtgaggtgagtgtgcacattggggttttaggtaattacataaaaaaagagtcctatgtgaaatatctcctaattatcatgagaaatgaagggtaagatttagaatctctgcagctattatatctcaattcaagagtaacctgctgaggataggcctgtgaatatgatcctctttgaagcttaagtcatccagtcgtctagttagtggttgatactgtgtgttcatgataaattctgtttaggccaggtacaaacatcaactggaagtcagaattgtaaaggttgaggattggtagcatttaatcattcattctgtatactgaataagtgaacaaaatgcttttcaaaaaacaattaatttcatggctcctgaatcctcatacctgtctcataattaaggaggaggaataaatgattaagaaggcaccttcttgcactgatttctttagtataagatagcttacctcattgtgaagatgaacgagagatgactcagggcttgtgtttagacattttgttggtgaggaatacctcattttcttgtcagtgtagcattatagactgtgcaaaactccagctccaattcctaccttaaaaagatgttttcagagttagtgaggaacatatacgtaaggggggagggaagaatgatggggaaataaatcatataaatgtagttattaccactgaactgtagacttaagaatggtaaaggtggtacattttatatgtctaatgacttccagaaaaaatttttaaaaattaaaaaggaattactgacatattgatagcagagagagagatggaattctggaatattctgtaagtggaccccattaaatggcagttttagtttttcctaattcagccatgtgttctgcagaggttgtcactgttagtgtttttagtattaaaatttttgtattttacatttctttgtgattaaaagctaatgacccagttttgagattatggcttacaattaatagatttctgccgtggtgttagatagttgcatcgtcgtaggcagagaaagatacacaattgagtgttgatgattatgcctatatttttgtgttcatctgtacagaacagggacttgtcacaattgtttttcatggtgttattctgcatgatctgtaagttggcacgcccctttcaaacccttgccttcaatttggtaatagcacttattctttgtgtcttatgcttgcactcaacatgtgttaactgaacacttactatgagctaggtcgtgtgctagaatgctgggagaaggaggggtacaatggtgaggaaaagcatcctgccttctctcacagtttaatcagagagagaaaaagagaggctagactgatattaatcagggagagaaagagaggctagacagatattaatccaacatgcacccaactatacaatgacaagctgggatgaatgttgtggagggaaactgtttccagttggaggtggggcctgatttcttggcaaacgcgtaatagttgtcaaagcagctactgtctaccttacagatatctttgagtatctcccattgattatctcacatgatcaatgtgcagaatttttactttatggtcagccatctacccttccttgcatgctcttgggtgcctagcatttgtgccctcagcaggactaagtcggaggggctctatttccttccagggtatcttactgggagaagtgggacaaagaagggggaagaaaaaaagaaaagaaactgaggcccttagcccttcctggtacctgtctcttttgcggggctttaagacttagttcagagcgaattatccagttccagatgcacttttattcaagtgcaaaggaaggggatgctttaagaagctgatttgacactttggattatctgtagttcttcttccctccattgcagtgaatgattgggagttgcttcttctagcttctctgtagtggaggaaaacttctgtgtctatgagcataaaaatctctgctatcctttttagcacttttccatagtattaagtattatgctgtacctatcaatattttttaaattaacaaatacttcaataaacctttgttttgagaaaacctactttactcagtgagctagtagttaatagcactgagctggtagcaacacttgaaggacaactaagctactgattactctaagaaatgtgctattaaagatgaatatggttgactttttcacttaatttaatctgaaaaagattaccttttattttcagcgttaaactagtgtacaattttttttttttaattccctagaaggacagaggcctctgtctttctttgggagtttattgtgttgacttgttgctcgtgaataatagggtaggtttcctttaacatgttatgaccttgtttaattttctcatcattacagacatttttaggaagtacacatttataaatcattttgtaacttctcattttcagcctggtcttccattaaagcccttgaaatggaagaagaagaagtgaactacatgtatctccttaagtgtcagaggaggaataatttttaaagtattgaaaacctagttatacgaaactaatttctttgatgttacaagatgaatgtgctattatgtgcgtttagcgtgaccttgaggtttcctacagggtacaaaagagaatggggctagatcaagccaccagggcctgtagaactaggattgaacatggctgaattagaaaattggaccactgtctgagtcgtgttagcaggtaatcttttaaaatcaaaactacatgagaaaatatttatactaatttgctattattctcttgtaggctagttcgttaaaagatctattaccatagtgcttcagtctcatgatctgtaagaaggcaataggaatgttgtctaccccatagatttgtagtgaggattacacgggaaagtttatctaaagcatgtagaatacaccctaacatatagtaagtgctcagtaaacattatttcatcaagcaattctatgatacgaaagtggaatcataattataatacggttatgtttttctgaaaacaaatgtctttttaaacctttactgcatattctttgtgatgttgaatcaataataattggtcaccactttttttctagttggaaatcagttgaatattagtgtgtaggatatctttgaggacagtgacagattctttaaatactgaggcatgcagaagaaaaatgaggctttatcacaggtgataaactctgcctaccaccctgttcccccaactcctacaaagagaattttctaattacatggcctctactttactagggttcctggcgattatgaggcatctctcacagtactttagggtacatttttccttaagctgtggttaggagcccataccaggagcagccctgcctattgcttaaaagtttagccatcagctgggtgcggcggctcacgcctgtaatcccagcactttgggaggccgaggcgggcggatcacgaagtcaggagatcgagaccatcctggctaacatgttgaaaccccatctctactaaaaaatacaaaaaattagccgggcgtggtggcaggcacctgtagtcccagctactcaggaggctgaggtgagagaatggtgtgaacccaggaggcagaggttgcagtgagccgagacggcgccactgcactccagcctgggcgacagagcgagactctgtctcaaaaaaaaaagtctagccatttcagaggttcagtttccagagccgttgacatcagccaaaactaaatatttaattctcccttccacatctttcagtgggatgtataccagcagaactattcttgtgttatttatttgtgacattttttggaaacatatgatactaatgggcacatactcagaatctctgaggcaacactagaccagatatagtcttcattggagtgttcgattttctttctgaaagttcttttatcctgtctctctctctctctctttctccttaccaaccgctattataagtaaatctcaggactgtgatattttgcaggttgaaccttcctggcataatgtattggccaacatagtggtaggaaatgtgtttttatcctgtttagataccagaatgtcagaatagcctgccgttggcacagttttcagtaaaaagagcttgaaaagcaaagcagtaatattgagacatttattttaaatacaagctactccctgattcttctcattttttttaatctcttacagtttccccaaagattatatactttattacagctttcctctacaggattttcatagatgaaagaaaggttctaatgtaattctggacatttgagtgtaggaactattgtaaggataggcgaacacaatgccacagtgattagatttatttgattagttgagtgtcaaaatgggggagaccagaagtttcgagaaaagtcctgtttaactggattgaccagttacttgagtcaaaatgtttgaattgattttctctataagaaacaacccaagtaatttgcaaatgcctgctaatttcatatccctgggcattttttaaaattacccgattacctatcttaatgctcaaaggtccccttaaatgacagtaaaaataactcaagtggtttttgaaagcttaggtttccagcaaaacagaatggatacttcagataccagcacatttgaatgttgtttcagtatattataataacacccactgggggataaagtacattcagtttataatatagattgtaaaatatttgatttaaaactttatgtaatgaggctgtttatagcctatgttaaagtaatctaaataaaatggcctactgaagtcttcttttgagttcagcttacacgggattttcatatttagagatttctttccctcctcagccctgttgttgattactttttataccttctgcaaggaagatgggtagccatgcacatacctttgcttgtgaacattgctaaatggaagatatgaattgacgttggccttggaaattgggaacacacactgtctttagtttgagtaatttttagttatgagtaatctttagttatgaataattcctatcatgtccccttacatgattaggttaattatactcctcccttcctacataagcataattagttctaaactcttttgcatgtaggaagaagccagtacaaagtttcttgttcagtgttttgttttgttttcaatcaatggaaataagtgcaggcatcagcccttaatattgatagcatgtgtctgcaattaaacgatgcagataactacatatgaaaatttctcaggtggcaggagacgctgtagcagctgctgcattcacaccaggacatttctttaggtgcttagagactgtccgaagtcagctcaaacagtagaccaatcacaaactcttgggtgttaattttgaaccaagaaaaagggacagttgtgtttgttttgctttgtttttttaagttttgagacacagcatgaagttgatgtgagtacatgtgcgcaccaaccagcaatgtcatcaaactgaaggaatccaaagaatgatgggaggagttgatatctcttgtcatagagtagactgccacctggcctctatctcttagaataacatggcagaattaggtccaaggacagggcatgcttgcgaggaaagtcacacagagcaccaattcaatggctaattgtgtgcgtgcttttaactattttatgacttgcaaatgtattggcatctatttatgatagtaataatttaatgagcatatgctaagtggaaagcattgttctttgtgctttcattcattccttcaatcaccaaatatttatttagcgccttttaagtgcctgatgcccttgtaggtgtgggaatgcaccagtgaacaaaacagacagaaatccctgacctcatagccttatgaagcagttttcaggaaatgcgacagccagtcttaattatagacataaaagaatagtcctcggactatcgttgacccttgtacaatgtggagattgaggctggcagcatatatattacttgtgacactggattgtacatgtcgaaatagtgacccagtcctgcagaagccctaggaagcaggcactgtttgttaccgttcctattttatgcaagagggaactaagacacaagagaaaatgatttgctcaagatcatacagctaagatctcttgcacttaattacagagagctaagattgaaacacagtttgtctaattgcagaaccaaatgtcttaaatctaatattactctgtctctcagatttgatacctaatagaactttaaaaaatagttttggggattttaagtaatggaagggcctgactgtgaggcagtttttagataacaatctagctagtctataggacattcataggaaagtatccttcagaatagttttgattatattaatatatgatgcttcaagctttttaatgtactattcattgaagtggtgatccaattccacttgctagatatttgaaattggcttattcattgtctcatttgattttctttgttggccaaatttgtagcttatttctgatttgtatggaaagaatggtgggattaattgactttcccccattaattaaaacagtcccctgagtatagattctgagaactccaaaaatggaaccttcagtgactggtagcagggatggtttcttttttgaattcaagggttctgttattgttctctgtgtcactagcttagatgtagagcctgatgctctttgaagctgatcatttcttttgagcttataaatgtttttgctctgaggtgacataaatagaaatttaattacttggaaaatcataaccaaagggagcattttatttcctactgtttggtggaagctgcatttttttttatgccttgtgctttggcatacatacatacatgtatttttggtaaaaattgcattgtaaaggaacttcagaaatttcaaatgttgatggaactgcctttcttcactatgactaaaaggctgattttaaactttaagtgttttcgtaaaagctattttgtcaaaagacagttgagttattttcatagaaggcaaaacgtattatataatcaatatcttaagtaaacaaagttgagttcatattgattctgtatcgtcattttttatttttaattaactaaagagcccaagggaggaaagctcattttctcaagcatcttggcaaaggaaatgactaaagttttttttttttctctcattatactatgcagaaatagtatagctttccagcagtaactttaaaatagtgattctgtagaggtgattttaaagatttcacttgtcagagaggttatcgtaagtggaagaaattttagattattttgcgtatattacagatgagactttcttggatatgtacaagatatgacataaccttttgtttttttttttgagacacagtctctctctgtcacccaggctggagtgcactggcaccatctcggctcactgcaagctctgcctcctgggttcacgccattctcctgcctcagcctcctgagtagctgggactacaggtacccaccacaacacccggctaattttttgtgtttttagtagagacagggtttcaccgtgttagccaggatggtctcgatctcctgaccttgtgatctgcccgcctcggcctctcaaagtgctgggattacaggcctgagccaccgcacccggccgatatgacataatcttaacttttcatttaggttgtttttgaggcacatagaagttaatggactaagatattgggatatgtaagaaatggtagataggctgggaggccgaggcaggcggatcacgaggtcaggagattgagaccatcgtggctaacacggtgaaaccccgtctccactaaaaaaatacaaaaaaattagccgggcgtggtggcggtcgcctgtagtcccagctacttgggaggctgaggcaggagaatggcgtgaacacgggaggcggagcttgcagtgagccgagatcgggccaccgcactccagcctgggcgacagagcaagactctgtctcaaaaaaaaaaaaaagaaatggtagataacagaattcttcatcagataacaatatctgcataaaattttcccatttacacatggtagaaatactatatgttatgaaagaaatttaggtggaattaaaaacatccagggaactcataaagttttatattatgtttttatgcccatggagtgggtcatagcttattcttttttttgagggtgcaatttgatttttattatttcaacttttattttagattcaggggatacatgtgcaagtttattacctgggtatattgcatgatgctgaggtttgtgttacagttgatcctgtcacccaggtggagatcatcgtacccaataattagtttttcagctcttgccccactccctccttcccccccagtagtccccagtgtctattgttgccatcagagcttattcttgatggacagttttagaatgttgttcttcattgctgaagaaaacttgcttaggggataggaaagttgagctaattatgtcaggatatatcttcccactcccaaacacgagaagaacatttcagattctgaccactgtcttgatactgccctgtcttaagctctttgggtccctgaagacacaatctacaaatctctagtgtgctggcctcagatcacatctgactttaagtgactctctacaagttgctaataaaatacatgttttgtctcagcaaagacaaatgaacataaaaggcattaacacttaagctgtgttagataataatggggtaacaatgaaatcctattacataccggagagttaccaggaagtcctgtaccccatcatcttggttacttataacattcgacattgttgttttgcagaaggctgaggagaatgcagagggtggggagtctgccctgggaccggatggagaggtaagggatcgtcatcctttccactgtgttctgctgaatggagtctgtgccatacctggcagcaccattcttcatgcatactattttattcttactgtgctatttattttaagtaaaaatagtaatacattagcatagtagaaaattagaatgatacaaaagggatacagtgaaaaataatctacttacatgtcagactgtcaatcaactgatattaaatgtcttgcatagtatttcagaattttctgtgcacatataatcgtatgtttacaaatatataggatgtgtaagaatcttaaatgattctataattatattgcccaataaatgatacagaagcataaaatatgacacagtttctctagtttattattaatttgaaattataaaaatacataaactaaagaagtcacatatttggttaacttatttttgaaatttttaatgggcttgccatatagcttctatgtgtatgttcatcagcctagtcctaaaattcatttcctagtagttccagtattctcctgaattatgaggatacaattaatttattggatttttttctgaagaatttaaggttccctgccagcgattcacagtgaggaggcacactcccagagcaggatgggaagggagtttgtttcaaattatcttcattttgtctcataaattctgataaacctacttggaggatcagagtgggtgggaggttgccacttcccattctggtatcactctgcacacagttgtattgtgaactcaaaacagtggagatgcagaaaaaaaagattgaaccactattctatgcagaaacctcttgaacaataaatgttacttaaccactataatctatcacaaggaaagaaaggatcaggataaatatctgatgtgtgagaaactccgtgggctgttactggttaagaatactatatattagagcacttagaaacaactctaatttgcaaaagcccagccttacctaagaatgcctcccattggtctataattaacaagcacacagggagagatttaacatgttagcgtaatagtgttcttatatcttattatctgttgggaaaattagctactgatcttggacttgtgacatacatcttttttttcttttaatcttaacacacgtgtcctgtttgctaaggattgcaacgtggacatcttgctctattttgaaataatgttcttctgttctcattcagcgcttttgccaaggttctaattccatctacagtgatttccagttttcttagaggcatgacattagcacgaaaacaagctgtattataatggttcatgataacagaatatgatttttccaagggaaatgatagcaaattgtagatttccttctttatctcttgagtatatagatgaccttttacattaatgaggaagaagtgtcatgttggcatgcaaagtaatgatgttcaaaaccattgctttagcacacacaaaagaaaaaaacgacaatctggagtaagccttttctctttttaaggttatttttgtggataattgctaatatattgtttcttgtgctgacagtgctttgttgagggtgtaaggggaaagaaccattgggtatgtgtttttcattttgttttgttttttggtcatctcctgtaagatatagaagataaacgatagcatactgtaggttatcctatcatttctgcttaattccaattgaaaggaatctagcaaaaattttggaattcatgaattgtctaaaaattcaattttatcacttttcaggaaatagataacttgaaccactcacagaccaagcatttccaaacaaacattttacttagggatctgttcttagaagtacagatcaccctgcaatcaggagcagtcattcgttactgattatattcaagtgtgattcttgtgttccttggatatagtagtctagtaatgaactttagcttaatttttggtcaatttgccagatttcagaggagcagagcttttttctttgtcacactcaaagataccaatagttgggttagacccctggggggttagttttggaaaaaggacaccggttctgcagagaccaaaccagccactcatgagatgacataagttataataccttttctgtttttcattctctctgaaaccatgactttgcaatagttgttatctgggaagtattagttgaggaatggggattgttagttacaaatgaccaaaatcaagtctgcctgatttaaataaaaagggaatgtattgataggatactgggcaactcatcagatctccagggtttctgaagaacctgactcagggctatccccaatattatgctctacaactggtctagtgaggacaggctgtggctgggagcaggcagaagtttgacagagccattaccgctttcctggcattgttgccactgtggtctgccctgcaccggtgcacctgttggtttccacacccttgcacttctagctcttaactcacaaccttggtgatgtatctggttgctggagctgaggtttgtgcctgtatcttagctaccagggaggcagcattttcagtttctttagtgggaggtagcctctcacctcccaccaacactcattaggtagtgaaatttccaaacacagaataaggtttcacatgctgggcagcaaaaactgctgagaaacgtccagtacaggactgggggaaggtcttaacatgaaaccgtgacatgattttccctcctttgtagcccatagatgagagcagccagatgagtgacctccctgtcaaagtgactcacacagaaaccggcaaggttctgttcggaccagaagcacccaaagcaagtcagctggacgcctggctggaaatgaatcctgggtaaggcatgaaagcagcgttcatggtgttcttttagctttttagattttggtaatcttgtacgatctcgaaactaaaactacacagcctttgctgagatggttgaagttgttacttggatttgtcttttatacatagaatgtgctattaagtcatgtgtgtctttattgtattgtaatttaaaaaactaagccccctcctcttcctcactctttttccttagttatgaagttgcccctagatctgacagtgaagagagtgattctgattatgaggaagaggtatgtatgtatctctgtttggggtttattggtttgaaagttatcagaggaagaaattcttcctgaatgtaagcccgggccttgggtccttctatcatttcttcctccaggattggcctttgggtgacagctggggctaaggatttacaaatgctttgatggtgtatgcctagtggtacagtgatctcttggaaggccctggctaatggggaaacctggcacattctaaattcacttgaaaccaagagaagagaagctggcattaacttgggagatggtgaagtggtcttttgggggagaattggagatggattaccttgggaaaggccataccatacctctaagtggttcacattgagaattaactgccaaaaagttctgaagtccttcagaccttcgagtctatttgcaattctgagtctggaaactttgacaaaaagggcaacgtgggcttggtttaatgagtttattgatcctcttgatttctctccaaatgtgattcttgtttgaattccttagatatacttgttatccaaagagaagcgctcagtctcttggaaggaatacttagtataactagaacctaaaatgggaatcttgggtatttttaatagaaattgagaaataataaaatatttgttaaatcagccacaaaatgtttaatttaataattacaaatagaaggattaaaagaagatttgctctcctttattagagcattgttaatatattgtcattagatctgatatttttgagagtgccgtagtataggtgccgaggacaaggtaaaacatccttacccataatagcttaccagggtctcaagactatggctgttttccttctatttgtggacctgtgttttccaaattagtcttccatgagcacttaatgcttttgtaaagagaaagaataataaaagttacattacaaaaaagcataatcattaaaaagcaaacatggttgggcatggtgactcatgcctataatcccagcattttgggaagctgacgcaggagaattgcttgagtccaggagttcgagaccagcctgggcaacatggtgaaaccccatttctacaaaaaacacaaaaatcagcggggcgtggtgacgcatgcccgcagttccagctactcgggaggctgaggtaggaggatggcttgagccctgggcagagactgcagtgagctgaggtcgtgccactgcactccagcctgggtgacagagcaagatcctgttttgttgtgttttgttttttaaaagcaaatactttttgggggatgctatttgtttcccccagctactctatctctaagatatttaaaggtgtcttgtttacttggatgaatgggcttataagtgacctactggaagaagcttctctttgaataagagagccagagagggcagagataagatgaagtaggccgcctagccagcaggtgccatcctgagaatgacaagcacagacagcccttgtccctgggcccatcacatgggtctgctgcaggtgagcctgagtgacatgtggagagtcatgtcccctctcagcgttttatttcctggcctggaaaatgaaggagttgtgccaaggccttagctgactccctgcttctaaatgatctgctatcataaggtttgatttttctccttgatagtttgtcaattcatactttgtcatttaagagttatggaaaagcatagttctctttaaacctgcttttcaaggaaattctactggattgtctggaaagatttagccttctacaccaggttagtagtggacagaatggacacttacttagtgatgctttcaaatgggtcagctggatgtagcactcggttgagaagggagaggtattgaatccttcgccaagttattgagagtttttaatctcagtactctgcaatgtgtgatgtgaatcagccacagaaagcagtgtgtttacaagatccaggtcctctacagtggacatatatggggccatccaagtcccatgggcattatcagagagagtagattttttttgttttgttttgtgttttgttttgagacagagtttcgctcttgttgtccaggctggagtgcagtggtgtgatcttggctcactggaacctctgcctcctgggttcaagcgattctcctgcctcagccccccgagtagctgggattacaggcatgagccactgcacccagccatcagagagagtgtttttatgagaaggaggatcctaagagctctacaatctggctttggagagagtgctactgagtgagagcagatatgctctacaggactctgataaccaaataatgataaacatttatgtttctggcatgatatttacatactcattcttgggagcaagggttctaggggatcctcagagagttcccttcattatctcattcatgatttcaacagcagttgaattgggggtattaccttacttaagtagatgttacatttacttcctccctgaattttatgatcttacttcatttgtgaagttcaatactagtttatgtcttttcagaaaatgtcaatcatagagatttccttgttaaaatataatttcctccctatctttgttccttttccaggatgaggaagaagagtccagtaggcaggaaaccgaagagaaaatactcctggatccaaatagcgaagaagtttctgagaaggatgctaagcagatcattgagtatgtactgcatttttcccttggaaatgcattgcatgaggatgatgcttaatgaattttcttttaggaaattttgttcaaggaaggcaactaacttcacgcctacactctgcttgaaactgtgctcctagaggtcaccactgagttctgggttcccaggtctcggggtttcgtgttgattctccatccctccctcccttccttccttccctttcttccctttctttctcacttccttacccttctctctccttaatccccacctccagctcaggctcactcctgagcctcacacctgtcgtctgcacctcagtcccagattgaactaatgaaacttatctccctaactggctgcttcgctgcctttcctgcctcccttaatgatatcagcagtctccctgaaacccaagtgagagacaccattcctgtcttactcctgctgccatagccaagttgaccaccaactctcgcccagtccctgaaccctctccagccctccgagtgccctggctctgtgtctcatccattcctgtgggccctctacgtcaagggtttgcacatttctttgcttgtatagctgtactattaaaagaaaactgttgtgaaacaatataaaagatgaggtattttaaaaaccagaaatagacatcctaatatttctcccttttattccatggttgtgtgccccactttagggacccttcctcctagcagtctgccctgatccttcactgaaggtgttaagaatccccctcagtggttcctcattgcctgtagaatagcagagtagctcatgaacaaggcacagaagatagaagcctacctttcctccttcctacccctttaatgttgggttgccctctccccctgccctcccagctttagccagtgttctgtgaataggatttatctttgtatttaacagtctaggacagcttcatcggcaaagtattcatctgcaggtgtagggttcaggctcctatctgtcttttgggtcaagggggcctgcattcgaatcctggtttcactaccttttatgtgatccaaacaaatgaagtaacctctctaagcctcgggttctccacctgcataaaatggggatgatcatcataacgccttttgtgcttgctttttgagaggattacagatagtgtatattaactgcatggcaagtcgtggttgataaataaaacacacttattgcagctattttaggttctcaaatcattttttgagtgaagtaaaacaacacatgcacgcacatgtctgtgtgtgattctccactttttcctttcccaggacagctaagcaagacgtggatgatgaatacagcatgcagtacagtgccaggggctcccagtcctactacaccgtggctcatgccatctcggagagggtggagaaacagtctgccctcctaattaatgggaccctaaagcattaccaggtaattggcatggtttcagtttccttggcaagttgtaaccatttacctaattttgattgaagtatgtcgtgcacatgttgactaagggcttttgatgatattttaggccacatcacttataatactgaggttcctttcattgtgcctatgattcttcttttagagtactccatgttaccatctttactagggcatgccatacccattttctcttttatgcttttgggatgtattttaagtgatctctgcaaaatggaaaacccagccacattctattaaactggagaacatccacgcctcatcttgaagtatttctttaaaatttttaggtgtggctgtggttttatgccgtggattgaacccacctaggagtagctgtctactacatttatgctttttcttaagacacaataggccaggcatggtggctcactcttgtaatcctatcactttgggaggctgaggtgggtggatcacttgaggtcaggagctcgagaccagcctggccaacatggtgaaaccctctctctactaaaaatacaaaaattagctgggtgtggtggcacatgcctgtaatcccggctactctggaggctgaagcaggagaatcgcttgagctccggaggtggaggttgcagtgagccaagatcttagcactgcactccagcctgggtgacaaagtgaaactccatctacaaaaaaaaaaagaaacaataaaattgtgagcatctgtgctttgattaaaaattaggccattgttttatttgacctgtctggattgaggctaattccctgtgttctaccttctaccacttagtgacgtttcccaaattgtgttccatgagacatttgttctatgaattgttccttaaaaattaaaaaaggaggccaggtgcggtagctcacgcctgtaatcccagcactttgggaggctgatgtgggccgatcacaaggtcaggagattgagaccatccttgctaacacagtgaaaccctgtctctactaaaaacacaaaaaaattagctgggtgtggtggcgggcacctgtagtcccagctactcgggaagctgaggcgggagaatggcgtgaacccaggaggcggagcttacagtgagccgagatagcaccactgcactccagcctcggcaacagagtgagactctatccaaaaaaataaataaataaataaaaataaaaatagataaataaaaaaagaggtttgtggccaaatactgtaactaagattaccatataaattactatttctaacttgaatacttgtgaacgtgagtcgagggccttattattgataataggcataaccctggactgtccctgaacaacaaccagatacggtcactctggcagtgacccctcttgttaattcacatgcccgtcagcacacttgggactctgcggaaatcaacatgtaaccataaggcacatcagcatcctgcagtaaaatcacttctttaacattgtttaacttcatatttcccaaatttctttgactaccaaaccctatttttatatcaccaattaacatcctgtaagactagcgtcttctagaatacactttaggaagtgtacttgttggcatcgggtcctctctactcttaacacttggtactgtgtctgctgcccttgcataggagtgcagaatacaatgactgccagtacagtactgccttgatggtgctgaagcactagcagtttttacctaccaatgcttttgcatcatcagtgcagacgtcaacacagtaaaaatgacaagtaatgccttagtattgtcatgctaatcattttgacctcgtggatctccttccaagatcttggggggccctgagaacccctggaacacattctgaggaccactgctctggccttagtaagatcatgttttctccaaagaccatgtcatagtcctgagatccaaagcaggtttcatcttgtttggcctacctgacatagatgttaactcatagcatgctctaataaacttttgtatatctgcatatgggagatccatcttttgaattatttgtgaataacttgtaatgccacatgggttttcttctgtcatccaacacccttctaagcagtcttcatcactctcttttcaaacctagctccttcgacttccatatgtgagttttatgcctctgtgttgctgagctgctgtagattccccgaacatgccttgcagtgtctctctggatttataccttatgaagtgccttcttcctggaatcttgactagccatgtccgcctgatgagatctcacaaaccagttcaagttgtggctcaaaccttccctgatcatttctcataaaccagttcaagttgtggctcaaagccttccctgatcattctctccatgagtaagctttctatcttcttgcttcttgacaggtttttgtgtcattaatatttacttaaccttgacacggctgaggtcttcctgctactcctagagagccacataagcttggttcaatatctaatcctccccctggccaattctgccagtttccagcctgagaaaatccaaattacttcccacaatttttgtgaatttctagtctcctctgctgctagtttctacttctgttttgtattgaattagcacaaacgcaaaattcagcccctttaccaattttctctaaaaaaagcagaaacaaaaattcatcctacagagtatccaaaatgtttcttgtatttagttgacttttctaaagctctagtggtttcagggtcacgttgcaggctgacagcttctcctgtctaccactgtttgttttataataatttcccctctttccctctatcttttgcccattctcttggaggttcttcctgtttctctacttatctcttgttgttgttgtagttctgcagaaacctttaggatgactttggaatcttgcatggtacaaagtatctcaagtggcttatggaagtattgcttcaaaccaatgcggtgtctccctaagaaaaattcttgtcaatacagttttatatgtatacatatacattggcactgtcaaccacttcttggcatatgtgtatatgtgcctcattcattgttttctcttttatttgagcttctgaagggaggcatgcatcttacagaccttggtatctatctgctacagctccttgcatggccccttgcacagaatagtcatttaataaatattttaaataaactaattgcttgctttgttcttacataaaaaattaacatttccctaagcataacatcattagcaagctaaattgactttagcaaacatatatataattgatcctatcatttaaatccagcatctgtggtatgatggtggaagggaattgttggttgggggatggaggtttaacaattttgtattttcacccaaccttacttttgtacttaacgtggggaatctctggtacagtgtttttgtacttcagaaatacgttgagcatgtttccagttgccagcttccagtgtgcagcctttgggtagtactcacattacaaagttcccatgtgctgtctgggaactttctctctgccaggaagagaaatgtcctcctgtggtcccagcagagtgtccctggctccctggtcagtccgccttttcactgaggggacctcacccatattaccgaaaaggctgtttttaataatctgtgatctgtgtgccaaatctgccttcacattggctttttattcttggagtggttggattagattttggtttgggcttttttcatttataataattctagaatgtcacatactgtgaaatactaagtgtggatgagcggcaaagctgctttaaaactcacctgtcaagaagcaagaagagagaaagcttactcatggagagaatgatcaggctcatattttacattctttttgcattggtgcttcccccattttcctactgtgggaaacccaacatacagcagtgaggagttaagaagtcacaccctcacttgggttgtattaggattaattacctgtgcagatcttggataattgaagcaattactcttcatttcagctccagggcctggaatggatggtttccctgtataataacaacttgaacggaatcttagccgatgaaatggggcttggaaagaccatacagaccattgcactcatcacttatctgatggagcacaaaagactcaatggcccctatctcatcattgttcccctttcgtaagtaaagtcatttattccacagtcatcgttctgtatgttgtagagtgcccgattagtagcttgtccttgttttttacttaagacagaattgtagcatgtactaacctaaatccagattaccaagagcatgtaatctggatttaggttttaatttcccacccctccccatcctagcctgtttgattggctatttaagcacgggaccgagtgattttatcggccatcagcattacaggtctgcacagcctgaacactgcatagtgagtcttgctcatcattattccttaagtggctcacaaaggggaaaggtgtgtgtgtgtgtgtgtgtgtgtgtgtgtgtgtgtgtgtgtgtgatttctttgattttttccttattccatttccaaaagatgatatattcttaaatcttccttttaaaagcagaacataaatcaaaatcataactaggagttttgcattaaaataaccttagtatgtgcactgaggttatgtttcaatgagtttgctgcttagtaaggacaggagtttgctgttcgccattacgttcccagtgcctcacctgctaggtggttcttagaaggtcctaagtgggcactaccgagtgagggcacagtgattcacaagcaccctcctgggagttcagtgtgtctgcctgtgggtgttaggttgcagttttgttcaccaaggtcttgttatagatctcaatgctgatatgaacttggaccttcagtaagaagaaggattctattaatattattgtgttttctttgaattttttctaatgaagactatttgcagccctttttgtatgaattatctgtatctgtaatatttaggcatcttttgcatttgatctatattttgcgtgttctgaatcatatatacagtgacactttcagggctttaaaaaacgtcattcaatatttcttcccctgttccctcttgacagatttattttatgttttcatgtggttttataatttctttttcctgactcatcttttttctgaacctatatggctgtttatgaagattcctaaaatcattctggcaattgctcttttttaatatcatattacagtttagttttttgctctctatatttatgtattctttagtttttttcttacaagatttttttatgaaccacaaacttgtcatgttttgtgacagtttgttttgaacttatctcctatttccacacctttaaaataaatatcttgttgagatatgttatattctgtagcccctttcaaggtgaggcctgaacatgaataagaacatctttttaaccattgatttttatacaaatgtcttttttctgttgttttttttttttgttccataggactctatctaactggacatatgaatttgacaaatgggctccttctgtggtgaagatttcttacaaggtttggaatgctgtatttatatataaatgtggaaaagcaaaaaatagaccctattttcttcattccatatgcacatctgtgaactgtatttggttgtaaagttttgtcatgtacatcatgtactaaaccgtgagagttaatcatcccaagaagattactgtaataaaccataattactttcagatagtgaaatccagaagaaaattgttaattatcatattgcaaggtttctgatgagagtaatacaaatgacaaatatcacacattctgcgtgctgctgactggctcgctgtgggctgctactgtgctgactgttcctgcaaagtcaggcaattacccggatgcccggggctgcctgctgtactttctcaacagaactggagcctttcagcagagcggtgggttctctactaccacaaattcactgtcatttccatttccctttgccctcccactgtctttggtgggaaaacctttctaaaaaaagatctgaaaagggtcaagacgtgtaatttagccaaaacttcctcgtgtataatagatcagtctatgagaatgtgtgtctgtgcattcttcagtcacatgtctgggcatcattaagctatgaaaagtgagaaataatgcacatatatgtccataggatcatgcatgtatttttttctttccattcagggtactcctgccatgcgtcgctcccttgtcccccagctacggagtggcaaattcaatgtcctcttgactacttatgagtatattataaaagacaagcacattcttgcaaaggtatgtttttaaaaaattattttctctctaattaggaccattgcactggaatgtgaagtattgcccaagtcagtagtgtaattggatccttagatggcttgtccttttctttatttttctaaaattttttcagagtttgatatgttttggtcgtggatttgatttcatgcatgctgtgtgtgtgtgtgtgtgtgtgtgtgtgtgtgtgtgtgtgtgtgtgtttatgattgatcctggggcggctcttgtctatcccctcagagttgatgagtttacatctcttaaaaaaattcacctggttgcttctgcaactctttttctctctaacagctttgcacattctcgggaaagaaataattgtttttgttgccctgagatattgtcagtgtaccttcattcttcttgtgcctttgtttctccaaagatgagccagacacctctccatatggtctccaccatatgaacatttatccaagccgtggcttttgtggtagtcttgtttcaggtttttcctggtgaaataatagactatcattgttggagttacttaaggttgtttctgtgactatttatttgaaactgagaaagaggtgtttctgtgacttacatgtctcctaatttttatgtcctaagttgttccttagccacaaagtagaaatttttactcccatttatatcatcttgtcttctaatacgttcatgtagaaagatggaagtgctctctttcgtggaacaaataatgaaaactcttttttttcctctttagtaattttttttaccccatgacgttttgaaagcactataatctgaaactaagttatgtccaaaattgaatagaacctctatttctggggtctattctacctctgcattaaactcttgctctgggaactgagagttcccttcatggcatcataggaaataacaaaaaggaaatgtgttcccaggggaggtttaataattgagatggcactggcaacaatgctgtgttccacctcctcacactcagagatgaatttggcagtgtcaaaaatgatgtgttttcaatattactatgggaatgtgtttgtacatttgagatccttttaaaaaaattcatcttgtgtcttctgcatatgtatttcaagattcattggagacgcctggtaatgagatgcactgcttttatttgttactggagaaatggagctatagaaagaggtagtaattttgtccaagtggaagacctaattagtagtagaccccatagcaaaatcaaaggcctctgattctttattatttcataagctaaattaggcaggaaagtagatttaggatatttatttaaatatattttttgtctgcttgcctcttttcatgctgttggtaagtctgaggcagttgattgtacagctttaattatccttaaacacttttcttagaggtggtggaggaaggaaaaaaaatcgaggtagttttttttttgtttttttcttacctgtacatcccagcctgactgatgattttattccttttctcctggcacgtctgatgcttctaatgaggaaggcaaggactgtatagaatactttcctcatctcccttctctctctaggagctttgcagcaggatctggggacatctgagcaccagatgtttttcttggccattgccactgttttgtagagaatggataggcttagtggaacattcctcattgttctcactttaagaaactgaacagtgctcttaagaggactgaagggtaaggtatgagagagggaacgtactgtgtagacaggaaggaggactgggccatataagcattctggacgttttacgcaatcgtcatgttggggaagtatgctgtatttaattctgcagttttggcataaatacaggagtgatatttttgcagtgcgctttctgccagtgagtatattcttttattttagataaaacaggggtcctagatctggcaggtaaggaatgtgcgtgtattgagggagcttcgttgtcagacctaatttttgaaaagccgtatgtacaaatttaatgtgaaatagtccaacttttaaatgttagcaaccagtccaaacaaaacaaaacacacttccgggccaagccctaaaggtagtaaccagccaggctaatagaaaagaaggcatggagccaagaatatctcagctgtagaacctgctagggccatcttggaaaaatcagtcattaacctctgcttcaggtgggtacctatattctgtgcctgtcatgagctgaaatagcatgaccatgaaatctgggatagctgtcatgataacgtattaatagaaggggcattggatggggagaggcaggatccacacgtggaaacaaccatgtcattctggacccaaatttccttctttgtaaaaagttgatttggaattacgtggtctgtaaggaacattgttcctttaacattctattattataggatgtcttatgcggcctatcaggcatgagacattgttgagattcccttgtctcaaaggtaatcacagcatatcatataccaaggatgggttctttggttttccgcaccaccacttgcttgttggaaatagttgtattttcccttgcttactacacgtccgtccttcctcttgtgttatagattcggtggaaatacatgatagtggacgaaggccaccgaatgaagaatcaccactgcaagctgactcaggtcttgaacactcactatgtggcccccagaaggatcctcttgactgggaccccgctgcagaataagctccctgaactctgggccctcctcaacttcctcctcccaacaatttttaagagctgcagcacatttgaacaatggttcaatgctccatttgccatgactggtgaaagggtactggtctgagttctgttttttccactgcatgataatgacatgaaatgaaaggccctaaagctgagaacattagagccacagaacacccgcagggtggtttccactgttgtttattttatgaaacccatcggtgggtggacatggtcttgtggtggggttattttcccctcctaggaaaatggatctagtggtacatggggcatctaacccctttgtcttttgccagcatgtttaccaggctaccagaaaagggtcccaaccggtggaatgcttaactgcagtgggccacagggaggatgaacatggagatattttgctgaagtaatttaaccagaccaaattaaccaactaaatacaagctttaaacaaatctttccttgttacaaacttggagttccctttaaaatatttccacataaagattttgtgtgtaaaacagaactagttttcacacattttctgcagaccttttttttttctttttaggttttaatgtgattttcagttttgtgtgtacataaatagtcttcctggagtagccatttattttccaatttcttattgttccttccactgtcttctcttaagagctcatttatgaaaggtagacaatgggccattgcaagaaaagaaatgggcacgtgtcattttcatagtggttcctcattcataggaatgaaaagccactaaaaatgctttagtaaaatttcggtttgaattatttggccctttgatgttgaggcacatgggcccatcctcagtagttaactcactggcttgcccatttgtagaaagtagtgtcaatcaactctgccccctagaggttaataggagaaggtgaaagagaaagccgggaaatgtataagatggactaaacttgaactagatagggctgagaaaaggaaaggagtgaagttagagggacttagaggtccaccagcataaactggatcaactcttgttcctcttctttatatgttcaagcccctgttcctttagtatgtttctgtgtctgagacaaccaagacgaagggcctaacttaaggctgccactttaaatagggaacatattaactgttgcccaccttctcccattctatctcagtcttcagtctgtcttcattgacagtcatatcccatacaatttgaggctgttgacactactgataaaaaactggtggcttccgggctcttttccatagaactcagcgttaagacaaagacacctatttagaggtcattttgttaccacttggtaggcaggtgggaagataattcagaggtagatatgacaggcttaaacttggctttaatgcatgtaaaatgtcaatcatgtattgaaccacacatatgtaacataaagctttattgtatgaaacatccttttctttaaaaaatcaaattcataataagcctctcttacaggtggacttaaatgaagaagaaactatattgatcatcaggcgtctacataaggtgttaagaccatttttactaaggagactgaagaaagaagttgaatcccagcttcccgaaaaagtatgttgcacaaccaaaagttgtgggttttttttttcctccagcaaatatatttgaagtacctgcctctgaaattgtgtttctgaaacagcagactcatattgtagttaatagtatcttgaaaatatcttaaattgtttttttgtttgatagggctagagagagcagggaaattatagcatttgcagcctgttttccaaaagcgttcgagcagattttatgaaattatcaaattcactgagtctcatttacattttaattttagatttttttttttttttaaattacggacctcattgagttcactgttgcaggcactgtagaattgttctgcttataagtgaatggaagatttggcgttggggacttgtatcatcattgaagggggtggtaaacatttacaggttttctagaaatttatttaacatttgatgtttgtgatgtcaccattctgacagtaagtgaaaaaacagagttcctgcaaattacagacacatcatctaattattcggggacattatttttgttctgtgccataaaaattaagttcctgtgaacattttaagccagtatttttcggactaacttttggaaacttagtctgcagtactttagaacaccttttgagcccttagctccatgttttggggttggatgtgagtattagtttaatatgtttctgatcatattttggaaccatgcatatttgaattctaagtttcattctcttagagacagtttagcattgtgtactatttgttgcatgaggtattttgatttgagttttttctttagaagagccaattctgaaatgtcttatttttgcatgattgtggtttttctgggtaacgactctatttttggacagattggttcatcctgttcatttttaagtgggagttttcaagatggtgtacaacgttgatcactttgttacgagagaattttattccctaatcctttatgcattgtcagaaaaaaaattaaaaatgacttttataccatgcatatctcagtgaaagtctacgtatttaagtattttattctgctggtgaatgcatcttggacgtagaatgctattgatattcccttggacttcctgtggtcttggcctctgggtagcaccctttctgttcaagatggagtttagttactgagtagataaattgatgaatgggtatagtggccatgtgggtgccttgaattacttcctagcatacagttacggtgattctgcaagtgatagagatggaatgttaaacattttaatttatcccataatttctttttttttctttacaagaggtataaaataaaaggcaattaactgaagcccttcatttattcattccataagacttcttaatcttgttcattcacttctaagactggctaatacagatagatttttaaaaatatattgcaagttgaaagtatgttatgttgtaagaaaatccactagagaaaaattgaaatttttgcagcttaagaattatgaggtgattaattatatcacaaaggaattctttgctttacaaaaatatttattttatggttctttctacaagagaagttcctgagtatacttgaaggatgataaaatttttatgaaatgtatctaccctcaaatgatcacgagtatgtctcttgcataaagtatacatgtatttattaacacccagagtattatatttgaggtatgtaaaatactcagtgtgtggtattatctacctttagaggtatctgtacctttaaaggcagccatgttatcttttcctttggtggagtggaaatcatgggctctgcctgtcagagtaggcatgggtttcaccacattctccatctcttactatgtgaacttgggcaagtcttctaacctaaatgggtcttagtgttctcatcagtgaactggaagataaatgactctgtcttatggagttgtgaggattaaatggatgtagggctcctaataccatgccaggcacatggtgggcacccaaccaatgacagctgcttctaccatattccttattttccactcatgttgctgtctctgttattaatgggagttccattttcctattcttgaagctctttctcgctgttctcttgtctttgctctccttccggtagcccccccacatctctctccataaacttttcaagcactttgtgtgtgtatgtggttgttgttgatatggctgggctgatgttcattttctactttttcagccttgttaggattttttttttttagcctggggaaagttggaaatttcattgccatcctagggggtaaagaagtttcttctaccaccactgcttacagccacaaacaatgtgcatgtcttacttagcctggataaagaatccagaaaggaactacggcagatggaggaaaaggctaaatactttcacagcagtaattctcaaatgtttttatgaaaaaagagccatataggaaagaggcttttacttttattttttaatttatgtgtagcaaaatttgcttcttttggtgtatagttttatgagttttgacaaatgaagagttgtgttaaccatgcccacaacaaaaattcaaaacaatgtcatcatccaccgctccaaaaaaaattccctggtatttcctggtactacttcttttcagtgaaaccctccctcggctcgtagcctcatgcagccactcatccgttctccatccctgtctatagctttatcttttccaaaatgtcgtatgagtagaataagacaatgtataaccttttgcatggttttttttaatttggcaaaatgtgtttcagattcatccatgttgttgcatgtatcaatagttggttcatttttattgttgaataatattccattacatggatgcagcgcagtttgtttgacatttgggtcatttctagtttttgacaattatgaatagagctgctataaacttttcatgtacaggtttttgtgtgaacataggtttgcatttcacttgagtaaacgccttaggtgtatgactggttgatcgaatggtcagtgtatacatagatttgtaagaaacttctgcattgtttcttagactgtctgaagcactgggtagtcccaccagaaatatatgaccattccacttcctccacattctcattagtgcttgctattgtcagtttttcaagatttttagccattcaataggtgcatagtggcgtcttgtaactttgattggcattctctaatgtctaatattgttgagcatcttttcatgtgctttctcaggtgaaactaaagcaggtatataaaggccacggttttgtaaatgcaggtcaaccgcataggaaaattagctttaatattggtctaatgaaacacttaatgagcaagggaatatacccaccccccaaccaccaacaccgctaagctgttgtcataaaatccatttcttatgcacgggaaagaatgccaaatagaaaagggattatgtaagaacaacttaagctaaataaaaattgatttttctatttagctcacatttaaggttgtctgtgctgactcttttgttaattcatatcatcaagaaccaactatacttgcaattggctgggtatttctttatgtctaaatatgctaaaatatgtatataatatctgaggttaactagaggttgacctacctaaagtcaatcctgattctgatataatttgtgaaaaacaccaacaaagctttgcttacattggcaatttgtgtattcattatatcaaattttggaatgcttagcataattaatcattactttgttcctctctcatgccatagcgtttagaacctgagacttttcataaattgaatcatattgtttacaaatctcagttcacaaagtggtgatatatcgcatgtagtttaggtttattattccaataaaacatcctttaggaaggggaattataaatgaagaatagtattcaggcagaagctatgaacaaagttcaaatacttctacaggggtatataagaagaaatgctgagccagtataatgacctgatttgcctacttttagggaccagttaggccatttactaacataagaatgcttaggagataattaccagatacatgaggtcagcacctcagttttacatatgatttaaacaaaaatagctccagtggctctcacaaacctaacttactgggagaatcacccccgtgaagaagtcacaagtttcatttgctatagcaccagtatattttgaggagctttcacatgcatgctctaaacagatgccatcacattttttgggggatgaactcagactcaaccgagttggagttggcacgattattataaaaacttctctcgtcactgtgattcagtgtggtggctgtataattgttcagtccatcccttttcttgatacaaacctgttgattgcctgctctgtacgttatactgcactcaggccaggtgggaggcacaatataccagaaatgggaggcatggtgtgagaagtcaacagacttggctgtggagatgagggctactcacactgaataatagtgtggcaatgcaaggcagcatcatcccaccaatcagaatgaatgatccaggcattcaggtggtttaggacggggtacccagtatgtacacatgggtgtatgtgttgggaaatgcttattagtaatccattgggtggagaaagatgttagaacaatcatatatattgatctttctcttaaaaagtcttcactgatatttaacatgcagattgacaccggtacctatgtggtaggtacgtatgtgtgtctgaaagcgagtcatgtgctgggagtacactgtgtaggttatcccaggggaagcgtgagagtgggtttccaccactgggagcatctgatagtcctttcatccttgctgcctggttcatttactttctgccaatggtggtgaggccttatgggtttccccgtgcccagataaggagatttagtatgatctgattttaactaaactaacaaccctggtccccagaagggcttaaaaggattctgcaaaggagtgcaggttgaaatcacataggcatagatgaacaatgggaaaaaggccatcttgtctcttttcccattcctagtattggttttcaacagccacatcatggtacatgggctggagttgaccaaaaatattagatatcatcaaattggcagctttaaatagggctataaatcaacttcctttaacattgacctgtgccctgaaagtaactcatatcttgagacattaatattagtgcatgcatatagtaattcataaataaatatgtagatatatgttgggaaaatgagcttcagcatgtcttcttgtttttcttctccattatcaaaaaagcttactgacaaacttctttgagagaaatgaaagggaaattggctttggctgatagtgtgattgggaaagatcttaaaaagggccctcaaggtagagtagagttagaaaggagccttaagactaagtaaggatatcccaggcctggagacaagtacaaaaacaaaccaagtggcctggagttgtttgagaaggctctgggagcaaacataaaatatagttgtgaagcttatgtgatttttgatcctgtgtattaaatgccctaattgtgtgaggaaagagactctgccttctaagagcagccatcaggtccttgagagctgcttgcctctctgctgacttgacctcgttgagaatgcttggcatccctgtctccatggtatcaccatgcatgcctcctgctttccaaactccttcacctccagcccttagcagtggctgactcataggaaggtgcttgagtgttctttcctgaattctggatctgccttccccaaatgcacaagcatcagccatgccttccacattcaggaaggtttgatcaagccagcagggaggtgcaggtgaaggtgaacctcaggcagagctaggaagcctgaaaatctcctcagtcgtcctaacgtggagcttaaaagagtggattctatttcaaatcctaactgtgccattcaatagctatatctttgggtctgtagctaaacctctgtggtgctcagattcctaatctataaaatgagaaggctaaatgtggctatcccagtacagctcttttgtgtgttaaatgagagaaggctcagaaagcactttgcacagggctttttctgtggaaagagcccagtaatattagtccttgatgtcattttaccattatacttgttaaagtctaaagttgttcagcgtttttctttacccttatcaaatgtcactgtgagttaacagctcttgaaaaagtagaagcctggggtaatcatttcacaatgtttatgtatatcaaaatctcacagacgtaccttaaatatacacaatatgtatttgtatatattatacctttctaaaatattataccttgatacaattatacctcaatacagctgaaaaaattagaatttttttttttttttgagacagagtcttgctccgtcacccaggctggagtgcagtggcgtgatctcagctcactgcaacctccgcctcccgggttccagcgattctcctgcctcagcttcctgagtagctgggactacaggcacacaccaccatgcctggctaatttttgtatttttagtagagctggggtttcattatgttggtcaggctggtctggaactcctgacctcgtgattcgcccgcctcggcctcccaaagtgctgggactgcaggcgtgagccaccgcaccgggccaaattatagaaaattttcataaaaaaaaaaggagaagtactttaaacatataacatgatcagttatctaaaacacaaagcctgtattaaatgcatacttattgattaatcttggagatttttctttcacctggccaagaaggtcattaaagactaaagcatgtttttggagggttacttacacttctcaccttttctgtaaaagagcgggcttttctctaatctcatttgatgcatgaaaatgtgtggccagaaactattaattgtgcttccatgtggcctgacagattccttcgaaagcaacaattgaaggaatattcaagttctctgtgacctcttctccctggccctcagagctgcaaataatgtgatcattttacatcagtttattaaacattagtcagcttgctgatttatttttaatcacattctgcatatttctaggcattaatctgatgtttattatgagaattgcacatttgtattattgggattctttattaaataaagaaaaacaatggtaggaaataaggcagattttatgtattaggagtatgataaagtacgcaggtgtgtgataaaccatgagcatgtgaattccccttaactttttaaaattggattttgatggtatttcaactggaagcacaggaggccttgtcctcctttagatgaccataaagtagtgtccaattgtggattaatttctcttccacctgttctcactatgttgtatcctgccatttctcaagagtagaagttaatggtagtcaccctagggatgttataaatctcactttaagactagtattagagataccacatgtgaccatttaaaaatgtttcacaaagaaaatcacaggatatttgccagtcactgcttttcctaaaaccgttctgttcataggaaaggaagtataattgggaagtgaaggaaatgctaaaaatatcccatattgaacatttttgaatttaaacagtttgtaagccattctctctctcaaatttggaaccaagtcaagaaatttcagaaaacatgaccctagcatcaattacatataaccttcagaaggcccccccatcataatggtaccttgaacaatgatgactctggtctcttctaacacccccatctcaccccgcactccgtgaatccaagaaagagaaagagagacacctttgtgcttccaggagtgacactgactcagcagtcttcttagaacaggcgccttctccttcctgctcttgcctacttactgttctcttttctgcaggtggaatatgtgatcaagtgtgacatgtcagctctgcagaagattctgtatcgccatatgcaagccaaggggatccttctcacagatggttctgagaaagataagaaggtacgttgcgaaagatgatgcaactcaaggtgctgtggtatgtcttctcatggctgtgacattgaatattcacaggaagcatccctgctaatgctgatttgttaaagtaaatcactggacctcctttgagctattatagctgccaaagataatctcttcacctcttagctgcattggggcaggaagaaagaaggaatatgttcatattaccaaaatagttagctctgcttatgtcaattttgcagcaaatatattctgcctaagtgtggggtggcattttagaaggttttaaaatccagtttcactccctgggcatctgttctgtgcttctgtgttccggagtcctacggaatagacagtcatgtcgcaaagccatatccctcactggacataatccttcaaggttcactttttttgtagcgtaagttaatcacaagaaagacattatgtctaactcaggaactgctatataattaaagaagctttgaaccacaaggtgtgtaggtgactgaaaaaacctatggtcctttgtcctttgtataagaagcccagtgtgaaggatctgaaatgtctgaccagttaataattaattctatttttccctttccactggttcttaggggaaaggaggtgctaagacacttatgaacactattatgcagttgagaaaaatctgcaaccacccatatatgtttcagcacattgaggtaagtctgtattgtgtgttttgagcctgattgtgctaatcatactaatgctagttaaaaaaaaacaaacaaacaggaaaaaaaaaaaccaaaatagatttaaaacatgttggcggaagttgagatgacatgatctgatagctcgacaagcagaattaacaaacatgagtattctggttatgggttctcattggcaccaagcatttgagcatctggaattttataaccaagagtttaaagtggagagaaaatgctataagcagtggctcacttgtgtgggtttgggtaagtactgtgaattaccttgttatactgggagctctgaaattgaagataatgataaatacattcagcttcctcaaaagaatgccgtgaaaaatattaaaataatgtatttatgacttctaaagagaaagattttctttaaatattttctttgtaaaagacactggctttagtctgttactttgatttatatttttcctttcttgtttctgtcatgatgatgtaaatccaggctttcatcctgatgctcaggtctctagaaagctattttgcagggagaggacattttctgccatgttcactccatgtgaataagtttcctaaattgacagtgtttcaccccagtgggcacaattgactggagcaggaatgaaaaggagccacaattgtattccccatttgtcattgctgctgctgaggcaaatggtcctggtggcaattatttgccagggtagtgcctacttgctactgcaaaatgggcaggagacatagagaacatcactgcagggctccagtgtctgcaccaatttattttaaaaaatacatagaaacttctggaaagttaaaacttaatcacagtataaaaattgagttggagcactcttcttttcgaatattcttttttgtcccttatttttggcagttgtacatgtcaggtcagagtattcatggcacaccatatccacatgtcagagttctttcctagctgtgctggataattggaaagatttctgacactgctattcaaggagtccataggagaaagtatccatagacactggggagtgtacccatagacataggagagagtgtgtgggcatagaggagaatatccatagacacaggggaaagtacctacatactggagtaaaatgacaacagcaacaacactttaaaaatctcatggctgggtgcagggggctcacccctgtaatttgggaggccgaggcaggaagatcacctgaggtcaggagttcgagaccagcctggccaacatggtgaaaccccgtctaaaactacaaaattacaaaactacaaaaattagctgggtgtcgtggcaggcacctgtaatcctagctattcaggaggcttaggaaagagaattgcttgaacccgggaggtggaggttgcagtgagccgagatcgcaccattgtactccagcctgagtgacaacagcgaaactcggtctcaaaaaaaaaaaaaaagtcacagttatttacatgtattgttgaaggggattgcaatggccaaaaatgagttcatatccaccaggtcactttgtaactgtgaactggggtaagtcactcagattcttcatgttgtaatgtgcttgatttgtaaacaggggtaataatagattatactactggcaggaggaccagcttttaaacaataacatgtgaagcatcttgtaaactgatgatgtgttatatcagatttaaggtaccaatgaattggtggtgggtgtatgttgggtttagaggagctgggagaaaagtctaatgatgaaactgtctgggattttcttagtctaacctgttacccactgtcccatttcttctgaaaacaattttaagacatgtatggtaatctatgggggcctttggggacttaggaattttgaagattaagcatcaatcttacttcgttttggttatcataaaagaaatgaccccttgagccatgctgggctaagtagggaaggaagggagatgatgccttctgtcctccagcttcagtttggagggtgggaaaggatagaggaggagggggaatcatgaaggaagcgaagttgaaggtgcatgcccttcagtacctacatattgacattttaagtagcagttggtagaaaggcagactctgatttgcaatttttttccctagaaacaagagatccacagttattggcaaatctattgacaactatggaacagagatggtgtgtgaatttgcctttgttgtgtttcgtttgccacacaccctttgttcatactaaatcctgccagatgctgagcctcaaaatgggtctcactgcctccctggggaccttaaactcaagaaatggaaacagtatttttaacatgatgctgttacctaatctccaacagcatccattgacccctctctttggttttgacaatcctgcttggagacagggatggagcctaatggagaccatgggtttggttctaggggatggagttgaggtattggccacagtccatcctgttaattttgccacagggctggagcctaccgtagatcatgagcatatttgagagtactgttgagcccagagggaaagatctggtccccttggaggaaaagctgcagggatccagggaggagggaaagcagaaaagggagacagccggtggctgatgtgaaggtgtaacctgctctccattcccacagatggttgccattcccataaagtcagaatggagaaatgcatggagaacagtcatttctcataaagtcagaatggagaaatgcgtggaaaacagtatttctttctgtgaatcagaaaggacggagttatgtgattacgttctgtattctgaaaacagaagctggacacttgaggggtgctgtacatctaggaccttatttgtaatggaggaaagcacagtatagaggagacattaggatttcacctaactggccaggtgtggtggctcatccaataagcctagcactttgggaggaaaaggcaagcaggtcacttgaggtaaagagttcaagaccagcctggccaatgtggcgaaaccctgtttctactaaaaatacagaaaattagccaggcatagtggcacatgctgtagtcccagctattcaggagtctgaggcaggagaatcgcttgaacctgggaggcagaggtttgcaatgagctgagactgcgccactgcactccagcctgggcaacagagtgagactctgtctcaaaaaaaaaaaaagaaaagatttcaccgaactaagaattataatatattatataatcacaattatttttattgctgaatttttccacaatacatattgtattagtccagtttgatgctgctgataaagacatacctgagactgggtaatttgtaaggaaaaagaggtttaatggactcacagttccacatggttggggaggcctcacagtcatggcagaaggtgaaaggaacgtcttacatggtggcagacaagagagaatgtgaggcaagtgaaaagggaaaccccttacaaagccacgagatctcgtgagacttattcacaaccatgagaacggcatgagggaaaccttcctcatgattcagttgtcgcccccagggtcccacccacaacacatgggaattataggagctataattcaagatgagatttgagtagggacacagccaaaccctatcacatatatttatatatacatattaattctgatttgcttgtttttgtgctcttctagtctagactctgtattccttgagttcagatccaccctagagggtggatctccagaaataactattggctccgctatgggccagtgggtgctcagtgactgtgtgatgggtaaagtaaccctgttacctgagagtctagagactagaagaaactcacaggatacttttctgatggaacaaaagtctgcatgcaatatactgaatttctggttcattacgtgtgtaagttttttgcttattcattaattattttgttttaactatagaaataggtaggataacctcactaaaagagtaatcattaagttaataattacattttttaaaatcattctttctatctctctcttttaaaggaatcctttgctgaacacctaggctattcaaatggggtcatcaatgggtaaatctcaaatttttttttcttttaaaaaaaaatgttgttggccttacataaactcctcctctctacagtgtttcctcttgtgcaaatacttacttccagccctctaagggctcttcagtggagaagttaaccaaaacggtatcagcattctcttttactttactggcataaattattacttgccttaagcaagtggctattcaaaggacatagatcttatttaatagacttaatagtatcctagtgttctaaaaatgttgttgaagcttcaaaggtttgttatttataaccaaaagtatagcttgggcttgaagagaatgtatcttattaaatttcacattgacatttgacattaatgtacatgagaagaaatctctcatctgctgtaaatgtttacagttttatagtcatttgttagagtgtggtctgtgtaagaaatgtttttcattagcaatggatgttaaagagaatcttgacttgcaacaacaaaaatgaacactgcattaacagaaagtgtgtgtgtgtgtgtgtgtgtgtgtgtgtgtggtgtgttgcaaatttgaaaaatttattattaggatgtagtcatcatttttggatcccaagggcggacagtttcactgggctaagaaccaggaactatagcctatcctgaagcagtgctgctaagctttccctacctctccttgcctctctcttctctgggttctgtggttactcctgctcccatcagcttggccagggttgcatggtgaaaacacagctatagggtatttggagaacagttctcagagtggaggatggatgtcgggaagacatacgtttgaaaatgtgtctgttagaaataacatatcctgtgtcataaaatcaactaccagatgagacatttcagtactatagattacatgtgcactggcaagttttccaaacagtatcacctaaaattcggttttaattgtataacacagtcctgttgataaaagaaaaaaatgggtgaactgtagagggaaaaggccctgctttagcctttctccatatcacagcgtcttctgcatctcatactcccaacgtgtttttgacttgtacttgtatagtaatccaagactcttgtcctcccccagtccattcttacatgacgtccatagtgatcttataaaaaggtacgccagacctcatcactcccctgcctaaaacccattgatgacttttcattgcactgaaaatgaaaacgttcagtatagctcatagagccctaaagagtctgttcctagtcccatcctccacctctctgtcttcatctcccagtattctctcctcacttctgtttaagccatgctccctgttttttttttttttttaatttctcaaatccttctgtctcctttacccctcaggacctttatgcatgctctttcctctccctggaatctcctcctctccaccccagtgcccccgttcttcacctggatgcctttttactcattatttcaggttttagctaaaatgttatctcttcagggaggtcacctctgacactctaaaccagtggttggcaaactttttctgtaaagatccagattgtaaatattttcagctttgtgggccataaagtgtctgccacacctgctcagcgtagcctctgtaacatgaaagcagccagacaatagagatgagtgagtgtggctgtgttctgataaaaccttatttgtaaaaacagacagcagacggggccaaaggttgccaaactctgctccaatctaaagaaggcatcacatgttgctgtgagttttctcattggaacttgttattttaatagtatttattacagtttgtaattatccatttagtaaaatgagactctgttttgcttattattatatacatggctagcactgtgcttggtacatagtaggtgatcagtaagtatttagatgaatcttaagtgccctgaattaaaaatttaaaaagttatatactgaataaggagaaatttacagaaacatggcatatacgtgtgtgtgtacgtgtgtgtgtgtgtgtgtgagagagagagagagagagagagagggcgaggagaaagaataagttgagagaaagattgaaagtattaagaacatgaaagtggttccttttctttctagaacctgtcatcatacctagtatatgcaatgcacttttcatgggttttatatgaatatattttccttttctgctaaatattctcattgctttggagttcatattcattcattcattcatttcacagttacttattgaatgtttacagtgtacaaaggactatatgaaaagcaaagcaatgctggattttttaagaagacagaagtaatactgtcttcttgtttttgcatttttttgggttcagggctgaactgtatcgggcctcagggaagtttgagctgcttgatcgtattctgccaaaattgagagcgactaatcaccgagtgctgcttttctgccagatgacatctctcatgaccatcatggaggattattttgcttttcggaacttcctttacctacgccttgatggtaagtgcataaggcattaggctcggaagccatactactgaaaatgaagggataatgggcacttaggtccaatctcagccaaaaagaaggggtaaaattgaagaattgactagaagcattgggagcagtttttctagatagcaattttttgcatccttaagctttaaataagctgacctcatttgtgcagctgcatagcccacaaataaaccaacacaaatgagtttagagtcacttttgagtacctaaataaaataaaattaaactaaattaaaattttaaaatcttccttgctcttaaaattgttacctatgtgccaaagatatagaatacattgacacacctttaactttttcagttaaggcatattttaaggggataaaggctatgtcacttttctttgtaacaatacaatcattcatttaagattatatttatcattaaagcccctctcttcctaaataagacgtatccacatgttacattgttaagagacatgacttcattgtttcctggattttgcaagctatgtaaaatatttgcctggttttaagagtgaaatttaaataccgtgtcctaaaaattagtaagaaaaaatattaatggaagcaaatatgattttagggataatccctaaattattttacgatttggatatcagccataatatattagaggttgaaagtatcagcatttatacacaatggaagggcataaatatggcattcaaagagtggtaagtaggaatagaagttaatttacagtcaggatgctatttaatccctgggtagaaaagaggatactgctaataccagtatttatctaatgcagtattactattgttaaatgggaaacttaaggtttatgtttcattaaaagactttattttcaatccaacattttgttttcctttttgccttaattgaacgtaattggatagataccaaactcagacttgccgtatgttctctgacttaaggttttaggtgaattctctttatggtctgcctcttagggtattgttaattaattttcaaattaattataccatttgacaaacttgatttttttttttattttattttgtttttattgagacagagtctcactctgtcacccaggctggaatgcagtggcatgatctcagcttactgcaacttctgccacccgggttcaggtgattctcctgcctcagcctcccgagtagctgggattacaggcatgtgccaccacgcccggctaattttttttttttgtattttttagtaaagatggggtttcaccatgttgcccaggctggtcttgaactcctgacctcaagtgatccacctgcctcggcctcccacagtgttgggattacaggcatgaaacttgattatttaaacccctttacctctcccctccaaattacgtaatgaatgaatggttacgtaaatgtcaatttttagttcacaaatacagtcctattaggcacagtttttctctgttggcaaatttcctttattgtctcttctcctttgactggatccctgcctccggaaagtggaaaattcggcactcttctttcaaaagacaaactgtcctttacctgctgctcaagtgaggggctgtggggttgtggggctgtattttcccctgaactgacagccccacacaggcgtcctgcgtaactatcacttgctactttgtggcatttcatttccaatctcaggtctctgacgactgcttctttggaatatttgcatctctcaaactggtaagaagtgacttcctggaaacaaattgctttcatgttttcttgtcagcctgagaggttctctttaatagtttattggctgctgtccttttgttagtaatagtcacaacagctaacatgtatggaacaccgctctgtgggactctgggccaagcatttcatgtgcattattaatttgattttctcaataaataagctactgttatccctgtcaaccagtgtgccattatgatgcccatttaaggatgagaaaacaaaggcatatggtgaaaccaggttctaacttggatctgtttgattccaaattagctgagttttagcagatccagagaggtataccccatctctgaaaatacaggcttcttctctagctacaacattttaagaatgattttgaacttatttaaaattttcaattgactaattgcaaaccaccatgcactagagatacaggatggatgaactattcactgttgctgctctcagagtccttaccattaggtggaaggagacagacaagcaaattgcctaacatgttacagatggaattgaaaaggcatctttccgtaagaaacagcagccgttccttttaagggtggacagaactaatggacaaacaatcaatatacacacagataagttagtacacacacttccagctgaacccccttctgtcccccatcccaccacccaattattaaaatgacttcagattttcattgctgaatttgatgtctcagattttgactttaggggaaaaaaaatcacttgatgagagctaagcagaaattttctgcaaagattatggttgcatgaatagatttagctaatactccatatagtttttgtttttactggaaatgatttttgagtgagagcaagtggtgaggttttatcccagaagacaacactttctaaaagcaggtgaaactaagttaaaattagtcatcatgaaagtctgacagtgtgttccttctggtaaattctgtaattttcaacacagttgccctattaattgtatgcttataattggttatgtatcaaagtctgaaagaccatgaaactgatgttagtattgagcacgttaaatcttgagccacttgcaaggagtgtctgcctttggatagactgttacatggtagtttgaatctccctgaacaatattaaaaattttgttaaatttaacaaaaaaaatttgttaaattaaaaaaatatttgttaaaaagattgctgatgatcagcaaagatcttgacttggaaatctttgacagaaagggttttctagcaaattgcaaaccttatgataagaaatgccatagctttgtatgaaaaatgacttaatcttttttgttttttataatttgtcaagttcccagctatttatacagtttatttaatttaattttattttattttattgagacggagtctcgctctgtcccccaggctggagtgcatctcggctccctgcaacctccgcctcctgggttcaagcaattcttgtgccccaccctcccaagtagctgggattacagatgtgtgccaccatgcccagctaacttttatatttttagtagagatggggttttaccatgttggccagggtgatctcgaacccctgacctcaggtgatccgcccaccttggcctcccaaagtgctgggattacaggtgtgagccaccatgcctggcctatacagtttaaatacacaccagatatatggttggcctgtttgcttgaggtatctgttatagggtctgaaaactcctttgaccaaataattttatttggatattccttttgtttttcagtgctttattttgcatgtaaggttatttcatctcagtaggcattggttgtatttatatcttatgttactggccagatccacgtctgttctgcccacgtgcagtaaatcaatcactgtgacacaggttttgcaaaagagaaaagatttattcacaagggcaccaagcataaagatggaagaacagccctcaactctacctccctgaagataaggtttagggatatttatggggtaaggaagtggggtggtgtaaggcacagggaaaggtgattggcagtggggaaaaatgaaatcacaggttcgttctacacaggcatagccagggttcatggcatttcctaggacatacatacaaaaaatggcagagtcagcatgatctgatggtggagtttttggctctccaacatcaaaaggccatctcttgggcacttgcgctcaggcccagtttaagggccagtggtgtcaacgggtttgaactggacagagctggccaaagttcctgaaaaacaactgaagtaaccattaccatggtgacgtatgcatgttatctgtaaagtagccagtgaaggtcaagtttgagcattcagcagcaagaccttcagctactgcagccttcagcttcatggaaaaagagaaaacaaaaataacaaaaagcaagaaaccagaagcatgcagggcaggcagacctgatgagattaactgttcggtttcattaatccgtgtgattatcagctggttcattttcagttaatcaaaactgttgaacttctatcctgagagtatagatgtatagttttgtacccagttttcagaaacacagatgccatgatctcttctttccccattttagaagaaatttttggtttctgtggcttttcattcagtttaaggcagaggagaaatatgtgaaccattaatacatgtaatttttaaaaaaattcttgtagctggaaagatgatagagaaggtttcacagcagaaatatcttccattagcgtcgtgaacagatagttcaccatgatctcagaaagccaattctgaaaataaaagaaaaaagagccccacatttgaaagctcactcatcaaagcatgtttagtgctcctcctccaaaatctaaactcggacagttgattgtgtcggacctcaccagttcactaaactcgaaacctaatgtagaaagtaaattgggtctaaagaatgtgactccatgtgactcaatgagagcccccagacccctcatttctaatccctgtttgctgtatgcagggaagtgatttgttttctccctcttcaggagggcatttgttatttttattaaatgtgaaaaacagatcgtccccatgtttgagaacttggccctcatagtaacttgatttcattagctatagtcacttaaatgttttaatcctctgaaataaattgctggtatgttttcatccttaaaaaatgaacagcatgtaggaataatatttgaagagcagctggtcagagactgtcatgtaacatgcatacatatttcccaacagcctaaagccttctttcaaaattcagcatctaagcttttctggtgaaatatgttcagatggaatactggtgttttttttttctccccttccacaatgcatttcatgaaatggctgggtaaatacattacacatacataatatgtgattgagtggctcatctggaatgcaagaatgccccaaaagtgagcaccagactctgaggtttggtactgtctgcttattcactattttggagcctcttcagaccccatggggcaaaccacccccaagtatttaacatgctgctctgtttagttggttttgatttagcatgctttgaactcaacagagcaggatgataacgtgagaaacagactaagagatttcttgtggggggggtggggattattgtggctttaagtcttgctaacaaggataaaattattccatttgcaaaaaaaaaattacattcataatttatgtttttaccaaccagaccatacttagtagtaatccaaatggtaaagtagtagaaaagaactttccttttttttctattttcttggcataaaaaaagtccttgctttaatgcctattggcaaagtttatttaagccattattacagttttaagtgcacgtgtgtgtgtgtgagagtgaaagagacagggcgagagaggaagtgtgtgtgttcttaaaattatttgttgttataaatcacagaaataaatccaaaaaatgttttcattgttttaggtgagagtaaggattttgtaaaatttgtgaagttgcgaagatgcctcattagaaatgtttaagctgtttctttctttttttttgtaattgcaaaatgttcaaagttttttatccagaagtacaaagccctggaaattacctcaccagtgttaggaggagtctgggtatatttcttgaaggaagcaagcctttttgtctcattctgtgccattttcagacaagagttaattggcaaattaatttttctgatcccctcaggcaccaccaagtctgaagatcgtgctgctttgctgaagaaattcaatgaacctggatcccagtatttcattttcttgctgagcacaagagctggtggcctgggcttaaatcttcaggcagctgatacagtggtcatctttgacagcgactggaatcctcatcaggtctgcatgtcccactcaggtgcccaggcctccctctggagagcaactaaaagatgatcagtttcattattcacatttacaggacagagcaaatatctaaacgagtgggctgttgctttcttggagccaattcttctggctgttttcttatctcccttagagcatagatacgaggtcccacatatgcctttgaagagctgaagtttgaggtagaaaaacttagtttctccttagttttaatcccatctcttgggattgccattgaacaaagtatatttaatgagggataagtcaaaaatgttgcaaaatcatagcagtaagaacaatagcaaccatcattcatgggacccttaatctgtgtcagcctcttgggcattttttcattcagttttacgacaaccctgtcagacggttaatatgatttgaatctttgcagtcaaggaaactgaatcctaggcagggtaagtaacttccccaaggccaaatagtattacagtagttaaccttttattttgtgttttatttaaagtcatcatcaaaacatattctaatgagcatttattgttgtaaagctcttttagccaggtaagttcagggctatccttttaaagcagtactttgatgttttgtttttttttttttaaattgagcgggtctctcatggcactggttttctaagttactctgggaggtgaggcatctgcctgtaggatattgggtagggtgatattggtcaagatttgattcatactggagcaaatgttgaattcaaaacagtaaaaatgaagagcaggtgtggtggctcatgcctgtaatcccaggactttgggaggttgaggcaggaggattgctttgagcccaggaatttgagaccagcctgggcaacatggtgaaaccctgtctctacaaaaaaatacaaaaattagctgggcacagtggcacttatacctgtagtcccaagttcctcagcaggctgaggtgggaggattgtttgagtctgggaggcagaggctatagtgagccatgatcgtaccaccgctctccagcccgggcgacaaagcaagaccgtgtctcaaaaaaaaaaaaaaaaaaagaaaagcaaaaagaaaatggcaactctcacgtaggagtatgaattctacatcagcttttcctaggctcctcgttgagccctggttttctcaacagtaacactatctcactgggttgcgaagactgagtggcagtgccatatagcgtcacttttggcaggaaatcacattcaggggtacattaaaattctaagggagcttttaaaaaatatgcatgcctgctttctgttccctgccaccgcaccccagttttcttattcggtggagctaggtgcttcctttgagcgctcccaggagattctgaaatagattctcatacagatgaggagcactgatttaaagactcaactgtgggcccaaaccaccaacactgggccactgcgtgtacttaccattctctgtaggtgaataaattaaagcgactattaaaattttctgatcttgaaaaggattgggagccaccagcacagaatatattagttctaagtcttcaggaaatatggttacgtatttttataaatacataaaagtgtttcattccaaggctcaggtagaattcatattgatgtgagagatctgcagtttttctttttagcattatggattcagtatttgttccacaaacatttattgagtgtctgttatgcagcaggcagtgagctaggctgggtatctaagggggaggaaggaagacgtgatgtgactcctctcctcctggtgtgctcaatgtgattggatgtttagattatccttgctggtacctaagttttgaggacgatctataggtgaatggagagtttgcttgtattctgtcttcggaggtaagcatttccactgactcttagatgtttaccttcactggcacagtatacagtataaatcctaaaagccactgttccaggaaatgctaatttaacatggaaaataactctgggacatctgtatgatgttccaccccaccccgcctctcatttttttttttcgtactttctctctagctctgttaaaaactttgaactctagaatcaaagtaaattgcagtaacatgaaatagtcccctcttaactacatgggaactaaagctttggcagcaaaacacaggatcccagattttctctggtggaatttacaacaggttcctcacactgggcctctgcaagtatttctagctgccataggtagattcacttccacctgattgaataaactgagctgaccagatttttcatctgtcactgaacaggaccattagtttggtcaaatttttctttttcaggaatcagtgattttatagtgataaagatccatctgagttttcatttaaaaggtacagaactctactaaatattgttaggcactttgcatacctcacctcaagtaatcgtcaaaatatcttctacataaggaaaccagtcaatgttagcttgctcatgaacaccccaccaggatcattcacacccatgtctgtttaggtttcagtttgaatactgggtttgagctctttctaccttgcacccttgcatatagattgtatagcccccaaaatgaaaattgtgaaggtctttgtatcaggttaattttagcatgcattgaagaaaaatttctataagtatgcattatcatgttgaatcaaaattaaggcacattggtgaaaataggggatttgacataggttgacagatagaggccttaatgttgctgattgaggaccctacctagaagcaaagatagaactcgtgattgtggaattaacaaaaaaaaatcccaccaggttgccaagactctcatttccaaactgttggttagaaacatgggttggtactcgtgttttttacccacggagctgcagctgtgttgctattacggaatggtcgaccacatttagcttgcagaccctcttctcagaccaggctcttagcttcaagtattatcagacctaagtcagtgtgatgaagtttcagaccaccgaagtgagacttcagaaacatggagtaatccatccaataattacagcacttctttgtcacaatattccccccacattttggcttagggactcaagctcacatttcgttaaacttctccagtctagaaatattagcttttgtgaccttccaattagatgggtgatctgaaacacacaaacacacatcactgctaccaccaccaaacgaaacttaactgcagcaaattcccaaggattctgtaatgtctgaccgtgttcctatgttgtccatgtgccctgcttcctcacctgttctattcatattctgctgccccctgcaattagtatttttgtggttttgtcagcatcttcttggacaagggtcagcaatctactactcgcgggctcggtctgacctgccccttgtttttgcacccccacaggtgaagaatggttttcactttttaaagtataaattagaaaatggtggggaaaaagagtatcagccttctggtgagaacagttgttcaatttttgcttctgggccagacagcatagaacgtttactattgcgcccattttagagactgttggcctctgttccaggagatagtgaaaagcctcatgacacagaccttgtcatcttgactgctgttagcaggtctggtgaagatatgtggagagggacggttgaactaaaagcttaaaaaggaactttagggtgttttaaagttctttgtcccctttggatttgaatgatccaccttacattctcctctttagaaaataatctaatatagatgggtcccagctgttttcatggcttagcactatgatatttatattaacttattcaaatatctgctgaggaagaagttttgcctaattggctacttaattttaaaggtccctgcagcaaaaacaaatctccagggcagccttttgtctgctgccttcttgtgctacaacaggtgaccaggagacttgtctggtcaagaagacgggtttacagctgttgagcatcagctctctgcaaattatggtgccagggatgtgggatacagaaaacagacttttgccctaaaaatctatacagtctaatgggagacatgtgtaaacaactaattagcatcagctcagataacatcacctggaccacaatgaataaaagccacatgccaagaagtttttattacgtagctataaaattaggtaattctgtttcatgggctcatgaaggtttttatttaaaaatttcatgaaagcttttttcttattattaatatatgagtaacatttaaagcagaaaaaaagcaaaatgtaagcaaagtgaagaaagtgaaataacacctataatcttatcacctggagatttacgttgttggcatgttggtgtttgtcctcctacctttttctttatatagataaatttgcacatgtaagatcagaatgcacatggttctttttaacttgttttcacttatgtcattaaatctcctacaatataatttttatgatactttgttgatacatacatatagtaggatactataattcatttaactaattcagtattgttgaatgtttctttttttaaaaaaaaagccattagagagagtaaattattctgagaaggtacatccttatttctaaatctttgtgcatatctatgattatttgtttaggatacatttccgaagtaatatttctaagtcggagacatagacagttttaacacttctaataggtagtgacaaatttctgtccccaaaagtttaccagttgactacccaactagcaatccttttgatttcccagattgaagaccgagtctttgataaggttaggttcgaggaaattgactacctgagcagtcagtgtgtgtgtatgtgtgtgtgtggtaagatgtatgtatgtataatacctgaatgtacaactcagtgatatttttaaaataatcttcaacacatcacgccaaagaaaaggcgtttcttgcaacttaaggttagttgtaggtgttatgtaagtcataattctgacattggacattggtgtcttgtttaatgttttagatatggactaaatctgatggggaacctagagacagataaaaacaatatgatacctggatttcaaaacctgagatgtatttcagttggcttggttaatttcaacccaggtttcttcaactaggaatgacactgaatttttccaaacgtagcttgtgtgtttttaaaatgtaggcaaaatcttaccttagtgaaggtgaaatacagaacccttccatatttccctctggggtggggtccggttttggatgcctatgccaggcatctcagtcctcatagcatattgaccccccaaacaggatctgcaggcccaagaccgagctcaccgcatcgggcagcagaacgaggtccgggtactgaggctctgtaccgtgaacagcgtggaggaaaagatcctcgcggccgcaaaatacaagctgaacgtggatcagaaagtgatccaggcgggcatgtttgaccaaaagtcttcaagccacgagcggagggcattcctgcaggccatcttggagcatgaggaggaaaatgaggtattagagaaaaccccaagtttatgaaatcaaacagtggccttttgtctctgaaggactcagaataatccctttgtttaaaaaacttcctgggctggcgttaatgaaaatgaagaaataaactgctgttttagatcccaaccacagaaagcctgtgaaagttatccaaaatgagtgtcttaagagctatggctggaggaacctcaaggcttggcctgtgggtgctccctcctttttcccagggtggtaaaaccagctgctttattgaaccatcagtgctgccttctgtagggcagatgcagaactgcagaggatactttagggacaaaatcagccggacgagacaagacataaatggaaggggtcctctttctctcatcctccagagccctctctctcttttgccttttgatcagggatcacagagcttcaacatgtttgtaggacaaaccttcacagccatcttccgcacatttagctatacttgctcttggcaagcatgaccttaacctctactttaaatttttctttaagagctgtctgaaggttgggaaaaattctggttaaacagcctttatttgtcagggttgtatgaagaatgctttgattaggtttttattaggggaatgttgagttgtcatttgtattatttttagattgccaacgttagtaattttataaatattcattccaaaatggatttgaagtagatttttaaacgtatgtatatagtgtagcaaactacagaatatcagcagaagtccttatcataaaactggctgaacatattttttgtgtgtgcccatacttacaaattgaagagtaagtctaccttacatattcatgtggaatactgttttcattgcatatattagatataatttgatttttgccttaaataggatatgtgtttttgcattcttctataaattatgaacctgagtcttttaaaagtcttataaacaaatactcctcaacttacaatggtgttacatcccgacaaacccatcataaattgaaaatatcaggacatggccccatcttaagtctagtggagtactgaatgtgcatcccttctgcaccattataaagtcgaaaaatcctaagtccagccgttgttaagccagagactgtctgtatattttttttttctaaaatgaatctaaatgtgaagtaaagcttgatatttaaaggtatcctgaaatgaacccatttgcaaattgaagagcacttttgtattatgcctcctaatttgtccgtcttatatttttgattagctatccactgagtttcatgaaaatgataaaaaatacatatgcacatgcactcactatccacacacatttgaaaaatttttatctcaaattatttatagtttctgattctagaaattgaatatttgatacagatatggagtcaaattgtcaggtgatttttttgttgtttatacacttggacatttccaaaaaaacaatatggcgaatgatctatcctgtgacaaagcagatatgagcagagctgacactcagccagtcgtctccactacatttttgcttatttgttttgtgtaggccttgtgaaaatagtacctgatctggttgagaatcaaggaagcctggagttttccagccttcctaagccgcagggagttgctgatctcccctgggcccgagggctgccaggtggggcctgacgtgcactcccctcccagttgtcctgccatcaaggctagctgtgagcagccttgctgaggctcctgtcccttcttggctgccagtgtgtcagaaaaatgttgccgttttcaggggggcttgtgtcctgatgtaaagatgtttgaaaacactgagagactgcagtcaaagactctggttctgaaagatcaggacaacataggctggcatcaccatggttgatgcgaccatgaagtgctggggcagagaacccaggaagatgcattgtgcacttccagctatttggttttccatgaaggtcataaacaggcactttacaagagaatcttttcatcttgggcatggggttgtctgagcccctcatggtccaggtgatgagagggtaatgtgtggttctaggaagaaagtaggcgaagcgggacatctgttcaccagaggagacattaccagatattttgtactgtgaagtctgaacccactcccagctagtctgtgtccttgtcagatgaaatgtaatatttagacaaattgactttaatcaatctggaggcaaactagaatagaaggtttttcagaaataattattatcttgaattgtagatttttttactttattggattgcattcatttctaacacttattaagttcctgcagtgcgaaagacgttatcttgggagccgacacagtcatctcatttgagccgctagtacactcatactctttctctaaaacaagttgcctctttttgaatatacagtttgtgaaattggatgtctgaagttctgtgttttcctctttcttccctcgactgatacactgaaattccccgtttgatttctaacaagcccactcagcatatgataaaaataatttgggaagataaatcctgaagcatatgccgaacttttagaatcattttttaagtgtggaagtcatcctataacattgctaaaaatcctgagaactgttggtttctgaggaattagcagagagataatgtacctttgtttgacgtaatataaaatgaaaattttagcccatatgtaattgctttctctgcttcatttaaattattttaattaagtataattttgaggttttttttgttactgatacctttttagttatcccatccatagctccaatcattaacaataatacccatagaaatatattaagcacacttcaaaggctatatatttggcacatatatgtgatttttattgtcttagtgcctagtaacaaggtttcacatacttatgtcttttgtaaggatttgaaaataagcatttatatttggcataaggtaaatggaattaaaagaattaatgttaggtactttttcatatggatgtccttattaaaatcttacatccccatgtgaaatagatgtcatacccatatgaccaatgaggtcactgaagcgaggagacattaagtaacttgttgtaaaagtaacagaatcaagatacacatccaggagaactcatatttcttatagtggaccacagtttcctccctgaacggattttgctctgggaagatggttttaatagcttgtaaaatagtaacgtcaaggactaaatccagcaaccccttcccttttctttctgccttgagaaatgggacccctctggtctggaggacagatcacttacttgtttgtaccttgccccagctgtccactggttaaaatcactctgtttttaaccccaggaagaagatgaagtaccggacgatgagactctgaaccaaatgattgctcgacgagaagaagaatttgacctttttatggtaatgttacagaaaatcatgaacacaaatgctttatacctctgcccctttttctgttaagcagaatgtctgcagcctgcgtgcctggcagaacttcatacgtaaagcctatgcctttccttcagttcagaggctgcaaactggctggagttagcctgcagacatattttgtttggcccacgcagcgttttttaaaatttcccattcattgccaacataaaaaagaatcctgatttctggcttctcttgaaaaatgagatcgggcagtaccaggcccacatctttgtgtggaaacagttggccagggctgagtagcagctgcactctctggacaggcctcgcggtctacagcttaccatagacgccctcctgttcccagttcgtttctaatcccaggccagtgtcattcaggaagcctacctggctcctataggcattggaacttacaacagtgcccttacaggatcaaagcaacatgctccttagggagataggtggacaacagctaaaacaacacacaggtgtgccttttcagattgttgacgagagcccgcttaaatgaatgggaaccttcaaggtgtgcataaaggtgtagccttcatttaaggatcaaaatcagttggatttccctccagtatcttcaatcattttcatcttcatcaaagactgattacagagagagtaaatataaaccacaactttggtggttgaatgctgcagaaccctaaagttagaggtcccaaatttgagtgccatcagatcttacatgagtaatttgcatgtgtgaagcaagtgggcagaggtaggtgtactggagatgctttgtctatggggggacagagctgcaaggtagaaatgtgagacctatgtcgccagaaatgacaaattttcaaggggagcttgaagtctaggtgtttatatgtgatctctagattttaaaacactgcaactaaatccaactttttaagttattatataacactgcatgccaaacaaaacacaactgtgggccaaatatgatccacagggtttgcaatctccacctttttagcctagacagaatggaactccacctgtttggagaggcttatgttattttggggttttgtgtgtgtggcttttttctgggaacattgattgtatataatgatccacaataaaattttctttctcaggaaaattcaggtagctgtgggaataactactaaactggaatatgcacttccctttctccctccgtccccgaaatgaaccacccttcccaagtaaagctatttcagagaggaatggtagtaagaaagacttaacagcatggtcaatgtagggcattcataaattctagatttcttaactcacactggtcagcataatcaaagattacaatattctctatttagggaggttaaagatacccatcagaaatagacatcccctctgtccttgaagaactaattaaaaggcagattactggggaacacccaagcaagagaccctcagctgatcactgttaggcaataattcagcactgctaaagccgcagatactctcaagacactggagaggacaagctgcttgccattatacaaattagattaggcccagaaatggagaattctttcagcagccctgaagtaagggggtgagtgtactttcctgccttacttttgggctatttgtagaaattgaaaacctcagaaacaatctgaatatcttggaaggcacagccttcttttaatttgggactattttaccactcaaagaacagggttgtaacacaggtgaggctggctagggaacactttttgtaggcactttgattttgtgaattttctgctggtttcttaaaggctggcttctattttgaggattagacagattttaggtttggtttagtttcttctaatccaccggccgcctttggccaattcactattccttagcacctccacttgggggcaggaaataaaatttgaatctctgccagctacagattaaaaagtttggtgagaaacaagaatggaatgtgagctagaataaatctttggattttaaatatttaaactctttatccattattagaaaagagtgagttctctataaaattaatgcaaacttattatttgattttgtatcatagacactggtttacacatgataagcagatagtttatttaaaataacgtcttttaaaaaaagcatgtcttgctttctctttctaaaaatgtaacttcagaaaaagtatcttttataccaattacttaggaattttaccaaaaagatctttggcagctcaagataagtatctgagacattttgctaacatacaaacattaagcttttagatgctatgttctctacaaagggaaaaagcacttttaaaactaattgtacttgtaagcataaggtttataatcagatctcaagaccaattattcacaaagtaactaaatagtaagtttgacttttgccataatgttgaattaattatattggtattttgtgggtagaaatctctagggagagatgcagttattaagactttaagcttatcttatgtaatcgaatctacctttcctaaaagaaaaaagaaacaagtgcataatttacttttctatcagaaaagatttattttctcaattgtggacaaatatattagcaagaataatgtccatagaaatgtattgattaaacatacctcaaagactatttttttgctgtagataatagtagcagtaaaaccaaagattgaaatggcagtattttgtcatctcagtataggacattaacaaaatttttttttttcttattagctctctaataactattaggaggttgcctcacgaagcccctctttagaactgctgtaaagaaggcaacctctgtgtgccagttatgaaggtttggtggatccatttctcataccaaaacattagaagacaggattaagcatcgtgagctaattatagcacatgacagagcaaggtggccacatttatcaaccaatgggacttcacctgtctcccaagcctaccagattctggaaatgtaatgacttccaaaaatgtggaagaagtataaagagctttgaccactaattttaaaataggttctagaggtcatatttgggtttgcgtagatatttcatttacattttatcagttttagcttttaagaaaacataatattgaatcccaatttcagtatggtagaaccaaaggaagaaggaaaaggtaaataacaatgattgatggtccatttcattgtggaaatgacacatctcgctagtgtttatacataatgtctgtggatatatagtggcagtttattttttaaaaatccatcgtattgtttagttatagattaaagccaagtgtgtggcccttagtttatttacaagtgaactggattattaaaaagagagaaggtggggaagtaggcattttgaaggctctgaaaggaatccatttcacttacaactagatcagttgggatgcgattacctctgagctctctctgtctgtggtataatatattcataagagcacaaagcttaaaatgtaaagaattgaagagcattcctcagacagtgcccatgaggtattgaaaagttagtatattcatggccaatggaccagtgaaaccatttatgtctcttccacatgtccaactgtgggtagctattctgtactgataccttgggtcccccttttttttttattgctgattgtttagttcaaagcacaagccctattgaataaaaggaaactttcttaaagatctttcttttctttactcaaagaataagtttcttccagggaagcaatgagttgtatagccaagaggtcatatttttaaaatgcattcccaatttgaatttttccactattactttaaagagggagctttgcatacacacagactaagaaaaggaaattctttaaaagtactttttaaatgtatgaataagtttcaaaaggtgggtacagaaaaagggaggggattttacttaatggaatctctccctagatatttagggatgagctagaacaagccaaccaggatgagagaggttgaaagggaccctgcagccataggaagtgacttggggaagttgtgtagtgctgggaagtctgcaccatacagaagccctgactttcggtgaccctcttattaatgtctccagcggatggacatggaccggcggagggaagatgcccggaacccgaaacggaagccccgtttaatggaggaggatgagctgccctcctggatcattaaggatgacgctgaagtagaaaggctcacctgtgaagaagaggaggagaaaatatttgggagggggtcccgccagcgccgtgacgtggactacagtgacgccctcacggagaagcagtggctaagggtaagcctagcttttctaacccgctctcactaggtggagggtttttggtggcttggagaaaccaggggcctagagctgggattttctgaggaggttgagttcgcaatcaaagggaaggggctcttgaacacagaagataaagtcattctgctgttgcataaagtcattgtgctgttgcatgaaaagataaaggcgttctgcacttcaaggcggtgcttggaaaaaaatatgcaggattttttgctagagcagaattggccattggaagcacaggattatgagagctgtgagacccactaatgttttatgaccaattgcctggtcatgcctcactctctccaactgagaatcaaaatgtggaagggcaccttagagatctttgtaattgttccccctcatttcatggtctttaatcgaggttttacttctgcaaggtctgcacagatctttcagggatagcagagttttctaactgtgtggttattttgtagacccatagtacttttagagctggaaggtacctttgtacttgcttggtttgatgaagaaactgaggcccagagaggtgacatggctatgcaggacacacagctggttagaaagcacagaatcggtgggtgcttatgtgtctacatgctcgatttccgcttgatctgatctgtccatcacaaacacagattagagcctgggactcgggtcaccgatgcttggaaatgttattcgggagtttgtttatgttgtttttcctttggaaaatggtgatttctcattttgattgaggactgaaatctcagaacgttccttgggcagcacagaaatatcaaaatgctcttaggagtcgcgtgtgtgaacactgaggcccgggtgactgttccattcgaagggttagctctacttcccatttttgggagtagacttagtacctagaaaggcacagcaacctgggacgtgcattccagacacagccagtaaatgcatgctggaaatagtaagtgtagttgggtaaaaaccaagcaaaacaaaagctttggggagttgttcgttttatccaaggggagtacagttcccaagctttgtgaaacatggattttaggacccaggttttaagtaggtagatgggtagaaaggcagtgggcagaattttgagattagataccaaaggcaaacaggtcttgtcaccgcttgagatgtgatttactcaggatgttgtatgatgagagggacgtgacctgaattttgaaaagatgggagcctgtgattctgaaaagcactgctcattatttgtggttgaaccgttgtgtataaaatagaacatttgtgtgattagtttgtcgtatccagtcaaatattccagtgacctcagaatatgattctgtgaattttctttgtttcactattgagattatagttatgtaattaggattcattctgtaatatgaagagttttttggctaatatatatttctgagcttgggaaaattttatttaagcaataaattgagtacctggagtattattacagcagacgggacaataataatctgggccaaccttttttttttttttttttgagacagagtctctctctgtcactgaggctatagtgcaatggtgtggtctcagctcagtgcaacctctgcctaccgggttcaagggattctcctgcttcagcctccccagtagctgggattacaggcgcataccaccatgcccggctaatttttatatttttagtagagacggggtttcaccatgttggtcaggctggtctcaaactcctgagctcgggtgatccacctgccttggcctcccaaagtgctgggattacaggcgtgagccaccgtgcctggctgggcgaaatttttaaagctaagctttgatatgtttactctccagacattaaaagcaaaataaccttgtttattgggcaagaatgtagctgaacttaacttcttaaaaataatcattgggaattgccctgaagaaaaacaacattatgaatctcaggaagttttccagtttaagatatctgttttgttttctctttttcattcagtttctatccctgatgagtagcacagtgcctagcatagagactcagtgaatgtttgtgaaaatgaaagtacagccttaagaggaaagggaaagtgactaacttatcagcagtgttaggagagcatttctctttttagcgcatctagtaaacttccttttatctacaacatgcacatttaaaaatggaaaattagtttgttttacaaaactgcactgtgaaaattggtttttataattgtaattctgaaatgccatgcaacatactaagtcatccaagaaggacgaaactgttctcaaaacagctttattgtaacgtatctttttgtaatctaatcttttgaaaactgcatgcccgtatttggtatgaaactggcaacttgttattaatcaggacatgcaatggatgcaacatgccccatggcctaagcattcaaagtagatgcttcctgtctttggtgttcaccagcagcaatcctatttaatatcagcctgttaagcttttttgatgatgaaatgatattgaaggggttctcatgggcctggatgtgaagagatgttctctactggcttatgatttaacacattgcatgaacccagccaacatggccttgaaagggagtttggcaaggaccgtcgtgtaagaaatgctgtaacagggctaaaataattcttctttttcctggcaatgttgtacaaagcctgcttggcagagacatccaagatgcatagcatcgcagagctgcaaggcagttgaatggaacagcttcccatcccttgcttaaatcacattctctgcaatctctgtgccaggaggctggccacccctagaaggaaaaactacctttttgtctgtgtagcccctccagttcgtggtcagctctgattaatagttcttcctccctttgaatagaatcttctttccttgtagcttctcctcagaggccttgattcttttccctggggtgaagagaacaatatttttttctgtctttagtatcacagccccatggataaaatgtttgcaaactcatgttcagtctggattgaacttttctaccttcctctttctcttgaactttcctcctccttcaaggcccaattcagttgctacccactctgtggcaccttcacagataaatattaattgcacttttaattgtcctcttaacatttctttgcttgtccttcagtcacagtttcctagtattctcggtagttgccaacctgcctgctttcctgactagttgataagctggctctgtatcttgttagaatttaggacttctgtgtacaaatactaagtgcccatcagttgccaaattaaagtagcactggattgacagataagggtatagatagatgtgggtaatgttcactgctgccacagatggacctcaacatgccctgtccatggacctatccagaaaaataaacagaagttgatttctcccttccctgaaagtccatggtaggtatacctgattggtgaggacagctgttctccatagggtcattcagggctccaggctgacagagcctctgccggcttggtcatctccatccacttgagctggaagtggggaaaacaacatggagtagtgggtatggaagtctaatgggccctgcctaaatggttataattccattggctagaacttggttatgtggccactccagctgcaaaggagatggaaatgtggtctggctgtgagcccaggaaaacaaaattttggtgaatatttagccatccctgcctcagcacacttaggttaaatttaagtgttatggtcactttctttgccagtggtgttttggtttcattgaagacaggttttattttgtaatcatggcatgctaaaaattcctacagtgtcctttagagaatctgtttagttggtggaatttagaatttttttctgcattcataatgttgtttttaatcaggaggatcacaatttcaaacacagttcagttttttttttaaaggtattttacattgttttgaaattcctatttccactgaggaaacttagatgtccttagtgttagctggtctcaagttaacgtcatctttttgtggtagtcagttcaacattcacattctgggctgtttctctcttccccgggggctcccatcccatcctcctctccaagatggcatctggataaagacgtggtgtttaagcacggcattgggggcgagcatcctggttctcttgctccccatgggtctccactgatctcagcatatagctgctggtctgctcctttcctgggaggtctttacatgcagctggtttcatctcattcttttagagaccttgatgacatgtgctgttgaaatctggagcctggccacaggcactagttagaagctgtcctgcattctcacatctcataagacccctggcctctttggcctcatggtgctcttagctcttttgtgttctgtttggaagtctgcaaactctccttgactgatttctccacactgtgctagaagatgcagggtgcagtgaaactggccccttcctcagcagttgctaagtcagcaccaggggttgtcaaacctgaagcttctagaatctgatggttgggtgggtcctggagctttttaaagaaagcaattcaaaaatgtcttaagtttgcaaattgtataaaagtatgtaactgtatttgggatcacatagtgagggatcctcccagagctttggaaggagcttgtgagtgagaggcctaaagcctaagcttcattagctttccaataaatccacctttagcagttttagtgcctcattatcttcctttccactcagtctctgcctccaaacaccccaggcttggattctgaagggggagtattgcatttcaacccagctgcaattcagtgctggcacaaactagccagacttagcacagactccataagttaaagtgcatagtccccaacaacactgccctgacttcagacaccagccacactttgggggtcccgaggccccccacacttttgactgactacaaattcaggggctcgtgaaccctttaggcttgataatttgctagaacaactcacagaactcaggcaagtgccattagcaacggattcggattgattatgaagtatacaaatcaagagaactaggccaggcactgtggctcatgcctgtaatcccagcagtttgggagggcgaggcgggtggaacatgaggtcaggagatcgagaccatcctggctaacacggtgaaaccccatctctactaaaaatacaaaaaattagccgggcgtggtggcacgtgcctgtagtcccagctgcttgggaggctgaggcaggggaatcgcttgaacccgggaggcagaggttacattgagccgagatcgtgccattgcactcctgcctgggcgacacagcgagactccatctcgaaaaaaggggactagccaaatgaatggaagcacagcatgcagtctgggaggaaacctgggctcctctgcctgctcctcgtggagtcagcgtccatcagtcctccagctcatcagtgtgttcactaaccagggagtgctactgggccttgatgtccagagttttcaatggggtttcacaatgtataggcatgattgattagattgtcggccacatgcttgaactcattccccagcctctctgccctccctggaggtcagactggctcacatccccaaccctgtagtcatgtggttggtctttctggtgaccagtgcccactggagctgtttaggggcctatcacacatgacacttcattatcagaacaaagttactcctgtcactacggagatgtcaaacatagaaactccatgtcaggaacccaggacaaagaccagacaaattcttttttttgaggcaggatctcactctgttatctaggctggagtatagtggtgtaatcttggctcacagcagccttgacctcccagactcaagcaatcctcctacctcagcctcctgagtacctaggactacaggtacatgccaccatgcccggctaattattgtacgttttgtagagtcggggttttgccccatttgccaggctggtcttgaactcctgagctcaagtgatccacctgcttccgtctcccaaagtgttgagattacaggcatgagccaccatgcccagcctgacagattctttattatataataggtagtaatatattcaatcctgaccttacttctcttcctcttgactcctgtttcctccatggaagaaggctctcctccttgccttcctgttacgtggcaacttcctgttcccgagtgatagcaccatgtgctgtggcttcttgtgccattctgagaatcccggtcctcggccttgtcgtggccgagtggttaaggcgatagatgagaatcccagtccgctaggtttggctcttgatcgtaaaggtggtgaagggaattcactaccctattttggaagtcaaagatgttgtcatttttgtctctgcataccaactttatcctaattctgctcaaggcaatgagtaactctcccgaacaagggatggttcttttagaaagtgtgaaagagaatattaacatggttaacaaatattccatccattacagttttgtaactttcctatggtgtgtgcccaaggtgataacagtgctatattacagattacatatatatatatttgcatgcgcaattatacatatacatacatacatacatatatatatacacacacatatatatatgtataattgcgcatgcacaagaaatggcccatcttgataatttcttggaaacatatatttgcttctttatatgtaagagcaggcattccatctacctaaattctgacaagcccagtgggccacggttttccggtttaatgtcagccctgtgtacactggctgtgctgtttctagcaccgtaaggcagtcatcattattgagccaggggctgtgcaaaattggggtgattgcagagccaaaaagcgggctggagggtggtttctggaatgcctgttttactattcttcctgttcctttgctctttctgccaacttcagaatccttcctttccttaaaacatttagtgatatccctgaatttatttaattgctacgtgagtgatttttgccccacagcactggaagataattggaatgcattctagcttatgtgctctggataatatgcaattaccagccagctggagagtagtatgcctttgtctcacaatgtcttgtaattttaaaaagtttctcctatttgctgttgtctcttgttacctttcttacggcatcatatatctactatggggtccttatgtctgaatgtgtagacacttcttttggtatactgaataagctcctggcgatgagggaagacaccgctttgttttgtgatgtcctgtgcagttgggccccagtaaacgctacggaaatgatggtgatgagacttatttctttagatctttaaattgggcgatagataacagcaagttttagaagccatagaatttcaagaggaactttgcagaaagacagaacaccctccttttccttgaccagtctgtcaccaggtgacagcctgagcttcccatggtgaacgtgcctaaatggggttcccatcatcatgtctgctgactactaaaataattttggggttttcttcttcccaaagtttaacaaagtagaaaaaacaaaatgaggccgggtgcggtggctcacgcctgtaatcccagcactttgggaggtcgaggccagcatggcgaaaccccgtctctactaaaaatgcaaaaattagctgggcgtggtggcgggtgcctgtaatcccagctactcgggaggctgaggcaggagaatcacttgaacctggaggtggagcccgcagtgagccaagatcacaccactgcactgcagcctggcgacagagcaaaactccatctcaaaaaaaaaaaaaaaggaaaaaaaaatgacatttataatactaggtgctaatcagtaaggtaaaatgtgatacatctcaagagaatttagtgtctttaaaaagctatgtaataccaagctgcaataagctctctgagttggcctctcatcacaggaagccaaactgctaaactctacacaggaaaaggcagacaaatccatggtgaattagtcacaaaaaactggggaaaatacgaagaaaggttaaagaacaagacatttgaaattctatggaaatacacaatgataaataaaatatttcctcacaggtgtaaagaggtgggctgtggatttttctctgcctagagatcttgcaagcaagctaatgcctcctctgggctgcattaagtatgatccagcctgaaatcacagacaggggcttgaatcactcagctctgtttctcctgacagcattagtgattctcgtagatgtacttttcccctaaaatattttattttacactggcaagatttttttgtggggggggatcctttttctaaagcataatgtgcatcatgtttgtaatatttggctgcatttgttttgtcatttaaaatcttttcttgctttttctgtaagatgtgagaagctttgtaaacatggacccttttttcttttatcaaatggcattttaacttttaaaattgggtcatcagatttactggggcaaaattttctagcaagctgcatttccgtttttatacacaagggagagctaaggccctagaaaatagatttgggttttgccaaattccaagtccttcaatactttagggtggaagtactgaactaattaagagtggtaacagttgtgaattgtttggaaaaatacttgatagtaataaaataattttaagaactttgtataatttttatttttttctgaaatccagtgactagattgctttaacagcccattggacttaacatttcccattttatataattcgggagaataaatttaatagaatagtcctcagaggtctgacggaaatatactaaaataaataataaaggttgaggagtgagagggaattgttttgatttttaaaactttgatgcttggaatttctggtcttgagttatgtcctctgaaatcaggattcagctctgaaatcaaactcactcatcgaaagtaaccatgggaaactgaatttggcctacagatactaaggagccccaataatttcacattatcttttaatggagcaaactattacagagaaatcagagaaataatattttaggaatttgtattatattttagaaataatattttaggaatatgtctatctgaatcaagatttggtttttgacgttttctgcttttttactttatttttttaagagacagggtctcactctgtcacccaggcttactgcagcctcaaactcctcggctcaagcagtcttcctgcctcagccttttgagtagctgagactacgggtacgcaccgccacacccagctaatttttgtatttttttgtacagacaggatgtcactcaattgcctaggctggtcttgaactcctgcccctaagtgatcctcctgcctcagccctgcaaagtgttgtggttacaggaatgagccaccgcgcctggccagtttttgttttgtttagtttttttaatcaataaggctacatgtttcagtcagtgtcaggatttaaaaaaaaaaaaaaagtctacatgaagaaaatcttcattaattttcttaataaagaaatgtaattattcattaagaggtggaggtggggaagagtacagtttaggaagcagagacagggcagggtggcctcatcatcattatgcctcagggttcgggtgagcatcagaaaaataactgtgacttttgatggctctctatatggagaatttaggatagtgattaagagtacatgccttgaaatctgatgaaccaagaggttcagattccggttccatctaattagctgtgtgaccttggggaccttgtgtcacctctcatagctttgctttcctctcagggaaaatcatagaacctacttgatagggtattctgagaattaaaggtgtgtaacatagaatctggcatataagaaggactcagaaatacgtattagctattaagaaaaagtttccaattatttcgaagaagacctaatggataatagaaaaagaagaaaatacgttctcttttgtgtcttgatgacaagactcggggtgacaagagaaaggtaaaattgggaatcaagaagctcctttcgcttactcactaatggaattaaaagagaacaatatgtttacttgctgaatatgttacaggaccacaggtttattcatctcaagcactccagcttgtactcattcagccattaagtctctttaccctggtttttacctaggtgcacaccaacccaaactcgctcggccattaactctctttaccctggttttctccctagctgtacaccaacctatactcacttggttattaactctctttcccctgactttctacctagccgcctgccaacctatacccacatggctattaactgtctttactctggttgtctctgtaaccctgcatgcctgcaggaaaaacaatggggctagcagtcaggagcctggaatgtgagttctgacgctgctaccagataaatatgtgatcttggacaggtctcctgaccttcctgggttgttatggactgatgtttatgtccctgcaaaatggatatgttgaaatcctaacccccaatgtgatattaggaggtgggccctttggaaggttattatatcatgagagccctcatgaatggaattagtactcttataaaggggaccctagagagccccctaggtctttctacaacaggaggtcagcaatctgcaagctgtaagagggctctcacagaatccaatcatgctataccctatcctcagacttccagcctccgaaactgagaaataagtgtatgttatttataagctatattagggttatccacagaaacagaaccaatgggatatgtggagccatgtataagaggagatatattatgggccttgactcgtgcaagaaatcccatgagatgccacctgcaagttggagacccaggaaagccaatggtataattgagtccaagtctgaaggactgagaaccagagcagggaccactgctggtataaatcctggattacgaagacctgagaactaggagctccagtgtctgagggcaggagaagatggatgacacagctcaagaaaagaaggaattcccctttcctttgcctttttgttcaagtccagccctcaatggattggatgattcctacctatgtgggtgagggtgaatcttctttactcagtatgtagattaaaatgcaaatctcttccagaaacaaccttacagacacatcacaaaataatattttaccaactatctgggcatcccttagcccagtcaagttgacacccaaaattaaccatcacatcagtcacccagttaatggtattttgttatggcagcctgaactaagacatgcgttgagttatattatctttaatttgcaggatcccttccatctatctaacagtaacctctaaagtcacctgaagcagctattatggagtacagtggtgcaatcttggctcactgcaacctctgcctcctgggttcaagcaattatcctacttcagcctcccaagtagctgggattaaaggtgtgtgccacaatgcctggctacttctttgtatttttagtagagatggggtttcgccatgttggtcagcctggtcttgaactcctgacctcaggtgatctgcctgccttggcctcccaaagtgctgggattacaggtgtgagccacagcacccagcaacttttactttttttttgagacagagttttgctctgtcgcccaggttggagtgcaatggtgcaatcttggctcattgctacctccgcctcccaggttcaagtaattcttctgcctccgtctcccgagtagctgggactacaggcgagtgccaccacgcccggctaatttttgtgtttttagtagacatggggtttcaccatattgaccaggctggtctcgaactcctgatctcgtgatccgcctgcctcagcctcccaaagtgctgagattacaggcatgagccatcgtgcctggcaacttttactttttattgtggtttttaataataattatcccctgcttctttttttttttttttttttaagatacagtttcaatctgttgccccggctggagtgcagtggcacgatcttggctcactgcaacctgcgcttctcgggttcaaacagttcttgtgcctcagcctcccgagtagctgggattacaggcacgtgccaccacccctggctaatttttatattattggtagagacggggttttgccatgttggccaatctggtctcgaactcctgacctcaagtgatctgcccacctcagcctcccaaaatgctgggattacaggcgtgaacactgtgcctggcctattccctgctttctcatcttacataaatgtactgtgagtacatttatggagactgttgaaaagcaaattcaattcaacatccatttgtcaaacattgattaggttgagcaacacactgtgattggcctggggaaagcagtattgtttggattccttacagtcatggcagacccaccttattagtgcaattaggagccaggtttgagagagactattaatagaggtaatattgcacagttagccaccttgaagagcttgtcaagagcaggtctccaagtagacacagatggaaattgatagagattaaaataaaccatgagatgctctgacttttaggggtttaagctgttcaggaaatcagagtttaaacaacacatattttacttaatgctagaattttagtgtcattcctctggatgttatttgcaattttcccttccaccatcctcaaactcccagaggaggaaagggccactgcctctttggccaagcagtctggcctccagacacagcaggcaactgggtttatcttcctggctactgctaggagactcaagccttcttaacaccgccagtgctcgtgtggtttctaccaaccctacttggcccaaaatgaaaaaaatgtacagcacggcccattcattcacctttgagaaaagaaagattcagggctttaggtgccaaagtaataaggatctggagtgtgtaacatttgcagatttttggcattttggaggatacctttcctccttgttcacaacagttcaggtggctacccagagaggtcagtgggaacttaccaaaaatcggccctgatgaccactcctgcatcaggttctccagtggccttccattgtaagtgttattaaatctaaacacttgaacatggtctataaggccacaatcaccaggcctttgcctggtgctggattcccacctccttgccttcatctctgggtggctgctccactgaactctccttctagtccttggcacttctagaagtctttctcccagatcttcatgtggctgtctcctccttgtcccaatgatctccttccagagagacccttcctggccaacctagctgaaatagatagcccctggcaacaagtcatatcacctatgttaactttcttcatggcatttgccactatctaaaagttatcttactgttcctctccactggtataaagctccgagaaagccagggcctttgcctgtcttcttgactgctatgtcctggtacctgcagcaggactttatgcgtggtaagcactctttaaataggtgtggataagtgaatatactaggcagaaatgagggctaggagggatttggttgcagccccatatccttttggcaaagacagtagaatcatgtttttgtaactaagatgagtcaaggtagaaattgtcattctctcctgttagattccagtgatctgtgacttcatgtaaagatttcttaaaataattctaaaggcacacatttttgatgtaacagtgcttttgagctttaagaatttgaggcctccattctaatttcagaacatattaatgaagatttaaatatgcattaaaaagtacaagcattgcttcaacccagctatttctcagaaattctcattgattttataacagctgtcttctagcttgagccttctaatttcttcagcaaaaaacaacaacaacaacaaaaaaaaaacagataaatacatctaacttaaaattaagacaaaaagtattaattggatttttagttttgtgtgttttccgtatttaaaatgtgatgctatatgccttttagatggcttcccttcttttatgttaaagaatgaattgtcagaatcatggaaaattgtggatgacaccatgaccctttccttgaggcaaaacttgattcttgttctgtcactcctattgaaataagtatttaaggtcactatgctcttaggaaagcatattcctaacatatcatgctgttttgaagaatatctttcccttggacctttgtagaacctttttccaaagattattttaaatatcatcctcaaaagttgagctttaccaaggcttcccatagtccgttcttctcttccacagctgcctctgataacttatgaaagatgggctcaggtgtcagatgatcagggttctgcttgcagatttgtcactgatttgctctgcaacatcttggataaatcactttacgtctcagagcttcagtttccctgtctctacattggagtcatgaaatggggaaaggggtagggaagagagcaccatctctcctgctctgtcattctccagctttgcctttggcggccctagaaagaaccatgtgggtttgccttggcctggctccatctccgagaactcagctcttaggaactgaaactcagaggggtgtgggtgagagatttttctccctgttcttagccggagtttctttcaaaaatgcaggcttgacaactaagggttccctagcatatctgtagcttagtaactggtcaaatgtgtagagcttttggggactcacctgtagcatctgaagagttgagatgagtcctgtgtgctgacaggatgtgatggacaggacaccaacatggtggagtcagtaaagggagcacctgcttgccacaggggtttactgtaatcacccaacaggtcttccttctggctgcacaaagccaggccactgagaccgtggcattgcggtaaagaaagagtttaattgacgtgaggccagccacaccatgtgggagatggagttagtactcaaatcagtctcccctaagacttgtaggtttgggtttttcaaggattgtctggtgggcaggggggtagagaatggggaatgctgattggtttgggatgcagttatagaagtgtggaaaatggtcctcatgtgctaagttagcctctgggttggggagccacaagactggtcgagtcaccaacactggtgtgatattgtgaagtccagcacgctagagtgtcaacattccaggaagatcactaaaatgaacatttgttctgcaagagaattaataagtccagcataggggttactggtatatatttttatttgctgccattttatctgatgattgactgttttgttctatttttctccatttttataatcactacccccaatatagtcgctactccaggtggagtcagctggtcgtcagaaatgcagaagtctgaaaagacatctcaagacaccaatcttaggttctacgatagtaatattgtctacaggagtaactggggaagtcacaaatcttgtaacatcagggaaaaatacctggttattgtttcacttacatacatcttagcagaattcaggcccctctcataatcctaacctgtgggctttcattagttttacaaaggttgtttagttttgggaagggctattatcatccttgcatgaaggttaaactacaaactaaatttctcccaaagttagcttgacctattcccaggaatgactaaggacagcttggcagtcagaagcaaaatggagtcaactatgtcagatttctcttactgtcgtaatttgcaaaggcggttttgctactttccttgcaaattagttggggacttcagcagttctgcatagaccaagaccaagagttttctagtactctcatcaagaagtcacctactctggtcccattttaaatcccctgcatactgtctagaagaatatataacagatgctaacaggatccaatttgttcattgaaatatatcaagttttctccaaattttcaagaccatattcatttgcatttaattttaaaatatcaatacactgatgtcatggtatatcatttgaaggcacagggaaggatattgtgaagtccagcagactaccgtgtcagcattccagggagatcaccaaaatgaacatttgttctcaaagagaattaataagcccagcataggggttcactggtatatatttttatttgccaccattgtatctgatgattgactgttctgttctgtctttctgcatttttgtaatcacaacccccaaacgcctgaagctttgggtttttatgtaaactgtaaagtagaatttatttggaaccagttccctgtcataaaagctaatgaaaaatacagaagagtgaagaaagaaactagaatcacctataatctcattgaccttcaggtatccaaggttggagaaggagccaagaaaaatcatggacagagttattttggtgccattacattcagaattatttttaacctgttgtgaggctcagctcttttgtagtagcttcagtaaatcctaacccatgatgcacaccgagcccctgcccgttagtggtaatgtgcatgtctttccatttggttgaggacaccttgctgtactgggcacttggtgccagaggacttttttttttaagggccattaagggatacatggagacctgcattctcacactggcgcagtgatttttgcttcctctctgaaccggcctttggtatttcacaggctgaacccttctctttttaagtgacctgtcacctctcatgtagtcaatgctttcttactgccaccaagttggcaagtgtcatgccattcctctccccaggggagagaattcttaatccaaatttattcccataggattttcaggacttttcctgagccaaattagaccctttctctcagtatatctactatctcctcctttccctgtattggccagtgctagactggaagctataaatagacagcaccttttaattcctttaaagatttggagtaggtattatttctcccattctgcaggtgaggaactaagcctcacaggtagggagtatggtttttggatttcgcatttagttaatgacaggtgtatttgtttagtgaatgttgaatgtagagttttaccttctttcatatattgaagatgcacatctatgcaagtctgtgcataatgtaatggcacaaggtaatggcaccaatggcatcctataaattttatttaatagcctgtagcattgaacgtcttcaagccttttaactctcaaagctaaaataagaagattgtgtagttactagttaaaataaaggcagtttaaagaggtggggctggagtagggcaaactgagaacaaagatcttcattttgccccatttctctggcctatttttattcactatgtagttgtgtggccctcccttgagcagtaatcatccaagccatctatgcaagtctgtgcatagatgtgcctcttcaatatgttaaaaaaaactataggactatgggagtttagtgaaagtcaatcacattttggtttacagcccctttgcccactgagctgatctcctggcttgtgaattctggcctcttcttgaaaaggtgcaggagcgcgtcagatcagcaacaggtggtctagctggcccggggaggacttacgcagaattgaagacagcctcctgggggcaggtgatgagacctgcccgtgaaggctgttaacctggctgctcctacagtaggggccccttagtggacttccttctgccaagcagttaggacctacaggttagtttccctctccttggttgtctctgtttctcgagaaggtagggcccttagctccatcagtcaagttgcagatgctctacctttgggctagtgggtgttggtcttcactgctcctcgtcttgggtgacactggacaatcttttttactttgtcaaaagacaaaatgacatcaaatttaaagtttttaattggcttttcctgttctagaatcagacaacgtcaagcaaaggaagttggcttaatagaaagggtagaggaaagcagaaatagaacaaaaagcatattggttgtttcaaagttaattttccttttaaaggttaaagcagagggaacttccttatgcttgcttaaactggcctgtttggggatttggtattctctctctctctcctgatttctcagaaggtcagataaacagcttggtttcagtggtgtgtaacttcagcaagggtgattccattttggtttggcctgttgagtctagtgcaggaggtcagtccaaaccaatggcctcctataaatgttatttaatagcctgtagagttgaacgtcttcaagccgtttaactctcaaagctaaaataagaagattgtgtagttactagttaaaataaaggcagtttaaagaggtgaggctggagtagggcaaactgagaacaaagatcttcattttgccccatttctctggcttatttttattcactgtgtagttgtgtggtcctcccttgagcagtaatcatccaagccagttgttagaacgcgtttccctagcaagctctgtcatcctgagaaagcagaggggtaaacaataaagacttaattgtatctcaccaacatttttgggtcccattggtgagtaaggcatcaatttaagtgcctttggaagcaattttgattaaaatgattttttttaagttcttaaacagatttcgggatgataggagattcggacatattttaaaacaaaagaaacttatacaagcagaattaaacaacctggataaaagtacctgtaatgtattgtgctagtacaggggaaggagggaactgcgtaggatgttataaaagaaatggtattttagccttttcttaaaagatgaggatgctttgggtaggaaagagggatcaggagagttccaggccaagggaagtatacgaaggcttggaaatacgtgacccctgcttactgtgactgtgtgtttgggacagagtttttaacagcagtagatgtggggaggaaaaatgaggctagggccagaccctggaggctatgcatgaccaaagaatctgggctttattctatttcacagaaagagttcaaagcctgagcgtatttgaaatgagagaatgaatgatgtgtccctctgtttaagcagagagaagataccatttgcattgaaaaagattaattgaagaggtcaaagaaagggcagggaaaacccataagggaggccatttaaatgtgaccctgtgggagaggagacccgtctgacagcagggagaccagggggaaaagattgcttatgaactatttcagaggtaaactatatgatgacaaataggactgcagggatgtgataagggaaactgatcattttaaagatttccaggagagataactgggaaatgatgctacctttagatgagacggacaggatccacaaagagaaatatttgcttggttgaaggaaaagtgataaacttgatttgtgatgtttggtaggacagtggaattcccaattggtgactccaaaaagaagcgcaggctagagagataatttttgaagccatatgcattgaggttgtaggggccaagggagaacttccccctttcccctctgaaggttcgttgagaatcaactgacaaaggcagattaataggagaaaagacatacaaaatgttaacaggcatagcacggggaactcacaggaggatgattacccaataacctagtgcaatacagaatcgtacatacccttttcataggggagccgggagatggggggggatgtaggcatttcttttgagggggcagtcaatcattagaggaaatgaatggacagaagttaacttggaaatgatttttttcttgaaatttgaatgagcccaagaggcaggtattatcttgtgaaggagtcttctcagatgtggttgcattcctcagtcttcttttctgagataatgaaatttcagggaggagagggaaacagttgtgtttctgttggcgacccctgcgtactgtgactgttaaactttcttggccagatgaggaaattccagagcaagtccgttcttgtgtccttcgctggatgggggtgggacaagaccaggttagaaggacctcgattctgaggcagcttctaaggcctctaagcatgtcaaggcaccagtctttggggtatcactttctgagtcccgacaaggtcatgtttgaagccatagaggcaagtaggatccccatggcagattttacacccaggaggagggcttagattcctctcaggagcctcccatcagtagttaggagctgctcagaaagcactcaccatatggaggattggagtctatgatctaggctgctcagcaggagtccacaaaagagccccgtctccaggatcagtaaaattaacagccttcctgaattatggcttatgtcccctccaggggggcagtagtgaagttctctagcagcgaggcctccagaggtcattaagagactcacctgttgctgatcacaactcatctcacctgttgctgatcacaactcatctcacctgttgctgctgaggacatccagacttagtggtgcctggtgtgtactcagaaagggtaattgcccacatttgactgcatattcacaatctgtgaatgaaaaaaaagccaaaagaaaagttggggaatctgcatttaaggagagaaaaatgtggctagtaaacttagagaaacgcgctggttatggtggctcatgcctgtaatcccagcattttgggaggcctaggcggacggatcacttgaggtcaggagttcgaaaccagtctggccaaaatggtgaaacccatctctactaacaatacaaaaattagccgggcatggtggtgcatgtctgtagtctctctgagctacttgggaggctgaggcaagagaatcatccaaactcgggaggcggaggttgcagtgagccgagactgcaacactgcacttcagcctgggtgacagagtgaagactcttgtctcaaaaaaaaaaaaaaaaaaaaaaaaagagagagaaacccaaacactatagaaaggagagatattttaagtaaggagcaatggaggagaaagtaaataaaagagtttagacaagactaagatgtgggaattagaaagcttttggcaatttttttaaaattattaatgtccttctgggtgccaaggaattttattgtgtggtcaggcggtagaatcatccgtaaaggtgaccctagacacctgacagtaaaggggaggtgaggaatgggtgaagtagggaagatgcagagagtttgatgctagagtttatcctttagtacttgaagattgacattaaaatacctagaacaaatacatttggacttagtgttccataaatggatatatatctaagagcagttttcccagggaaccctatctaaaagttgaattcgttctcaactgtttttacagtatctagtttccctccctcattactgtgctatgtactctaggggaattgagaaaaacgaaggcattatctttgtaagaagaaaaaatcaagatgatctggcacacctggaggggtagtggaatgggagttgatcactgaacatgtatgcaatgtgcataggcacaggggattgggaaagacatcccaggcgagaagtaagagtaatcacaaaggtgggaataaatatggcatgctacagtgtggcatgggctttgtgttcctattatttgcagaatgttcgcatagctgggtgtcttagtctgggatgctataccaaaataccatagattggtttataagcagttctagaagctgggaagtccaagatcaaggcaccagcagattccatgtctggtgattctgtttcctggttcatagacagtgcctttttgctgtgtcctcacatggtagaagggacatgggagctctttggggtcttttttaataagggcactattcccaatcatgacctaatcacctcccaaaggccccacctcctaataccatcaccttgtgggctaggatttcaagacaggaattctgggaggacacaatcatttagtccacagcaataagaaatatgcgaaatcaggatgggtaggatcagatcatagagatctctagattctgttaccggatcccaccacttactcaaagttagcctttggatcgggggtttcctaggtattgtggctttgtggtcaccagaaagatgttgctgaaaaggggtccggatccagatcccaagagagggttcttgaatcttgcacaagaaagaatttgaggcaaatccatacagtaaagagaaagcaagtttattaggaaagtaaagggataaagaatggctactccataggcagagcagcttcgagggctgctgcttgcccatttttgtggtggttttttgatgatatgctaaataaagagtggatttttcatgcctcccctttttagaccacatagggtaacttcctgatgttgccatggcatttgtaaactgtcatggctctggtgggagtgtagcagtgagggccaccagaggtcactctcatcaccatcttggttttggtgggttttggctggcttctgtactgcaagctgttttatcagcaaggtctttatgacctgtatcttgtgctgacctcccatctcatcctgtgactgacaatgccttaactgtctgagaatgcagcccagtaggtttcagctttattttacatagcccctattcaagatggagttgctctgcttcaaacatctctgacagttccatacaggcaggcttggaattgatagactgaggagaatttaagaaactgagtagtgcagtagctcatgacagaagtgtttaagatctgagcaataggatacagctcataactatttagaaaaagtacagaatcgagctattaagtattattacagggaaaaaatgtactgagtgaccaaaaaaaaaaaaaaaaaaagcaaggtactatgaccttctttatgacccccattccaaaaagatggaagagcagatgccttcttaaataatcagaaccagtggtgcattggtcaacgaaaacattcattagggtgagaaaataatatgtcacttatatgtacatgtaatttttaattaaaagcatatatctgtttgccacatctttcattgtaaggctgagattgcctctttgagtaaactggctagaacacataaatcacacataatctttctcacagaaataacacctataatgtttcacttaggatttccagttttggtgttctgggcgtggtggctcacgcctgtaatcctagcactttgggaggccaaggcgggagtatcagctgaggtcaggagttcgagaccagcctggccaacacggcaaaaccctgtctctactaaaaatacgaaaattagcggggcgtggtgggcgggcacttgtaatcccagctacttgggaggctgaggcagggaaaattgcttgaacccgggaggcagaggttgcagtgagctgagttcgcaccattgcactccagcctgggtgacagagccagactccgtctcaaaaaaaaaaagtagggcctcacccaggctgtagtgcagtggcatgatcacagctcattgcagcctccctcaaactcctggccttgagtgattcttgcacctcagcctcccagagtgctgggattacaggtgtgagccactatacccagccatgaatgagaatcctaatattaacaaagcatgctgagtttacacttgcctcccagttgtaacagtctagccctcaccaaattcaacagcatttctgttgctgctgctgtttttaaagaaactcacctttttggagtcttgtattgttcatgctctttatagaactaagaacccaatttcttctcacagttacattatattcatttatgtaagtactatctaattgaattaatgtagctccagtaataagtataactaacatcaacatgtttgggaacacagctgatcattggtatttgagccaaatggcaagagcagagtggcctagacaaggaaggtctgtgcagtaggcagagtcataagtagtggtcagttgagtgagcctcttaatttccttcagatttattgactatcagttgtataccctctaaaattttctctagatactccatttctggaacacattttaggagatgttttcttttttttattattattttattattattatactttaagttttagggtacatgtgcacaatgtgcaggtttgttacatatctatacatgtgccatgttggtgtgctgcacccattaactcgtcatttagcattaggtataaggagatgttttctttcttttttctgagacacacgcacatgccaccacacccaactaatttttttattttttgtagagacagagttttgccatgttgcctaggctggtctctaactcatggcctctagtaatcctcctgcctcagcctcccaaagcactgagattacaagtgtgagccacggtgcccagcttgttttctatagatcttgccattgcccattagagcactttgtgtttgtctctcctgcgcaaatccttgactgtgcttcagttgacctggtctcctggtgtctccttctctgccaccaactcttcatgttgatttcagccatgtattaccagcaaaacggctagtcctggcttggaataccaaaaagctatggaagccatgcagggccagaatatagctaagtgtgtttattagtaaaacctgcctgtattagtctgttttcacactgctgataaagacatacctgagactgggaagaaaaggaggtttaattggacttacagttccacatggctggggaggcctcagaatcatggcaggaggcaaaaggcacttcttacatggtggcggcaagagaaaatgagagagatgcaaaagtggaaacccctggctgggtgtggtggctcacacctgtaatccaagcactttgggaggctgaggcaggtggatcacctgaggtcaggagttcaagaccagcctggccagcatggtgaaaccccatctctacaaaaatataaaaaaaattagccaggcatgatggtgggtgcccgtaatccaagctactcaggaggctgaggcaggagaattgcttgaacccgggaagcagaggttgcagtgagccacgaacatgccattgcactccagcctgggcgacagagtgagactcctcaaaaaaacaacaacgaaaaaggaaacctcttataaaactatcaaatcttgtaagatttattcactaccatgagaacagtgtgagggaaaccgctcccatgattcaaattatctcccaccgggtccttcgcacaacatgtgggaattatgggagtacaattcaagatgagatttgggtggggacacagagccaaaccatatcactggccatgttcacctttgttcttatttttttgcgctttagagattcattttaaagtttattggatagcaaattgtctttgccacatatatgtagctcttagctaaccaaaactcaaaattttgtttggcattttagctttattaaataaagtgggatagacatacttgacaaataaccttttatatacttagcaacagcatacagactagatctgaaatgagtgtacatttcaagttggcttctcaaatttaactagaaccagacactcctctactgacactctaataatatgtgtatgtgcatctatgtgtatttatgtatgtgtacatacatgtatgtgagtacatgcgtctgtatctattgctaaccaaatacttgtgtgtgagtgcaaggaattagaacctccctgggaagagctagtcatctccttcacacagtatactttttgcatatctcctgttggcctctgcttattcatggcttctactcataacatccctagcataatgctaccttatgacctatattcaacttctactgccataccaactggacatgttctttcacacctgcagctcaaattcttaatagaagtgaactgactggctcagccaatcactctggctgtgcttgtgcagagaccctcatgccaggccccacacaagcttagagacagggtactctgagaacaggggaccattcccatctccatcaggtgagactaggacagatagcagctgcctgatacagaagatggcagcgcaggcagcagatgctgtgggtggggcagtttcccttagattataggatggcaggaaagataaagcctgccatcgtttactccgaggggtgtgttatatgtgcctacgagcagcgttgctttgcaccattccatatgtacacattcccatcactggatatagttaaacaccagtccctcaatgacaaattttagttaccctatgtaatagtttgctaggactaccattataaaataccacagattggtggtttaaacaacagagatttattatctcactgtgtggaggctggaagtctgagatcaaggtgtcagcaggtttggtttcttctgagacttctccccttggcttgtaaatggctacctccttggtgtatgtatgcggggttgttcctctatgtgcatctgtgtcttagcagccttttcttagaaagacagcagtcatataggcctagggccattttaactctttaaaagccctatctcaaaatacagtcacaatctaaagtcttggggattaggacctcaacgtatgaattttagagggatgcagttcagcccataacactatagtatagcaattccgagtcactgcacataaaacacagactttggggctaacttcacaaatcactatgtacatgacacatgtacattttgatcaataactaatcatatcacttctcagaatctggtggtgatttgttagcgagcgtctgttcagttcatgcacagacagcaaagtatgtagctgtgttgctgccttctttctcagtgaaaacatgacatttaaaaaaatagttcatcaggagagggaattggccaccaaagatgaaagtgctgcaaagaaacaggtagtagtaatgaaggaagtgaaatttgttgtgatgagatgtgaaaatggcaacaggataacgaagacagtacaagacccaggccagcatgaagctacagtacgaaccagattgaggctgggcacagtggctcatgcctgtaatcccaacactttgggaggatgaggcaggtggatcacctgaggccacgaatttgagaccagcctggtcaacacagtgaaaccccgtctttactaaaaatacaaaaaaattggctgggcccagtggtgcctgcctgtaatcccagctattgggaggctgagacatgagaatctcttgaacccaggaggtggaggttgcagtgagccaagattgtaccactgcattccagcctgggtgacagagcaagacttcgtatcaaaaataaaaaataaatgaattaaaaaatatgtaaaccacactgaaaatgtccaatgaatataaaaaaaacacagtaaagtcatctcaatatattttaatttaaattgcgctaggaacagaaaaccacttatgattgaaattgagtattttactttagttggaagaccataatgatttttaaaaatcctgcttagtttgactagtattcaggccaaagtgtttaaaaatcctgcttagtgtgactagtattcaggccaaagaagttagtttctgaattaaaagaaaatggtaattacatggagactataggaggacttttgactgccagtaaagactggtttcattgtctcagaagtcaccatgaactgatgaatgttaaatagtctgatgacaccactagtgtaaatcaggatgcttccgtgaaatttcaccccgaatcccaagatggaggcaggtttatgatgatcctggaatatttcatgctgatgagatagatcttttggaagacaaccctatcaagaacttaagtgaagaaaggctgggtgccatggctcatgcctgtaatcccagcactttgggtggccggggcaggtggatcacctgaggtcaagagagtttgagaccagtctggccaacacgatgaaagcccatctttactgaaaatacaagattagccaggtgtggtggctcatggctcatgcctgtaatcccagctacttggaaggctgaagcagaattgaacctgggaggcagaggctgcagtgagctgagattgtaccgctgcactccagcctgggcaacagagcgagactccatctcaaaaatttaaaaaataaggtcgggtgcattggctcacacctgtaatcccagcgctttgggaggctgaggtgggcggatcacctgaggtcagaagttcaggaccagcctggccaacatggcaaaaccccatctctactaaaaatataaaaattagccaggcgtggtggtgcacacctttagtcccaggtgcttgagagtcttgggcaggagaatcacttgaacctgggaggcggaggtttcagtgagccaagatcacgccactgcactccagcctgggcaacagagggagcaagactgtctcaaaaaataaaaataaaagaatttataaaaaataaaaataatttatgtgaagaaagatgtaggatcacaaagtggccatacaggagagattctagatattcagctagggaaattggtgaaggtgaacttacccacataaatgagggatgtggctatcaagaaaacgatgaagatgacccagaggaagtgataatggcaaaaacaaacaaacaaccaaaaaaacaaagtcacagtaaaggaactcagctaattcacgacatggaaatggcaaaggataaaatgttggaagccaacccaaatttaggagtatggcagttttccaaggcatagaaaagacgtgtattctatatcgtaagttatttgacaaggagaagaaaagcactgttcaaagtgccctcgataggccagatgtgattgcttgctcctgtaatcccaacactttgggaggccaaagcaggaagatcgcttgcgcccaggagttcaagacaagtgtaagcaacatagcctgaccctgtgtctacaaaaaagaaaatcgccaggcatggtggtgtgcgcctgtagtcccagctctcaggagggtgaggtgggaggattgcttgagcccacaaggttaaggctgtagtgagctgtgattgattacaccactgcactccagcctgggtaacagaatgaggccccgtctagaataaagcaaagcaaagcaaaatactctggatagagtttttttaatagggaaatgaaacattttaatgctcaatgtttttaatattttaaattactgtactaagcaaatgcttattttactagttttttccttcccctctacatgtatgagtttttaatgtttagacataaattttttaaagttgtggaacagtaatcccattgactattaaaatgacttgtgcactttgaacttgcatggttacttttatggttccacacgtctatgcaaagctgtgtgtgtgtgtgtgtgagagagagagatctttgctggaagtgtgtttatctgtgtgtgatatttgctgaaagtcttcctaagatgtatcatgttatgtttttacttgaattattcacctaagcctggcaagtataacttagccttttcctgatggtgcagtcatcattaataataagttattgttttctgctataacgtagaataatctggtagttaagggtgtggtttatgcagccagactttgagttcaaatactgagtcatcttcttactatgatatgatctggaacaagatgctatatctctctgtgcctcagtttcctcatctgcaaaatgcagagagtaagagtggctacctttttagggttattgtgaggtttaaatgactaaatgtacccaaagcactgagtggctgtcaacacagcaaacgtgttagccaatgttgctgctgctgctggcagggacaatggtggtagtagtagtagctgcatcagtagtagtagaagtattaatgatgctgtgtatctgaagatttattaaaagttggggctatttttttctatattagacagcctcaaactactatgcttttgatttcttctgttgctttatttacgtgttttcctctctcatgattgggctgttaatggacattctttgcattggactcaagcagaacaactacctcattctcatcagtatcttattaatttgatgttcttaaccacgatgggtgtagtgaccttctgaaagtctgacttgttgcagtagcgtggaaccctggattgtggctcagttcctcatggacggcctatcccagggaagcccaggaaagaaccatttattttgctcaggcctcacacattcatgaaggtgcttccaagttgccatttgacattatctccaaatgaggttatgctcttaatagttgtattttatctgtcttaaaagcattaagttattctttgtgagcattaaaactatattagtttgttaccctcttttggcatttcatttttaatatactgagaacctcgaagtacagaagtggcccaggcccttctgaggagccctgctcaacagacaagcggatggagttcctgactgtgttaagagtagatagtaacttgattgatgatctcggaataggaaagcagaactgtatttcttgcagttctccattgtttctctaagtctcttgcagggtctcaagatcatctctcaggttttttgattctaccccagggagctgagcatagcatgtgcctaacttgtaagaacaaaggtgtaaatacttcacatttataatttggtagggtcttcagggccagacgttttgtgggccaacctccactaaatctaaaagtcattctccaaatagagtatattgtgtttaatactgcttaaaaggtatcagtcacatgggttcttagttgaacttatggctaaaataatttttaaactcatttttctttttattttttttttgagactgagtcttgctctgttgcccaggctggagtgcagtggggcaatctcggctcactgcagcctccgcctcccgggttcaagcgattctcctgcctcagcctcccgagtagctgggactacaggcacgtgccaccacacctggctaatgtttgtatttttagtagagatggtgtttcaccatattggccaggctggtctcgaactcctgaccttgtgatccacccgccttggcctcccaaagtgctgggattacaggcgtgagccactgcgcgggggcattttcgtttttccaaccaaattgcttttaactaaaatagtgtattaattgaccttgtctcagatgtgtaagccgtagctccccttttcccttcgtcacccacctccatgtgcttatggcatatggggaaagagaaaaccttttttttttttttttttttttttttttttttttttttcaaaagtgatctgaagttattgatcagtaaactggaaaacccactgtaccagaaattcagttaagctgaggctcaattttcttcccctttatggtcagctttgtacatttcagtcccaaatcagggagaactgggagtgcagttggcatcccagttgcctgacgaatgaaatacgaaacagagtctgttgagttaatgtcatcagtttattgaaccacagtcggaggaaaaaaactatatccaaaaaagtccttacccttcagaccaactcatctcttgaatatgggaagcatttgttaacaaaagtataaggtttgtgaatagttatgtgctatacacagttattttcttacatcatggatattcccttaataacctttgaaagaataaatggggattttcgaaaagttctagccattggccagggcaagactgaacgtgctatctgttgaaatgttaaaagggttgaattctgaaggaaagctagcaagatttcgctaatatgtgctgtgtgtcgaggtggtaagaacatgtatgaacctgcagttgttggggtttaattggggttaagttgttccttaatttagataatttggtttcttttcagggacttttttttcttatgggaaaataccataaagtttaccattttagacttttttttttttttttttttttttttttttttgaggcagagttttgctcttgttacccaggctggagtgcaatggcacaatcttggctaattgcaacctcagcctcccgaggttcaagcaattctcctgcctcagactcctgagtaggtgggattacagggcatgcgccaccatgcccggctaattttgtatttttagtagagatggggcttctccatgttggttaggctggtctcaaactccctacctcaggtgattgacctgctttggcctcccaaagtgctgggattacaggcatgagccaccgtacctggcccattgtagccatttcaagtgtacatttcagtaacatgaagtacatttacattgttgtgcaaccatcaccaacatcccatcttcagaacttttccgttatctcaaactgaaattctgacctcattaagcactaactccccggccgtcttccctcagccgctggtaaccactgttctcttttctcatctctgaaatcatacagtatttgcttttttgcgtctagattatttcactttagggaatttgattttatgtcagtttttctaacaggttgcttgtggagacagtaataatcaaaaacttactgtccaaagccctgagtctgaaattctgcatccaaaagtgctacagtatgcattaaaccgattaaaataacgtagcatctttgaggacgagctctatgttcaactttgttgttgtttaatatttatattgtaagaaaatctctcatatatgaaaatgtaaatgtaaaattccactattttgttgccattgcttttcttgtttgggggatcattactgtattattggcttccaggatctcagcagggcacgagggaggtttagcctgtctgaattttggaatcaagtttggtgatgtgtgagctgtccccagaaagcaccagtataatgtagtggtcttaacctgcattctttaacgcttagccctgggctctataaccccacccccatgcccagcagcatcccagggtcctcttctggaatccaactagagttcctaagggaggaggatccccagagggagaaagagaaagtgtcagtggacagaaatatgatactgaaatagcagagttagtgtttgagtactgacttttcccgttgcacatggaagaaatttccatgccttgtctggatatctttagaattgctcctttcagtgttaagatggtttgttccactgtaaggactgtgccacatggcttggtggacttggagaggcggaggtggaaacgatgcgcaggagttggcttggggctttttgtttgcgtgtccctgtttacctattcataatcatggatcccctctgctttgtgatactgtgaaccacgcataacagcaattctttacaccaccgggttgagaagaaggcgcctgaggctgactttctggacctgccgtcacgcagtaaagatgtggttggtgtgtgtcaggatgagagggccacgactggacccacgtgtggctgcttcccgggtctgccatgtgctttgctagctgcaaagcgtttcctctagaacagctaatcctgcatgactgtctcctgcattttgctgtcagaacgtgcacgccgctttcataagaactaattgaagagggagctagtttgcatctcgttcttgcacaccctgagtcatgaaaaacattttcttttaagcaaaattgaaagaaagaaaagagagaaagagggccaaaaaaaaaaaaaaaaaaaaaaaaggttgccatccagtggaggaaggagtaattcccagcttcattcaggaaaaacgcaggctgaaatatgcttaactgttttaacttgactggggatttttaccaccatatgggttttttttcgtgacatccggagccaaacgggtgcagtgtgcctttaagaaaagaggtgcctcactaaacttcgctgtagttgtgtttcaccgtctcggtagggaaggaaagtgttaatttcttatttgtacactttttttccccgacttccttcctattcacttcattaaatctagaggcagttgagcatgggagccgtctgtatgttgaattagggctcgcactcttgcgcaacacgtcaccagtcggaaactgggggtttgcttctgtgatttatttcattattgtgctggtaaaaggtttggaagggaattctttttgggggtagtactttagcattgtgtagcaagttttggggttttttttgtgtgtgaccccccagcccccagcgctgagtttgagtcagttgagccagtttagtaaataattttttaaaataaaagaacagtttaaaatctccatgaataattttacttacatgcaggagtaatcttactctactctttatgtgcgaaaagcattgggaagtgtttagtgaattgatttccattagaaaaagacccttagaaatcacagaacataaagcactgcatatggatgtgtttggggtctttggggaggagggaagatgttttgtagctctctgcattcctgcataaaaccttagtttgaggggaataatggtcagtactttgtgtgtttccttgtaattccaaaacctaaatttaataagaatctgcacgtattagcagttgtaataaaaattgtttcagttgttctgtttgcaatttaatttgttttcgcttcttagctactcacagatttagagatttaaaagtccaaatatcacaagcttatgctttttccctcttttcaaaattaaggcggggtgagagtagaaatatcctttttacaactccccccaacttcatttatctcagtgagaagaaaaacaatgaaaaattttacagcatgaaactgaaagtgaagtgttacagttgttctgaattttctttaaagagcagaataacatattatgttaaaatatttgaatcttagaacatgtacattgcttttatttacatgcatttaattttcttaaaaaattgtcttctgttttaaaaatcatttagactgctccaaaccatgctgctattttttaaaaaataaggtacaaaataccaaaaaagcataccttgttttaattttttaaaataaatttgaatattaatttgttttcttactggcttaattgttaaaatgcgagcaagactcagaaaccatctgagctaaaatctctaattctgctgcctggaataataaggggaaaaaaatatgataagtagttgtcaatattaaaacaggctgaaggaggaagccttcgggccttgtagtaggtagcatgaaacagcagatttgagtatccacaatcctttcagtattaaattttcagtaaaataaaattacttgtatatgaaaacacagcctttccccagctctcttttttttcctgatcagctgatgaagagactagcagctcgctgctttgctggcttgttaattttatccccactaactgtgatttctgatagccggcctgctgatagtggtaaggtaaatatcctttttcgttgccgtcttgaagattcagtagaactacatcccaggcatgtgtaggtgtgtaacacatcaaaagccggtgttctccgtatttctgtgtgcaactgggtgcttgaggagagcaataccattaactgttgacagccttgcatgctgtatttattattagcatgttcagcctccaagatatgcgggacaatttccttttcttaatcttcgcttagctgcagtgtgtttagctgtatagtgggtgtcctgcttttaggcaaatgaatattaatgcaataatgtgaagctttttttttttttttccttttcctttttcctcataactcatcagatctcgcctccttcatagtggttatctgaacactgtaggatcgtgatggaaattgtcttttgattattaaggcctaggctcagactgtcccttaggaatctgtctgtgtgcatgttgcttctatggatttgcacattggaggatgcgtagaaaacatatttttttaaaaatcccactggcatttttaacatgccaggttgttttgtgttctgcaccaggttttcttcacaaaacctttctttttcaggaagcgttgtacataagtgaaagaaattatgtctgagctgtaatcactctttatattgattgttatactcacatgatcataaatattagccatgtttggtgctgtggagaaatgaaggtaattgccttatgattagagctctttcagctacgagaaaggattgattatcatttgcatactggaggcaatattgagaggcaggaggaacaaataacaacattagttattttgtgtgagagagcaatctcaaaacgtaatttaaagatcttttttttagaaaatttcttttctatgttcaaattttatgttctttttttttttcttccttggcagtatttttcccctttatttttcttcttttgagttttgagatttaccaggaaaaaaaaaaaaagtttaaacttctgggtctgtcacatggatcttttagccctttatgaacaggtttcttttatacctggttgtttttatagctattttattatctctctgcattagtgctgctggctctggtttaaagcaagcgtttgcaggtaattggaaaaaagtgtttattgtttgtgagtgtgtgtgttctttatcgaatgatttaagtgcgtttaccaaggaatgtggctttgtactttcccgtttccttgtttactaattgccacgttagaacaaaaatctagatggaattttgatttatattgttaatcatgaataaattgatgcaaaacctgaagaaaaggtcaaagcttatatgatcgtgtggatgtattcttagggattgttttttcataacccacccctcgttttgtttaccttttccttcccttacagtaatgagaacattagggtcttgttcttaaactgagctaaaatttcatctggattcagttatcatcaaacacttgaatttatagatcttttaatatttgtcatattctcctttactgtgagtgttttgggccttcagtgtgttttgctcatgaaacagacttgagttaaagatgcatttatccaatatggtagcattccttttttcttcttcctccactgattactgttaacttttacttttttttggttaatttctttcattttattctaattgttggagctatatataaatatacacatactttttttgtcttggttattctcttgtcttgaatttgtctcctttgtttccaacgaacaggccatcgaagacggcaatttggaggaaatggaagaggaagtacggcttaagaagcgaaaaagacgaagaaatgtggataaagatcctgcaaaagaagatgtggaaaaagctaagaagagaagaggccgccctcccgctgagaaactgtcaccaaatccccccaaactgacaaagcagatgaacgctatcatcgatactgtgataaactacaaagataggtgagtgtttggttccttcaccttgatcatctctcaccaagacgccgagtggcgctccctgaggagcaggagttgttaagttgtgcacttaggttttattaaggttctttctagtttgtgttttatggctttctctctttgtacctgtttaaaatggccactgttgctattttgtatgatgtttcatcaggtattatttaagatagttgaaaatttagtttagattacaaaatgtttaaaatatctaaattagaaaaggattcttaaggacctctgaaataccatctgaaaatgaagatatttcagatgaaagggattggaatgatggagtaatattattttttttaattgaattgttagagaaagaagaatcctaatatatttaaggtaaatatttcatgttaagactgtttaaagcaaaatagatgcttcatagtaaatacagtttctcctttccttccaacacttgaatttctgagcacttgaattatctctttcttcctaataccacatagcatggtctttcagcaaatttaccttttttatgggatttttggatacagactttgagaaagctgtgtgggacactcctcccaggccattttaaacagtgaataattatttgaaaaccaaagacactgtagggtgtcagtcagatccatacagtagatgaactagttaggctacgttttggttagttgacttggtttccactttattatcacgtggtaatgtttttatttgaagatggcttctcgtttcagttgaaaaattgacaggaaacagacattttcactctctcagttgcatagtggtattaatcagttttaaattaccaattgaaaaaaattttttcttaagggggtgcatcattcctggaggtactgcagtaccaggtcgatgtgtggagtggatgaagcaagctcctcttctagctccctgcttccaaaatccacttaacatattgtcctcggatagaggaggaatcagatattaagcatgtaacaacagatactacacttggtcttagccaaaaggctgagaagcagttagttaccagaaatgtttgacattaactagtgttgctctgtgcacctgtaagtgtttaacatgaataactacgttgtgtttttagtttttcattatggctttgccgaatacatcatagcaggaaggaaaaattattataaaaattaagtggtataaaaagcaagaaactttccaccatggggttttgctgttaagcctttcttatctgcaaggagttttcagaaatggggaaagaggagtaggtggagatgtttgtcaaaaggccagaaattcttgctccattttgcccaccaggtctctcagattttgtgctgttttccttttttcaaaaaataaattaagttcatcccctttaaggcttctgaagtggccagaatggcaaccccattctcttgggtttggaccaacggttcattaatgaaggggaatgacagcaatccaggaaggaaattttgaccgcaagtccatgctttgtccagagtgaacatgaaaacttccttgtaaaccatgaatcagtaactcttacgtttttactcacccccaagctccccttgtatatttgccacagaaagggacagtattctagaagccagcttggattattatgccgtattttctcagtccctggtttaaaacaaaatattactaagtagagagcatcactctgtatatacatcaaacttttttgcctttgtctcaggagagtgtaacgtctgtttctctgaccagatgggctgtgatgtgtgacaggcattttgttcattacttgctgagtgagtcaagggctgttataacactgagaagggccaggaaagtccttggaggttgagaagctaggcctttcattctaaggtgatggctgtgtctttctctagtgacataaaagaagtcaccaacaggtggatgacatactgccaacagattcgtttggttttatgcttttttttgaggggtgggggtcagcaaatacataaacctggagagatttcttgcaaaaatttccctcccatcccccacccttttgaaaaaccagaaactctggcagcactttgcccagctctccctaaggtaacaatcagtaggcatgaagaagtggccatcctttcattggcagcatcgcgctctaggttgtcacaatccccgtcactctgtatcatctcttcagtatggagtgcgtcgccatttaccgtcatgctttttacgcccgactcacttcattcatttgtattacctgctggctccatagcctttgagtttatgacttcctggtatacatggctgtgggctgaccactttcctagaaaccatccagaaatacagattggtaggagattatatgccttaaacctcagctaagcattgcacagtggtttagagtacagactcttgtctgtcttgatttaaccacctgcatttgaatcctgactctgacattcaccagctatgggacagtggagtaatttacttaatctccctgagcctcagtttccccatcccagaatcccagaaatgggattcattactgcgcctgcctctagagtccttatgtgtcatcagggatttaatgtaactcaggttctttgaacagtttcattattaataatcagcctttcagactttgaggctaatcaggtaggtgaatggacctctgagcaccagaaagaaagtgtccttgctgatgctgatttccatggttttatttctagaaacagaaaatgccgtttcagtggtactgtttattgagtatgtctttagttgtactgtaggtaaagggtatttgaatatttcagcatgcagatattgagtagctttttctttttaacttttccttgttttatttgcttacttacttttccttaaaaagtgtcctggtaagacacattctattagctcccacatttagatgctttctggggagtatgtactaaatttcccaccactgaatgtttcatatacatttcatgttggtaatcctctagaaaaagcactaactatttatattttgtatttctctttaacttggtactcaagtaggaggcactcaaattaggagagccttaattagatacagtatcatatttttggttggttttagaaaatgctgctgggagatgtacatttcactgtgttgagatttttgcaccattaaagtcagttatttaaatagtaactcaacccacctgtgaaagaaataccacctgtgctaacctccttcccatcactttggtcaaatgttagggctctgacagtcatgtagattaacagcatattaaataagaacagccatttatatttaactgatatataagaagaattaattagctctcatgagacagttgtttaaaagttcatgcatgattctggggcagatgtatcttgatattttcataaagaactaagatcatttatgtgtgtgttcgttgctttttagtcagtgataggacttgttttttaaaaaggagggtgcttctaaaaatctgacccttaagagccagatattgtgtgattcataagtgtaaaatccttctgcagtaactgctgtctattcagaggtagtactggtctgttgctactggcccccatcattttatttgcacaaatagaaaactttatatcaagtagtaccagaaggctttgttttgacttgttaagcatgctttgttttctgcttatttagaagcttttaaaaactcatattctcaatttatgtaatatttaacctaagactgttctttgagaagggattcatcctgtttgcatttgtacccttgaaaattgcccctgggatgagatgtagagttagacggtaaagagtagagtctgtggagctattccaactctagagacatggctttcagttacttggataaagaagagagagagattcctagaggcattattaacgttggaagggatgtttagctctgcctggcccaacttcccacaatacaagtaagtcccctgtaccacctctggcatgtggtcatccacctccatgtggacactgcagtgtgagctccctgcttcagagcagttcttgacaccaaaggacagttctaaacacatcaccgtttcctacattaaaagggatgcagccatctgacccctagctatttgttctacttggaacctcagagaataaatctgctctcattctgagaaatatcctttaaaatgtatcaacaagcatatctacctcaggacattgtccttcaggtaggaacacttcactttgctctctgagtgatgcctagacattggccacattccagtctcctagtgtcccatttaaaacatggcgcccagaactccaatggggtattctgatcattaattacagggtggttagcttccttattctggacaccagaattctgttagcataactcaagggtgtgtttactttttccagcaaccttgacacagagttaactcattgaatatgtgacttcttgaaataacccccactccccaccccagaacacacatacactgctctgatttcacatgaatactgccaaggttagctttcagataagcataatttttttgaacctatgcctactgattgtattcttatccctgtgtagttccagccttactacttttcatcccatcatccttgcatgatgctgtacctctaaatttttatctatgcaaaaattatgtttactatgtttatgtatctaagttttttcatccaaagatttgatataagctattgcttagaaacttagacaaaggacaggtctctgtgacatacccctgaaaatcacttcaggttgacatgaatatattaaccagtaatccttaaggttggctacccatcagtgaactcatatagccatgctctcatgtcacccacatttatcctttctcagcattttcaagaaaggctaaaaccatgatacaccatggctagcctttccaatctatcaccctagaaaccctgccgaaaaagtgaaatgaggttagcttgccatggctttttctaagagacctcctgctggcacttaatgatgttatttatcttttctaaatagccctgtggcatctgaataatttcttctaaatttttaccaagggtcaacatgaagtgtaacagtctcaaattttctttctcatattgaaattgggacatttgcttatctctagtgttctgattccatttttgtgctctgtgatttcctaaatgttattagcagaagcaatctacatgcttttccaggatttaattagtttgcaactggacacttgaacttacttgaagtggcttgatatgctctttctatcaactctcttatcaggagcttttgttcaccatttattatgccttgcttttcaaatctgagcattgttctcattcctgaagaatctggaaacagaaagtgaatagggtgtcatcacctcctccctctctatcaattgctcgaagacaattctttcagaagcagtccacaggaagaccagccctgctgtctgtaaaagggtttggacatcaggtctagccttgaagatggatgcagcaaagtgatggaggaggcaggagatacctgcctggtcagtgcctggtcagtccaattggtgaacacagctccggggcacagaggggtggcagccacgtggctcctgcctccctgacactcttcagtctctgctgtcttcccctagctgggaatgtgttcttcttgcccctaaatttaataagcccttgtggtatctctgggaattgtggtaagactcagcttgtcctgaatttttagttctcacattggagaaaatcattcattctttttagaaatatttccttcattgtttgcttatttccaggactgtactcttgaatataactatagggtaaaaaattccaggtacttacccccactagctcatcatgcagtggaagagatagctttcctatgggatattactgaagaggtagctggcctggggaattcagggaagtaggggtcgtatgagccccattatctgaggatacaatgacattacctggtacttataatcaattcaggacattataataatatttacctggggaaatgagctttttttctttttcttttttttttttttttgagacagtttcgctcttgttgcccagctggagtgcagtggcaacatctgcctcccgggttcaagcaattctcctgcctcagcctccagagttgctgggattatgggcgtgcaccacgatgcctggctaattttgtatttttggtagagacagggttttgccatgttggtcaggctggtctcgaactcctgacctcaggtgatctgcctttctcagcctcccaaagtgctgggattacaggcgtgagccaccatgcccggcaaatgatctgatattttattacattatagcagctttccaagaatttggtaaactacaaacctgtggactaattgtagttaattcctatacacactgaaaagtaatttgcagcatttttctcattttgagtagggctgaaactctgctgaaggcctacatataatcagcccattctccagaagggcagggctcttatttcaaaatattgccttgttccaaggcttcctctgtctcggaagttctttatttgaagtgcgttaggtatttactgtgtcgaaggctttcccacactgattatcatctcaaggcttcatcaaaccaatggatctttacactggcattcatactgtctttctaaaataagttttcgtaacaaacattttaaaccaaatctatgtcattatgtagttattattttttgttgctttgttttagggttttaaaaatttgtatttttttttccccaagccaatcagatggctgacctgcatggactccttatcagaatgatttgaaattattctgctgggatctttggttgttcagaacccactgagttgtaatctgcgtcagtcttagatgacagagccatacctagcttctctgagttagttacaatctactctgtaaagcgttctaaggtagcacttgttgttttcaggttttcctcccgtcactcgtgcacattcggagatgacatggtctacttaagcttctgtgtctccttggtcagcaccatcattagcccaaggtcctaaaagtgaaataatgaagactttcccaattctgtgccttattgctgacactcagagtcccctcaacctgctcctaattgtccctacaagacacccgttcacctgcctcctcaccccctgtcttcctgaggcccttgcactgctggcctggcctggcaagctgcagggtggactgcgtgccctgcctccagtctcttcctgtcctggcttctttctgaggcacctaacttgtcatttcatattgtaactttagaactcttcacagcggccccgttgcctacccgatgatgaccagacttcttagcgtaggagaattgttatcttccacagtctgaaccttcggatcttcccaactttttttttccaacgcacccctcccagctctccttatatttcagacattccaaagaattctgtgtattttgaagcgagcacatgcgcttccacccctctgttgatttgtttatgggtttatgcttttccctctctgcaacaccccgaccccacactgactctagagcagaatggggcagtgactccgagaagggatttatacatggacaggcacaggctgtgaatcccaaagggtgctgtctacacctggagggagatctagagggtatggagggtcttcccaggatctgtgaaacctggcagctgcttccctgccaccacaaaaagggacaccaacctagcagtttcaaaaatcctcaactgaagaggcttttctagaggcttgtagaagtgcccaagacgccaggtggtggtttattttcatttcccactaaagatcatgaattcagtgatctctcgaaaaggaatagagctcttggaagcagagctgactagttagatggtgttcagacccaaattggctgatgcttggaataccttctttgtgcaggctactgtaagaaagcactttatcctatttaatttttataactttctgaggtaagtatttcccccattttacaaatgaggggctaaactcagatagactagcactaccttcccaagatcacacgcacctctagccagtggctgcctgtctcccaccttctggaacagtgaatggaacatgtaagagggaacagggtaacaggaatttctgtgtgtgacggaagtaggaaaatcccagaaaggttaggaaatccacttcccccaccatttttagagaactatgttatttttccgaatgaggttccaaacataaccccgtgtaatcttcctaacaaggcaggttggtgaggagactgaggcttggccaggtcacccagcctaggaagaaggagccgggcggggacgagaacccaggtcttctgactctagtgttctttctactctaccgcaggtgtaacgtggagaaggtgcccagtaattctcagttggaaatagaaggaaacaggtcaggatctgtcttgtattcccctatctctaaatacaggtatccctcgttacgtgaaacagattgaatcatataatcggcctttggaagcaaatttcttcggtcacctcctgatcacccctacttggagagcgggatagaggcacagatactcttaaacagctgtcatggcttctgaagttgttggtgctgtattttaatctggctgtgccaattctatacatgcactccttacaagggtattgggagctcctggaaagcccgccctaactgcagcatcttggagatgggtttctgttgcttccccctcccttccagcgcagcttctgttgtgtgttccttgggcctctttaactctgtgctgtcttggtttagaagcttaatgcgaagcacctgtagtgacttgatctcctgcgagacatgaagaagcgtgcatgttcacgctgtgtctgcattccagagttgatcatggcatgcagagggcaattgctgagttgtctcttgtttgtgtgatgggttgggtttcagggtgtgactcctgggcctacctgttgtgtgtagtcccaagcatctaaagccagtctctttcaacctctgtgtgcctttgcttctgttgggtagaaagcagaaatgcttttctcctagagatagtgtgaatattagatgacattggttgagctggagagatggatgtgcatgatacgccaaggctaggagctcttattaatataagagagataacatttctttctccatttcggtcaacctgtacttaaaaaaaaagttgatacagtgctttaccatccatatttcaattttttcagctgacccaataacatcctttacaacattttttccaccagcataggatccaatctaggactggagagagagaattgaggatgtatcgtgtatttcagaatgaacacagatcactggttgtggtattcttctttatataaggagattaaaaactagtggtgatttatacttatacttatccatttttgtgtggagatgaaactgtcgggcaagattgtcaatttttgatatttcttattgcagtccagtaaacaatgactaatcctggaaaccaggcacataaactataatgtccattgagaaacgtcaggaattttgttctgtatttgccattatgagaaagaactcagtctggattctgacttggagattgaaacaacgaatagttgacttcataagtttgggcagtttaaataatcaaccaacttgggtaaatatttccacccctaatacctagaaaggaggccttagggctaaatggcatgatgtaagctgaatgccctgcccaggaaacagggacaggcaggagctcctttccctctttgccctcttccatcaagatggtcatgtgaccacgaaaagtagatcaataactttaaaggctatttattgcaagtccccagtgccatgcccaggctttttgaggggagatgacatgacggtatatggttaggaactagataaattcattttcactaaagacacatttaaactgtaatttccagagacatagcagtgggcttggtgtttgcttcctcagggctctctggagtctgtcttgtcccttgctgtctgtcacacacacttccttttcccataagcctgagctgacaacatggtgtttggagattgtattatgatgaattaaccctttcttcagaagagcttttaaaaaatctttgcttcagtggtcaaaagaggctctttgtaagtactgaaaatggaaaaaaaaaatgaattaaatggagttatttaaagcacaattggaaagagctatgtcaacaaataatgcaagtacctggtatgaatgacacatatacacagggtgggatgggaagggatggggcaggagtggagagcgcttctaactggctgtgagcagagtgggagtttctcaaacagacgggtgaagggaggatgggtgggggggcagagcttgtggtggtatgtgcgggaaggggggatgtagccatggaggcttggaaagcagtccccctctagagactacaccatctcagtgtgaccagctcattggagacgaaggaagactgggacagcctggatgaggctggagaagcctgagaacagatcccggagggccttgaacaccaagctgaggaggaattttgatgtactcttctaggagtggggtcttgagggcaacctgtagtcatcgcagtgactgttacgagcactgtttcaaggaggccaacggagagcacacctgagttctgggcgtgagcgtgcagggaccagtgtgtgatgtctgtcatgtgggagaggggaggtgacacccagggagccgacatcagccctgcagtggaaagtagatagccaagagaggatcatggggatacagtgagatttgtgttcggaaatgtcactgatagtctgtgaaggatggatcgaaggaggcatacattaggagaatgttgtctggtagtggaggaagatagtccttaaaataaggaagtggcagtaggtaggaagacaagggaataaatattcagaggtaggattgacaagacttaggggcagtctggatgtgtgaggaggaggaggaagagaactcacatagctcttggatctctgccctgagtgatcagtgaacagcggtgccatttacagaaatgaaggtggcaggaaaattggtgtgtgtgaaggcacttgtatgtatgtgtgtgtgcacatgtgcatgtatgtagttacctggccctgcatacaccatcacaggacagctgaactctctacttggccattagcagcccttgatgttgtgatgatgttgttagtattgactgctctttcaggaccagccaacaggctcttgtaggcaaaatttatttttagtaccagacatcttgtattactgtaaatgagagcgagaggctgtaatgatagctctcaagcccaagcacctctgcatttagtggctctaatttcattatagagaaattaccatgaaatagcactgtgaccaattagatacacttctgggaaattttaggtgtaggtgaagcaccttttttcattcattctctgtttctgatagtttggacccagaatcaagctgacagtttatcgtgtagagtagcagcctcctgaggtcagcgcctcatgcattgtctggtgcatgagcatttcatgatgcttcccagagtgagttcttccaactctagccttcctactccttcttggagattcccagtacagccagaggccctgggaagtcttgctgtaaagtcagctgtttaactgcttaacccagcatctcccaaacttatttgaccaaagaacccccttttcacgggacactgttactaacccattaagctagtgttctgtggagcacactttgggaaatgctgaattggagcccaaatattacattcagtgcagagagaatctgctcggagatgtggactccagtgctttattttgccctcattacaaatattgatactttaaatgtttgctgaaacaggctgctttgactcttggtgactcgatacttatttatggtgctcagccagttatgcccacgtacagcaggtgggcagcccacaaataaacttagtccgtctgtttcccgagacacagaatgcttccccttatcttccagcattagcctggccctgaagcccctctttcgagggtctctgcctctcaagctccaggccaacaccagcataaaggacccctctctgaaggaagggaaggaggctcagctggaaataacaaaatggattttcttacagggctgacacactgttttccaaaccactctataaatgttttgattaaaaagggattgctctgagagatcgcagttatttggatatatatatgtccttgtatattaatgggggaaaaaaatctgtctcactcctttagaatcacacctaatatgtcttccttgaggatttattacaacagccattgtaggaaaaagcagcactccggtgcctgccacgacccacccatttaacaaagcagcgtgtgggacttgggcacagatggagaggcatttgattttgtctgcagaagctttgtgctttttaatttggtcgcaattcagccaaaagctttgaagcattttaccattaagaaatggggaagcaaaggccaggcacagtggctcacacctgtaattcccagcactttgggaggccgaggcaagcggattgcttcagctcaggagtccgagaccacccgaggcaagatggtggaagcctgtctctacaaaaatgcaaaaatgaggtgggtgtggtggcatactccagtagtcctggctacttaggaggctgagatgggaggattgcttgagcctgggaggtcgaggctacagtgcagtgagccctgatcgtgctactgcagtccagcctgggggacagagtgagaccctctctcaaaaaaaaaaaaaaaaaaaaaaaaaaaaggaaatggagaggcaggaaaggatcaaaggacaggatattcagtggaagggaacgtgtgggttcttacattttgaaagacagggacgctttgagtcatgagcgcgtgtaacgtctacaaggtggggtgggaaaaactgtagaaatggccggctgaggaagctgcgggtcatccggtgtctgtctgtcatcacatttagtcagatgggcagtggagttctgtacctgcacccactgttatgaaacgtcaagtgagaaagagggcagaaaagtagccatcctaagacagtatttccgaaagctgagtgatagctgacaaatctactcaaaaacattttttgaaaatgcttgtccccgcccccccccaacaatacacacacaaaaacactctctggatcttacgtcctgtttcaagcacctttgctttgtagtaataaatgcaatgggagaatcctgaagaaatgcatagaaactgaaaagaagattctgatggttgcaacatacaacatattaaaacttttgtgtaatgattgtattcttttatttgtttggctttatgtgatagttaaaaagtgccagttggtaaaacgtgctgcttatggtgataaaaataaaggttggtcctatgatccttaaacgtaatttctttggaaaaataaaattagatatttgacttatggcatgttttcaatgaaggtaacccaattaaagtgtttgttgggacctactttatttatggataaaatggttctaaattacattcaatcataaatcttcattttggtgaataagagtaaattattatttttcattatgctttggcatatctccttctggtcgtttttcaatgccttttttaaatccttgttgaggttgaagttgtactgcactacaatttgatatcctctgtttttttgggttttttttgtttgtttgtttgttttttgagatgaagtttcgctcttgttgcccaagctggagtgcaaatggcacaatctcggctctctgcaacctccgtctcctgggttcaagcgattctcctgcctcagcctcctgagtagctgggattatagccatgcgccaccatactcggctaatttttttatttttggtaaagatgaggtttctccatgttggtcaggctggtcttgaactcctgacctcaggtgatccacctgcctcggcctcccaaagtgctgggattacaggcatgagccaccgcgcccggcctgtttttttttttttttttttttataatgacgtaacaagcatttttttctttaaaatacataattttagactctcttgcattctattacttgttctttccacaactgttaaacgttttggctgctttgaattatttattttaagtaatcctgtcatgaacatcttgatgattatctgtttcagcccagagtagattaccaaaagtaaaatcatgcatcagagtgtgattgtttttaagactctagacaaagtcttggtgcctttcttattgagctgagtgaacccttttcagtctcctaacagggaactttgtttgtcttggtaactcagggacattatttatctgtagtcctttatgactattagggtgatttttttggtggtgttgagacaacaaagtcagctctgttgaccaggctggactgcaggggcatgatctcagctcgctgcgacctctgcctcctgggctcaagcgatcctcccacctcagcctcccaagtagcttgggactacaggcacatgccaccacgcctggctaagttttgtatttttttttttgtagagatgaggtttctccatgttgtctgtgctggtcttgaattcctgagctcaagtgatcagcctgcctcagcctcccatagggcggggattgcaggtgtgagccactgtgccccaacattggggtgattttaacagctactgattccatgacccaaacaagtctactcccatcccaccctgttttgctcttgcttcaagggtctaggtgatgatatttccaaacttgttaggaattatcaccaatcaatactcaataaggctgagatgagagatctcagtagcaaatatggagtgcagatgcttgtaacacctgcttcatgactggctcaggtttctgcaggctctcttgataagtaggggcactaatgtggtctagtaaattcttcaatgatatatattaaacttcataggaacctgaacctgaaaagaagcagcacacttcacctactgccctgcagtgttgacccaatttttaaagttttcttcccaaacgactttgctatgtttgattcctttaggattgtgatagcttttctgtaaaatccttccaacctcaagtgagtattctaaatcatgacatcattttctaattgaagtaagatagtcacattttaacccttattaatataaacaatttatttcagctatggttaagatttctttctcagcactatctattagctattctggtcaacagatcagcttgagcaggtaaaaaagaaaaaccataaaagaccctcactaattatcaattttcgcctggtcctggaaaaaaaaatcattaaaagacataaccagtattctagaagtagatttctttttttttttgaaactgagtttcgctctcgttgcccaggctggagtgcagtggcgcgatctcagctcaccacaacctccgcctcctgggttcaagcaattctcctgcctcagcctccctagtagccgggattacaggcatgcgccaccacacccagctaattttgtatttttagtagagagggggtttctccatgttggtcaggctggtctcgaactcctgacctcaggtgatcggcccgcctctgcctcccaaagtgctgggattacaggcatgagccaccgtgcccggccagaggtagatttcttaactagcatatcagccttcagcatgtctctaagcttaaaatataatcaaagctaaagctgacatttatgtaaagtttataatgtatgcagtcaccagtttagagtcatagtatagatgtggaattatacttttggtttgtggaggtaatgaggaaaaaaaaaaacggttcccagatgaagcataacaaattaaatctcagtagtgtgtgtatataagccatgcatacaaggagttcaaactaccacttgcacagtggtatgtggctcttttccatctcgtactctatggtctcatggccacgtcgcggtgtctgttcacttccatgaagaggagaggttcttgtccaccaacacctgtgaaccgtaagaccagtgagtgcctttaaacttgtatttcaagttgtacccctcccccacttaccccatgtctcatcagagccatactttctagcctgaggtagatcattacgtaaaatccatccgatgtcacaggccatcagtggtcttggggagggggtctcatttttgtctgggaagtagtgtactttccaagtagcaatttatatttaggccttaaattgtgtaaaatgacataatggaaaacaccttgggataggataagcatttcaacagaaacagaacaccttctgtaaacaacattgtacaattagactattgttctgtacttaaaaaggaaaaaagcaactttctggccgggggcagggtggtgcatagaaaaccaccacatttcaaaaggtgtattaaaaaaaagccctcgctcagctcaaattagagaaagaaatgacaagaattcagaactgtcaggctgcttcacttccttttttgtttgtttgtttaatttttttttaacctgtaatttaggaaaaaaataaattactccagcaagtgttcgtgctacctcaaggtattctataggtaatgtggaagctaggtactgcccaggacctcctggctttgtctgccgaagctggccatccaatcccttgctctgggagggagggggatgagtttgatgtctgtaagagaaccccaccctgtagggaggagagaaacaataacacacatggaagaattaggttctgagcaatgaggggactgagagtaagtgtagttgatatttaggagaatttcctccatttttctggacaatgggttcttggtcctggagggataaggaaatacttggagaggagaaaaaaccacagagaactttccagctatggagtgagagctaggtcaggcctggagacctggaatgggtcataaagaggggattgcccatgaaagttgggggtacacagtagagactctcctgactagaagagaagtgtgttggtggtaccagttgggtttttagagaatgaggacatatcctggagggctttggaagccagactctgagatatttcagtgatgatggaaacaacatatcccaagaagtcttccatagataacttccaggtcctccttttgcctttgtcctttattcttaggagaggaaaaaagaaagtgggataggtatattaagtccagtgagcctttattttttaaacagaataaatgtgtttatttctcattttacagtattggccccaagtctcagagcattcagctctcttctttatgagcatagagaactctggacttgtctccttatggggtttagaatttttagggtcttactgaagacagagatgtcatacctagattgttttagccttaccccttagagctttgttgcactatcggtcacaaactgcttatttcttatttatttcaccagtagcacagttaaaattaagagtgtgtccatcacaggtgtacagagatgtgcctgtactccatcaatagaaacatgacaccatctgttggcaatgtagaagatgacaccatgaagatgtgtcattgagggagagtcaggtgttaaatccgcgattacaaagaggtctgacttttggggtgctgctagctgataaagaatgctcattaagcaagttgctctctacaaattactctcactgcatttcataggcaccttttcaaacagataaaatgtttgggtgtataaatctcagaatggtaggaaaagaacagaccagaaattgtggccctgtgttgaaggtgatggtaaataaaatagttcttgtcatatactatgtggcataagttagggaaggacgaacgcatttctcgaagagatgtgaggggtaggggtggttgagagggtgagagaacaccttggttcttgctgcagtgatccagaaatccaattcctgagtaaaatgcagtcttcttaaacctcttgcgttcacccctgattttcgtactcatgaggcccatatgtgcattaaaaaaataaaacaaaaacgatgacccagtgtagcagaaaaactagcccatctctttgacagaggattttcggttaatttatgcaggcctctttgcacttcagcattttttccactggttgctaagcaactgtttgagttactgcgcatgccttttttatgataactgtcattatgattactttttaattatacaactgggactgtggattaaaacagcatgttgtcactcatgctttgccactgccttctagtattctcacaagctcctagaatcaccttctgaaatttttatggcagtgatttatgattaatttttgatatttctggcttcacaaaggaagtagagatggcatcctaaggggagtgagacttttgtgaaaaatgactgtatcacacaccacaagctgttattgatctgagtagaccaaatcagatattaggatcttgaacctaaattgtgaatctctttgtgcctttaaccacatctgcttccaacagtaagtgatgttaaaagctcatatgctttgtagcctgtgtttgaaaattacaaaaatgctaagcatttgttgctatagaacctgctttcaaataaacttaccatgaatttgggggagggtacagcactaatcccagaaatgtgaagcatccacacttaagcttgtgaaacatgttcttctttcacccaaattgctctacacacacacaatggcagttaggcaagatgaaggggttcctctggcaagtaataataaaggtatgtgtggtttacagtatgtaaaataccctaaaggaataagaccaagaaaatagtcagccagtaggtacgtacgaaggcttttcacttgagtttccagagtatattctaacatattattttgagttactttctaaatacaatgtgaatttgaaaacaaaggagtatggaaatacaatgtttaaatatgtatatttttatatatatgtacgtgtatatatatacatatatatttaccaattcatttaaaaatgaaatttttagctaacaaggatattttaaatatttctatcaaaataaagagatatatttgctctgaaagggaaatccacagtgtatttcccatattgtatgtgtgacagaagtggggaccaaattgtattttactgtgatcttaaaattatcacaagggacatcaatctccaatagagttgtgggatttttcctgacttaaaaagctccatatctatatgcgtggaatataatttactttgttgacatgttctgaaataaaaataaaacctttcctcagtaatgtttgatgtggcttgtttttgtttgtttcacccatcctacagttcagggcgacagctcagtgaagtcttcattcagttaccttcaaggaaagaattaccagaatactatgaattaattaggaagccagtggatttcaaaaaaataaaggtagatattttgtttaccaactttattcttcaagtaaataaaggagcagtgtaaagtcattgaaatggttgtagttggagctgaccgccactgatgggtacccagggctcgcgacaggaaaggttgggatgtccttgtgcttttccgtgagtgttacttaatcatgttgagagctcacgttatattgcattttattgaagagctttcaggctccctgctccccctgcccacattattgctcttctcatttcccatcatatgctcctccttggcaaaaggaatgacagtgggagtgggagggtggacttggtgaatggcgccccctggggttatgcagtcccagatccctggcagggctttatgctgtgaagacaaagggaaaatcaggctttgttaactaagtaatgatcgctgaattttcctctccctctttttttctccattttctccaaaatttccatcaggaaaggattcgtaatcataagtaccggagcctaggcgacctggagaaggatgtcatgcttctctgtcacaacgctcagacgttcaacctggagggatcccaggtctgtcttgttaagttgtctaaaagttcttaactgtaaatgtgcccgttgttctttttaagtagaatatttcattttctacctcttgaatcattgctactggcagaagaaaaataactaaaagcctatgttccttgctacctgtgtaagaaaatagaggctttggcatctgcagattacctttgtaccacagctgtgctagcgcctactcacacttcttttcaaagcttatagtctttttttttttttttttttttttttttccttttttgaggcagaatttcactgtctcccaggctggagtgcagtggtgccttctcggctcatggcaacctccgcctcccaggttcaagccattcttctgctctcagcctcccaagtacctgggattacaggcacccaccaccatgcctggctgatttttgtatttgtagtacagatggagtttcaccatgttggccaggctggtcttgaactcctgacctcaggtgatccgcctgcctcggcctcccaaagtgttgggattacaggcatgagccaccacacccggcctcaaagcatatatttttttttcgagatggagtctcgcccaggctggagtgcagtggcatgatctcggctcactgcaacttccacctccctggttcaagggattttcctgccttagcctcctgagtagctgggattacaggcatgcgccaccacgccaggcaaatttttgtatttttagtagagacggggtttcaccatgttagccaggctggtcttgaactcctgacctcaggtgatctgcccacctcagcctcccaaagtgctgggattacaggtgtgcaccactgtgccaggcctcaaagcttatattctaattagaggagggattcaagtaaatcattgaaggtaatgctgaaaggctggcagtaccatttttgcttttttggagattgtatatttgattacatttacctttatttttctgattgtactcttggatcaggagctgaaactactagaatttaagacaaaacaaactataggaaaaaaagaaacactgcctctaattgctttttaatcctttttatcagcccaattgcaattctctttttactccccttctccacagtcaacaattctaagagtatataaatgaactgaatttattaaaataaccaaggtgagggcactttcattttaattacacctcaattactgtcaccttatgtaggacttttttcctgtctgtatctctggcctgatgccttcagacccatgaagaagaaagaaaaagacaaagatgtacccctcaaccaatgcagtgtcaagccacctttaaattccttaagcctgtttcctgatcatacccttctgcctgcacatcaatttcagtcatcaaacaaaggaatagagaaatcttcagaatgtgccttgaacagacttaaacatttccaagttaatagtagtagtatcttgaccaaaacaggaggtcttgaaagcaagccttctgttactcaagacactctccaaggaaaaagttaagcttgaagccaggacatagaaagtagaagaatcaacaaagaaggttttgtgaatgacagacagtttgttttagtctctggatagatatttggattctttatgtcaatatggcattattatgatatacatccagtccttttttctacaaccttcctatctgttttcactctgactgtctcacaactgaaataaggtgtcaagtggcctcttgactggtttctcccccaggccatctggacactaatttgtctttctctagccaccttcctatcctgttcatttactgcctctcttcccaacctttgaggttcccctgcactgtctagaatgaatttcatctctgtagactgcttgctgaggtctaaggtaagtctttagcttcatctgctactcctcaacctaaacagcccatagtccattcggactgatactatgatcacctgcttccccagttacatttcttttactctttaagatcattcgagactcacatgcagttgtaagatacagtacagagagattctgtgtaccttttacttctcaagatggaaacatcttgcaaaacataggataatatctttttttttttttttttgagatggagtctcactctgttgcccaggctggaatgcagtggtgccatctcggctcgctgcaaactctgcctcctgggttcaagcaattcttctgcctcaccctcccaagtagctgggattaaggcatgcaccaccatgcccggctaatttttgtattttttttagtagagatggggtttcaccatgttggccgggctggtctcgaactcctgacctcgtgatctacccacctcagcctcctaaagtgctaggattacaggtgtgagtaccacacctggcccaagacaatatcttaagcagaaaattgacatcaatacaattcccagatgcatctcttttattcttgcctcctcatctttctcatttgttttcctttctaaaaagcccctcatttctcaccgctgaaggaattcttctcatctttaagatccagctcaactctgatgtctctggaaaattctgccctgatggtcccatcctctctctctctcgcgcgggtcctttgttctgtcatactcatactcactgcctatattgttcatttggtgcaaactgcaatttgcccagtcacttttttgaacgagtaggtcttctttacaataaaaggaatatgttcctaaagagcaggggttgttttattttcctttaaatttttaaaacactgtgattaagagacataacaaaatttatcataatttttcagtatacagttcagtagtgttaagtatattcactttgatgggcaacagatcttcagaactttttaatcttgcgactctgaaactgcgtgcctacgaacaactaactctcatctccttcccccagcccctggcacccaccatggcactttctgtttctatgaatttgcccattttagatacttcgtatgagtagaatcatagagttacttgtctttttgtgactggcttatgcacttatggacttagcgtaacaatcctccagttacaggatttccttcctttttaagactgaataatattccattaaatgtatagactatagttgcttatccactcatttgtcgatggacccttgggttgcttccacctcttggctattgtgaataatgctgctaggaacatgggtgtgccaatgtcttttcaagacgcctcttgtgattctttatatacccagaagtggggctgctggatcatatagtagttctattttcaattttttgaggaaaaggaccatttttgaacttatatctttcagcacctacccagggccatgctcagacatgtgtatccattaaaaatagagtgttgattggataagtgttggctgctactgataaactcagtaataaggaatagcccccacttgctaggggcactgcattttaaagaagattgctttaccacagtcttactacagcatttggtagaacaattcaaataagaaatgtagttttaaaaaataccatttttgttgtccaatatgaggatattttatctactattctcctccaagcatccatgcactttgaaataatttcaactgcatttggactttctgtatgaatgtgtatgtctgtatttgggcttggcgttttgctactgggttttcatgtgggcataaaagctaacggtgacatttctactaaaattcatgtgaattaagctggtgtattgcataaggaagagttcactgccatggtaactcagcttgcagttttaacagatgcccctttgaccatttagatctatgaagactccatcgtcttacagtcagtgtttaagagtgcccggcagaaaattgccaaagaggaagagagtgaggatgaaagcaatgaagaggaggaagaggaagatgaagaagagtcagagtccgagggtaagcccagacattcgggtcctgtacatctttgcccctcctcacctgcatagctgtctccacagatgttcacagaagagactttagagtggggcagatctggattggagcccttattccactttgtccttgctgggcaaccggtggccatgtccttatccctgctcatgctcagttttctcatctataaaactgaggtcataaaaatgacctccaagcatgtaaaatgtttagcataggctgggctcctagcaaacactgttttatttcttttatttatttatttttgagacagggtctcactctgtcacccaggccggagtgcagtggcatgatcttagctcactgcaacctccacctcccagtagctcactgcaacctccacctcccaggttcaagtgattcacctgcctcagcctcccgagtagctggaattacaggcgtgtgccaccacgcccggctaatttttatatttttagtagagatggagtgtcaccatgttggcaaggctggtctcaaactcctggcctcacgtgatccacccgcctcggcctcccaaagtgctgggattataggcacaagccgccacacctgacctgcaaacactttctgaatgttgcctttgaatattagcctactacacattttctacaacactgctgctaatgccacctatcttctcttctttaccacggtggttctcaaacctggagggcttgaagaatatgccagtgtttgatccagtagcctgttgtggggcctgagaatccgtatttctggcaaattcccagggggaactgatgatgttgggccaaggaccacaccttgagaatcacagctttagaaaataattgtcaaactgagttggaactgtgaattttgcatagaattttttttttgccaggggaagaggggtgatgacagggtactgtctttatttaaaatgttgatagtttgtttatcatggatttggggggcattaatttgggtttttaaaatatttaccttttaggatattagttgatgactgggttttggtgcccccttaaattttacacccaaggtgcgtgcctccattgtttgaccctagttccagccgtgactgataacccaaatattccaaaggttaagggtgctgtcagaagttgaaaatttcttcttagtgaggcatgttggctcacaaatataatccgagcactctgggaggctgaggcagaaggatagcttgaggctaggagttcaagaccagcctggataacatagcaagaccctatctctacaaaaatgtttgtaaaaagaaatagccaagtgtggtggcacgtgtctatagtcccagctactcaggaagctgaggcaggaggattgcttagtctaggtgttcaaggctgcagtgagctatgatcataccgctgccctccagcctgggcaacagaacaaaactctatctaaaaaaaaaattttaaatataattaataaatattttaaaacataaaatttcttctttcctatgtgtgtcttcactgtatacctctcaaagtaaagtactaaaacctatgttttacagtataagccgagcccagtggctcatgcctgtaatcctagcactttgggaagctgaggtggctggatcacttgaagtcaccagtttgagacaagcctgatcaatatattcataataagtacaaaaatcatgccacaaaactgtgtgctatgtttttataaacaaatttatgtgtggaaaaaaaggtaacgtctcaaaatattacatgtatgtagagatatagaattacacgtttttttaccttttcaaatatattaaaagtatagacatgcttttaaaaaattattaggtttctaattacatgttaatacagatgatttggaaaatgtaatgatttttgtacttattatgaacactttccatggcaatgagggtttttctacagcaacatatattaatatgtataatacatattaacattcattaatgaatgtgaatgtactgtaacgtgttaattatactctgttaatcaggctgtttcccaataatgctgtcctgagcattttacccatagattattgaaaagatgtaaaatgtatgtgatgtatacacattgccacagagaatggagtttccattcttgccctcaacatacataagcgactctttcccaacgtcattttctattatctatattctagttagtgattctgccttttatttgttatgaatttaaactgtcccaaattcatgtagttattcctttgttctagcatttgaatacatctgcatcatctattttagctatatatttcattattttagagccagatttctaatctgttttcaaagagctgctctcttcccccatccatcaatttagtttaatttctcaagccttggtatattaattttctattgctgctataacaaattaccacaaacttagtgactttaatacaacacaaacttattacctcttgtgggtcacaggttcagcatgagtctccctggactaagatcaatgtgtcagcagcctgcattcctttctggatgctctagataggaatcccatttccttgttcatcctggttctcagcagagttcaatttcttgtgttcgtagaaacaaagttcccactttcttgctggctgccagtggaaggctgttcccaacagtgttctttggctcattgcctccccacccctaccccttgttccaactttaaagccagcaatgacaggtacagtccttctcatgttgtgtctccatgaatctctcttttgcgtcttcctcttccacgtttaaggatgcagatgattagactgggcccacctggataatccactataatttccccatctcagggtcagacaattaacagtctaaaattccttttgccatgtaatataacatattcagaggttcttggattaggatatggacatcttgtgaggagatgatattctgtccaccacacttggtagctagaacttttcataggtgcatatggttgtatcataaaaccttaggaagctgtttatagggctcacctaatggtccagtttattagcctgcttgtccatagtctgtcaggtgggttttttggagagacatatccactgacgcgtgcctgacagcattagagtgcgccacactccaaggcacatttagattacgctctcaaggatacattttcttacccctattgcctgtcttctgcccattcatttcctatactcctgtgtcagggagagcccacacctgtataaatggacatatttcactttgtctttccacactgtttagaaatgcccattatgactggcttctttcatctatttctttcgacgtcctacatctttccgttttcattagtaggtaggccttttcatctccagtctcttcttcttctcctaagttactccacccaaagcaaagggccccaccttatagcttgtcaagcagcagacagtgggtataatagatagaagccttggctaggagtactattttgtgtattaggatttggaatggaggaaaggcaaatgctaccaccacacttgagttcataacagcacagctctcatctcttctgttagcttatgtttctgtgggagattttagttttggaaatattttgtgggtccctctacttcacaaaagaagtctgaaacaaacactctcatctccttctggagctaggattccctctatgacaacatctatttctagatgccttgagacccaccaaacatggtagtctagcaaagctagcttgaatgagagacccacattttcagtgccttcatcatcaaataccaaaaatggtattctgtggatacctatcccaaatagctaccatggagcagattgtctttgttctttgggatatgtacctatatttgtgatattgtaccttgaactcttccaagtgtttccattggtatccccagaatagtcctgtgaaggtggaaatgggtgcagtctttctgaaggctaatctgacagagcactgttagaaatttaccatatggacatgtccaccatacctccctcaaattctaccaagatacattgaaataggttcacagcagctttgtttatactagcaaaaaaagcagcaaacttcagccctcatcatatgagactgattaaataaattatagtataccatacgatggaaaattttcaggtatttaaaagaattaggcaggtatgtatgaggctttacctataaatatcccacatatgtgagtaaacagccaagtggagaatatggacatgtataaagatccctttgggataactacataaagaattcaagtatgtacgtgtgtatatatacttacttgaattctttatattctatagagagacttttatagaatatgtagaatcacacacacacacatacacacagagagagagaaatggatgtacttgcaggtatggacataaacattttttttcctggaaataattagttatagggaaagagactaggaatgggggaaaggaagcattcaaaagtgttttctttttactttccattttgtacctttatgttaaaaatatgattgaactatttgaattttctttttaagtcaatggagttatctacatggtaaaattattggcctttaaactctttacatttatttatatttttaatgtaaaagttctattaaatgattgcccaacacctccacaagtagaaaacacttcccctcattatccttggtcagccattgggagacaggtgaaggatccagccccactgtcctccatgagacctcggtccctcttgttgcctgtttagcgtgactacatcatttatcttccaaaagaagacaggttggcaagtgaaaggggtcgctgactagacagaaccacacagaacaggcataaaccgggaatgttctgggcactctgggatgtttgtgttgtctgtaggttggtgatagtcttaccacctatacaaagatctccttgtgattactcactggtgtctatttcatttgctttggttttagcaaaatcagtcaaggtgaaaattaagctcaataaaaaagatgacaaaggccgggacaaagggaaaggcaagaaaaggccaaatcgaggaaaagccaaacctgtagtgagcgattttgacagcgatgaggagcaggatgaacgtgtaagtgtagccgactgggactgaaggcggagacgccctctcccctgcttgctggcctcttgcatttccatgccccttcagccttttcgctttattacccaggactggaaatgtcaggatttagtgagatttcattattgagggatgtgaacggagctgtatgatttagaacaaaggattgggggctttgtttattgcctttttaatagtcttcaaaaatgaaaaactaccaaatcaagcttccctttccccctttttaatcttcccacatgctttcttataaatatggttttccccgtagccagttaatattttacatgcatcacagccagcatggtgcctggtacataacagtagattagatggaagaatgagaaaaactgttatgacgtcagagttattggctcgagttgctacttaagggaccatgtaaaaactcaaaggactttttaagtacctaggggtgttatttttcatatttgaaaaattcagtgagcatctattgagccaggccttgtaataggtacctccctgtgtggtagcatattaaatactgatgtcggcatcgtgagatagatgtcactctcaaataacatgttatggaaatggttaggttatgggtgttttgctacagatcacgtcgcagaggtggaattcggacccgagtagcataactcagtctagtgtgctatgtctgactgggcagcaaggtagaagagtgggcagaagctcctaataccttgccttttgaaatacccttcatttcatccattcactttccagaggatggtttcctggcacccacattattttctttctggaaagataaatttcccttttcctagtttgattttccttcaacacattaccattttggtaaaatttcagaattatgatctcaccccagccttaacccttgatgcaagcattttcaaatacatttttctttaggagatgtcaaatgcagggtatttttttttctcccatgaattttctaaaacctggggctgaaaaagagaaaatattagccataaacctttcaagccaaagtgaaagggcacaaacaatagcttagagggtgggctttgctttggggtttttcagtaagaaaaacttctcagcttagagactgtcgatgcctctttaatgtgtttctgtcctcccacggaaagagatttggcgagttgtttccaaaatgtcattttttcctactggcctcttgatggtttgttgttatatcttctttttcttgcatgtgatgttacttcattttatcttcttatttttacttttaggaacagtcagaaggaagtgggacggatgatgagtga

SEQ ID NO: 5 - DNA, 4719 bp, Homo sapiens
  CDS (1)..(4716)
atgtccacgcccacagaccctggtgcgatgccccacccagggccttcgccggggcctgggccttcccctgggccaattcttgggcctagtccaggaccaggaccatccccaggttccgtccacagcatgatggggccaagtcctggacctccaagtgtctcccatcctatgccgacgatggggtccacagacttcccacaggaaggcatgcatcaaatgcataagcccatcgatggtatacatgacaaggggattgtagaagacatccattgtggatccatgaagggcactggtatgcgaccacctcacccaggcatgggccctccccagagtccaatggatcaacacagccaaggttatatgtcaccacacccatctccattaggagccccagagcacgtctccagccctatgtctggaggaggcccaactccacctcagatgccaccaagccagccgggggccctcatcccaggtgatccgcaggccatgagccagcccaacagaggtccctcacctttcagtcctgtccagctgcatcagcttcgagctcagattttagcttataaaatgctggcccgaggccagcccctccccgaaacgctgcagcttgcagtccaggggaaaaggacgttgcctggcttgcagcaacaacagcagcagcaacagcagcagcagcagcagcagcagcagcagcagcagcagcaacagcagccgcagcagcagccgccgcaaccacagacgcagcaacaacagcagccggcccttgttaactacaacagaccatctggcccggggccggagctgagcggcccgagcaccccgcagaagctgccggtgcccgcgcccggcggccggccctcgcccgcgccccccgcagccgcgcagccgcccgcggccgcagtgcccgggccctcagtgccgcagccggccccggggcagccctcgcccgtcctccagctgcagcagaagcagagccgcatcagccccatccagaaaccgcaaggcctggaccccgtggaaattctgcaagagcgggaatacagacttcaggcccgcatagctcataggatacaagaactggaaaatctgcctggctctttgccaccagatttaagaaccaaagcaaccgtggaactaaaagcacttcggttactcaatttccagcgtcagctgagacaggaggtggtggcctgcatgcgcagggacacgaccctggagacggctctcaactccaaagcatacaaacggagcaagcgccagactctgagagaagctcgcatgaccgagaagctggagaagcagcagaagattgagcaggagaggaaacgccgtcagaaacaccaggaatacctgaacagtattttgcaacatgcaaaagattttaaggaatatcatcggtctgtggccggaaagatccagaagctctccaaagcagtggcaacttggcatgccaacactgaaagagagcagaagaaggagacagagcggattgaaaaggagagaatgcggcgactgatggctgaagatgaggagggttatagaaaactgattgatcaaaagaaagacaggcgtttagcttaccttttgcagcagaccgatgagtatgtagccaatctgaccaatctggtttgggagcacaagcaagcccaggcagccaaagagaagaagaagaggaggaggaggaagaagaaggctgaggagaatgcagagggtggggagtctgccctgggaccggatggagagcccatagatgagagcagccagatgagtgacctccctgtcaaagtgactcacacagaaaccggcaaggttctgttcggaccagaagcacccaaagcaagtcagctggacgcctggctggaaatgaatcctggttatgaagttgcccctagatctgacagtgaagagagtgattctgattatgaggaagaggatgaggaagaagagtccagtaggcaggaaaccgaagagaaaatactcctggatccaaatagcgaagaagtttctgagaaggatgctaagcagatcattgagacagctaagcaagacgtggatgatgaatacagcatgcagtacagtgccaggggctcccagtcctactacaccgtggctcatgccatctcggagagggtggagaaacagtctgccctcctaattaatgggaccctaaagcattaccagctccagggcctggaatggatggtttccctgtataataacaacttgaacggaatcttagccgatgaaatggggcttggaaagaccatacagaccattgcactcatcacttatctgatggagcacaaaagactcaatggcccctatctcatcattgttcccctttcgactctatctaactggacatatgaatttgacaaatgggctccttctgtggtgaagatttcttacaagggtactcctgccatgcgtcgctcccttgtcccccagctacggagtggcaaattcaatgtcctcttgactacttatgagtatattataaaagacaagcacattcttgcaaagattcggtggaaatacatgatagtggacgaaggccaccgaatgaagaatcaccactgcaagctgactcaggtcttgaacactcactatgtggcccccagaaggatcctcttgactgggaccccgctgcagaataagctccctgaactctgggccctcctcaacttcctcctcccaacaatttttaagagctgcagcacatttgaacaatggttcaatgctccatttgccatgactggtgaaagggtggacttaaatgaagaagaaactatattgatcatcaggcgtctacataaggtgttaagaccatttttactaaggagactgaagaaagaagttgaatcccagcttcccgaaaaagtggaatatgtgatcaagtgtgacatgtcagctctgcagaagattctgtatcgccatatgcaagccaaggggatccttctcacagatggttctgagaaagataagaaggggaaaggaggtgctaagacacttatgaacactattatgcagttgagaaaaatctgcaaccacccatatatgtttcagcacattgaggaatcctttgctgaacacctaggctattcaaatggggtcatcaatggggctgaactgtatcgggcctcagggaagtttgagctgcttgatcgtattctgccaaaattgagagcgactaatcaccgagtgctgcttttctgccagatgacatctctcatgaccatcatggaggattattttgcttttcggaacttcctttacctacgccttgatggcaccaccaagtctgaagatcgtgctgctttgctgaagaaattcaatgaacctggatcccagtatttcattttcttgctgagcacaagagctggtggcctgggcttaaatcttcaggcagctgatacagtggtcatctttgacagcgactggaatcctcatcaggatctgcaggcccaagaccgagctcaccgcatcgggcagcagaacgaggtccgggtactgaggctctgtaccgtgaacagcgtggaggaaaagatcctcgcggccgcaaaatacaagctgaacgtggatcagaaagtgatccaggcgggcatgtttgaccaaaagtcttcaagccacgagcggagggcattcctgcaggccatcttggagcatgaggaggaaaatgaggaagaagatgaagtaccggacgatgagactctgaaccaaatgattgctcgacgagaagaagaatttgacctttttatgcggatggacatggaccggcggagggaagatgcccggaacccgaaacggaagccccgtttaatggaggaggatgagctgccctcctggatcattaaggatgacgctgaagtagaaaggctcacctgtgaagaagaggaggagaaaatatttgggagggggtcccgccagcgccgtgacgtggactacagtgacgccctcacggagaagcagtggctaagggccatcgaagacggcaatttggaggaaatggaagaggaagtacggcttaagaagcgaaaaagacgaagaaatgtggataaagatcctgcaaaagaagatgtggaaaaagctaagaagagaagaggccgccctcccgctgagaaactgtcaccaaatccccccaaactgacaaagcagatgaacgctatcatcgatactgtgataaactacaaagatagttcagggcgacagctcagtgaagtcttcattcagttaccttcaaggaaagaattaccagaatactatgaattaattaggaagccagtggatttcaaaaaaataaaggaaaggattcgtaatcataagtaccggagcctaggcgacctggagaaggatgtcatgcttctctgtcacaacgctcagacgttcaacctggagggatcccagatctatgaagactccatcgtcttacagtcagtgtttaagagtgcccggcagaaaattgccaaagaggaagagagtgaggatgaaagcaatgaagaggaggaagaggaagatgaagaagagtcagagtccgaggcaaaatcagtcaaggtgaaaattaagctcaataaaaaagatgacaaaggccgggacaaagggaaaggcaagaaaaggccaaatcgaggaaaagccaaacctgtagtgagcgattttgacagcgatgaggagcaggatgaacgtgaacagtcagaaggaagtgggacggatgatgagtga

SEQ ID NO: 6 - protein, 1572 aa, Homo sapiens
MSTPTDPGAMPHPGPSPGPGPSPGPILGPSPGPGPSPGSVHSMMGPSPGPPSVSHPMPTMGSTDFPQEGMHQMHKPIDGIHDKGIVEDIHCGSMKGTGMRPPHPGMGPPQSPMDQHSQGYMSPHPSPLGAPEHVSSPMSGGGPTPPQMPPSQPGALIPGDPQAMSQPNRGPSPFSPVQLHQLRAQILAYKMLARGQPLPETLQLAVQGKRTLPGLQQQQQQQQQQQQQQQQQQQQQQQPQQQPPQPQTQQQQQPALVNYNRPSGPGPELSGPSTPQKLPVPAPGGRPSPAPPAAAQPPAAAVPGPSVPQPAPGQPSPVLQLQQKQSRISPIQKPQGLDPVEILQEREYRLQARIAHRIQELENLPGSLPPDLRTKATVELKALRLLNFQRQLRQEVVACMRRDTTLETALNSKAYKRSKRQTLREARMTEKLEKQQKIEQERKRRQKHQEYLNSILQHAKDFKEYHRSVAGKIQKLSKAVATWHANTEREQKKETERIEKERMRRLMAEDEEGYRKLIDQKKDRRLAYLLQQTDEYVANLTNLVWEHKQAQAAKEKKKRRRRKKKAEENAEGGESALGPDGEPIDESSQMSDLPVKVTHTETGKVLFGPEAPKASQLDAWLEMNPGYEVAPRSDSEESDSDYEEEDEEEESSRQETEEKILLDPNSEEVSEKDAKQIIETAKQDVDDEYSMQYSARGSQSYYTVAHAISERVEKQSALLINGTLKHYQLQGLEWMVSLYNNNLNGILADEMGLGKTIQTIALITYLMEHKRLNGPYLIIVPLSTLSNWTYEFDKWAPSVVKISYKGTPAMRRSLVPQLRSGKFNVLLTTYEYIIKDKHILAKIRWKYMIVDEGHRMKNHHCKLTQVLNTHYVAPRRILLTGTPLQNKLPELWALLNFLLPTIFKSCSTFEQWFNAPFAMTGERVDLNEEETILIIRRLHKVLRPFLLRRLKKEVESQLPEKVEYVIKCDMSALQKILYRHMQAKGILLTDGSEKDKKGKGGAKTLMNTIMQLRKICNHPYMFQHIEESFAEHLGYSNGVINGAELYRASGKFELLDRILPKLRATNHRVLLFCQMTSLMTIMEDYFAFRNFLYLRLDGTTKSEDRAALLKKFNEPGSQYFIFLLSTRAGGLGLNLQAADTVVIFDSDWNPHQDLQAQDRAHRIGQQNEVRVLRLCTVNSVEEKILAAAKYKLNVDQKVIQAGMFDQKSSSHERRAFLQAILEHEEENEEEDEVPDDETLNQMIARREEEFDLFMRMDMDRRREDARNPKRKPRLMEEDELPSWIIKDDAEVERLTCEEEEEKIFGRGSRQRRDVDYSDALTEKQWLRAIEDGNLEEMEEEVRLKKRKRRRNVDKDPAKEDVEKAKKRRGRPPAEKLSPNPPKLTKQMNAIIDTVINYKDSSGRQLSEVFIQLPSRKELPEYYELIRKPVDFKKIKERIRNHKYRSLGDLEKDVMLLCHNAQTFNLEGSQIYEDSIVLQSVFKSARQKIAKEEESEDESNEEEEEEDEEESESEAKSVKVKIKLNKKDDKGRDKGKGKKRPNRGKAKPVVSDFDSDEEQDEREQSEGSGTDDE

SEQ ID NO: 7 - DNA, 71 bp, Artificial
  shBRM origo sequence
cgcgtccccggaggtgctaagacacttatgttcaagagacataagtgtcttagcacctcctttttggaaat

SEQ ID NO: 8 - DNA, 69 bp, Artificial
  shBRM origo sequence
cgatttccaaaaaggaggtgctaagacacttatgtctcttgaacataagtgtcttagcacctccgggga